(19) United States
(12) Patent Application Publication
(10) Pub. No.: US 2026/0152497 A1
(43) Pub. Date: Jun. 4, 2026

(54) MIDAZO[4,5-c]QUINOLINE-4-AMINE COMPOUNDS AND CONJUGATES THEREOF, THEIR PREPARATION, AND THEIR THERAPEUTIC APPLICATIONS

(71) Applicant: SANOFI, PARIS (FR)

(72) Inventors: MARIE-PRISCILLE BRUN, Paris (FR); STEPHANIE DECARY, Paris (FR); ALEXANDRA FERRIER, Paris (FR); MARC FREDERIC

(21) Appl. No.: 18/699,087

(22) PCT Filed: Oct. 6, 2022
(86) PCT No.: PCT/EP2022/077804

§ 371 Date: Apr. 5, 2024

(30) Foreign Application Priority Data
Oct. 7, 2021 (EP) 21306412.4

(51) Int. Cl. C07D 471/04; A61K 47/68; A61P 35/00

(52) U.S. Cl. CPC C07D 471/04; A61K 47/6803; A61K 47/6849; A61K 47/6851; A61K 47/6853; A61P 35/00

(57) ABSTRACT

The present disclosure relates to compounds of formula (I) (I) wherein n is an integer from 1 to 50; R1 represents a hydrogen atom, a —(C1-C6)alkylene-O—(C1-C6)alkyl group, a —(C1-C6)alkylene-NH—(C1-C6)alkyl group, or a —(C1-C6)alkyl group; R2 represents a —(C1-C6)alkylene- group; R3 represents —O—, —NH— or a —N((C1-C6)alkyl)- group; R4 represents a hydrogen atom, a —(C1-C6)alkyl group or a —(C1-C6)alkoxy group; R5 represents a —(C1-C6)alkylene- group; and R6 represents a -L1-RCG1 group or a —RCG1 group. The present disclosure also relates to conjugates of formula (II), to processes for their preparation, to compositions comprising them and to their therapeutic uses, especially in the prevention and/or in the treatment of a disease or a disorder that may benefit of an activation of the immune system, such as cancers.

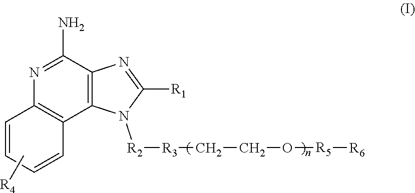

The present disclosure relates to new imidazo[4,5-c]quinoline-4-amine compounds (also named payloads), new imidazo[4,5-c]quinoline-4-amine conjugates, to compositions comprising them and to their therapeutic uses, for instance as Toll-like receptor 7 agonists. The present disclosure also relates to processes for preparing these conjugates.

### BACKGROUND OF THE INVENTION

Over the last decade, immunotherapy, mainly immune checkpoint inhibitor (ICI) has led to impressive clinical responses in patients. However, the percentage of ICI-responding patients remains low. It is noteworthy that ICI efficacy correlates with an “inflamed” tumor microenvironment (TME) and patients with “non-inflamed” tumors tend to respond poorly to ICI.

Therapeutic activation of TLR (Toll-like Receptor) such as TLR7/8 shows potential for immunotherapy, by converting non-inflamed or poorly inflamed tumors into immunologically inflamed tumors and (re)initiating T-cell mediated anti-tumor immune response. The innate immune system contains several families of germline-encoded pattern recognition receptors (PRRs), including Toll-like receptors (TLRs). These receptors recognize danger signals from either released internal cellular components termed damage-associated molecular pattern (DAMP) or microbial components termed pathogen-associated molecular patterns (PAMPs). The PAMPs are highly conserved molecular structures on a wide range of pathogens such as viruses, fungi, bacteria, and parasites. TLR7 and TLR8 are both located within endo-lysosomes and play an important role in the immune response during viral infection by their ability to recognize single stranded RNA PAMPs, as well as synthetic small molecules. Their stimulation leads to intracellular signaling and downstream activation of genes coding, among others for co-stimulatory molecules, pro-inflammatory cytokines and type I interferons. The activation of TLR such as TLR7 and/or TLR8, by an agonist can induce secretion of type I interferons such as IFNα and IFNβ, Tumor necrosis factor (TNFα) and interleukins such as IL6, IL12, which are important actors in the initiation of innate and adaptative immunity. The secretion of these cytokines, associated with the expression of co-stimulatory molecules, are known to induce the maturation of dendritic cells, monocytes and macrophages, facilitating the presentation of antigen and the stimulation of the adaptive immune response.

Various small molecules agonists of TLR7 (commonly named TLR7 agonists) and/or agonists of TLR8 (commonly named TLR8 agonists) have already been described as potent antitumoral compounds. for example, imidazoquinoline compounds such as Imiquimod (R-837), Resiquimod (R-848, also named R848), and Gardiquimod. For example, Imiquimod, the lead compound of the imidazoquinoline family, is a TLR7 agonist and is efficacious against many primary skin tumors and cutaneous metastases and is marketed as a topical formulation (Aldara®). It has been firstly approved by the FDA in 1997. Resiquimod (R848) can act simultaneously as TLR7 agonist and TLR8 agonist and has antiviral and antitumor activity. It is currently tested under clinical trials as a topical gel for the treatment of skin lesions such as those caused by the herpes simplex virus, multiple actinic keratosis and cutaneous T cell lymphoma, as an adjuvant to increase the effectiveness of vaccines and as an adjuvant to immunotherapy in allergic rhinitis (AR) patients.

Systemic administration of such imidazoquinoline agonists is not well tolerated, limiting their clinical use to local administration such as topical or intratumoral administration. Intratumoral delivery of TLR7/8 agonists has shown encouraging preclinical and clinical anti-tumor benefit. However, current intratumoral delivery approaches demonstrate poor tumor retention, limiting anti-tumor benefit and promoting treatment-related adverse events.

TLR7/8 agonists delivered by intravenous administration have also emerged in the clinic, as BDB-001 which shows activities in combination with pembrolizumab, but also treatment-related adverse events in around 78% of the enrolled patients under monotherapy (Journal of Clinical Oncology 2021 39:15_suppl, 2512-2512).

TLR7/8 agonists conjugated to antibody for intravenous tumor-targeted delivery have recently been described. However, the TLR7/8 conjugates currently under development show several adverse events.

Therefore, there remains a need for new immunotherapies based on TLR7/8 agonists with less adverse—events for the treatment of diseases, in particular cancer.

The purpose of the disclosure is to provide new immune-stimulating compounds suitable for systemic delivery, especially suitable for conjugation with an antibody and presenting less adverse-effects.

The present disclosure describes new imidazoquinoline compounds suitable for conjugation with an antibody and conjugates thereof showing potent TLR7 activity but no TLR8 activity.

### SUMMARY OF THE DISCLOSURE

The present disclosure relates to a compound (also named payload or compound/payload in the present text) of formula (I) or a pharmaceutically acceptable salt thereof

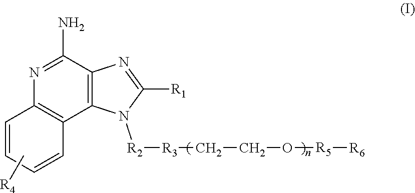

wherein
n is an integer from 1 to 50, for instance from 2 to 30, such as from 3 to 25;
R₁ represents a hydrogen atom, a —(C₁-C₆)alkylene-O—(C₁-C₆)alkyl group, a —(C₁-C₆)alkylene-NH—(C₁-C₆)alkyl group, or a —(C₁-C₆)alkyl group;
R² represents a —(C₁-C₆)alkylene- group;
R³ represents —O—, —NH— or a —N((C₁-C₆)alkyl)- group;
R⁴ represents a hydrogen atom, a —(C₁-C₆)alkyl group or a —(C₁-C₆)alkoxy group;
R⁵ represents a —(C₁-C₆)alkylene- group; and
R⁶ represents a -L₁-RCG₁ group or a —RCG₁ group;
   L₁ representing:
      —NH—;
      —C(═O)—NH—, for instance the —C(═O)— being linked to RCG₁ and the —NH— being linked to R⁵;
      a —(C₁-C₆)alkylene-R₈—C(═O)—NH— group, R₈ being a —(C₃-C₁₀) cycloalkylene- group or a —(C₃-C₁₀)heterocycloalkylene- group comprising 2 to 9 carbon atoms and 1 to 4 heteroatom(s) selected from oxygen, nitrogen, sulfur, —S(O)— and —SO₂—, for instance the —(C₁-C₆)alkylene- group being linked to RCG₁ and the —NH— being linked to R₅;
      a —NH—C(═O)—(C₁-C₆)alkylene-C(═O)—NH— group, one —NH— being linked to RCG₁ and the other —NH— being linked to R₅; or
      a —(C₁-C₆)alkylene-C(═O)—NH— group, the —(C₁-C₆)alkylene- group being optionally substituted by a —NH₂ group, for instance the —(C₁-C₆)alkylene- group being linked to RCG₁ and the —NH— being linked to R₅;
   RCG₁ representing:
      (i) a RₐZₐ—C(═O)— reactive group for which:
      Zₐ represents a single bond, —O— or —NH, such as —O—, and
      Rₐ represents a hydrogen atom, a —(C₁-C₆)alkyl group, a —(C₃-C₇)cycloalkyl group, a —(C₂-C₆)alkenyl group, a —(C₆-C₁₀)aryl group, a —(C₅-C₁₀)heteroaryl group comprising 4 to 9 carbon atoms and 1 to 4 heteroatom(s) selected from oxygen, nitrogen, and sulfur, or a —(C₃-C₇)heterocycloalkyl group comprising 2 to 6 carbon atoms and 1 to 4 heteroatom(s) selected from oxygen, nitrogen, sulfur, —S(O)— and —SO₂—, said —(C₆-C₁₀)aryl group, —(C₅-C₁₀)heteroaryl group and/or —(C₃-C₇)heterocycloalkyl group being optionally substituted by 1 to 5 atoms/groups chosen from a halogen atom, such as a fluorine atom, a —(C₁-C₆)alkyl group, a —(C₁-C₆)alkoxy group, a hydroxyl group, an oxo group, a nitro group and a cyano group; or
      (ii) one of the following reactive groups: a maleimido

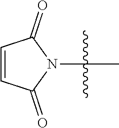 [SMILES: *N1C(=O)C=CC1=O]

group; a substituted maleimido group such as

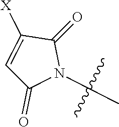

with X=Me, Br,

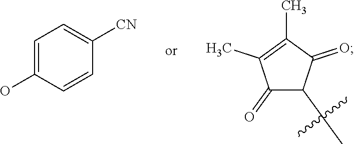

a haloacetamido

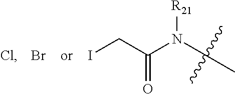

group with R₂₁ representing a hydrogen atom or a (C₁-C₆)alkyl group, such as a methyl group; Cl—; N₃—; HO—; HS—; an activated disulfide such as

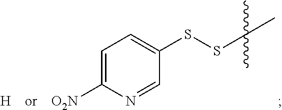

H₂N—; HC≡C— or an activated C≡C such as a cyclooctyne moiety for instance a DBCO-amine

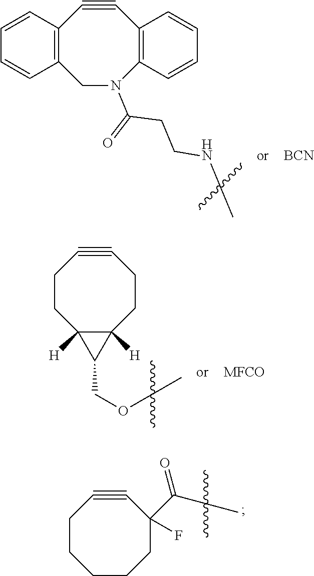

a phenyloxadiazolyl methylsulfone group (PODS) such as

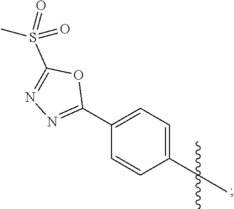 [SMILES: *c1ccc(-c2nnc(S(C)(=O)=O)o2)cc1]

a

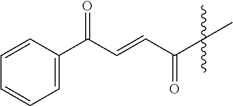 [SMILES: *C(=O)/C=C/C(=O)c1ccccc1]

group; a

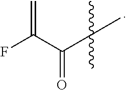 [SMILES: *C(=O)C(=C)F]

group; an O-alkyl hydroxylamine or a Pictet-Spengler reaction substrate such as

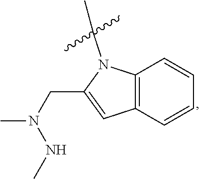 [SMILES: *n1c(CN(C)NC)cc2ccccc21]

for instance RCG₁ is N₃—; a maleimido group

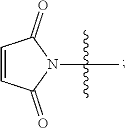 [SMILES: *N1C(=O)C=CC1=O]

a substituted maleimido group

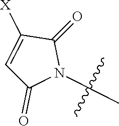

with X=Me, Br,

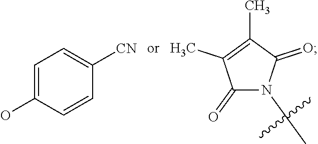

a phenyloxadiazolyl methylsulfone group

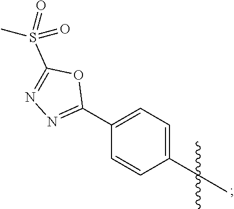 [SMILES: *c1ccc(-c2nnc(S(C)(=O)=O)o2)cc1]

a

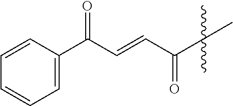 [SMILES: *C(=O)/C=C/C(=O)c1ccccc1]

group; a I—CH₂—C(═O)—NR₂₁— group with R₂₁ representing a hydrogen atom or a (C₁-C₆)alkyl group; or a

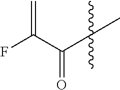 [SMILES: *C(=O)C(=C)F]

group.

The present disclosure further relates to a compound (also named conjugate or compound/conjugate in the present text) of formula (II) or a pharmaceutically acceptable salt thereof

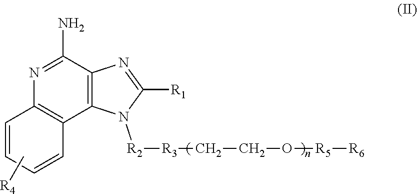

wherein
n is an integer from 1 to 50, for instance from 2 to 30, such as from 3 to 25, for instance 3, 7, 11 or 23;
R₁ represents a hydrogen atom, a —(C₁-C₆)alkylene-O—(C₁-C₆)alkyl group, a —(C₁-C₆)alkylene-NH—(C₁-C₆)alkyl group, or a —(C₁-C₆)alkyl group, for instance a —(C₁-C₆)alkylene-O—(C₁-C₆)alkyl group such as a —CH₂—O—C₂H₅ group;
R₂ represents a —(C₁-C₆)alkylene- group, for instance a —CH₂—C(CH₃)₂— group;
R₃ represents —O—, —NH— or a —N((C₁-C₆)alkyl)- group, for instance —O— or —NH—;
R₄ represents a hydrogen atom, a —(C₁-C₆)alkyl group or a —(C₁-C₆)alkoxy group, for instance a hydrogen atom;
R₅ represents a —(C₁-C₆)alkylene- group, for instance a —CH₂—CH₂— group; and
R₇ represents a -L₁-G-Ab group or a -G-Ab group,
   L₁ representing:
      —NH—;
      —C(═O)—NH—, for instance the —C(═O)— being linked to G and the —NH— being linked to R₅;
      a —(C₁-C₆)alkylene-R₈—C(═O)—NH— group, R₈ being a —(C₃-C₁₀)cycloalkylene- group or a —(C₃-C₁₀)heterocycloalkylene- group comprising 2 to 9 carbon atoms and 1 to 4 heteroatom(s) selected from oxygen, nitrogen, sulfur, —S(O)— and —SO₂—, for instance L₁ is a —(CH₂)₂-piperidinyl-C(═O)—NH— group, for instance the —(C₁-C₆)alkylene- group being linked to G and the —NH— being linked to R₅;
      a —NH—C(═O)—(C₁-C₆)alkylene-C(═O)—NH— group, for instance L₁ is a —NH—C(═O)—(CH₂)₃—C(═O)—NH— group, one —NH— being linked to G and the other —NH— being linked to R₅; or
      a —(C₁-C₆)alkylene-C(═O)—NH— group, the —(C₁-C₆)alkylene- group being optionally substituted by a NH₂ group, for instance a —CH₂—CH(CH₂—NH₂)—C(═O)—NH— group, a —(CH₂)₂—C(═O)—NH— group or a —(CH₂)₃—C(═O)—NH— group, for instance the —(C₁-C₆)alkylene- group being linked to G and the —NH— being linked to R₅;
   Ab representing an antibody, for instance a monoclonal antibody; and
   G representing the product of reaction between RCG₁ as defined in the present disclosure and RCG₂, a reactive group present on the antibody, for instance G is selected from the group consisting of:

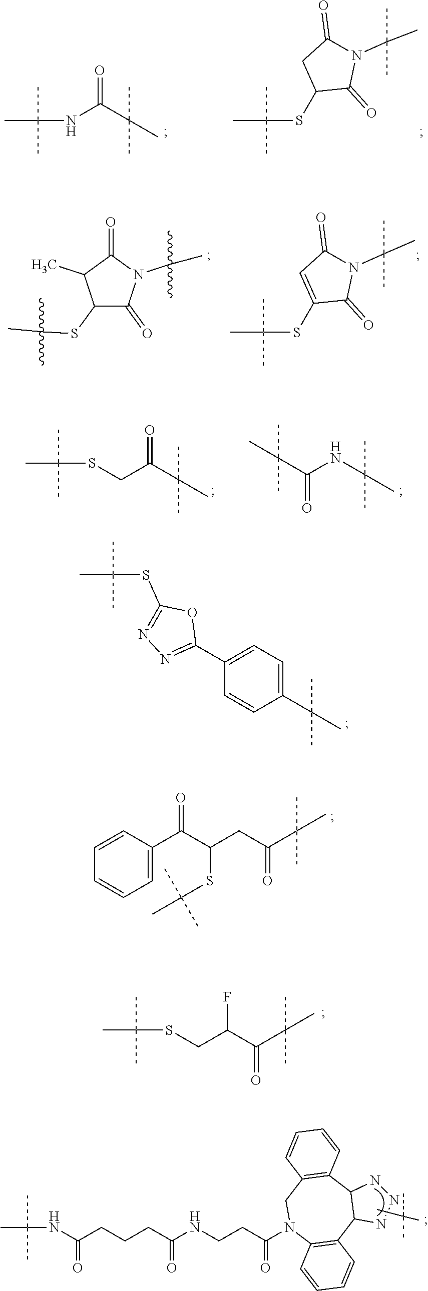

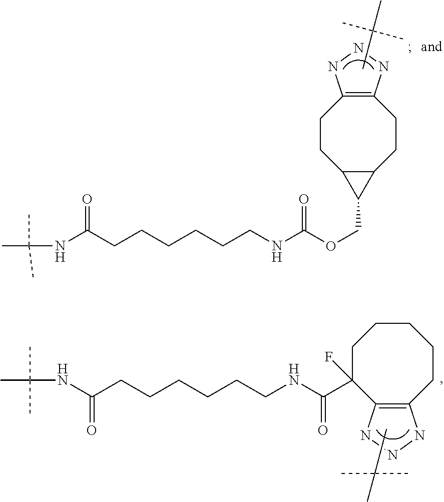

for instance, the left side of the G groups being linked to Ab and the right side of the G groups being linked to L₁ when R₇ is -L₁-G-Ab or directly linked to R₅ when R₇ is -G-Ab, for instance, G represents the following groups:

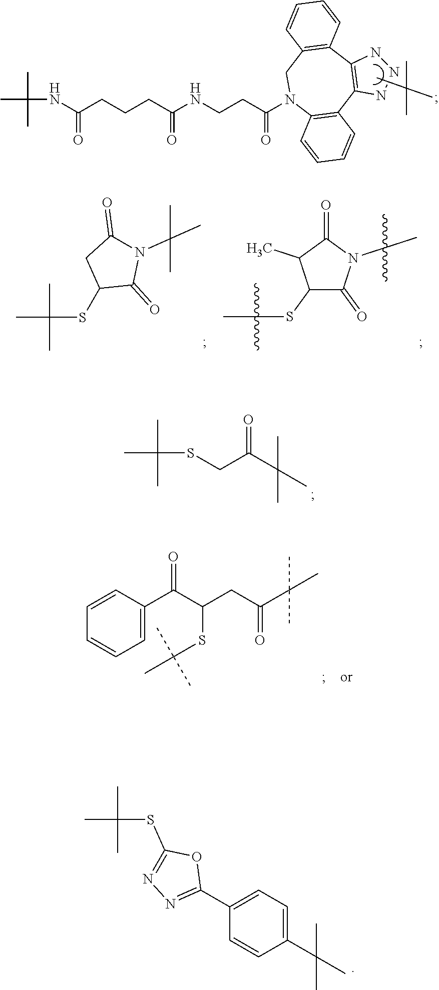

The compounds of formulae (I) and (II) may be present as well under tautomer forms. Indeed, it is to be understood that the present disclosure encompasses all isomers of formulae (I) and (II) and their pharmaceutically acceptable derivatives, including all geometric, tautomeric and mixtures thereof.

The compound/payload of formula (I) may exist in the form of bases or addition salts with acids or bases, in particular pharmaceutically acceptable salts.

Pharmaceutically acceptable salts of the compound/payload of formula (I) do form part of the disclosure.

The compound/payload of formula (I) may exist in the form of bases, acids, zwitterion or of addition salts with acids or bases. Such addition salts, bases, acids and zwitterion form part of the disclosure. Hence, the disclosure relates, inter alia, to the compound/payload of formula (I), or to pharmaceutically acceptable salts thereof.

These salts may be prepared with pharmaceutically acceptable acids or bases, although the salts of other acids or bases useful, for example, for purifying or isolating the compound/payload of formula (I), also form part of the disclosure.

The present disclosure also relates to processes for the preparation of the compounds/conjugates of formula (II) in accordance with the present disclosure.

Thus, according to one specific embodiment, the disclosure relates to a process for preparing a compound/conjugate of formula (II) as defined in the present disclosure comprising at least the steps of:
(i) placing in contact and leaving to react:
   an, optionally buffered, aqueous solution of an antibody Ab, comprising a reactive RCG2 group, optionally modified by means of a modifying agent,
and
   a solution of a compound/payload of formula (I) as defined in the present disclosure, comprising a reactive RCG1 group,
RCG1 groups of compound/payload of formula (I) being reactive towards the RCG₂ groups of the antibody to form the G group by a covalent bond and forming the compound/conjugate of formula (II);
(ii) and then optionally separating the compound/conjugate of formula (II) formed in step (i) from the unreacted compound/payload of formula (I) and/or from the unreacted antibody and/or from any aggregates that may have been formed.

Another subject matter of the instant disclosure is a compound/conjugate of formula (II) in accordance with the disclosure selected from the above and below definitions/lists, or a pharmaceutically acceptable salt thereof, for use as a medicine.

Another subject matter of the instant disclosure is a compound/conjugate of formula (II) in accordance with the disclosure selected from the above and below definitions/lists, or a pharmaceutically acceptable salt thereof, for use in therapy, especially as a TLR7 agonist

Another subject matter of the instant disclosure is a compound/conjugate of formula (II) in accordance with the disclosure selected from the above and below definitions/lists, or a pharmaceutically acceptable salt thereof, for use in the prevention and/or treatment of a disease or a disorder that may benefit of an activation of the immune system for instance for use in the prevention and/or treatment of a cell-proliferative disease, a cancer, a chronic myelogenous, a hairy cell leukemia, a dermatological disease such as a skin lesion or a skin cancer (for example an external genital and perianal warts/condyloma acuminate, a genital herpes, an actinic keratosis, a basal cell carcinoma, a cutaneous T-cell lymphoma), an autoimmune disease, an inflammatory disease, a respiratory disease, a sepsis, an allergy (for example an allergic rhinitis or a respiratory allergy), an asthma, a graft rejection, a graft-versus-host disease, and an immunodeficiency, for instance in the prevention and/or in the treatment of cancers.

Another subject matter of the instant disclosure is a compound/conjugate of formula (II) in accordance with the disclosure selected from the above and below definitions/lists, or a pharmaceutically acceptable salt thereof, for use in the prevention and/or treatment of a cancer.

Another subject matter of the instant disclosure is a compound/conjugate of formula (II) in accordance with the disclosure selected from the above and below definitions/lists, or a pharmaceutically acceptable salt thereof, for use as anticancer agents.

Another subject matter of the instant disclosure is a compound/conjugate of formula (II) in accordance with the disclosure selected from the above and below definitions/lists, or a pharmaceutically acceptable salt thereof, for use in an anti-tumoral vaccine.

Another subject matter of the instant disclosure is a method of preventing and/or treating a disease or a disorder that may benefit of an activation of the immune system, for instance of preventing and/or treating a cell-proliferative disease, a cancer, a chronic myelogenous, a hairy cell leukemia, a dermatological disease such as a skin lesion or a skin cancer (for example an external genital and perianal warts/condyloma acuminate, a genital herpes, an actinic keratosis, a basal cell carcinoma, a cutaneous T-cell lymphoma), an autoimmune disease, an inflammatory disease, a respiratory disease, a sepsis, an allergy (for example an allergic rhinitis or a respiratory allergy), an asthma, a graft rejection, a graft-versus-host disease, and an immunodeficiency, which comprises administering to a subject in need thereof, for instance a human, a therapeutically effective amount of a compound/conjugate of formula (II) in accordance with the disclosure selected from the above and below definitions/lists, or a pharmaceutically acceptable salt thereof.

The present disclosure also relates, in another aspect, to a method of preventing a disease or a disorder that may benefit of an activation of the immune system, for instance of preventing a cell-proliferative disease, a cancer, a chronic myelogenous, a hairy cell leukemia, a dermatological disease such as a skin lesion or a skin cancer (for example an external genital and perianal warts/condyloma acuminate, a genital herpes, an actinic keratosis, a basal cell carcinoma, a cutaneous T-cell lymphoma), an autoimmune disease, an inflammatory disease, a respiratory disease, a sepsis, an allergy (for example an allergic rhinitis or a respiratory allergy), an asthma, a graft rejection, a graft-versus-host disease, and an immunodeficiency, in a patient in need thereof, for instance a human, which comprises immunizing said patient with a vaccine comprising a compound/conjugate of formula (II) in accordance with the disclosure selected from the above and below definitions/lists, or a pharmaceutically acceptable salt thereof.

The present disclosure also relates, in another aspect, to an anti-tumoral vaccine.

The present disclosure further relates to the use of a compound/conjugate of formula (II) in accordance with the disclosure selected from the above and below definitions/lists, or a pharmaceutically acceptable salt thereof, for the manufacture of an antitumoral vaccine and/or of a medicament for preventing and/or treating a disease or a disorder that may benefit of an activation of the immune system, for instance for preventing and/or treating a cell-proliferative disease, a cancer, a chronic myelogenous, a hairy cell leukemia, a dermatological disease such as a skin lesion or a skin cancer (for example an external genital and perianal warts/condyloma acuminate, a genital herpes, an actinic keratosis, a basal cell carcinoma, a cutaneous T-cell lymphoma), an autoimmune disease, an inflammatory disease, a respiratory disease, a sepsis, an allergy (for example an allergic rhinitis or a respiratory allergy), an asthma, a graft rejection, a graft-versus-host disease, and an immunodeficiency, for instance a cancer.

Another subject matter of the instant disclosure is a medicament comprising as active principle an effective dose of a compound/conjugate of formula (II) in accordance with the disclosure selected from the above and below definitions/lists, or a pharmaceutically acceptable salt thereof.

Another subject matter of the instant disclosure is a medicament comprising a compound/conjugate of formula (II) in accordance with the disclosure selected from the above and below definitions/lists, or a pharmaceutically acceptable salt thereof.

Another subject matter of the instant disclosure is a pharmaceutical composition comprising as active principle an effective dose of a compound/conjugate of formula (II) in accordance with the disclosure selected from the above and below definitions/lists, or a pharmaceutically acceptable salt thereof, and at least one pharmaceutically acceptable excipient.

Another subject matter of the instant disclosure is a pharmaceutical composition comprising a compound/conjugate of formula (II) in accordance with the disclosure selected from the above and below definitions/lists, or a pharmaceutically acceptable salt thereof, and at least one pharmaceutically acceptable excipient.

### Definitions

In the context of the present disclosure, the terms below have the following definitions unless otherwise mentioned throughout the instant specification:
a halogen atom: a fluorine, a chlorine, a bromine or an iodine atom, and for example a fluorine or a bromine or an iodine atom;
a hydroxyl group: a “—OH” group;
an oxo group: a “═O” group;
a cyano group: a “—C≡N” group;
a nitro group: a “—NO₂” group;
a —(Cₓ-C_{y})alkyl group: a linear or branched saturated hydrocarbon-based aliphatic group comprising from x to y carbon atoms, for example from 1 to 6 carbon atoms, By way of examples, mention may be made of, but not limited to: methyl, ethyl, propyl, n-propyl, isopropyl, butyl, isobutyl, sec-butyl, tert-butyl, pentyl, isopentyl, hexyl, isohexyl groups, and the like;
a —(Cₓ-C_{y})alkylene- group: a linear or branched saturated hydrocarbon-based aliphatic group being bivalent and comprising from x to y carbon atoms, for example from 1 to 6 carbon atoms, that is to say a —(Cₓ-C_{y})alkyl group as defined above which is bivalent.
a —(Cₓ-C_{y})alkenyl group: a linear or branched hydrocarbon-based aliphatic group comprising at least one unsaturation (double bond) and comprising, from x to y carbon atoms (x being an integer of at least 2), for example from 2 to 6 carbon atoms. By way of examples, mention may be made of, but not limited to: ethenyl, propenyl, butenyl, pentenyl, hexenyl groups, and the like;
a —(Cₓ-C_{y})alkoxy group: an —O-alkyl group where the alkyl group is as previously defined. For example, a —(C₁-C₆)alkoxy group. By way of examples, mention may be made of, but not limited to: methoxy, ethoxy, propoxy, isopropoxy, linear, secondary or tertiary butoxy, isobutoxy, pentoxy, hexyloxy, groups, and the like;
an azido group: a “—N₃” group;
a —(C₃-C₁₀)cycloalkyl group or a —(C₃-C₇)cycloalkyl group: a cyclic alkyl group comprising, unless otherwise mentioned, from 3 to 10 carbon atoms (noted “(C₃-C₁₀)cycloalkyl group”) or from 3 to 7 carbon atoms (noted “(C₃-C₇)cycloalkyl group”), saturated or partially unsaturated and unsubstituted or substituted. By way of examples, mention may be made of, but not limited to: cyclopropyl, cyclobutyl, cyclopentyl, cyclobutenyl, cyclopentenyl, cyclohexenyl, cyclohexyl, cycloheptyl, cyclooctyl, cyclononyl, cyclodecyl groups, and the like;
a —(C₃-C₇)heterocycloalkyl group, or a —(C₃-C₁₀)heterocycloalkyl group: a monocyclic alkyl group comprising, unless otherwise mentioned, from 2 to 6 carbon atoms (also noted “a —(C₃-C₇) membered heterocycloalkyl group”) and comprising 1 to 4 heteroatoms selected from oxygen, nitrogen, sulfur, —S(O)—, and —SO₂— (in other terms, one heteroatom replaces one carbon atom) or a monocyclic alkyl group comprising, unless otherwise mentioned, from 2 to 9 carbon atoms (also noted “a —(C₃-C₁₀) membered heterocycloalkyl group”) and comprising 1 to 4 heteroatoms selected from oxygen, nitrogen, sulfur, —S(O)—, and —SO₂— (in other terms, one heteroatom replaces one carbon atom). Such heterocycloalkyl group may be saturated or partially saturated and unsubstituted or substituted. By way of examples of heterocycloalkyl groups, mention may be made of, but not limited to: piperazine, morpholino, pyrrolidine, tetrahydropyrane, thietane dioxide, piperidine, thiolane, thiolane oxide, thiolane dioxide, dihydrofurane, tetrahydrofurane, azetidine, oxetane, thietane, 2H-pyrrole, 1H-, 2H- or 3H-pyrroline, tetrahydrothiophene, oxadiazole and for example 1,3,4-oxadiazole or 1,3,5-oxadioazole, thiadiazole and for example 1,3,4-thiadiazole, isoxazoline, 2- or 3-pyrazoline, pyrroline, pyrazolidine, imidazoline, imidazolidine, thiazolidine, isooxazoline, isoxazolidine, dioxalane, oxathiazole, oxathiadiazole, dioxazole groups, and the like.
a —(C₅-C₁₀)heteroaryl group means: a cyclic aromatic group comprising from 4 to 9 carbon atoms and comprising from 1 and 4 heteroatoms selected from nitrogen, oxygen and sulfur (also noted “a (C₅-C₁₀) membered heteroaryl group”) (in other terms, one heteroatom replaces one carbon atom). Such heteroaryl group may be unsubstituted or substituted. By way of examples of 5 to 10-membered heteroaryl groups, mention may be made of, but not limited to: pyridine, furan, pyrrole, thiophene, pyrazole, oxazole, isoxazole, triazole, tetrazole, oxadiazole, furazan, thiazole, isothiazole, thiadiazole, imidazole, pyrimidine, pyridazine, triazine groups, and the like;
a —(C₆-C₁₀)aryl group: a cyclic aromatic group comprising from 6 to 10 carbon atoms (noted “a (C₆-C₁₀) membered aryl group”). Such aryl group may be unsubstituted or substituted. By way of examples of 6 to 10-membered aryl groups, mention may be made of, but not limited to: phenyl, naphthyl groups, and the like;
PEG: polyethylene glygol, PEGn means, as defined in the general formula of the disclosure, a —(CH₂—CH₂—O)ₙ portion wherein n represents an integer from 1 to 50, for instance from 2 to 30, from 3 to 25 linked to a —CH₂—CH₂— group (R₅). For instance PEG4 means that in the (CH₂—CH₂-0)ₙ portion, n is 3, PEG8 means that in the (CH₂—CH₂—O)ₙ portion, n is 7, PEG12 means that in the (CH₂—CH₂—O)ₙ portion, n is 11 and PEG24 means that in the (CH₂—CH₂—O)ₙ portion, n is 23.
antibody: an antibody with affinity for a biological target, such as a monoclonal antibody.

The function of the antibody is to direct the biologically active compound such as a TLR7/8 agonist compound towards the biological target. The antibody may be monoclonal, polyclonal or multispecific; it may also be an antibody fragment; it may also be a murine, chimeric, humanized or human antibody. An “antibody” may be a natural or conventional antibody in which two heavy chains are linked to each other by disulfide bonds and each heavy chain is linked to a light chain by a disulfide bond (also referred to as a “full-length antibody”). The terms “conventional (or full-length) antibody” refers both to an antibody comprising the signal peptide (or pro-peptide, if any), and to the mature form obtained upon secretion and proteolytic processing of the chain(s). There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains or regions, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) influence the overall domain structure and hence the combining site. CDRs refer to amino acid sequences which together define the binding affinity and specificity of the natural Fv region of a native immunoglobulin binding site. The light and heavy chains of an immunoglobulin each have three CDRs, designated CDR1-L, CDR2-L, CDR3-L and CDR1-H, CDR2-H, CDR3-H, respectively. A conventional antibody antigen-binding site, therefore, includes six CDRs, comprising the CDR set from each of a heavy and a light chain V region.

As used herein, the term “antibody” denotes both conventional (full-length) antibodies and fragments thereof, as well as single domain antibodies and fragments thereof, such as variable heavy chain of single domain antibodies. Fragments of (conventional) antibodies typically comprise a portion of an intact antibody, such as the antigen binding region or variable region of the intact antibody and retain the biological function of the conventional antibody. Examples of such fragments include Fv, Fab, F(ab′)2, Fab′, dsFv, (dsFv)2, scFv, sc(Fv)2, nanobodies and diabodies.
biological target: an antigen (or group of antigens) for instance located at the surface of cancer cells or stromal cells associated with this tumor; these antigens may be, for example, a growth factor receptor, an oncogene product or a mutated “tumor suppressant” gene product, an angiogenesis-related molecule or an adhesion molecule;
conjugate: an antibody-drug conjugate or ADC, i.e. a polypeptide such as an antibody to which is covalently attached via a linker at least one molecule of a TLR7/8 agonist;
DAR (drug-to-antibody ratio): a number of TLR7/8 agonists attached via a linker to an antibody. DAR may be an average number or a number depending on the conjugation technology used
Fc receptor: a protein at the surface of certain immune cells such as B lymphocytes, follicular dendritic cells, natural killer cells, macrophages, monocytes, neutrophils, eosinophils, basophils, human platelets, and mast cells and contributing to the protective functions of the immune system. It may recognize the Fc (fragment crystallizable) region of an antibody.
linker: a group of atoms or a single bond that can covalently attach a compound/payload of formula (I) to an antibody in order to form a compound/conjugate of formula (II);
payload: a TLR7/8 agonist compound to which is covalently attached a linker. In the case of the present disclosure means compound of formula (I).
modifying agent: chemical agent that is used to modify amino acid side chains of the antibody in order to introduce a reactive group;
reactive group: a group of atoms that can promote or undergo a chemical reaction;
activated disulfide: a group comprising at least one disulfide group prone to thiol-disulfide exchange as illustrated in Bioconjugate techniques, Second Edition by Greg T. Hermanson.

By way of examples of activated disulfide, mention may be made of, but not limited to,

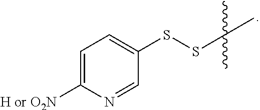

activated C≡C: a group comprising at least one cycloalkyne C≡C bond as illustrated in Bioconjugate techniques, Second Edition by Greg T. Hermanson. By way of examples of activated C≡C, mention may be made of, but not limited to a cyclooctyne moiety, for instance a DBCO-amine

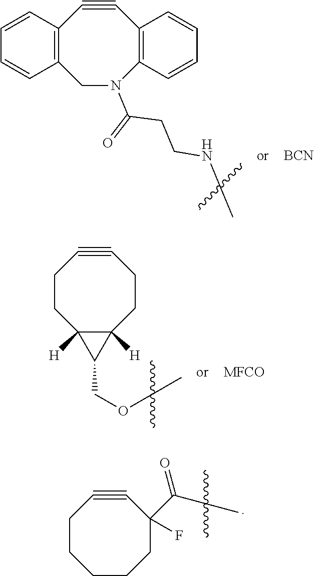

cycloalkyne: a monocyclic or polycyclic ring which comprises at least one C≡C bond, and from 3 to 16 carbon atoms and which can be unsubstituted or substituted.
heterocycloalkyne: a cycloalkyne group as defined above which comprises at least one to six heteroatoms selected from oxygen, nitrogen and sulfur.
“TLR”: the terms “Toll-like receptor” and “TLR” refer to any member of a family of highly conserved mammalian proteins which recognize pathogen-associated molecular patterns and act as key signaling elements in innate immunity. TLR polypeptides share a characteristic structure that includes an extracellular domain that has leucine-rich repeats, a transmembrane domain, and an intracellular domain that is involved in TLR signaling;
“TLR7”: the terms “Toll-like receptor 7” and “TLR7” refer to nucleic acids or polypeptides sharing at least 70%; at least 80%, at least 90%, at least 95%, at least 96%, at least 97%, at least 98%, at least 99%, or more sequence identity to a publicly available TLR7 sequence, e.g., GenBank accession number AAZ99026 for human TLR7 polypeptide, or GenBank accession number AAK62676 for murine TLR7 polypeptide;
“TLR8”: the terms “Toll-like receptor 8” and “TLR8” refer to nucleic acids or polypeptides sharing at least 70%; at least 80%, at least 90%, at least 95%, at least 96%, at least 97%, at least 98%, at least 99%, or more sequence identity to a publicly available TLR8 sequence, e.g., GenBank accession number AAZ95441 for human TLR8 polypeptide, or GenBank accession number AAK62677 for murine TLR8 polypeptide;
“TLR agonist”: the terms “Toll-like receptor” refer to a substance that binds, directly or indirectly, to a TLR (e.g., TLR7 and/or TLR8) to induce TLR signaling. Any detectable difference in TLR signaling can indicate that an agonist stimulates or activates a TLR. Signaling differences can be manifested, for example, as changes in the expression of target genes, in the phosphorylation of signal transduction components, in the intracellular localization of downstream elements such as NF-kB, in the association of certain components (such as IRAK) with other proteins or intracellular structures, or in the biochemical activity of components such as kinases (such as MAPK);
“Immune response”: An “immunological response” or “immune response” to an antigen or composition, as used herein, refers to the development in a subject of a humoral and/or cellular immune response to the antigen or composition.

Immune responses include innate and adaptive immune responses. Innate immune responses are fast-acting responses that provide a first line of defense for the immune system. In contrast, adaptive immunity uses selection and clonal expansion of immune cells having somatically rearranged receptor genes (e.g., T- and B-cell receptors) that recognize antigens from a given pathogen or disorder (e.g., a tumor), thereby providing specificity and immunological memory. Innate immune responses, among their many effects, lead to a rapid burst of inflammatory cytokines and activation of antigen-presenting cells (APCs) such as macrophages and dendritic cells. To distinguish pathogens from self-components, the innate immune system uses a variety of relatively invariable receptors that detect signatures from pathogens, known as pathogen-associated molecular patterns, or PAMPs. The mechanism behind this potentiation of the immune responses has been reported to involve pattern-recognition receptors (PRRs), which are differentially expressed on a variety of immune cells, including neutrophils, monocytes, macrophages, dendritic cells, natural killer cells, B cells and some nonimmune cells such as epithelial and endothelial cells. Engagement of PRRs leads to the activation of some of these cells and their secretion of cytokines and chemokines, as well as maturation and migration of activated cells but also of other cells not directly activated by PRR agonist. In tandem, this creates an inflammatory environment that leads to the establishment of the adaptive immune response. PRRs include nonphagocytic receptors, such as Toll-like receptors (TLRs) and nucleotide-binding oligomerization domain (NOD) proteins, and receptors that induce phagocytosis, such as scavenger receptors, mannose receptors and β-glucan receptors. Dendritic cells are recognized as some of the most important cell types for initiating the priming of naive CD4⁺ helper T (T_{H}) cells and for inducing CD8⁺ T cell differentiation into killer cells. TLR signaling has been reported to play an important role in determining the quality of these helper T cell responses, for instance, with the nature of the TLR signal determining the specific type of TH response that is observed (e.g., T_{H}1 versus T_{H}2 response). A combination of antibody (humoral) and cellular immunity are produced as part of a T_{H}1-type response, whereas a T_{H}2-type response is predominantly an antibody response;
A “humoral immune response” refers to an immune response mediated by antibody molecules, while a “cellular immune response” refers to an immune response mediated by T-lymphocytes and/or other white blood cells. One important aspect of cellular immunity involves an antigen-specific response by cytolytic T-cells (“CTLs”). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4⁺ and CD8⁺ T-cells;
An “immune response” associated with TLR7 and/or TLR8 is an immune response which involves activation of TLR7 and/or TLR8 receptors. Activation of TLR7 and/or TLR8 receptors may be determined in vitro using methods such as those described in the Examples.
“Induce”: “Induce” and variations thereof refer to any measurable increase in cellular activity. For example, induction of an immune response may include, for example, an increase in the production of a cytokine, activation, proliferation, or maturation of a population of immune cells, and/or others indicators of increased immune function;
Resiquimod or “R848” means 4-amino-2-(ethoxymethyl)-alpha, alpha-dimethyl-1H-imidazo(4,5-c)quinoline-1-ethanol (also called “R-848”), CAS number [144875-48-9], of formula:

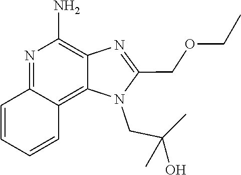 [SMILES: CCOCc1nc2c(N)nc3ccccc3c2n1CC(C)(C)O]

3M012 or 3M-012 means 1-(2-amino-2-methylpropyl)-2-(ethoxymethyl)-1H-imidazo[4,5-c]quinolin-4-amine, CAS number [642473-95-8], of formula:

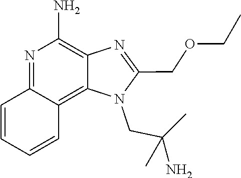 [SMILES: CCOCc1nc2c(N)nc3ccccc3c2n1CC(C)(C)N]

### Abbreviations

Ab: antibody; AcOH: acetic acid; ADIBO: azadibenzocyclooctyne; AP-1: activator protein-1; Ar: Argon; CH₃CN: acetonitrile; CRS: cytokine release syndrome; DAR: drug-to-antibody ratio; DBCO: dibenzylcyclooctyne; DCC: N,N′-dicyclohexylcarbodiimide; DCM: dichloromethane; DIEA: N,N-diisopropylethylamine; DIC: N,N′-diisopropylcarbodiimide; DMA: dimethylacetamide; DMAP: 4-dimethylaminopyridine; DMF: N,N-dimethylformamide; DMSO: dimethylsulfoxide; DPBS: Dulbecco's phosphate-buffered saline; DSC: N,N′-disuccinimidyl carbonate; EC50: effective concentration 50; EDTA: ethylenediaminetetraacetic acid; ES: electrospray; Et₂O: diethyl ether; EtOAc: ethyl acetate; H₂O: water; HCl: hydrochloric acid; HEK: human embryonic kidney; HEPES: 4-(2-hydroxyethyl)-1-piperazineethanesulfonic acid; HIC: hydrophobic interaction chromatography; HPLC: high pressure liquid chromatography; HRMS: high resolution mass spectrometry; IEC: ion exchange chromatography; IFN: interferon; ISG: interferon-stimulated gene; KD: dissociation constant; K₂HPO₄: dipotassium hydrogen phosphate; MeOH: methanol; MTBE: methyl tert-butyl ether; LAR: linker-to-antbody ratio; LCMS: liquid chromatography mass spectrometry; mAb or MAb: monoclonal antibody; MFI: median fluorescence intensity; MgSO₄: magnesium sulfate; NF-kB: nuclear factor kappa-light-chain-enhancer of activated B cells; NHS: N-hydroxysuccinimide; Na₂S₂O₃: sodium thiosulfate; NMR: nuclear magnetic resonance; NT: not tested; OD: optical density; PBMC: peripheral blood mononuclear cells; PBS: phosphate buffer saline; Pd/C: palladium on carbon; PEG: polyethylene glycol; PEGn: polyethylene glycol comprising a number n (integer) of ethylene glycol units; PES: polyethersulfone; PODS: phenyloxadiazolyl methylsulfone; PS: polysorbate; R848: resiquimod (CAS number [144875-48-9]); PVDF: polyvinylidene fluoride; RP: reverse phase; RT: room temperature; sat.: saturated; SEAP: Secreted Embryonic Alkaline Phosphatase; SEC: size exclusion chromatography; T: temperature; TEA: triethlyamine; THF: tetrahydrofuran; 3M012 or 3M-012: (CAS number [642473-95-8]); TLR: Toll-like receptor; UPLC: ultra-performance liquid chromatography; ˜: about.

### DETAILED DESCRIPTION OF THE PRESENT DISCLOSURE

The disclosure relates to a compound/payload of formula (I) or a pharmaceutically acceptable salt thereof

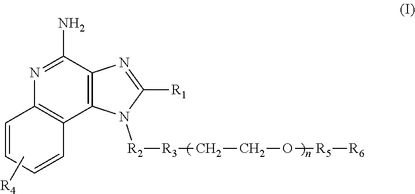

wherein
n is an integer from 1 to 50, for instance from 2 to 30, such as from 3 to 25;
R₁ represents a hydrogen atom, a —(C₁-C₆)alkylene-O—(C₁-C₆)alkyl group, a —(C₁-C₆)alkylene-NH—(C₁-C₆)alkyl group, or a —(C₁-C₆)alkyl group;
R₂ represents a —(C₁-C₆)alkylene- group;
R₃ represents —O—, —NH— or a —N((C₁-C₆)alkyl)- group;
R₄ represents a hydrogen atom, a —(C₁-C₆)alkyl group or a —(C₁-C₆)alkoxy group;
R₅ represents a —(C₁-C₆)alkylene- group; and
R₆ represents a -L₁-RCG₁ group or a —RCG₁ group;
   L₁ representing:
      —NH—;
      —C(═O)—NH—, for instance the —C(═O)— being linked to RCG₁ and the —NH-being linked to R₅;
      a —(C₁-C₆)alkylene-R₈—C(═O)—NH— group, R₈ being a —(C₃-C₁₀) cycloalkylene- group or a —(C₃-C₁₀)heterocycloalkylene- group comprising 2 to 9 carbon atoms and 1 to 4 heteroatom(s) selected from oxygen, nitrogen, sulfur, —S(O)— and —SO₂—, for instance the —(C₁-C₆)alkylene- group being linked to RCG₁ and the —NH— being linked to R₅;
      a —NH—C(═O)—(C₁-C₆)alkylene-C(═O)—NH— group, one —NH— being linked to RCG₁ and the other —NH— being linked to R₅; or
      a —(C₁-C₆)alkylene-C(═O)—NH— group, the —(C₁-C₆)alkylene- group being optionally substituted by a —NH₂ group, for instance the —(C₁-C₆)alkylene- group being linked to RCG₁ and the —NH— being linked to R₅;
   RCG₁ representing:
      (i) a RₐZₐ—C(═O)— reactive group for which:
      Zₐ represents a single bond, —O— or —NH, such as —O—, and
      Rₐ represents a hydrogen atom, a —(C₁-C₆)alkyl group, a —(C₃-C₇)cycloalkyl group, a —(C₂-C₆)alkenyl group, a —(C₆-C₁₀)aryl group, a —(C₅-C₁₀)heteroaryl group comprising 4 to 9 carbon atoms and 1 to 4 heteroatom(s) selected from oxygen, nitrogen, and sulfur, or a —(C₃-C₇)heterocycloalkyl group comprising 2 to 6 carbon atoms and 1 to 4 heteroatom(s) selected from oxygen, nitrogen, sulfur, —S(O)— and —SO₂—, said —(C₆-C₁₀)aryl group, —(C₅-C₁₀)heteroaryl group and/or —(C₃-C₇)heterocycloalkyl group being optionally substituted by 1 to 5 atoms/groups chosen from a halogen atom, such as a fluorine atom, a —(C₁-C₆)alkyl group, a —(C₁-C₆)alkoxy group, a hydroxyl group, an oxo group, a nitro group and a cyano group; or
      (ii) one of the following reactive groups: a maleimido

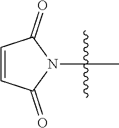 [SMILES: *N1C(=O)C=CC1=O]

group; a substituted maleimido group such as

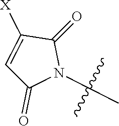

with X=Me, Br,

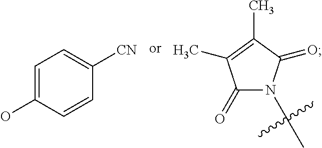

a haloacetamido

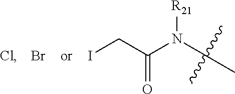

group with R₂₁ representing a hydrogen atom or a (C₁-C₆)alkyl group, such as a methyl group; Cl—; N₃—; HO—; HS—; an activated disulfide such as

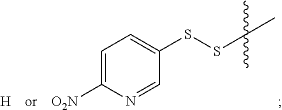

H₂N—; HC≡C— or an activated C≡C such as a cyclooctyne moiety for instance a DBCO-amine

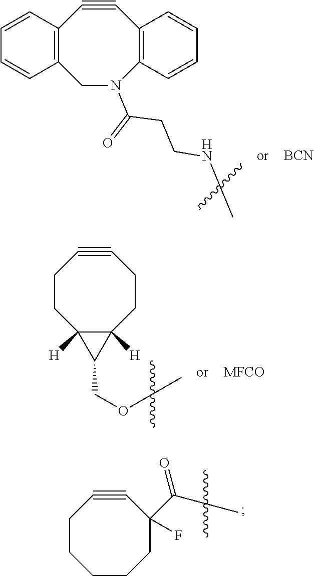

a phenyloxadiazolyl methylsulfone group (PODS) such as

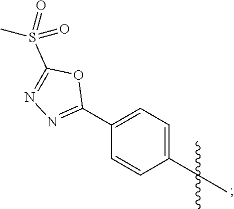 [SMILES: *c1ccc(-c2nnc(S(C)(=O)=O)o2)cc1]

a

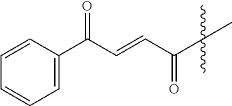 [SMILES: *C(=O)/C=C/C(=O)c1ccccc1]

group; a

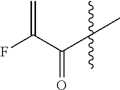 [SMILES: *C(=O)C(=C)F]

group; an O-alkyl hydroxylamine or a Pictet-Spengler reaction substrate such as

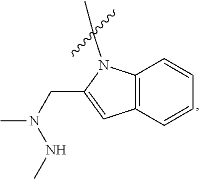 [SMILES: *n1c(CN(C)NC)cc2ccccc21]

for instance RCG₁ is N₃—; a maleimido group

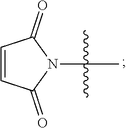 [SMILES: *N1C(=O)C=CC1=O]

a substituted maleimido group

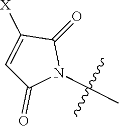

with X=Me, Br,

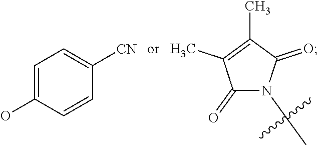

a phenyloxadiazolyl methylsulfone group

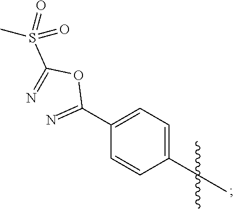 [SMILES: *c1ccc(C(=N)OC(=N)S(C)(=O)=O)cc1]

a

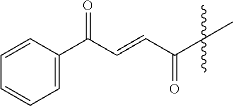 [SMILES: *C(=O)/C=C/C(=O)c1ccccc1]

group; a I—CH₂—C(═O)—NR₂₁— group with R₂₁ representing a hydrogen atom or a (C₁-C₆)alkyl group; or a

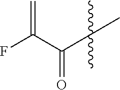 [SMILES: *C(=O)C(=C)F]

group.

As mentioned above, examples of RCG1 that may be mentioned include:
(i) a RₐZₐ—C(═O)— reactive group for which:
Zₐ represents a single bond, —O— or —NH, such as —O—, and
Rₐ represents a hydrogen atom, a —(C₁-C₆)alkyl group, a —(C₃-C₇)cycloalkyl group, a —(C₂-C₆)alkenyl group, a —(C₆-C₁₀)aryl group, a —(C₅-C₁₀)heteroaryl group comprising 4 to 9 carbon atoms and 1 to 4 heteroatom(s) selected from oxygen, nitrogen, and sulfur, or a —(C₃-C₇)heterocycloalkyl group comprising 2 to 6 carbon atoms and 1 to 4 heteroatom(s) selected from oxygen, nitrogen, sulfur, —S(O)— and —SO₂—, said —(C₆-C₁₀)aryl group, —(C₅-C₁₀)heteroaryl group and/or —(C₃-C₇)heterocycloalkyl group being optionally substituted by 1 to 5 atoms/groups chosen from a halogen atom, such as a fluorine atom, a —(C₁-C₆)alkyl group, a —(C₁-C₆)alkoxy group, a hydroxyl group, an oxo group, a nitro group and a cyano group; or
(ii) one of the following reactive groups: a maleimido

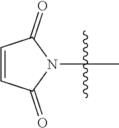 [SMILES: *N1C(=O)C=CC1=O]

group; a substituted maleimido group such as

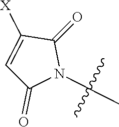

with X=Me, Br,

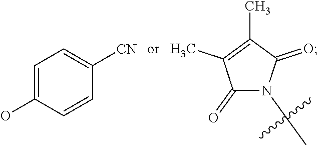

a haloacetamido

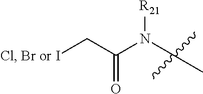

group with R₂₁ representing a hydrogen atom or a —(C₁-C₆)alkyl group, such as a methyl group; Cl—; N₃—; HO—; HS—; an activated disulfide such as

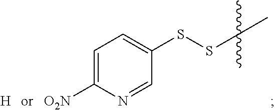

H₂N—; HC≡C— or an activated C≡C such as a cyclooctyne moiety for instance a DBCO-amine

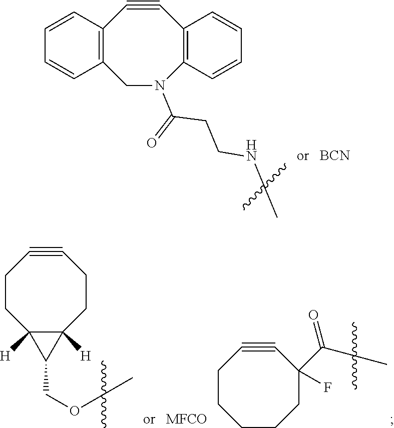

a phenyloxadiazolyl methylsulfone group (PODS) such as

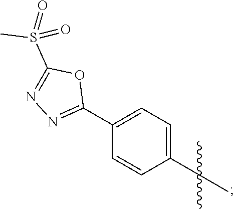 [SMILES: *c1ccc(-c2nnc(S(C)(=O)=O)o2)cc1]

a

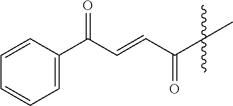 [SMILES: *C(=O)/C=C/C(=O)c1ccccc1]

group; a

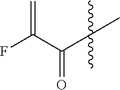 [SMILES: *C(=O)C(=C)F]

group; an —O-alkyl hydroxylamine or a Pictet-Spengler reaction substrate such as

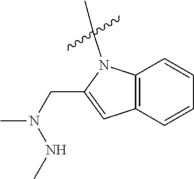 [SMILES: *n1c(CN(C)NC)cc2ccccc21]

described in Agarwal P., et al., Bioconjugate Chem 2013, 24, 846-851, for instance RCG₁ is N₃—; a maleimido group

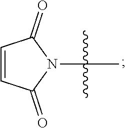 [SMILES: *N1C(=O)C=CC1=O]

a substituted maleimido group

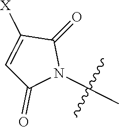

with X=Me, Br,

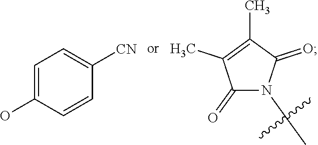

a phenyloxadiazolyl methylsulfone group

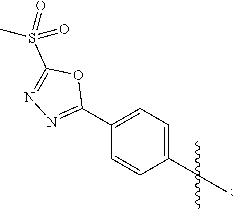 [SMILES: *c1ccc(-c2nnc(S(C)(=O)=O)o2)cc1]

a

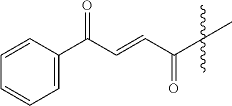 [SMILES: *C(=O)/C=C/C(=O)c1ccccc1]

group; a I—CH₂—C(═O)—NR₂₁— group with R₂₁ representing a hydrogen atom or a (C₁-C₆)alkyl group; or a

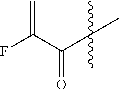 [SMILES: *C(=O)C(=C)F]

group.

For instance, RₐZₐ— may represent HO—, CH₃O—, CH₂═CH—CH₂O—,

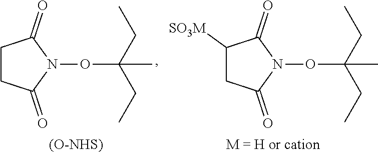

where cation represents for example sodium, potassium or cesium or

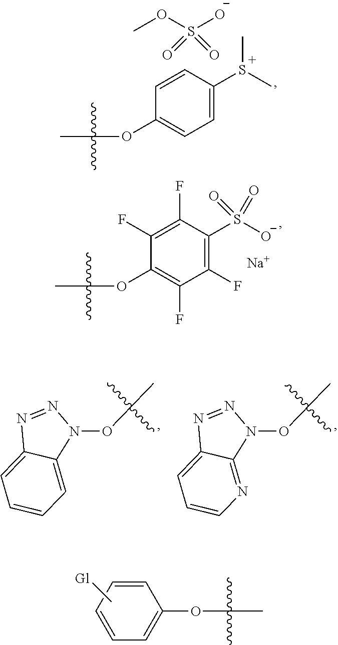

group in which GI represents at least one electroinductive group such as —NO₂ or a halogen atom, such as a fluorine atom (F). They may be, for example, the following groups:

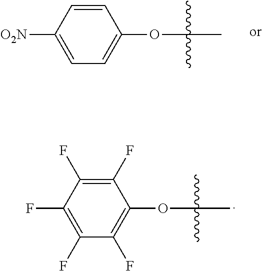 [SMILES: *Oc1ccc([N+](=O)[O-])cc1 ; *Oc1c(F)c(F)c(F)c(F)c1F]

Another type of RₐZₐ—C(═O)— group is the following

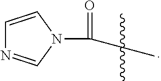 [SMILES: *C(=O)n1ccnc1]

Among the compounds of formula (I) that are subject matter of the disclosure, a group of compounds is composed of the compounds for which n is 3 to 25.

Among the compounds of formula (I) that are subject matter of the disclosure, a group of compounds is composed of the compounds for which n is 3, 7, 11 or 23.

Among the compounds of formula (I) that are subject matter of the disclosure, a group of compounds is composed of the compounds for which R₁ represents a —(C₁-C₆)alkylene-O—(C₁-C₆)alkyl group.

Among the compounds of formula (I) that are subject matter of the disclosure, a group of compounds is composed of the compounds for which R₁ represents a —CH₂—O—C₂H₅ group (also named a —CH₂—O-Et group).

Among the compounds of formula (I) that are subject matter of the disclosure, a group of compounds is composed of the compounds for which R₂ represents a branched —(C₁-C₆)alkylene- group.

Among the compounds of formula (I) that are subject matter of the disclosure, a group of compounds is composed of the compounds for which R₂ represents a —CH₂—C(CH₃)₂— group, for instance the CH₂ group being linked to the nitrogen atom of the imidazo[4,5-c]quinoline ring and the C(CH₃)₂ group, being linked to R₃.

Among the compounds of formula (I) that are subject matter of the disclosure, a group of compounds is composed of the compounds for which R₃ represents —O— or —NH—.

Among the compounds of formula (I) that are subject matter of the disclosure, a group of compounds is composed of the compounds for which R₃ represents —O—.

Among the compounds of formula (I) that are subject matter of the disclosure, a group of compounds is composed of the compounds for which R₃ represents —NH—.

Among the compounds of formula (I) that are subject matter of the disclosure, a group of compounds is composed of the compounds for which R₄ represents a hydrogen atom.

Among the compounds of formula (I) that are subject matter of the disclosure, a group of compounds is composed of the compounds for which R₅ represents a linear —(C₁-C₆)alkylene-group.

Among the compounds of formula (I) that are subject matter of the disclosure, a group of compounds is composed of the compounds for which R₅ represents a —(CH₂)₂— group.

Among the compounds of formula (I) that are subject matter of the disclosure, a group of compounds is composed of the compounds for which R₆ represents a RCG₁ group.

Among the compounds of formula (I) that are subject matter of the disclosure, a group of compounds is composed of the compounds for which R₆ represents a RCG₁ group, RCG₁ being a —N₃ group or a I—CH₂—C(═O)—NR₂₁— group with R₂₁ representing a hydrogen atom or a (C₁-C₆)alkyl group.

Among the compounds of formula (I) that are subject matter of the disclosure, a group of compounds is composed of the compounds for which R₆ represents a RCG₁ group, RCG₁ being a —N₃ group or a I—CH₂—C(═O)—NH— group.

Among the compounds of formula (I) that are subject matter of the disclosure, a group of compounds is composed of the compounds for which R₆ represents a -L₁-RCG₁ group.

Among the compounds of formula (I) that are subject matter of the disclosure, a group of compounds is composed of the compounds for which R₆ represents a -L₁-RCG₁ group,
RCG₁ being N₃—; a maleimido group

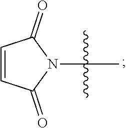 [SMILES: *N1C(=O)C=CC1=O]

a substituted maleimido group

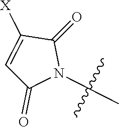

with X=Me, Br

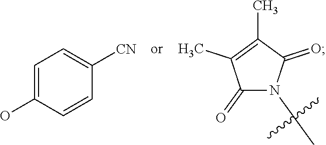

a phenyloxadiazolyl methylsulfone group

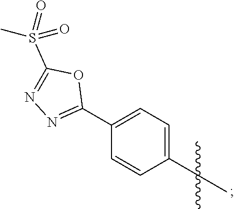 [SMILES: *c1ccc(-c2nnc(S(C)(=O)=O)o2)cc1]

a

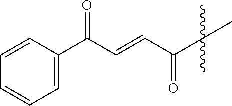 [SMILES: *C(=O)/C=C/C(=O)c1ccccc1]

group; or a

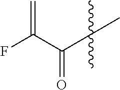 [SMILES: *C(=O)C(=C)F]

group, and
L₁ being:
   —NH—;
   —C(═O)—NH—, for instance the —C(═O)— being linked to RCG₁ and the —NH— being linked to R₅;
   a —(C₁-C₆)alkylene-R₈—C(═O)—NH— group, R₈ being a —(C₃-C₁₀)cycloalkylene- group or a (C₃-C₁₀)heterocycloalkylene- group comprising 2 to 9 carbon atoms and 1 to 4 heteroatom(s) selected from oxygen, nitrogen, sulfur, —S(O)— and —SO₂—, for instance a —(CH₂)₂-piperidinyl-C(═O)—NH— group, for instance the —(C₁-C₆)alkylene- group being linked to RCG₁ and the —NH— being linked to R₅;
   a —NH—C(═O)—(C₁-C₆)alkylene-C(═O)—NH— group, for instance a —NH—C(═O)—(CH₂)₃—C(═O)—NH— group one —NH— being linked to RCG₁ and the other —NH— being linked to R₅; or
   a —(C₁-C₆)alkylene-C(═O)—NH— group, the —(C₁-C₆)alkylene- group being optionally substituted by a —NH₂ group, for instance a —CH₂—CH(CH₂—NH₂)—C(═O)—NH— group, a —(CH₂)₂—C(═O)—NH— group or a —(CH₂)₃—C(═O)—NH— group, for instance the —(C₁-C₆)alkylene- group being linked to RCG₁ and the —NH— being linked to R₅.

Among the compounds of formula (I) that are subject matter of the disclosure, a group of compounds is composed of the compounds for which R₆ represents a -L₁-RCG₁ group,
RCG₁ being a maleimido group

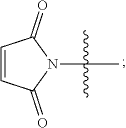 [SMILES: *N1C(=O)C=CC1=O]

a substituted maleimido group

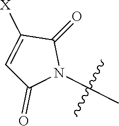

with X=Me, Br,

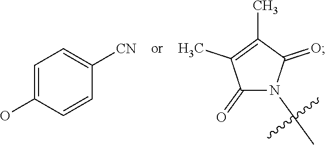

a phenyloxadiazolyl methylsulfone group

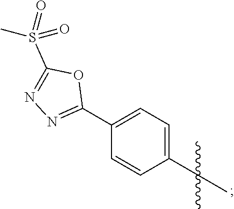 [SMILES: *c1ccc(-c2nnc(S(C)(=O)=O)o2)cc1]

or a

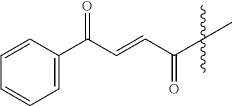 [SMILES: *C(=O)/C=C/C(=O)c1ccccc1]

group, and
L₁ being:
   —NH—;
   a —(CH₂)₂—C(═O)—NH— group, for instance the —(CH₂)₂— group being linked to RCG₁ and the —NH— being linked to R₅; or
   a —NH—C(═O)—(CH₂)₃—C(═O)—NH— group one —NH— being linked to RCG₁ and the other —NH— being linked to R₅.

All these sub-groups taken alone or in combination are part of the present disclosure.

According to a particular embodiment, the disclosure relates to a compound/payload of formula (I) or a pharmaceutically acceptable salt thereof wherein:
R₁ represents a —(C₁-C₆)alkylene-O—(C₁-C₆)alkyl group;
R₂ represents a —(C₁-C₆)alkylene- group;
R₃ represents —O—;
R₄ represents a hydrogen atom;
R₅ represents a —(C₁-C₆)alkyl group; and
R₆ represents a RCG₁ group, RCG₁ being a —N₃ group or a I—CH₂—C(═O)—NR₂₁— group with
R₂₁ representing a hydrogen atom or a (C₁-C₆)alkyl group.

According to another particular embodiment, the disclosure relates to a compound/payload of formula (I) or a pharmaceutically acceptable salt thereof wherein:
R₁ represents a —CH₂—O-Et group;
R₂ represents a —CH₂—C(CH₃)₂— group;
R₃ represents —O—;
R₄ represents a hydrogen atom;
R₅ represents a —CH₂—CH₂-group; and
R₆ represents a RCG₁ group, RCG₁ being a —N₃ group or a I—CH₂—C(═O)—NH— group.

According to another particular embodiment, the disclosure relates to a compound/payload of formula (I) or a pharmaceutically acceptable salt thereof wherein:
R₁ represents a —(C₁-C₆)alkylene-O—(C₁-C₆)alkyl group;
R₂ represents a —(C₁-C₆)alkylene- group;
R₃ represents —NH—;
R₄ represents a hydrogen atom;
R₅ represents a —(C₁-C₆)alkylene- group; and
R₆ represents a RCG₁ group, RCG₁ being a —N₃ group.

According to another particular embodiment, the disclosure relates to a compound/payload of formula (I) or a pharmaceutically acceptable salt thereof wherein
R₁ represents a —CH₂—O-Et group;
R₂ represents a —CH₂—C(CH₃)₂— group;
R₃ represents —NH—;
R₄ represents a hydrogen atom;
R₅ represents a —CH₂—CH₂— group; and
R₆ represents a RCG₁ group, RCG₁ being a —N₃ group.

According to another particular embodiment, the disclosure relates to a compound/payload of formula (I) or a pharmaceutically acceptable salt thereof wherein:
R₁ represents a —(C₁-C₆)alkylene-O—(C₁-C₆)alkyl group;
R₂ represents a —(C₁-C₆)alkylene- group;
R₃ represents —O—;
R₄ represents a hydrogen atom;
R₅ represents a —(C₁-C₆)alkyl group; and
R₆ represents a L₁-RCG₁ group, RCG₁ being a maleimido group

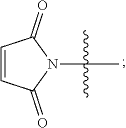 [SMILES: *N1C(=O)C=CC1=O]

a substituted maleimido group

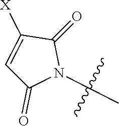

with X=Me, Br,

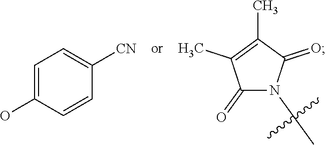

a phenyloxadiazolyl methylsulfone group

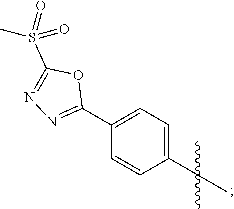 [SMILES: *c1ccc(-c2nnc(S(C)(=O)=O)o2)cc1]

or a

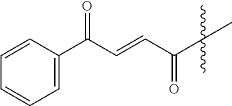 [SMILES: *C(=O)/C=C/C(=O)c1ccccc1]

group, and
L₁ being:
   —NH—;
   a —(C₁-C₆)alkylene-C(═O)—NH— group, for instance the —(C₁-C₆)alkylene-group being linked to RCG₁ and the —NH— being linked to R₅; or
   a —NH—C(═O)—(C₁-C₆)alkylene-C(═O)—NH— group one —NH— being linked to RCG₁ and the other —NH— being linked to R₅.

According to another particular embodiment, the disclosure relates to a compound/payload of formula (I) or a pharmaceutically acceptable salt thereof wherein:
R₁ represents a —CH₂—O-Et group;
R₂ represents a —CH₂—C(CH₃)₂— group;
R₃ represents —O—;
R₄ represents a hydrogen atom;
R₅ represents a —CH₂—CH₂-group; and
R₆ represents a L₁-RCG₁ group, RCG₁ being a maleimido group

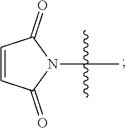 [SMILES: *N1C(=O)C=CC1=O]

a substituted maleimido group

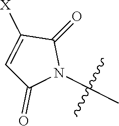

with X=Me, Br,

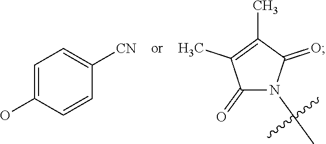

a phenyloxadiazolyl methylsulfone group

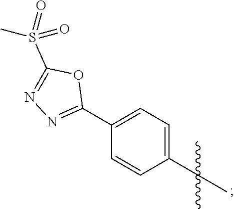 [SMILES: *c1ccc(-c2nnc(S(C)(=O)=O)o2)cc1]

or a

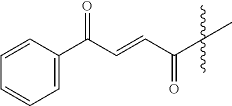 [SMILES: *C(=O)/C=C/C(=O)c1ccccc1]

group, and
L₁ being:
   —NH—;
   a —(CH₂)₂—C(═O)—NH— group, for instance the —(CH₂)₂— group being linked to RCG₁ and the —NH— being linked to R₅; or
   a —NH—C(═O)—(CH₂)₃—C(═O)—NH— group one —NH— being linked to RCG₁ and the other —NH— being linked to R₅.

Among the compounds of formula (I) that are subject matter of the present disclosure, mention may be made for instance to the following compounds

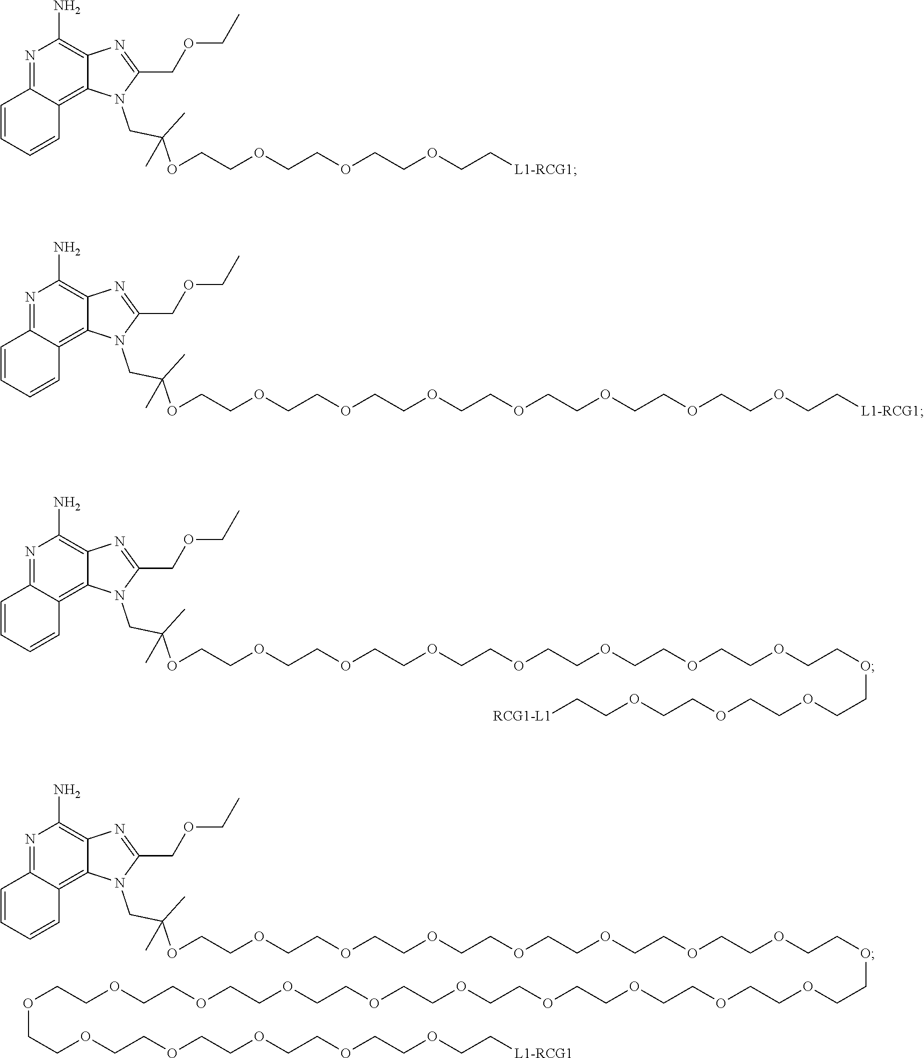

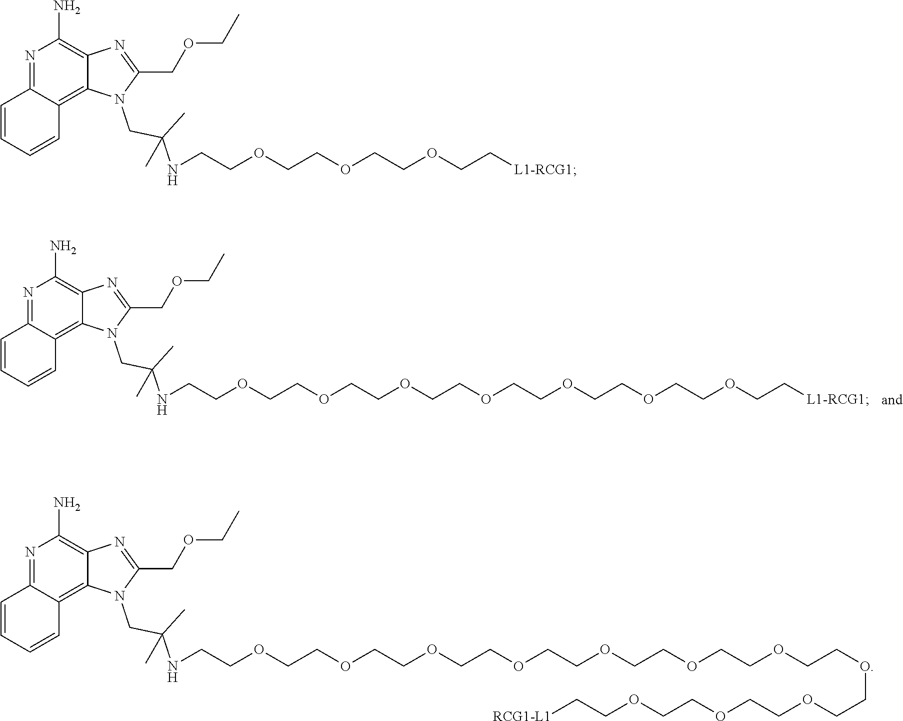

Among the compounds of formula (I) that are subject matter of the present disclosure, mention may be made for instance to the following compounds

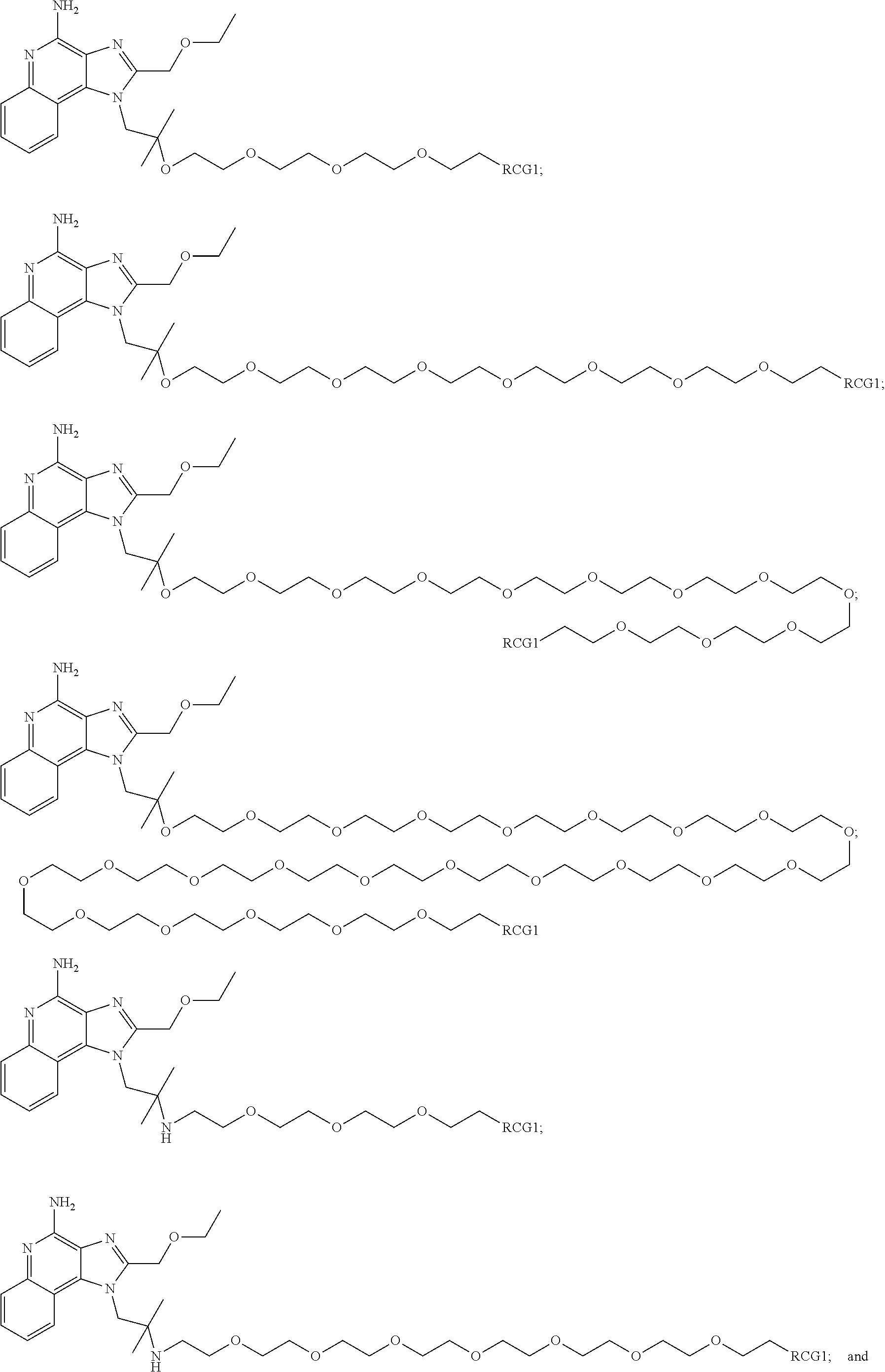

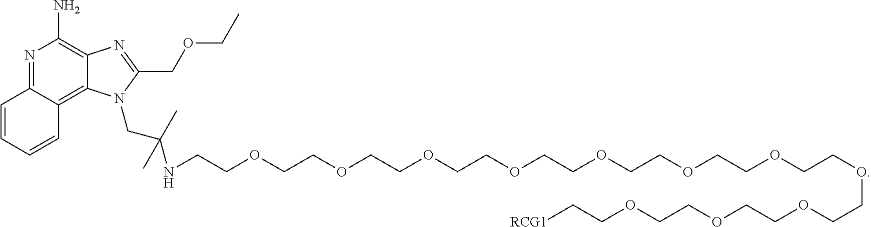

Among the compounds of formula (I) that are subject matter of the present disclosure, mention may be made for instance to the following compounds

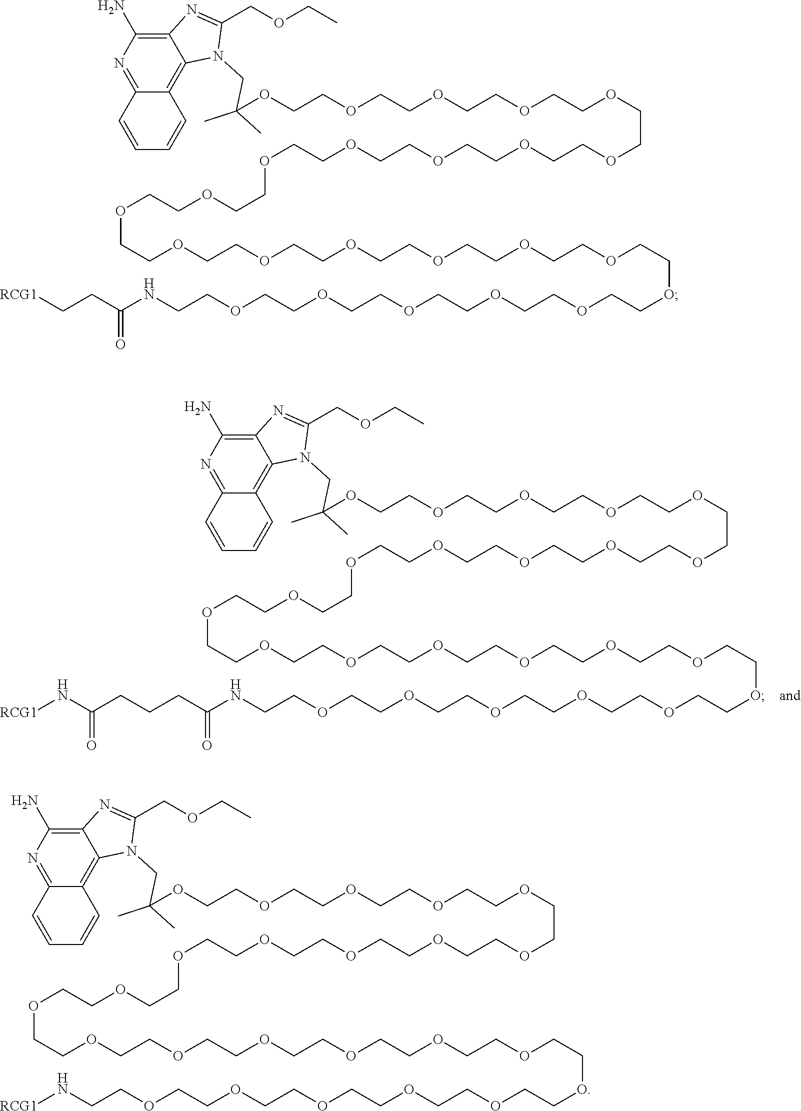

Among the compounds of formula (I) that are subject matter of the present disclosure, mention may be made for instance to the following compounds:
(1) 1-(14-azido-2,2-dimethyl-3,6,9,12-tetraoxatetradecyl)-2-(ethoxymethyl)-1H-imidazo[4,5-c]quinolin-4-amine (Example 1);
(2) 1-(14-azido-2,2-dimethyl-3,6,9,12-tetraoxatetradecyl)-2-(ethoxymethyl)-1H-imidazo[4,5-c]quinolin-4-amine (Example 7);
(3) 1-(38-azido-2,2-dimethyl-3,6,9,12,15,18,21,24,27,30,33,36-dodecaoxaoctatriacontyl)-2-(ethoxymethyl)-1H-imidazo[4,5-c]quinolin-4-amine (Example 13);
(4) 1-(74-azido-2,2-dimethyl-3,6,9,12,15,18,21,24,27,30,33,36,39,42,45,48,51,54, 57,60,63,66,69,72-tetracosaoxatetraheptacontyl)-2-(ethoxymethyl)-1H-imidazo[4,5-c]quinolin-4-amine (Example 19);
(5) 1-(14-azido-2,2-dimethyl-6,9,12-trioxa-3-azatetradecyl)-2-(ethoxymethyl)-1H-imidazo[4,5-c]quinolin-4-amine (Example 25);
(6) 1-(38-azido-2,2-dimethyl-6,9,12,15,18,21,24,27,30,33,36-undecaoxa-3-azaoctatriacontyl)-2-(ethoxymethyl)-1H-imidazo[4,5-c]quinolin-4-amine (Example 27);
(7) 1-(26-azido-2,2-dimethyl-6,9,12,15,18,21,24-heptaoxa-3-azahexacosyl)-2-(ethoxy-methyl)-1H-imidazo[4,5-c]quinolin-4-amine (Example 29);
(8) N-(74-(4-amino-2-(ethoxymethyl)-1H-imidazo[4,5-c]quinolin-1-yl)-73,73-dimethyl-3,6,9,12,15,18,21,24,27,30,33,36,39,42,45,48,51,54,57,60,63,66,69,72-tetracosaoxatetraheptacontyl)-3-(2,5-dioxo-2,5-dihydro-1H-pyrrol-1-yl)propanamide formate (Example 31);
(9) N-[2-[2-[2-[2-[2-[2-[2-[2-[2-[2-[2-[2-[2-[2-[2-[2-[2-[2-[2-[2-[2-[2-[2-[2-[2-[4-amino-2-(ethoxymethyl)imidazo[4,5-c]quinolin-1-yl]-1,1-dimethyl-ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethyl]-3-(3-methyl-2,5-dioxo-pyrrol-1-yl)propanamide (Example 44);
(10) N-[2-[2-[2-[2-[2-[2-[2-[2-[2-[2-[2-[2-[2-[2-[2-[2-[2-[2-[2-[2-[2-[2-[2-[2-[2-[4-amino-2-(ethoxymethyl)imidazo[4,5-c]quinolin-1-yl]-1,1-dimethyl-ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethyl]-3-(3,4-dimethyl-2,5-dioxo-pyrrol-1-yl)propenamide (Example 45);
(11) N-[2-[2-[2-[2-[2-[2-[2-[2-[2-[2-[2-[2-[2-[2-[2-[2-[2-[2-[2-[2-[2-[2-[2-[2-[2-[4-amino-2-(ethoxymethyl)imidazo[4,5-c]quinolin-1-yl]-1,1-dimethyl-ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethyl]-N′-[4-(5-methylsulfonyl-1,3,4-oxadiazol-2-yl)phenyl]pentanediamide (Example 48);
(12) N-[2-[2-[2-[2-[2-[2-[2-[2-[2-[2-[2-[2-[2-[2-[2-[2-[2-[2-[2-[2-[2-[2-[2-[2-[2-[4-amino-2-(ethoxymethyl)imidazo[4,5-c]quinolin-1-yl]-1,1-dimethyl-ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethyl]-2-iodo-acetamide (Example 50);
(13) (E)-N-[2-[2-[2-[2-[2-[2-[2-[2-[2-[2-[2-[2-[2-[2-[2-[2-[2-[2-[2-[2-[2-[2-[2-[2-[2-[4-amino-2-(ethoxymethyl)imidazo[4,5-c]quinolin-1-yl]-1,1-dimethyl-ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethyl]-4-oxo-4-phenyl-but-2-enamide (Example 52); or
   a pharmaceutically acceptable thereof.

Among the preceding listed compounds of formula (I), the following compounds which are of interest may for example be cited:
(4) 1-(74-azido-2,2-dimethyl-3,6,9,12,15,18,21,24,27,30,33,36,39,42,45,48,51,54, 57,60,63,66,69,72-tetracosaoxatetraheptacontyl)-2-(ethoxymethyl)-1H-imidazo[4,5-c]quinolin-4-amine (Example 19);
(8) N-(74-(4-amino-2-(ethoxymethyl)-1H-imidazo[4,5-c]quinolin-1-yl)-73,73-dimethyl-3,6,9,12,15,18,21,24,27,30,33,36,39,42,45,48,51,54,57,60,63,66,69,72-tetracosaoxatetraheptacontyl)-3-(2,5-dioxo-2,5-dihydro-1H-pyrrol-1-yl)propanamide formate (Example 31);
   or a pharmaceutically acceptable salt thereof.

The compounds/payloads of formula (I) or a pharmaceutically acceptable salt thereof according to the present disclosure can be prepared according to any process known by the skilled person, and for instance by the following processes.

### Synthesis of the Compounds/Payloads of Formula (I)

### Synthesis of Linkers

### Synthesis of Cyclic PEGn Sulfates

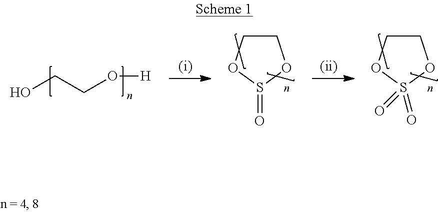

Step (i): ethylene glycol macrocyclization by treatment with thionyl chloride in the presence of a base such as, for example, a combination of DMAP and TEA;
Step (ii): oxidation of sulfite to sulfate by treatment with an oxidizing reagent such as, for example, sodium periodate in the presence of a catalytic amount of ruthenium(III) chloride trihydrate.

Scheme 1 depicted the synthesis starting with PEG4-OH (CAS number [112-60-7]) or PEG8-OH (CAS number [5117-19-1]) but may also apply to other PEGn-OH which are commercially available for n ranging from 1 to 12.

### Synthesis of Azido PEGn Aldehydes

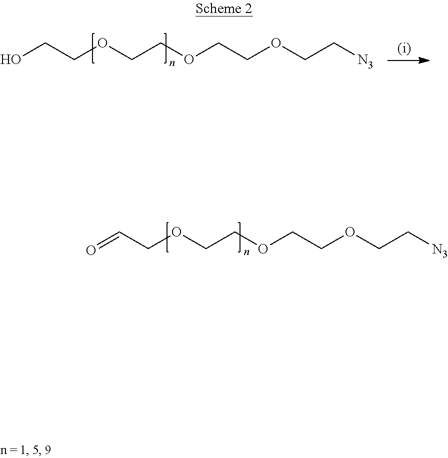

Step (i): oxidation of the alcohol in aldehyde using either oxalyl chloride in the presence of a base such as, for example, TEA or Dess-Marin periodinane in the presence of a base such as, for example, sodium bicarbonate.

Scheme 2 depicted the synthesis starting with azido-PEG4-OH (CAS number [86770-67-4]), azido-PEG8-OH (CAS number [352439-36-2]) or azido-PEG12-OH (CAS number [1821464-55-4]) but may also apply to other PEGn-OH which are commercially available for n ranging from 1 to 24.

### Synthesis of Maleimido Linkers

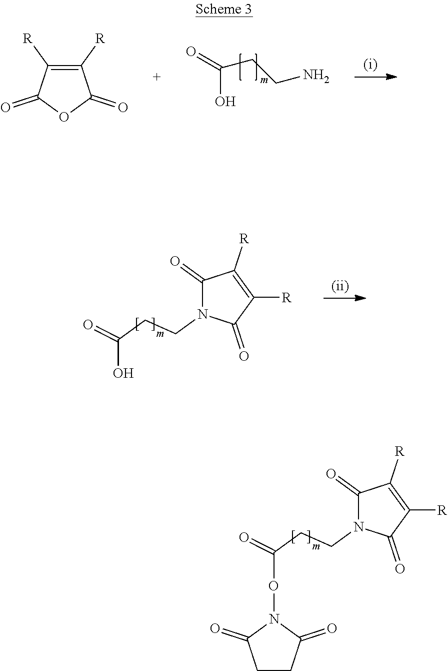

Step (i): conversion of maleic anhydride to maleimide derivative in an acidic medium such as, for example, acetic acid;
Step (ii): activation of the carboxylic acid as a NHS ester by treatment with DSC in the presence of a base such as, for example, DIEA.

### Synthesis of the Compounds/Payloads of Formula (I)

### Synthesis of R848 Compounds/Payloads of Formula (I)

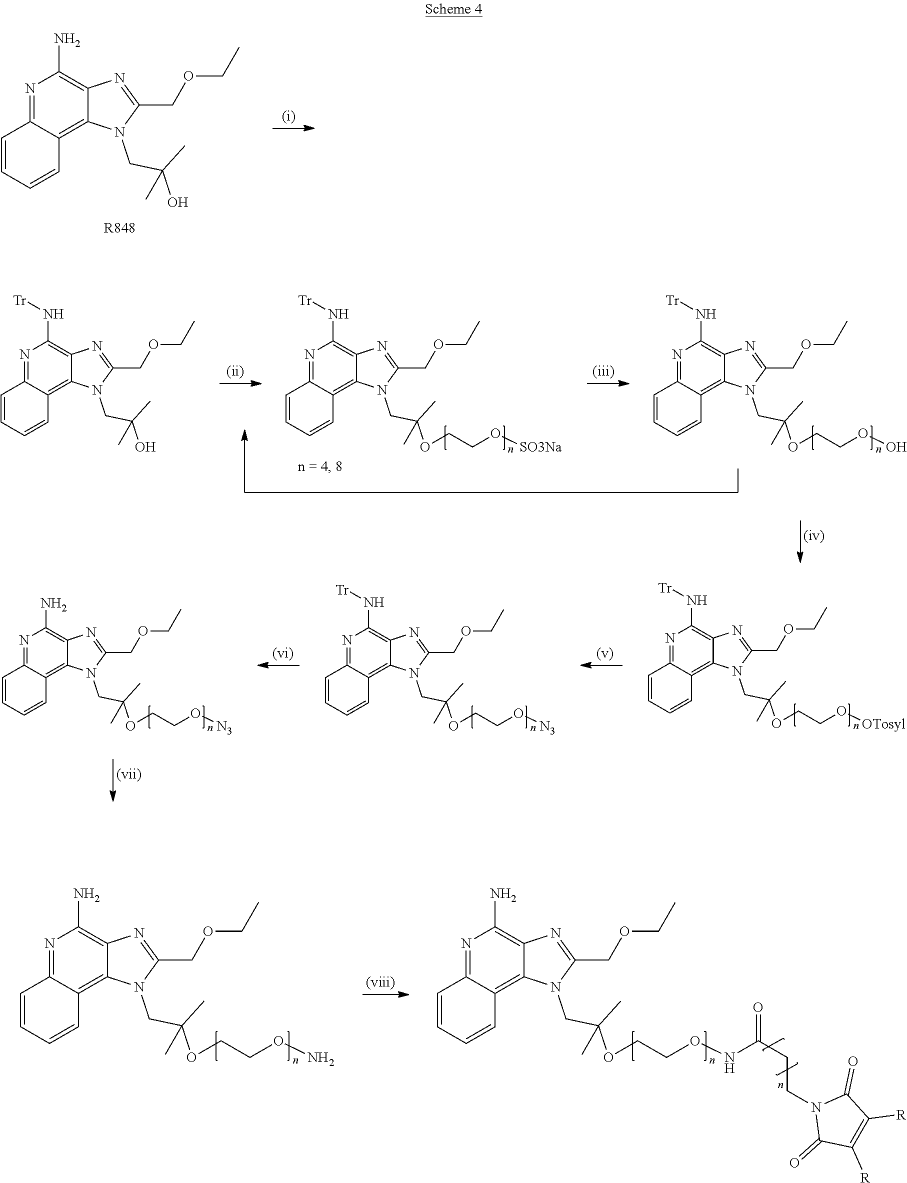

Step (i): protection of R848 primary amine with a trityl group using trityl chloride and a base such as, for example, TEA;
Step (ii): two-step O-alkylation of R848 alcohol consisting in generating an alkoxide using sodium hydride and reacting it with cyclic PEGn sulfate;
Step (iii): O-desulfatation in basic conditions such as, for example, a solution of pyridine (for example a solution in dioxane);
Step (iv): activation of the alcohol as a tosylate using tosyl chloride and a base, such as, for example, a combination of DMAP and TEA;
Step (v): substitution of the tosylate group by an azido group by treatment with sodium azide;
Step (vi): deprotection of the trityl group using a solution of hydrochloric acid (for example solution in dioxane);
Step (vii): reduction of the azido group using, for example, ammonium formiate and a catalyst, such as, for example, palladium on carbon;
Step (viii): peptidic coupling with an activated ester such as a NHS, and a base such as, for example, DIEA.

Scheme 4 depicted the synthesis starting with cyclic PEG4 sulfate or cyclic PEG8 sulfate but may also apply to other cyclic sulfates that may be prepared according to Scheme 1 for n ranging from 1 to 12. Starting from cyclic PEG8 sulfate, reiteration of step (iii) with cyclic PEG4 sulfate allows to prepare azido-PEG12-R848; starting from cyclic PEG8 sulfate, two reiterations of step (iii) with cyclic PEG8 sulfate allow to prepare azido-PEG24-R848.

### Synthesis of 3M012 Compounds/Payloads of Formula (I)

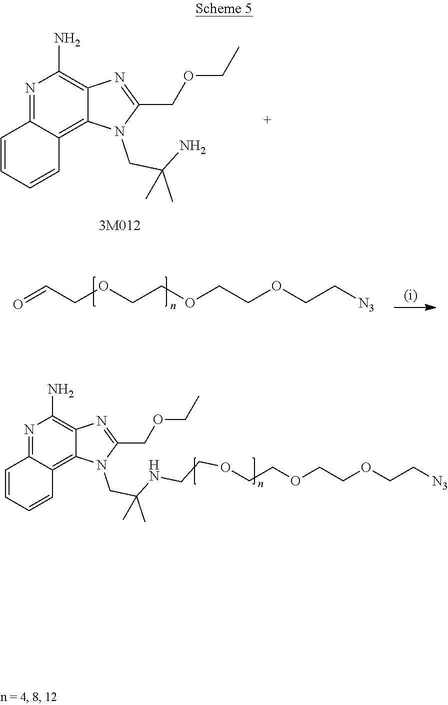

Step (i): reductive amination of 3M012 with azido-PEGn-aldehyde in the presence of a reducing reagent such as, for example, sodium triacetoxyborohydride.

Scheme 5 depicted the synthesis starting with azido-PEG4-aldehyde, azido-PEG8-aldehyde or azido-PEG12-aldehyde but may also apply to other azido-PEGn-aldehyde which may be prepared according to Scheme 2 for n ranging from 1 to 24.

The specific compounds/payloads of formula (I) as defined in the present disclosure are indicated in Table 1 (number, and formula) and are further detailed hereafter. In Table 2, ¹H NMR, and liquid chromatography/mass spectra are also indicated.

The ¹H NMR of Table 2 is ¹H NMR Spectra (400 MHz, δ in ppm, DMSO-d6) as defined in the Experimental part.

The liquid chromatography/mass spectra (LC/MS) of Table 2 were obtained according to one of the seven methods described in the Experimental part.

TABLE 1

| Number/ | |
|---|---|
| Example | Compounds/payloads of formula (I) |
| 1/ Example 1 | 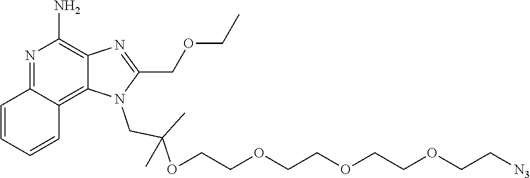 [SMILES: CCOCc1nc2c(N)nc3ccccc3c2n1CC(C)(C)OCCOCCOCCOCCN=[N+]=[N-]] |
| 2/ Example 7 | 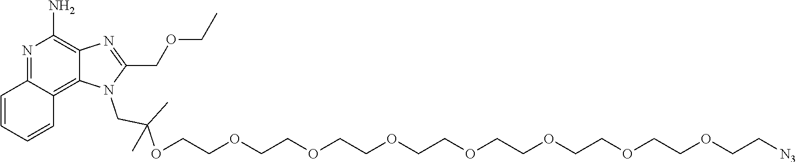 [SMILES: CCOCc1nc2c(N)nc3ccccc3c2n1CC(C)(C)OCCOCCOCCOCCOCCOCCOCCOCCN=[N+]=[N-]] |
| 3/ Example 13 | 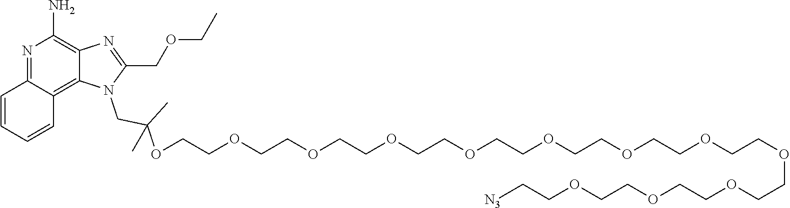 [SMILES: CCOCc1nc2c(N)nc3ccccc3c2n1CC(C)(C)OCCOCCOCCOCCOCCOCCOCCOCCOCCOCCOCCOCCN=[N+]=[N-]] |
| 4/ Example 19 | 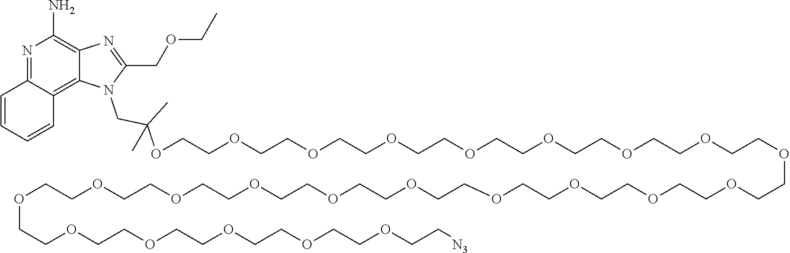 [SMILES: CCOCc1nc2c(N)nc3ccccc3c2n1CC(C)(C)OCCOCCOCCOCCOCCOCCOCCOCCOCCOCCOCCOCCOCCOCCOCCOCCOCCOCCOCCOCCOCCOCCOCCOCCN=[N+]=[N-]] |
| 5/ Example 25 | 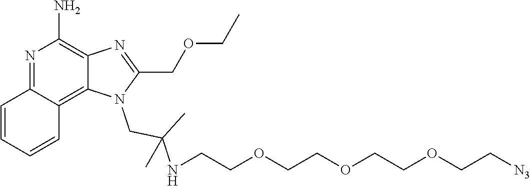 [SMILES: CCOCc1nc2c(N)nc3ccccc3c2n1CC(C)(C)NCCOCCOCCOCCN=[N+]=[N-]] |
| 6/ Example 27 | 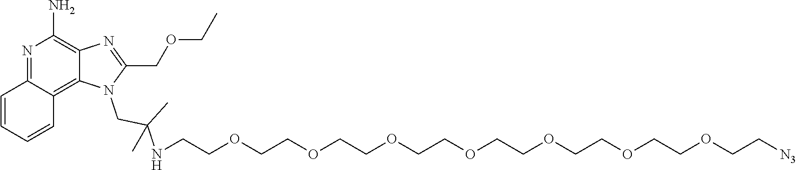 [SMILES: CCOCc1nc2c(N)nc3ccccc3c2n1CC(C)(C)NCCOCCOCCOCCOCCOCCOCCOCCN=[N+]=[N-]] |
| 7 Example 29 | 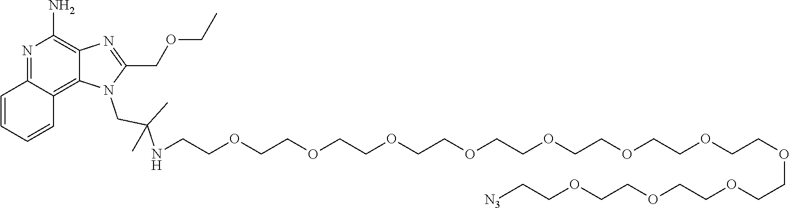 [SMILES: CCOCc1nc2c(N)nc3ccccc3c2n1CC(C)(C)NCCOCCOCCOCCOCCOCCOCCOCCOCCOCCOCCOCCN=[N+]=[N-]] |
| 8/ Example 31 | 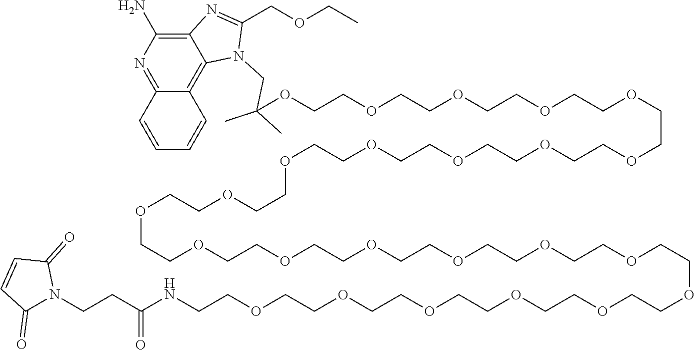 [SMILES: CCOCc1nc2c(N)nc3ccccc3c2n1CC(C)(C)OCCOCCOCCOCCOCCOCCOCCOCCOCCOCCOCCOCCOCCOCCOCCOCCOCCOCCOCCOCCOCCOCCOCCOCCNC(=O)CCN1C(=O)C=CC1=O] |
| 9/ Example 44 | 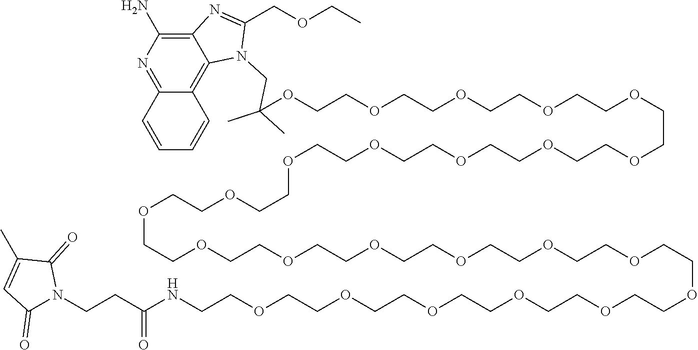 [SMILES: CCOCc1nc2c(N)nc3ccccc3c2n1CC(C)(C)OCCOCCOCCOCCOCCOCCOCCOCCOCCOCCOCCOCCOCCOCCOCCOCCOCCOCCOCCOCCOCCOCCOCCOCCNC(=O)CCN1C(=O)C=C(C)C1=O] |
| 10/ Example 45 | 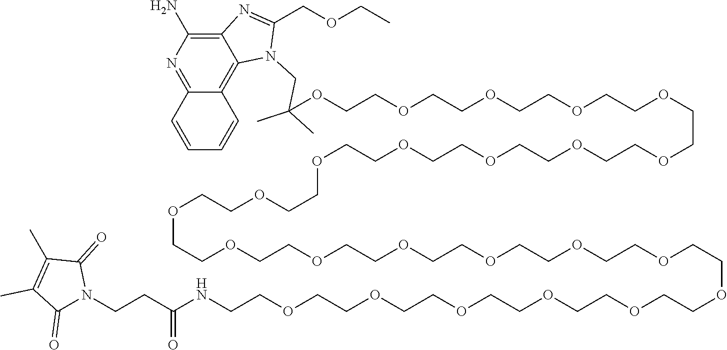 [SMILES: CCOCc1nc2c(N)nc3ccccc3c2n1CC(C)(C)OCCOCCOCCOCCOCCOCCOCCOCCOCCOCCOCCOCCOCCOCCOCCOCCOCCOCCOCCOCCOCCOCCOCCOCCNC(=O)CCN1C(=O)C(C)=C(C)C1=O] |
| 11/ Example 48 | 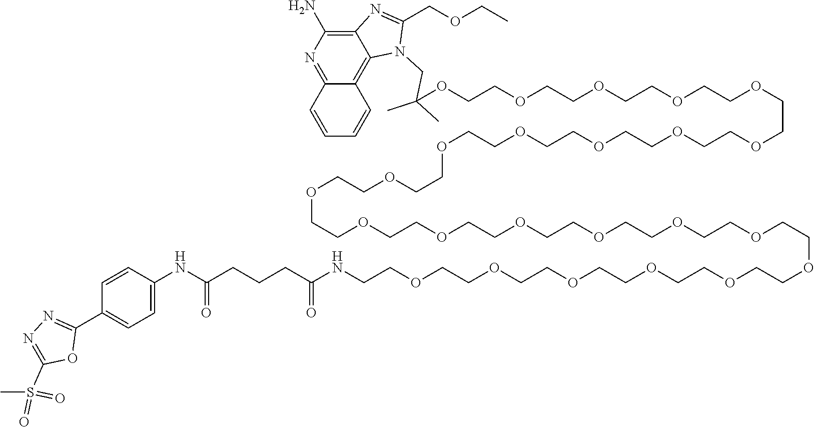 [SMILES: CCOCc1nc2c(N)nc3ccccc3c2n1CC(C)(C)OCCOCCOCCOCCOCCOCCOCCOCCOCCOCCOCCOCCOCCOCCOCCOCCOCCOCCOCCOCCOCCOCCOCCOCCNC(=O)CCCC(=O)Nc1ccc(-c2nnc(S(C)(=O)=O)o2)cc1] |
| 12/ Example 50 | 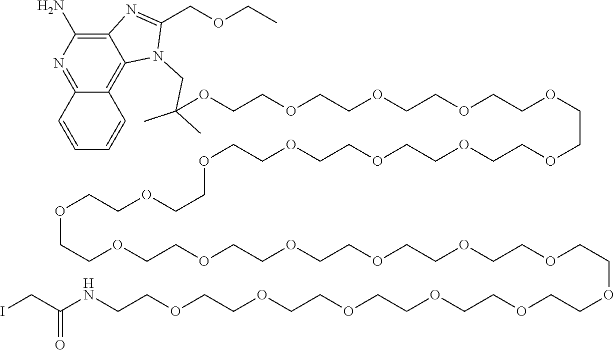 [SMILES: CCOCc1nc2c(N)nc3ccccc3c2n1CC(C)(C)OCCOCCOCCOCCOCCOCCOCCOCCOCCOCCOCCOCCOCCOCCOCCOCCOCCOCCOCCOCCOCCOCCOCCOCCNC(=O)CI] |
| 13/ Example 52 | 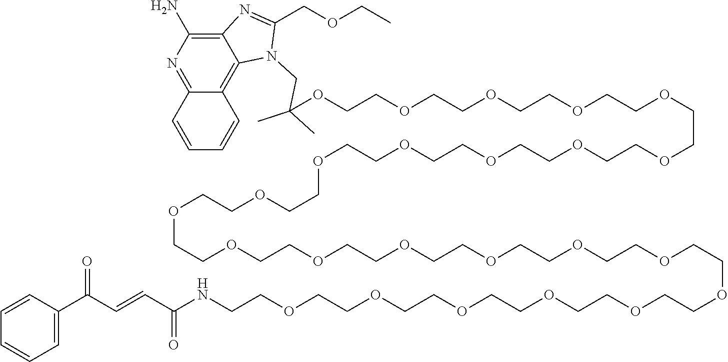 [SMILES: CCOCc1nc2c(N)nc3ccccc3c2n1CC(C)(C)OCCOCCOCCOCCOCCOCCOCCOCCOCCOCCOCCOCCOCCOCCOCCOCCOCCOCCOCCOCCOCCOCCOCCOCCNC(=O)/C=C/C(=O)c1ccccc1] |

TABLE 2

| Number/ | | |
|---|---|---|
| Example | MS | ¹H NMR |
| 1/Example 1 | m/z = 516 [M + H]⁺ | NMR ¹H (400 MHz, δ in ppm, DMSO-d6): 1.10 to 1.26 |
| | | (m, 9 H); 3.32 to 3.70 (m, 20 H); 4.77 (large s, 2 H); 6.54 |
| | | (large s, 2 H); 7.22 (t, J = 7 Hz, 1 H); 7.41 (t, J = 8 Hz, |
| | | 1 H); 7.59 (d, J = 8 Hz, 1 H); 8.29 (d, J = 8 Hz, 1 H) |
| 2/Example 7 | m/z = 692 [M + H]⁺ | NMR ¹H (400 MHz, δ in ppm, DMSO-d6): 1.10 to 1.28 |
| | | (m, 9 H); 3.32 to 3.60 (m, 36 H); 4.77 (large s, 2 H); 6.55 |
| | | (large s, 2 H); 7., 23 (t, J = 8 Hz, 1 H); 7.41 (t, J = 8 Hz, |
| | | 1 H); 7.59 (d, J = 8 Hz, 1 H); 8.29 (d, J = 8 Hz, 1 H) |
| 3/Example 13 | m/z = 868 [M + H]⁺, | NMR ¹H (400 MHz, δ in ppm, DMSO-d6): 1.07 to 1.29 |
| | m/z = 420 [M + 2H − | (m, 9 H); 3.35 to 3.66 (m, 52 H); 4.78 (large s, 2 H); 6.53 |
| | C₂H₅]²⁺, m/z = 912 | (large s, 2 H); 7.23 (t, J = 7 Hz, 1 H); 7.42 (t, J = 8 Hz, |
| | [M − H + HCOOH] | 1 H); 7.60 (d, J = 8 Hz, 1 H); 8.30 (d, J = 8 Hz, 1 H) |
| 4/Example 19 | m/z = 1396 [M + H]⁺, | NMR ¹H (400 MHz, δ in ppm, DMSO-d6): 0.89 to 1.03 |
| | m/z = 698.5 [M + 2H]²⁺ | (m, 9 H); 3.10 to 3.48 (m, 100 H); 4.55 (large s, 2 H); 6.32 |
| | | (m, 2 H); 7.00 (t, J = 8 Hz, 1 H); 7.20 (t, J = 8 Hz, 1 H); |
| | | 7.36 (t, J = 8 Hz, 1 H); 8.06 (d, J = 8 Hz, 1 H) |
| 5/Example 25 | m/z = 515 [M + H]⁺ | NMR ¹H (400 MHz, δ in ppm, DMSO-d6): 0.92 to 1.20 |
| | | (m, 9 H); 3.39 (masked m, 21 H); 6.56 (s, 2 H); 6.66 (large |
| | | s, 2 H); 7.23 (t, J = 8 Hz, 1 H); 7.42 (t, J = 8 Hz, 1 H); 7.59 |
| | | (d, J = 8 Hz, 1 H); 8.31 (d, J = 8 Hz, 1 H) |
| 6/Example 27 | m/z = 691 [M + H]⁺, | NMR ¹H (400 MHz, δ in ppm, DMSO-d6): 1.05 (large s, |
| | m/z = 346 [M + 2H]²⁺ | 6 H); 1.14 (t, J = 7 Hz, 3 H); 3.33 to 3.63 (m, 37 H); 4.66 |
| | | (large m, 2 H); 6.54 (large s, 2 H); 7.23 (t, J = 7 Hz, 1 H); |
| | | 7.41 (t, J = 8 Hz, 1 H); 7.59 (d, J = 8 Hz, 1 H); 8.31 (d, J = |
| | | 8 Hz, 1 H) |
| 7/Example 29 | m/z = 867 [M + H]⁺, | NMR ¹H (500 MHz, δ in ppm, DMSO-d6): 1.05 (m, 6 H); |
| | m/z = 434 [M + 2H]²⁺ | 1.14 (t, J = 7 Hz, 3 H); 2.59 (m, 2 H); 3.35 to 3.57 (m, |
| | | 49 H); 3.59 (m, 2 H); 4.53 to 5.06 (large m, 2 H); 6.59 |
| | | (large s, 2 H); 7.23 (t, J = 8 Hz, 1 H); 7.41 (t, J = 8 Hz, |
| | | 1 H); 7.60 (dd, J = 1 and 8 Hz, 1 H); 8.31 (dd, J = 1 and |
| | | 8 Hz, 1 H) |
| 8/Example 31 | m/z = 1521 [M + H]⁺ | NMR ¹H (400 MHz, δ in ppm, DMSO-d6): 1.05-1.30 (m, |
| | | 6 H); 1.15 (t, J = 7 Hz, 3 H); 2.28-2.37 (m, 2 H); 3.15 (q, |
| | | J = 6 Hz, 2 H); 3.25-3.70 (m, 96 H); 4.60-5.20 (m, 4 H); |
| | | 6.55 (br s, 2 H); 7.01 (s, 2 H); 7.23 (br t, J = 7 Hz, 1 H); |
| | | 7.41 (br t, J = 7 Hz, 1 H); 7.60 (br d, J = 8 Hz, 1 H); 8.01 (t, |
| | | J = 6 Hz, 1 H); 8.19 (s, 1 H); 8.29 (br d, J = 8 Hz, 1 H) |
| 9/Example 44 | m/z = 512 [M + 3H]³⁺ | NMR ¹H (400 MHz, δ in ppm, DMSO-d6): 1.05-1.35 (m, |
| | | 6 H); 1.16 (t, J = 7 Hz, 3 H); 1.98 (d, J = 2 Hz, 3 H); 2.32 (t, |
| | | J = 7 Hz, 2 H); 3.15 (q, J = 6 Hz, 2 H); 3.19-3.70 (m, 98 H); |
| | | 4.40-5.40 (m, 4 H); 6.60 (m, 1 H); 7.52 (t, J = 8 Hz, 1 H); |
| | | 7.69 (t, J = 8 Hz, 1 H); 7.80 (d, J = 8 Hz, 1 H); 7.97 (t, |
| | | J = 5 Hz, 1 H); 8.20-9.50 (m, 2 H); 8.53 (d, J = 8 Hz, 1 H); |
| | | 13.30 (br s, 1 H) |
| 10/Example 45 | m/z = 1549 [M + H]⁺ | NMR ¹H (400 MHz, δ in ppm, DMSO-d6): 1.08-1.30 (m, |
| | | 6 H); 1.17 (t, J = 7 Hz, 3 H); 1.88 (s, 6 H); 2.28-2.36 (m, |
| | | 2 H); 3.15 (q, J = 6 Hz, 2 H); 3.18-3.70 (m, 98 H); 4.40- |
| | | 5.30 (m, 4 H); 7.52 (t, J = 8 Hz, 1 H); 7.69 (t, J = 8 Hz, 1 H); |
| | | 7.80 (d, J = 8 Hz, 1 H); 7.96 (br t, J = 5 Hz, 1 H); 8.20-9.40 |
| | | (m, 2 H); 8.53 (d, J = 8 Hz, 1 H); 13.31 (br s, 1 H) |
| 11/Example 48 | m/z = 854 [M + 2H⁺]²⁺ | NMR ¹H (400 MHz, δ in ppm, DMSO-d6): 1.82 (quint, |
| | | J = 7.44 Hz, 2 H); 2.15 (t, J = 7.36 Hz, 2 H); 2.37 (t, |
| | | J = 7.40 Hz, 2 H); 3.34-3.60 (m, 98 H); 3.70 (s, 3 H); 6.58 |
| | | (s, 2 H) 7.86 (d, J = 8.80 Hz, 2 H); 7.89 (t, J = 5.68 Hz, 1 H); |
| | | 8.04 (d, J = 8.80 Hz, 2 H); 10.33 (s, 1 H) |
| 12/Example 50 | m/z = 1539 [M + H⁺]⁺ | NMR ¹H (400 MHz, δ in ppm, DMSO-d6): 6.54 (s, 2 H); 3.34 |
| | | to 3.60 (m, 98 H); 3.19 (q, J = 5.56 Hz, 2 H); 3.65 (s, 2 H) |
| 13/Example 52 | m/z = 1530 [M + H⁺]⁺ | NMR ¹H (400 MHz, δ in ppm, DMSO-d6): 6.52 (s, 2 H); |
| | | 3.34 to 3.60 (m, 98 H); 8.68 (t, J = 5.36 Hz, 1 H); 8.01 (d, |
| | | J = 7.20 Hz, 2 H); 7.59 (d, J = 8.00 Hz, 2 H); 7.70 (t, |
| | | J = 7.40 Hz, 2 H) |

The disclosure also relates to a compound/conjugate of formula (II) or a pharmaceutically acceptable salt thereof

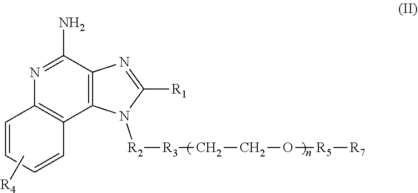

wherein
n is an integer from 1 to 50, for instance from 2 to 30, such as from 3 to 25, for instance 3, 7, 11 or 23;
R₁ represents a hydrogen atom, a —(C₁-C₆)alkylene-O—(C₁-C₆)alkyl group, a —(C₁-C₆)alkylene-NH—(C₁-C₆)alkyl group, or a —(C₁-C₆)alkyl group, for instance a —(C₁-C₆)alkylene-O—(C₁-C₆)alkyl group such as a —CH₂—O—C₂H₅ group;
R₂ represents a —(C₁-C₆)alkylene- group, for instance a —CH₂—C(CH₃)₂— group;
R₃ represents —O—, —NH— or a —N((C₁-C₆)alkyl)- group, for instance —O—or —NH—;
R₄ represents a hydrogen atom, a —(C₁-C₆)alkyl group or a —(C₁-C₆)alkoxy group, for instance a hydrogen atom;
R₅ represents a —(C₁-C₆)alkylene- group, for instance a —CH₂—CH₂— group; and
R₇ represents a -L₁-G-Ab group or a -G-Ab group,
   L₁ representing:
      —NH—;
      —C(═O)—NH—, for instance the —C(═O)— being linked to G and the —NH— being linked to R₅;
      a —(C₁-C₆)alkylene-R₈—C(═O)—NH— group, R₈ being a —(C₃-C₁₀)cycloalkylene- group or a —(C₃-C₁₀)heterocycloalkylene- group comprising 2 to 9 carbon atoms and 1 to 4 heteroatom(s) selected from oxygen, nitrogen, sulfur, —S(O)— and —SO₂—, for instance L₁ is a —(CH₂)₂-piperidinyl-C(═O)—NH— group, for instance the —(C₁-C₆)alkylene- group being linked to G and the —NH— being linked to R₅;
      a —NH—C(═O)—(C₁-C₆)alkylene-C(═O)—NH— group, for instance L₁ is a —NH—C(═O)—(CH₂)₃—C(═O)—NH— group, one —NH— being linked to G and the other —NH— being linked to R₅; or
      a —(C₁-C₆)alkylene-C(═O)—NH— group, the —(C₁-C₆)alkylene- group being optionally substituted by a NH₂ group, for instance a —CH₂—CH(CH₂—NH₂)—C(═O)—NH— group, a —(CH₂)₂—C(═O)—NH— group or a —(CH₂)₃—C(═O)—NH— group, for instance the —(C₁-C₆)alkylene- group being linked to G and the —NH— being linked to R₅;
   Ab representing an antibody, for instance a monoclonal antibody; and
   G representing the product of reaction between RCG₁ as defined in the present disclosure and RCG₂, a reactive group present on the antibody, for instance G is selected from the group consisting of:

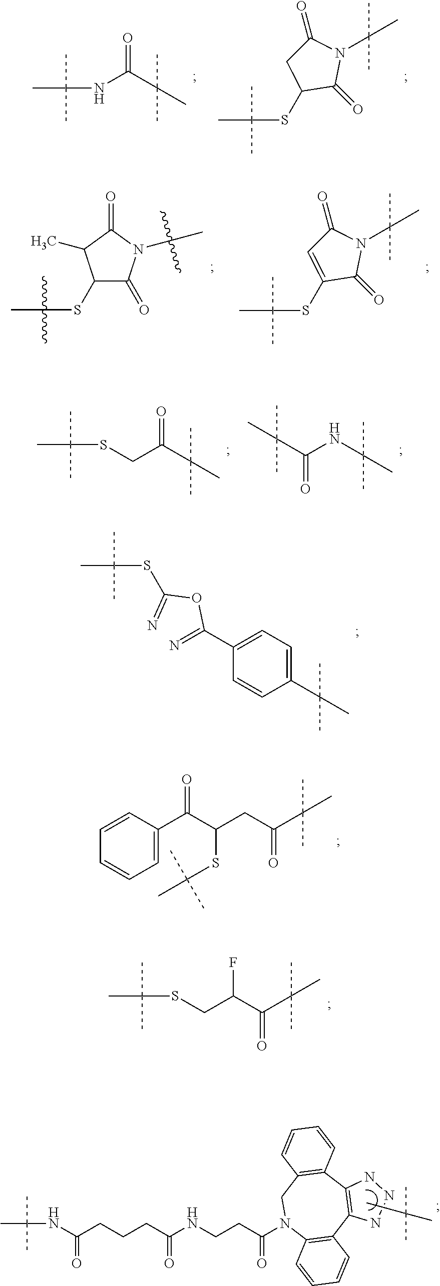

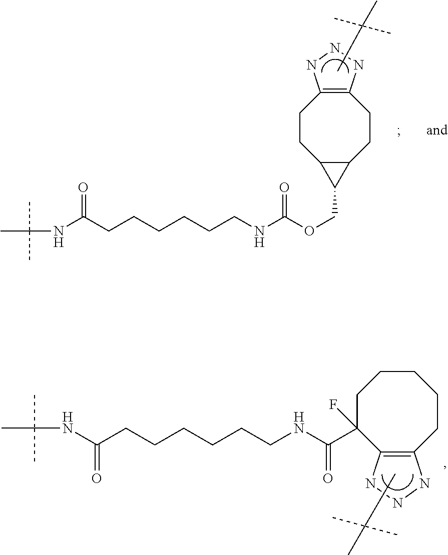

for instance the left side of the G groups being linked to Ab and the right side of the G groups being linked to L₁ when R₇ is -L₁-G-Ab or directly linked to R₅ when R₇ is -G-Ab, for instance, G represents the following groups:

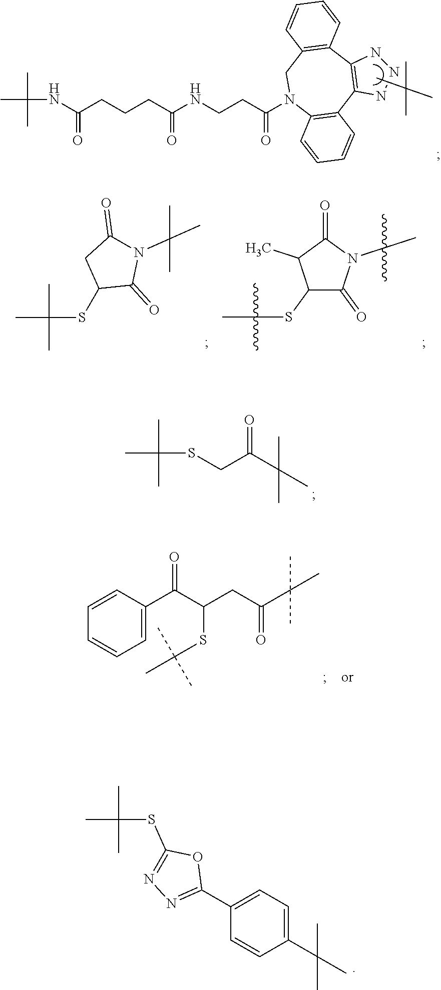

Among the compounds of formula (II) that are subject matter of the disclosure, a group of compounds is composed of the compounds for which n is 3 to 25.

Among the compounds of formula (II) that are subject matter of the disclosure, a group of compounds is composed of the compounds for which n is 3, 7, 11 or 23.

Among the compounds of formula (II) that are subject matter of the disclosure, a group of compounds is composed of the compounds for which R₁ represents a —(C₁-C₆)alkylene-O—(C₁-C₆)alkyl group.

Among the compounds of formula (II) that are subject matter of the disclosure, a group of compounds is composed of the compounds for which R₁ represents a —CH₂—O—C₂H₅ group (also named a —CH₂—O-Et group).

Among the compounds of formula (II) that are subject matter of the disclosure, a group of compounds is composed of the compounds for which R₂ represents a branched —(C₁-C₆)alkylene- group.

Among the compounds of formula (II) that are subject matter of the disclosure, a group of compounds is composed of the compounds for which R₂ represents a —CH₂—C(CH₃)₂— group, for instance the CH₂ group being linked to the nitrogen atom of the imidazo[4,5-c]quinoline ring and the C(CH₃)₂ group, being linked to R₃.

Among the compounds of formula (II) that are subject matter of the disclosure, a group of compounds is composed of the compounds for which R₃ represents —O— or —NH—.

Among the compounds of formula (II) that are subject matter of the disclosure, a group of compounds is composed of the compounds for which R₃ represents —O—.

Among the compounds of formula (II) that are subject matter of the disclosure, a group of compounds is composed of the compounds for which R₃ represents —NH—.

Among the compounds of formula (II) that are subject matter of the disclosure, a group of compounds is composed of the compounds for which R₄ represents a hydrogen atom.

Among the compounds of formula (II) that are subject matter of the disclosure, a group of compounds is composed of the compounds for which R₅ represents a linear —(C₁-C₆)alkylene-group.

Among the compounds of formula (II) that are subject matter of the disclosure, a group of compounds is composed of the compounds for which R₅ represents a —(CH₂)₂— group.

Among the compounds of formula (II) that are subject matter of the disclosure, a group of compounds is composed of the compounds for which R₇ represents a -G-Ab group,
Ab representing an antibody, such as a monoclonal antibody, for instance Tusamitamab (CAS [2349294-95-5]) or a variant of Tusamitamab, hu2H11_R35R74, trastuzumab, enoblituzumab or cetuximab, and
G representing a group of formula

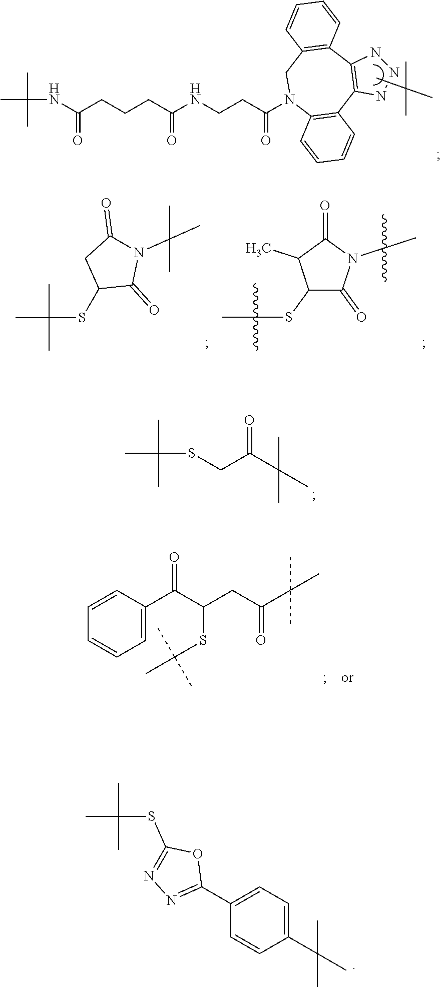

particular G being

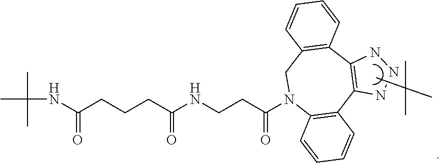

Among the compounds of formula (II) that are subject matter of the disclosure, a group of compounds is composed of the compounds for which R₇ represents a -L₁-G-Ab group,
Ab representing an antibody, such as a monoclonal antibody, for instance Tusamitamab (CAS [2349294-95-5]) or a variant of Tusamitamab, hu2H11_R35R74, trastuzumab, enoblituzumab or cetuximab;
   G representing a group of formula

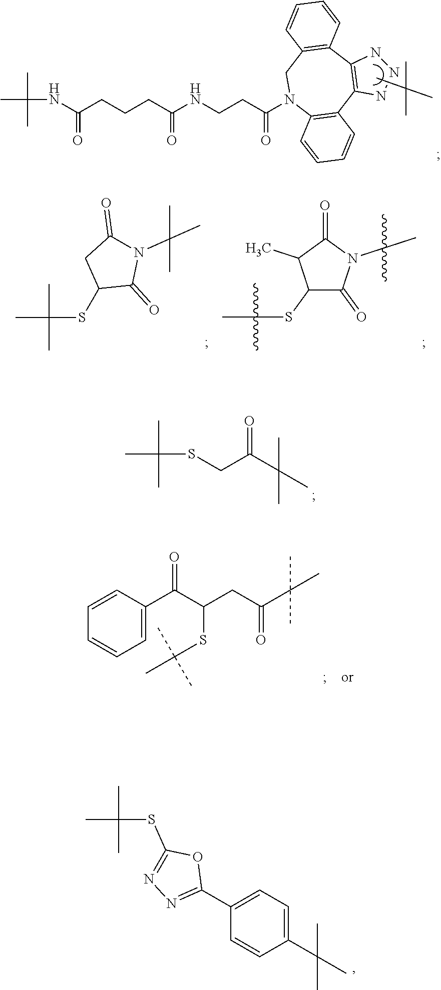

in particular G being

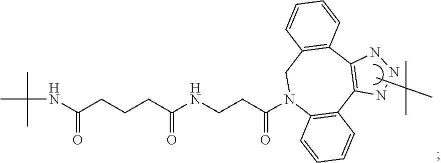

and
L₁ representing:
   NH—;
   —C(═O)—NH—, for instance the —C(═O)— being linked to G and the —NH— being linked to R₅;
      a —(C₁-C₆)alkylene-R₈—C(═O)—NH— group, R₈ being a —(C₃-C₁₀)cycloalkylene- group or a (C₃-C₁₀)heterocycloalkylene- group comprising 2 to 9 carbon atoms and 1 to 4 heteroatom(s) selected from oxygen, nitrogen, sulfur, —S(O)— and —SO₂—, for instance a —(CH₂)₂-piperidinyl-C(═O)—NH— group, for instance the —(C₁-C₆)alkylene- group being linked to G and the —NH— being linked to R₅;
      a —NH—C(═O)—(C₁-C₆)alkylene-C(═O)—NH— group, for instance L₁ is a —NH—C(═O)—(CH₂)₃—C(═O)—NH— group, one —NH— being linked to G and the other —NH— being linked to R₅; or
      a —(C₁-C₆)alkylene-C(═O)—NH— group, the —(C₁-C₆)alkylene- group being optionally substituted by a —NH₂ group, for instance a —CH₂—CH(CH₂—NH₂)—C(═O)—NH— group, a —(CH₂)₂—C(═O)—NH— group or a —(CH₂)₃—C(═O)—NH— group, for instance the —(C₁-C₆)alkylene- group being linked to G and the —NH— being linked to R₅.

All these sub-groups taken alone or in combination are part of the present disclosure.

According to a particular embodiment, the disclosure relates to a compound/conjugate of formula (II) or a pharmaceutically acceptable salt thereof wherein:
R₁ represents a —(C₁-C₆)alkylene-O—(C₁-C₆)alkyl group;
R₂ represents a —(C₁-C₆)alkylene- group;
R₃ represents —O—;
R₄ represents a hydrogen atom;
R₅ represents a —(C₁-C₆)alkyl group; and
R₇ represents a -G-Ab group, Ab being an antibody such as a monoclonal antibody and G being

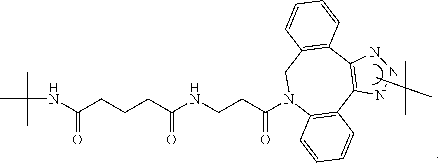

According to another particular embodiment, the disclosure relates to a compound/conjugate of formula (II) or a pharmaceutically acceptable salt thereof wherein:
R₁ represents a —CH₂—O-Et group;
R₂ represents a —CH₂—C(CH₃)₂— group;
R₃ represents —O—;
R₄ represents a hydrogen atom;
R₅ represents a —CH₂—CH₂-group; and
R₇ represents a -G-Ab group, Ab being an antibody such as a monoclonal antibody and G being

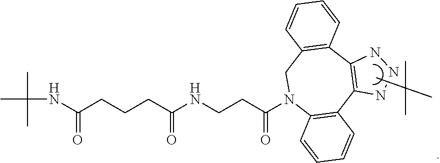

According to another particular embodiment, the disclosure relates to a compound/conjugate of formula (II) or a pharmaceutically acceptable salt thereof wherein:
R₁ represents a —(C₁-C₆)alkylene-O—(C₁-C₆)alkyl group;
R₂ represents a —(C₁-C₆)alkylene- group;
R₃ represents —O—;
R₄ represents a hydrogen atom;
R₅ represents a —(C₁-C₆)alkyl group; and
R₇ represents a -L₁-G-Ab group, Ab being an antibody such as a monoclonal antibody and G being

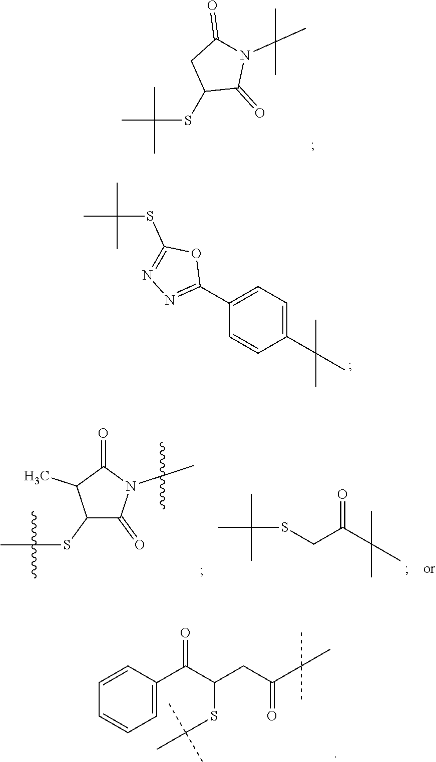

According to another particular embodiment, the disclosure relates to a compound/conjugate of formula (II) or a pharmaceutically acceptable salt thereof wherein:
R₁ represents a —CH₂—O-Et group;
R₂ represents a —CH₂—C(CH₃)₂— group;
R₃ represents —O—;
R₄ represents a hydrogen atom;
R₅ represents a —CH₂—CH₂-group; and
R₇ represents a -L₁-G-Ab group, Ab being an antibody such as a monoclonal antibody and G being

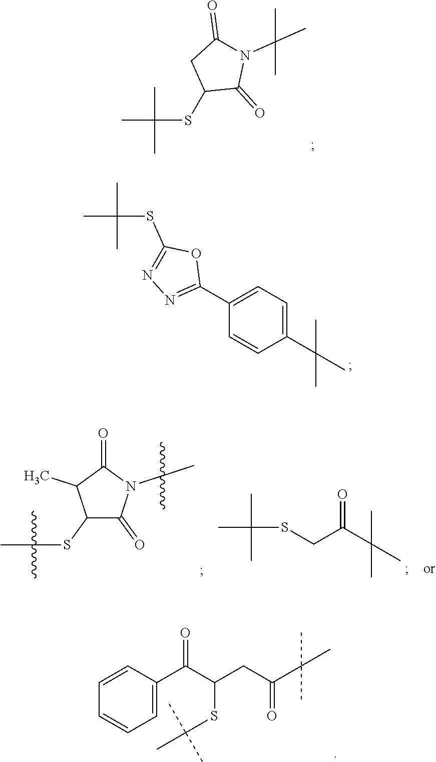

According to another particular embodiment, the disclosure relates to a compound/conjugate of formula (II) or a pharmaceutically acceptable salt thereof wherein:
R₁ represents a —(C₁-C₆)alkylene-O—(C₁-C₆)alkyl group;
R₂ represents a —(C₁-C₆)alkylene group;
R₃ represents —NH—;
R₄ represents a hydrogen atom;
R₅ represents a —(C₁-C₆)alkylene- group; and
R₇ represents a -G-Ab group, Ab being an antibody such as a monoclonal antibody and G being

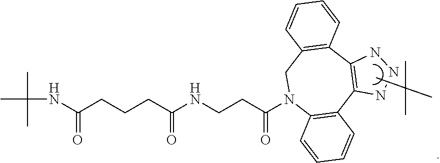

According to another particular embodiment, the disclosure relates to a compound/conjugate of formula (II) or a pharmaceutically acceptable salt thereof wherein
R₁ represents a —CH₂—O-Et group;
R₂ represents a —CH₂—C(CH₃)₂— group;
R₃ represents —NH—;
R₄ represents a hydrogen atom;
R₅ represents a —CH₂—CH₂— group; and
R₇ represents a -G-Ab group, Ab being an antibody such as a monoclonal antibody and G being

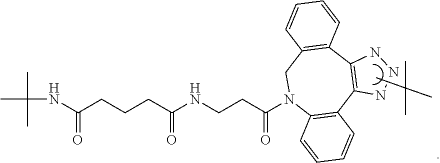

Among the compounds/conjugates of formula (II) that are subject matter of the present disclosure, mention may be made for instance to the following compounds:

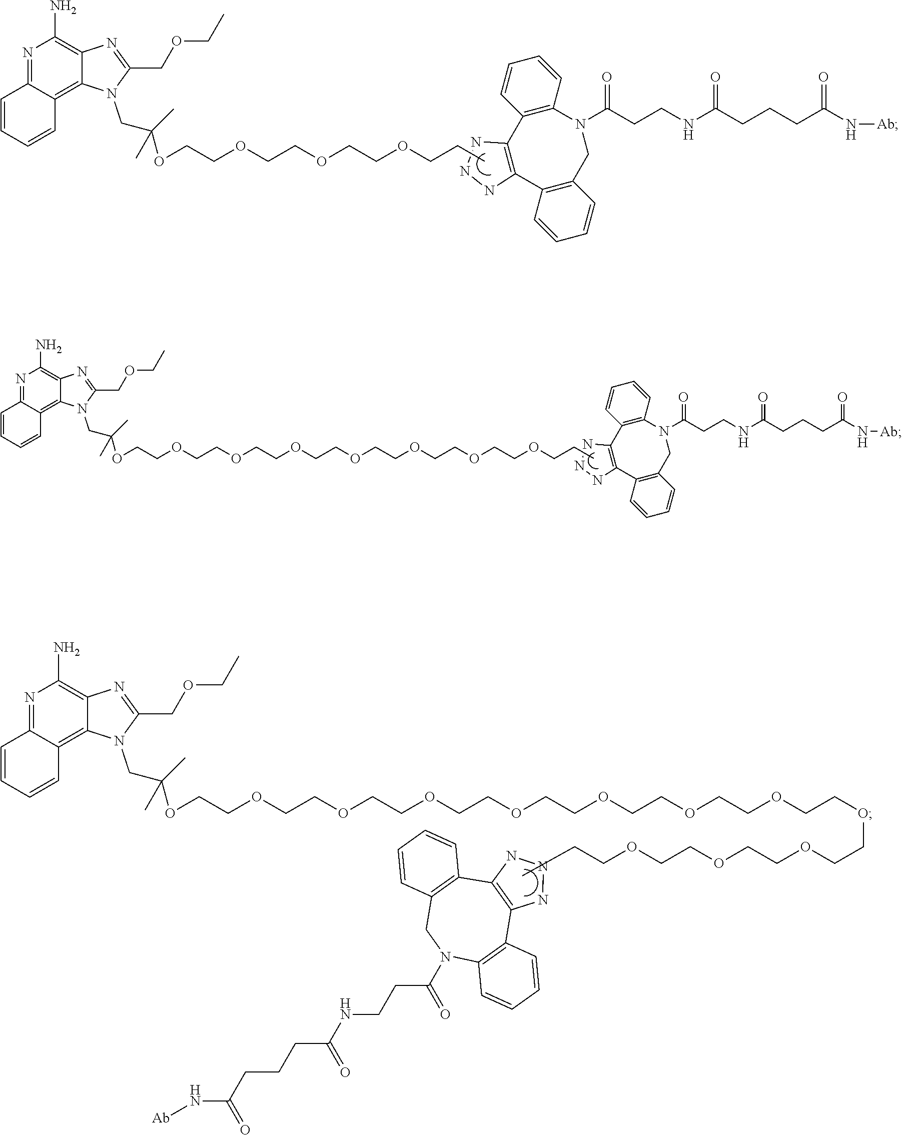

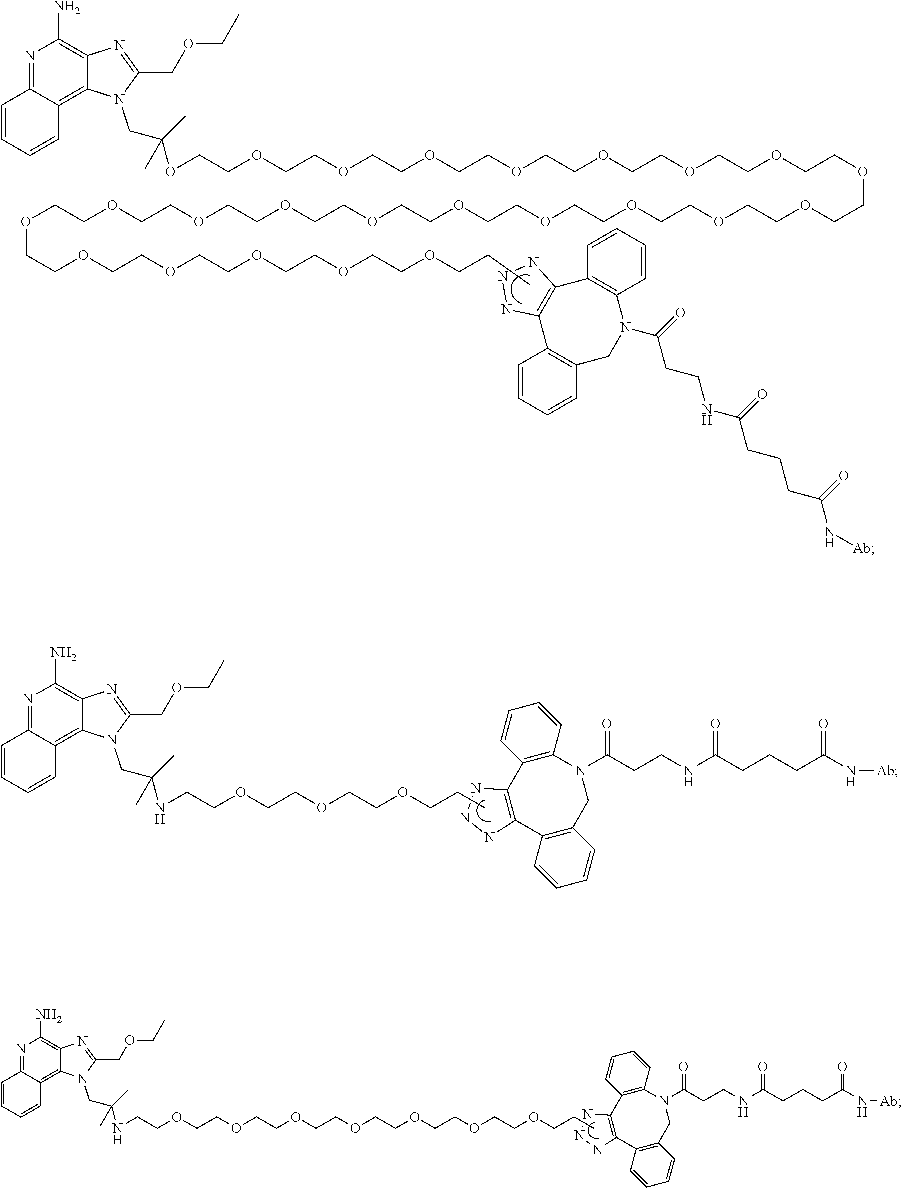

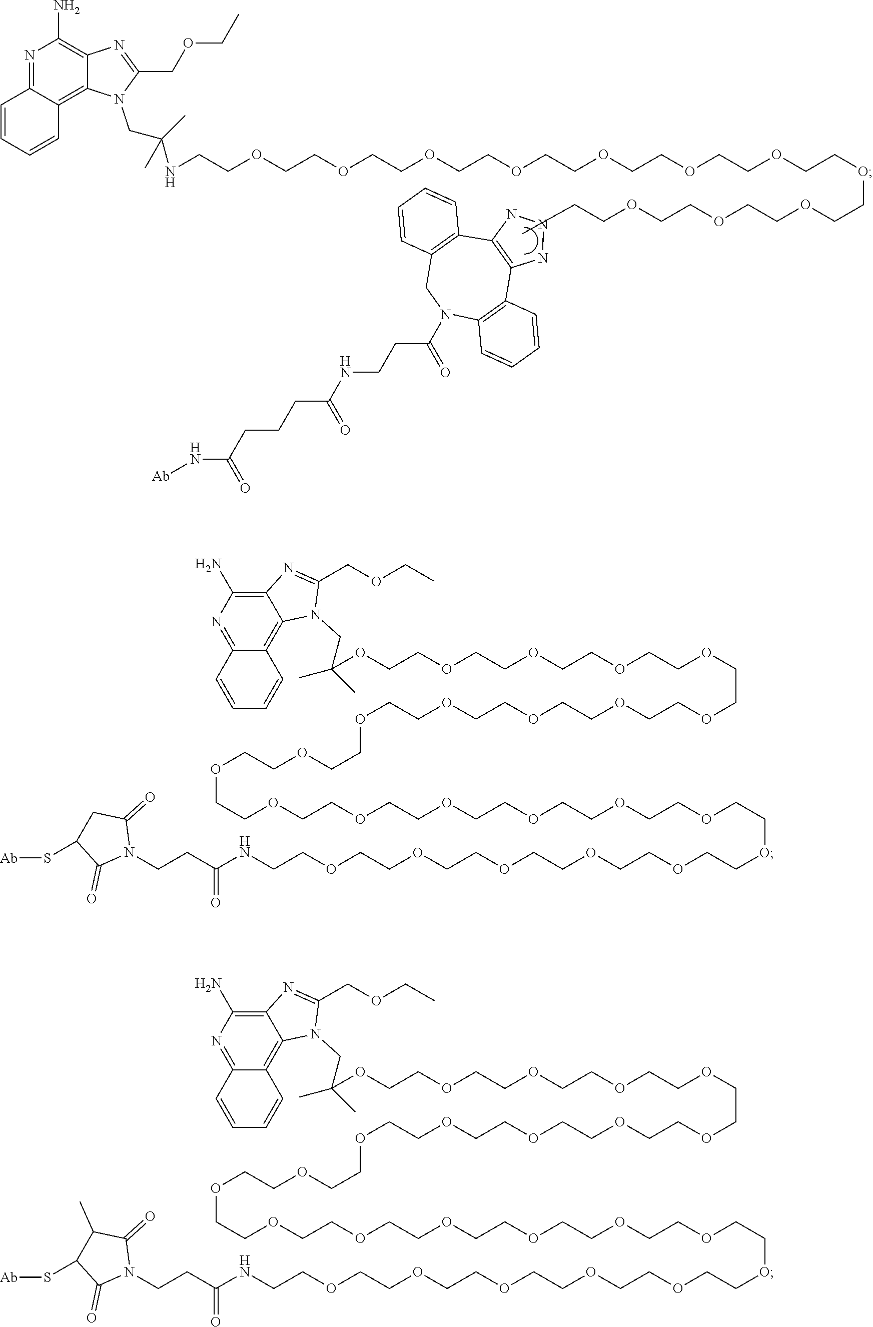

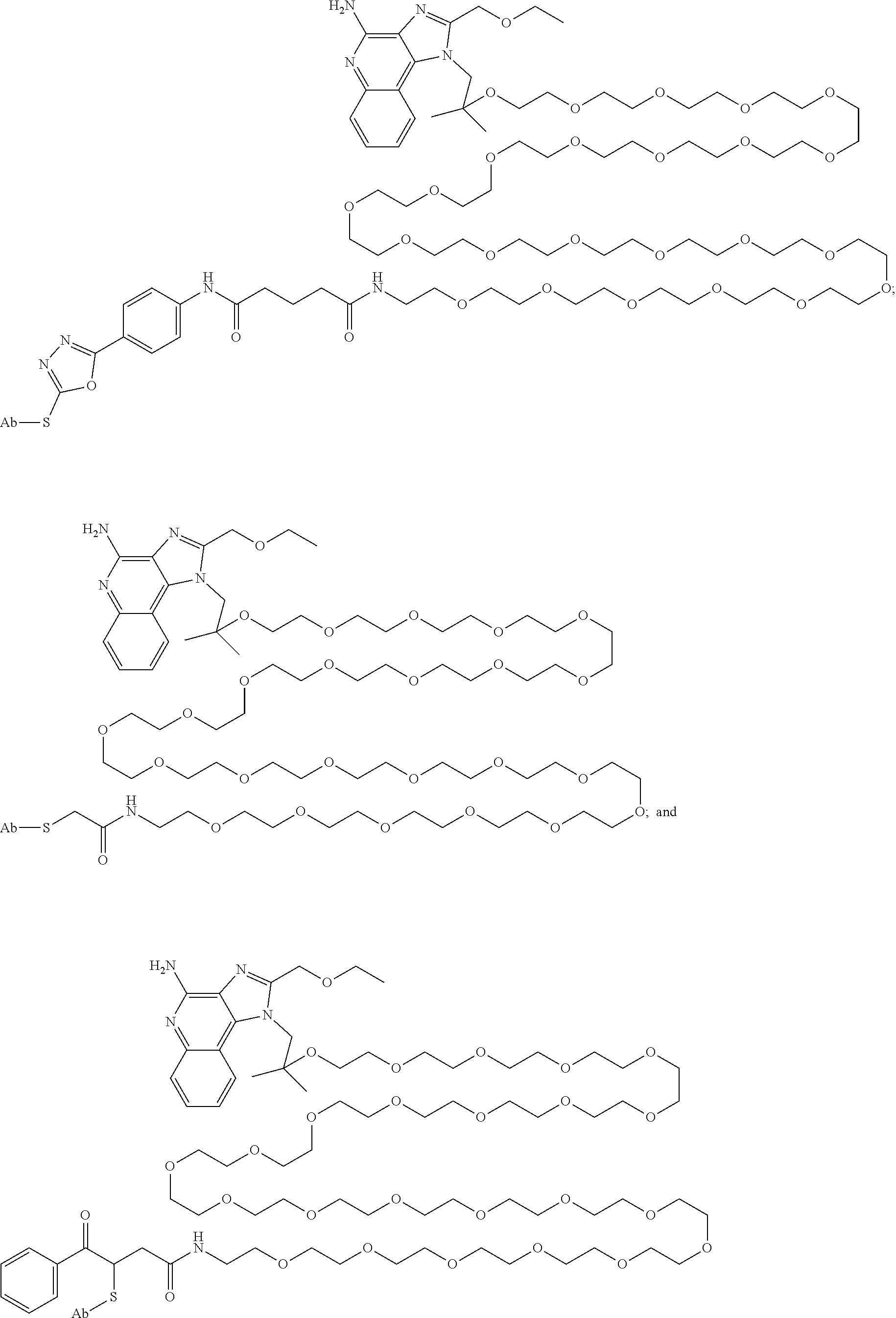

wherein Ab represents an antibody, for instance a monoclonal antibody.

The Tusamitamab variants are named according to the EU numbering in the below lists.

In particular, among the compounds/conjugates of formula (II) or a pharmaceutically acceptable salt thereof that are subject matter of the present disclosure, mention may be made for instance to the following compounds:
(c1) CEACAM5_Tusamitamab-PEG4-R848 ADC (Example 2);
(c2) EphA2_hu2H11_R35R74-PEG4-R848 ADC (Example 3);
(c3) HER2_trastuzumab-PEG4-R848 ADC (Example 4);
(c4) EGFR_cetuximab-PEG4-R848 ADC (Example 5);
(c5) B7H3_enoblituzumab-PEG4-R848 ADC (Example 6);
(c6) CEACAM5_Tusamitamab-PEG8-R848 ADC (Example 8);
(c7) EphA2_hu2H11_R35R74-PEG8-R848 ADC (Example 9);
(c8) HER2_trastuzumab-PEG8-R848 ADC (Example 10);
(c9) EGFR_cetuximab-PEG8-R848 ADC (Example 11);
(c10) B7H3_enoblituzumab-PEG8-R848 ADC (Example 12);
(c11) CEACAM5_Tusamitamab-PEG12-R848 ADC (Example 14);
(c12) EphA2_hu2H11_R35R74-PEG12-R848 ADC (Example 15);
(c13) HER2_trastuzumab-PEG12-R848 ADC (Example 16);
(c14) EGFR_cetuximab-PEG12-R848 ADC (Example 17);
(c15) B7H3_enoblituzumab-PEG12-R848 ADC (Example 18);
(c16) CEACAM5_Tusamitamab-PEG24-R848 ADC (Example 20a);
(c17) CEACAM5_Tusamitamab-PEG24-R848 ADC (Example 20b);
(c18) CEACAM5_Tusamitamab-PEG24-R848 ADC (Example 20c);
(c19) CEACAM5_Tusamitamab-PEG24-R848 ADC (Example 20d);
(c20) EphA2_hu2H11_R35R74-PEG24-R848 ADC (Example 21);
(c21) HER2_trastuzumab-PEG24-R848 ADC (Example 22);
(c22) EGFR_cetuximab-PEG24-R848 ADC (Example 23);
(c23) B7H3_enoblituzumab-PEG24-R848 ADC (Example 24);
(c24) CEACAM5_Tusamitamab-PEG4-3M-012 ADC (Example 26);
(c25) CEACAM5_Tusamitamab-PEG8-3M012 ADC (Example 28);
(c26) CEACAM5_Tusamitamab-PEG12-3M-012 ADC (Example 30);
(c27) CEACAM5_Tusamitamab_E152C-PEG24-R848 ADC (Example 32);
(c28) CEACAM5_Tusamitamab_S239C-PEG24-R848 ADC (Example 33);
(c29) CEACAM5_Tusamitamab_K274C-PEG24-R848 ADC (Example 34);
(c30) CEACAM5_Tusamitamab_K290C-PEG24-R848 ADC (Example 35);
(c31) CEACAM5_Tusamitamab_K326C-PEG24-R848 ADC (Example 36);
(c32) CEACAM5_Tusamitamab_K320C-PEG24-R848 ADC (Example 37);
(c33) CEACAM5_Tusamitamab_K340C-PEG24-R848 ADC (Example 38);
(c34) CEACAM5_Tusamitamab_S375C-PEG24-R848 ADC (Example 39);
(c35) CEACAM5_Tusamitamab_N361C-PEG24-R848 ADC (Example 40);
(c36) CEACAM5_Tusamitamab_K414C-PEG24-R848 ADC (Example 41);
(c37) CEACAM5_Tusamitamab_V422C-PEG24-R848 ADC (Example 42);
(c38) CEACAM5_Tusamitamab_E152C_S375C-PEG24-R848 ADC (Example 43);
(c39) CEACAM5_Tusamitamab_K290C-Example 44 (Example 46);
(c40) CEACAM5_Tusamitamab_K274C-Example 44 (Example 47);
(c41) CEACAM5_Tusamitamab_K274C-Example 48 (Example 49);
(c42) CEACAM5_Tusamitamab_K274C-Example 50 (Example 51); or
(c43) CEACAM5_Tusamitamab_K274C-Example 52 (Example 53).

According to a particular embodiment, compounds/payloads of formula (II) or a pharmaceutically acceptable salt thereof in accordance with the present disclosure are of formula:

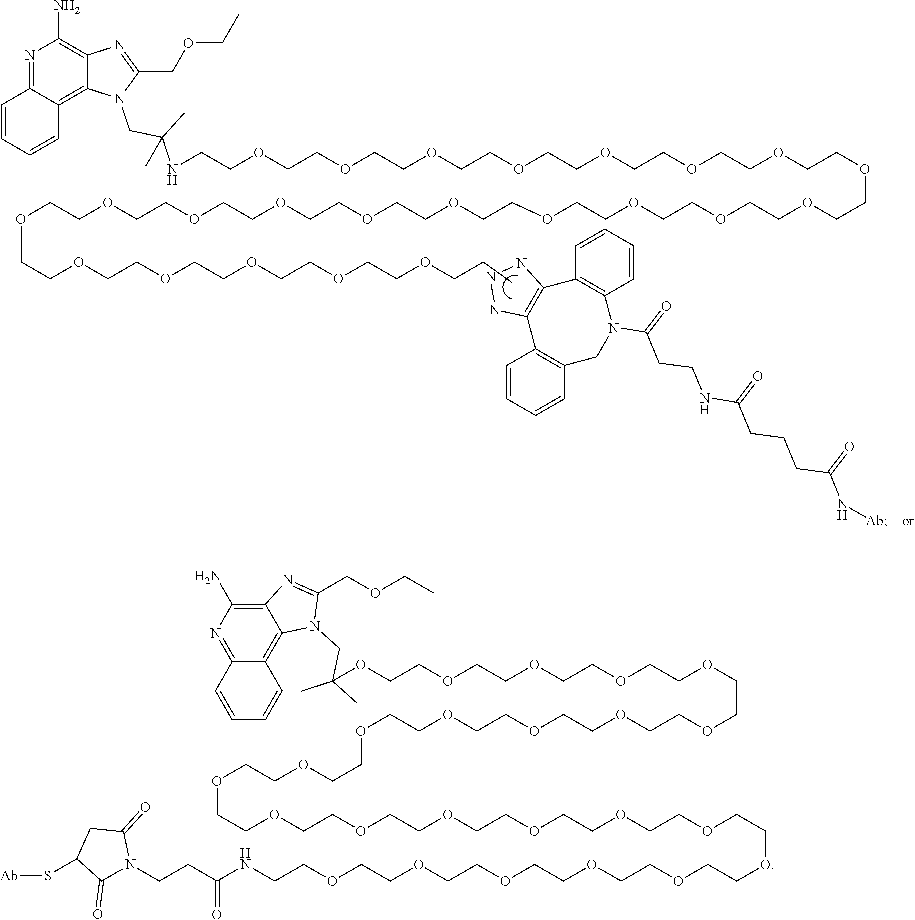

Among the preceding listed compounds/conjugates of formula (II) or a pharmaceutically acceptable salt thereof, the following compounds which are of interest may for example be cited:
(c16) CEACAM5_Tusamitamab-PEG24-R848 ADC (Example 20a);
(c17) CEACAM5_Tusamitamab-PEG24-R848 ADC (Example 20b);
(c18) CEACAM5_Tusamitamab-PEG24-R848 ADC (Example 20c);
(c19) CEACAM5_Tusamitamab-PEG24-R848 ADC (Example 20d);
(c20) EphA2_hu2H11_R35R74-PEG24-R848 ADC (Example 21);
(c21) HER2_trastuzumab-PEG24-R848 ADC (Example 22);
(c22) EGFR_cetuximab-PEG24-R848 ADC (Example 23);
(c23) B7H3_enoblituzumab-PEG24-R848 ADC (Example 24);
(c27) CEACAM5_Tusamitamab_E152C-PEG24-R848 ADC (Example 32);
(c28) CEACAM5_Tusamitamab_S239C-PEG24-R848 ADC (Example 33);
(c29) CEACAM5_Tusamitamab_K274C-PEG24-R848 ADC (Example 34);
(c30) CEACAM5_Tusamitamab_K290C-PEG24-R848 ADC (Example 35);
(c31) CEACAM5_Tusamitamab_K326C-PEG24-R848 ADC (Example 36);
(c32) CEACAM5_Tusamitamab_K320C-PEG24-R848 ADC (Example 37);
(c33) CEACAM5_Tusamitamab_K340C-PEG24-R848 ADC (Example 38);
(c34) CEACAM5_Tusamitamab_S375C-PEG24-R848 ADC (Example 39);
(c35) CEACAM5_Tusamitamab_N361C-PEG24-R848 ADC (Example 40);
(c36) CEACAM5_Tusamitamab_K414C-PEG24-R848 ADC (Example 41);
(c37) CEACAM5_Tusamitamab_V422C-PEG24-R848 ADC (Example 42);
(c38) CEACAM5_Tusamitamab_E152C_S375C-PEG24-R848 ADC (Example 43);
(c39) CEACAM5_Tusamitamab_K290C-Example 44 (Example 46);
(c40) CEACAM5_Tusamitamab_K274C-Example 44 (Example 47);
(c41) CEACAM5_Tusamitamab_K274C-Example 48 (Example 49);
(c42) CEACAM5_Tusamitamab_K274C-Example 50 (Example 51); or
(c43) CEACAM5_Tusamitamab_K274C-Example 52 (Example 53).

Among the preceding listed compounds/conjugates of formula (II) or a pharmaceutically acceptable salt thereof, the following compounds which are of particular interest may for example be cited:
(c1) CEACAM5_Tusamitamab-PEG4-R848 ADC (Example 2);
(c6) CEACAM5_Tusamitamab-PEG8-R848 ADC (Example 8);
(c11) CEACAM5_Tusamitamab-PEG12-R848 ADC (Example 14);
(c16) CEACAM5_Tusamitamab-PEG24-R848 ADC (Example 20a);
(c17) CEACAM5_Tusamitamab-PEG24-R848 ADC (Example 20b);
(c18) CEACAM5_Tusamitamab-PEG24-R848 ADC (Example 20c);
(c19) CEACAM5_Tusamitamab-PEG24-R848 ADC (Example 20d);
(c24) CEACAM5_Tusamitamab-PEG4-3M-012 ADC (Example 26);
(c25) CEACAM5_Tusamitamab-PEG8-3M012 ADC (Example 28);
(c26) CEACAM5_Tusamitamab-PEG12-3M-012 ADC (Example 30);
(c27) CEACAM5_Tusamitamab_E152C-PEG24-R848 ADC (Example 32);
(c28) CEACAM5_Tusamitamab_S239C-PEG24-R848 ADC (Example 33);
(c29) CEACAM5_Tusamitamab_K274C-PEG24-R848 ADC (Example 34);
(c30) CEACAM5_Tusamitamab_K290C-PEG24-R848 ADC (Example 35);
(c31) CEACAM5_Tusamitamab_K326C-PEG24-R848 ADC (Example 36);
(c32) CEACAM5_Tusamitamab_K320C-PEG24-R848 ADC (Example 37);
(c33) CEACAM5_Tusamitamab_K340C-PEG24-R848 ADC (Example 38);
(c34) CEACAM5_Tusamitamab_S375C-PEG24-R848 ADC (Example 39);
(c35) CEACAM5_Tusamitamab_N361C-PEG24-R848 ADC (Example 40);
(c36) CEACAM5_Tusamitamab_K414C-PEG24-R848 ADC (Example 41);
(c37) CEACAM5_Tusamitamab_V422C-PEG24-R848 ADC (Example 42);
(c38) CEACAM5_Tusamitamab_E152C_S375C-PEG24-R848 ADC (Example 43);
(c39) CEACAM5_Tusamitamab_K290C-Example 44 (Example 46);
(c40) CEACAM5_Tusamitamab_K274C-Example 44 (Example 47);
(c41) CEACAM5_Tusamitamab_K274C-Example 48 (Example 49);
(c42) CEACAM5_Tusamitamab_K274C-Example 50 (Example 51); or
(c43) CEACAM5_Tusamitamab_K274C-Example 52 (Example 53).

Among the preceding listed compounds/conjugates of formula (II) or a pharmaceutically acceptable salt thereof, the following compounds which are of more particular interest may for example be cited
(c16) CEACAM5_Tusamitamab-PEG24-R848 ADC (Example 20a);
(c17) CEACAM5_Tusamitamab-PEG24-R848 ADC (Example 20b);
(c18) CEACAM5_Tusamitamab-PEG24-R848 ADC (Example 20c); or
(c19) CEACAM5_Tusamitamab-PEG24-R848 ADC (Example 20d).

The compounds/conjugates of formula (II) or a pharmaceutically acceptable salt thereof according to the present disclosure can be prepared according to any process known by the skilled person, and for instance by the following processes. Processes are also detailed in the experimental part of the present disclosure.

The compound/conjugate of formula (II) may be prepared by
(i) placing in contact and leaving to react:
   an, optionally buffered, aqueous solution of an antibody Ab, comprising a reactive RCG2 group, optionally modified by means of a modifying agent,
and
   a solution of a compound/payload of formula (I) as defined in in the present disclosure, comprising a reactive RCG1 group;
RCG1 groups of the compounds/payloads of formula (I) being reactive towards RCG2 groups of the antibody to form the G group by a covalent bond and forming compounds/conjugates of formula (II);
(ii) and then optionally separating the compound of formula (II) formed in step (i) from the unreacted compound of formula (I) and/or from the unreacted antibody and/or from any aggregates that may have been formed.

Examples of RCG2, present on the antibody, that may be mentioned include (Garnett M. C., et al., Advanced Drug Delivery Reviews 2001, 53, 171-216):
(i) a ε-amino group (ε—NH₂ group) borne by the side chain of a lysine residue that is present at the surface of the antibody;
(ii) an α-amino group (α-NH₂ group) of an N-terminal amino acid of a heavy chain or a light chain of the antibody;
(iii) the saccharide groups of the hinge region;
(iv) a thiol (a —SH) of a cysteine residue generated by reducing an intra-chain disulfide bond of the antibody or a —SH of an engineered cysteine residue of the antibody;
(v) an amide group (a —C(O)NH₂ group) borne by the side chains of a glutamine residue that is present at the surface of the antibody;
(vi) an aldehyde group (a —C(O)H group) introduced using formylglycine generating enzyme; and
(vii) RCG2 group, optionally introduced by means of a modifying agent.

More recently, other site-specific conjugation approaches have been considered, for instance the introduction of cysteines by mutation (Junutula J. R., et al., Nature Biotechnology 2008, 26, 925-932), the introduction of unnatural amino acids allowing other types of chemistry (Axup J. Y., et al., PNAS 2012, 109, 40, 16101-16106) or the conjugation on antibody glycans (Zhou Q., et al., Bioconjugate Chem. 2014, 25, 510-520). Use of cysteine bridging dibromomaleimides (Behrens C. R., et al., Mol. Pharmaceutics 2015, 3986-3998) and bis-sulfone reagents (Bryant P., et al., Mol. Pharmaceutics 2015, 1872-1879) in order to cross-link antibodies have also been described and could be applied to the present disclosure.

Another approach for site-specific modifications of antibodies is based on enzymatic conjugation using for example bacterial transglutaminase (Jeger S., et al., Angew. Chem. Int. Ed. 2010, 49, 9995-9997; Strop P., et al., Chem. Biol. 2013, 20, 161-167) or formylglycine generating enzyme (Hudak J. E., et al., Angew. Chem. Int. Ed. 2012, 51, 4161-4165). For a review of site-specific conjugation strategies, see Agarwal P. and Bertozzi C. R., Bioconjugate Chem 2015, 26, 176-192. These conjugation technologies may also be applied to compounds/payloads of formula (I) described in the present disclosure.

It is also possible to chemically modify the antibody so as to introduce novel RCG2 reactive groups. Thus, it is well known to those skilled in the art how to modify an antibody with the aid of a modifying agent introducing for example activated disulfide, thiol, maleimido, haloacetamido, azido, alkyne or cycloalkyne groups (see especially WO2005/077090 page 14 and WO2011/001052). The modification makes it possible to improve the conjugation reaction and to use a wider variety of RCG1 groups, present on compound/payload of formula (I).

For instance, in the case where RCG1 is of the type (ii) above defined, that is to say one of the following reactive groups: a maleimido

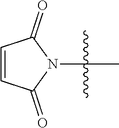 [SMILES: *N1C(=O)C=CC1=O]

group; a substituted maleimido group such as

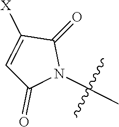

with X=Me, Br,

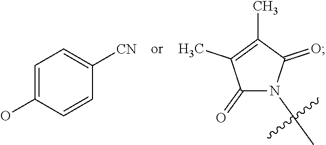

a haloacetamido

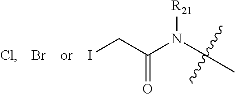

group with R₂₁ representing a hydrogen atom or a (C₁-C₆)alkyl group, such as a methyl group; Cl—; N₃—; HO—; HS—; an activated disulfide such as

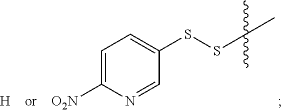

H₂N—; HC≡C— or an activated C≡C such as a cyclooctyne moiety for instance a DBCO-amine

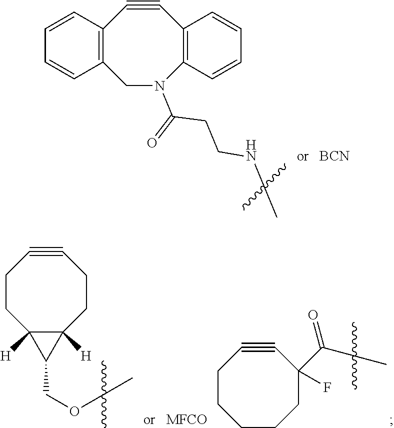

a phenyloxadiazolyl methylsulfone group (PODS) such as

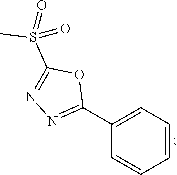

a

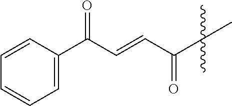 [SMILES: *C(=O)/C=C/C(=O)c1ccccc1]

group; a

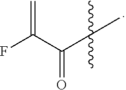 [SMILES: *C(=O)C(=C)F]

group; an O-alkyl hydroxylamine or a Pictet-Spengler reaction substrate such as

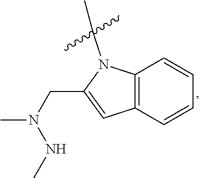 [SMILES: *n1c(CN(C)NC)cc2ccccc21]

for instance RCG₁ is N₃—; a maleimido group

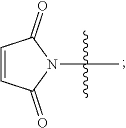 [SMILES: *N1C(=O)C=CC1=O]

a substituted maleimido group

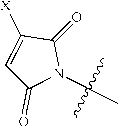

with X=Me, Br,

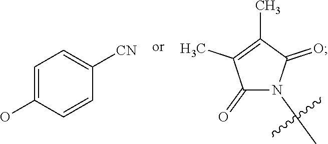

a phenyloxadiazolyl methylsulfone group

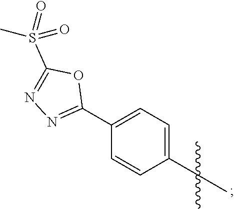 [SMILES: *c1ccc(-c2nnc(S(C)(=O)=O)o2)cc1]

a

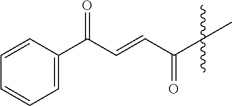 [SMILES: *C(=O)/C=C/C(=O)c1ccccc1]

group; a I—CH₂—C(═O)—NR₂₁— group with R₂₁ representing a hydrogen atom or a (C₁-C₆)alkyl group; or a

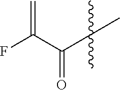 [SMILES: *C(=O)C(=C)F]

group,
it is possible to chemically modify the antibody using an adequate modifying agent or to introduce one or more unnatural amino acids so as to introduce the adequate RCG2 groups.

For example:
when RCG1 represents a N-hydroxysuccinimidyl ester, RCG2 represents a —NH₂ group;
when RCG1 represents a maleimido function, a haloacetamido function, a chorine atom or an activated disulfide, RCG2 may be a —SH group;
when RCG1 represents a —N₃ group, RCG2 may be a —C═CH function or an activated C═C function such as a cyclooctyne moiety;
when RCG1 represents a —OH group or —NH₂ group, RCG2 may be a carboxylic acid or amide function;
when RCG1 represents a —SH group, RCG2 may be a maleimido function, a haloacetamido function or an activated disulfide function;
when RCG1 represents a —C═CH function or an activated —C═C function, RCG2 may be a —N₃ group;
when RCG1 represents a O-alkyl hydroxylamine function or a Pictet-Spengler reaction substrate, RCG2 may be an aldehyde or ketone function.

As indicated above, examples of G that may be mentioned include

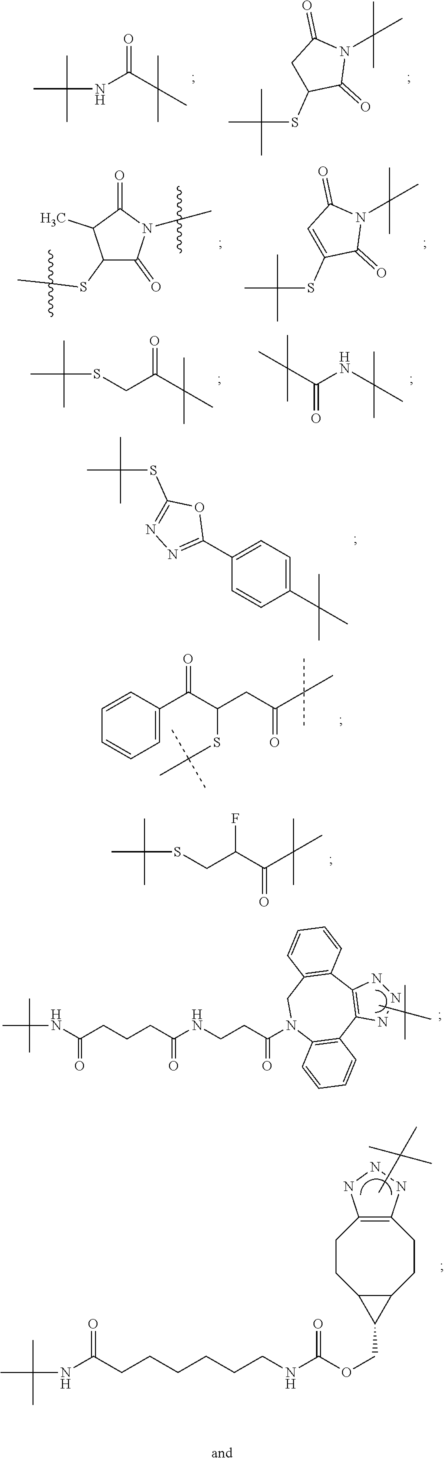

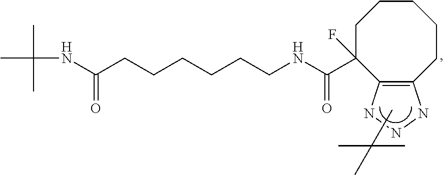

for instance, the left side of the G groups being linked to Ab and the right side of the G groups being linked to L₁ when R₇ is -L₁-G-Ab or directly linked to R₅ when R₇ is -G-Ab.

For instance, G represents the following groups:

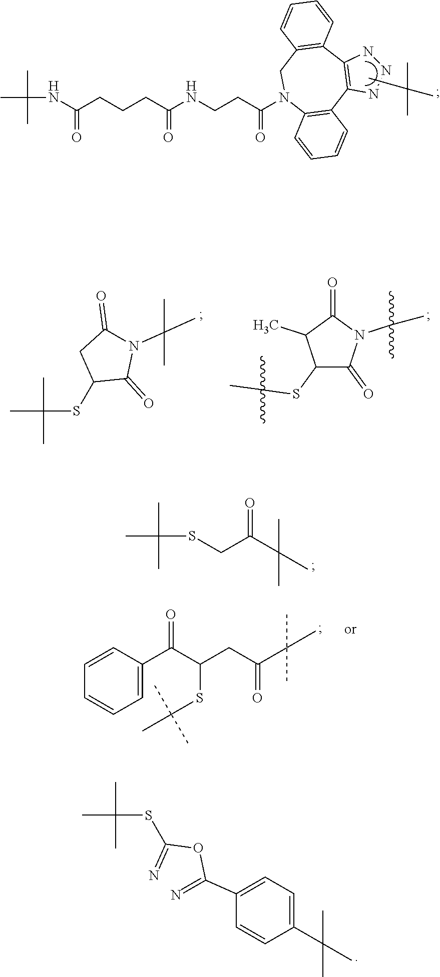

### Synthesis of the Modifying Agents

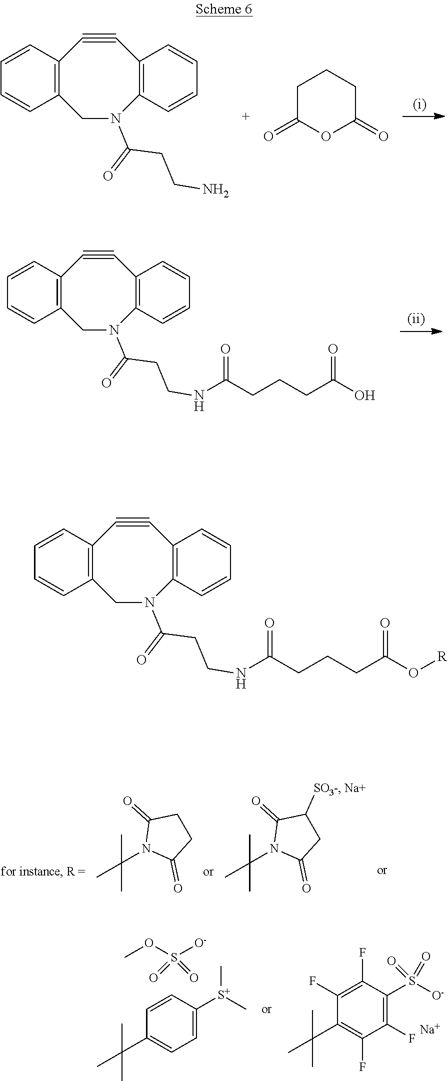

Step (i): coupling of dibenzocyclooctyne-amine to glutaric anhydride;
Step (ii): activation of the carboxylic acid as an activated ester by treatment
   with DSC in the presence of a base such as, for example, DIEA; or
   with N-hydroxysulfosuccinimide sodium salt in the presence of a coupling reagent such as, for example, DCC; or
   with 4-hydroxyphenyldimethylsulfonium methyl sulfate in the presence of a coupling reagent such as, for example, DIC; or
   with 2,3,5,6-tetrafluoro-4-hydroxybenzenesulfonic acid sodium salt in the presence of a coupling reagent such as, for example, DIC.

Scheme 6 depicted the synthesis starting with dibenzocyclooctyne-amine (CAS number [1255942-06-3]) but may also apply to other cyclooctyne-amines which are commercially available. It depicted the synthesis using glutaric anhydride but may also apply to succinic anhydride or alkyl diacids which are commercially available for n ranging from 3 to 10.

### Preparation of the Compounds/Conjugates of Formula (II)

The compound/conjugates of formula (II) or a pharmaceutically acceptable salt thereof of the present disclosure can be obtained via the process comprising at least the steps of:
(i) placing in contact and leaving to react:
   an, optionally buffered, aqueous solution of an antibody Ab, comprising a reactive RCG2 group, optionally modified by means of a modifying agent,
and
   a solution of a compound/payload of formula (I) as defined in the present disclosure, comprising a reactive RCG1 group,
RCG1 groups of compound/payload of formula (I) being reactive towards RCG2 groups of the antibody to form the G groups by a covalent bond and forming compounds/conjugates of formula (II).

According to one variant for example, in step (ii) the compound/conjugate of formula (II) from step (i) is separated from the unreacted compound/payload of formula (I), from any aggregates formed, and/or any unreacted antibody.

The function of the placing in contact is to react RCG1 groups and RCG2 groups in order to ensure attachment of the compound/payload of formula (I) to the antibody by formation of a covalent bond, such as,
when RCG1 represents a chlorine atom or a maleimido or haloacetamido group, the antibody may comprise thiol groups;
when RCG1 represents an azido group, the antibody may comprise a —C═CH moiety or an activated C≡C such as a cyclooctyne group;
when RCG1 represents a —NH₂ group, the reaction may take place on amide function of the antibody using an enzymatic catalysis, such as the amide groups borne by the side chains of glutamine (Gln) residues of an antibody.
when RCG1 represents a HC≡C— or an activated C≡C group such as a cyclooctyne moiety, the antibody may comprise azido groups.

The term “aggregates” means associations that may form between two or more antibodies, the antibodies possibly having been modified by conjugation. Aggregates are liable to form under the influence of a wide variety of parameters such as a high concentration of antibody in the solution, the pH of the solution, high shear forces, the number of grafted drugs and their hydrophobic nature, the temperature (see the references cited in the introduction of J. Membrane Sci. 2008, 318, 311-316), the influence of some of them, however, having not been clearly elucidated. In the case of proteins or antibodies, reference may be made to AAPS Journal, “Protein Aggregation and Bioprocessing” 2006, 8(3), E572-E579. The aggregate content may be determined via known techniques such as SEC (see in this respect Analytical Biochemistry 1993, 212 (2), 469-480).

The aqueous solution of the antibody may be buffered with buffers for example, potassium phosphate or HEPES or a mixture of buffers such as buffers A, B, C and D described later. The buffer depends on the nature of the antibody. The compound/payload of formula (I) is dissolved in a polar organic solvent such as DMSO or DMA.

The reaction takes place at a temperature generally ranging from 20° C. to 40° C. The reaction time may be ranging from 1 to 24 hours. The reaction between the antibody and the compound/payload of formula (I) may be monitored by SEC with a refractometric and/or ultraviolet detector and/or HRMS in order to determine its degree of progress. If the degree of substitution is insufficient, the reaction can be left for longer and/or compound/payload of formula (I) can be added. Reference may be made to the example section for further details regarding particular conditions. Particular embodiments are described in Examples 2 to 6, 8 to 12, 14 to 18, 20 to 24, 26, 28 and, 30, 32 to 43, 44, 46, 47, 49, 51 and 53.

A person skilled in the art has at his disposal various chromatographic techniques for the separation of step (ii): the compound/conjugate of formula (II) may be purified, for example, by steric exclusion chromatography (SEC), by adsorption chromatography (for instance ion exchange, IEC), by hydrophobic interaction chromatography (HIC), by affinity chromatography, by chromatography on mixed supports such as ceramic hydroxyapatite, or by HPLC. Purification by dialysis or diafiltration may also be used.

After step (i) or (ii), the solution of the compound/conjugate of formula (II) may undergo an ultrafiltration and/or diafiltration step (iii). After these steps, the compound/conjugate of formula (II) in aqueous solution is thus obtained.

The present disclosure also relates to compounds of formula (II) that may be obtained via the process(es) in accordance with the present disclosure.

### Antibody

The antibody can be a monoclonal antibody selected from the group consisting of a murine, chimeric, a humanized and a human antibody.

In one embodiment, the antibody is a monospecific antibody, i.e. an antibody specifically binding to one single target. Alternatively, it might be a multispecific antibody.

In one embodiment, the antibody is an IgG antibody, for instance an IgG1, an IgG2, an IgG3 or an IgG4 antibody.

The antibody according to the disclosure specifically binds to a target, thereby directing the compound/conjugate of formula (II) towards said target. As used herein, “specifically binds” or “binds specifically to” or “binds to” or the like, means that an antibody or antigen-binding fragment thereof forms a complex with an antigen that is relatively stable under physiological conditions. Specific binding can be characterized by an equilibrium dissociation constant (K_{D}) of at least about 1×10⁻⁸ M or less (e.g., a smaller K_{D} denotes a tighter binding). Methods for determining whether two molecules specifically bind are well known in the art and include, for example, equilibrium dialysis, surface plasmon resonance, and the like. As described herein, antibodies have been characterized, for example, by their specific binding to target and/or target antigen using surface plasmon resonance, e.g., BIACORE™.

The target typically corresponds to a protein expressed at the cell surface, e.g. a protein expressed at the surface of tumor cells.

In one embodiment, the target is the CEACAM5 receptor. The CEACAM5 receptor is a member of the carcinoembryonic antigen-related cell adhesion molecule (CEACAM) receptor family and is also referred to as “CEA cell adhesion molecule 5”, or “CD66e” The antibody specifically binding to the CEACAM5 receptor might for instance correspond to one of the antibodies described in WO2014/079886 (U.S. Pat. No. 9,617,345B2),

In one embodiment, the target is the EphA2 receptor. The EphA2 receptor is an Ephrin receptor and is also referred to as “Eph receptor A2” or “Epithelial Cell Receptor Protein-Tyrosine kinase”. The antibody specifically binding to the EphA2 receptor might for instance correspond to one of the antibodies described in WO2008/010101 (USRE47123 E) or WO2011/039724 (U.S. Pat. No. 8,668,910).

In one embodiment, the target is the ERBB2/HER-2 receptor. The HER-2 receptor is a member of the human epidermal growth factor receptor (HER/EGFR/ERBB) family and is also referred to as erb-b2 receptor tyrosine kinase 2 or NEU, NGL, TKR1, HER-2/neu or CD340 (cluster of differentiation 340). The antibody specifically binding to the HER2 receptor might for instance correspond to trastuzumab also referred to as Herceptin® (CAS [180288-69-1]). For the amino acid sequence of trastuzumab, it may also be referred to SEQ ID NO. 5 (light chain) and SEQ ID NO. 6 (heavy chain) disclosed herein after.

In one embodiment, the target is the EGFR receptor. The EGFR receptor is a member of the human epidermal growth factor receptor (HER/EGFR/ERBB) family and is also referred to as epidermal growth factor receptor, or ERBB, ERRP, HER1, ERBB1, PIG61 or NISBD2. The antibody specifically binding to the EGFR receptor might for instance correspond to cetuximab also referred to as Erbitux® (CAS [205923-56-4]). For the amino acid sequence of cetuximab, it may also be referred to SEQ ID NO. 7 (light chain) and SEQ ID NO. 8 (heavy chain) disclosed herein after.

In one embodiment, the target is the B7H3 immune checkpoint. The B7H3 immune checkpoint is a member of the B7-CD28 family and is also referred to as CD276 (cluster of differentiation 276), B7-H3, 41g-B7-H3. The antibody specifically binding to the B7H3 immune checkpoint might for instance correspond to enoblituzumab (CAS [1353485-38-7]). For the amino acid sequence of enoblituzumab, it may also be referred to SEQ ID NO. 9 (light chain) and SEQ ID NO. 10 (heavy chain) disclosed herein after.

The antibody may optionally be modified with a modifying agent so as to promote the attachment of the compound/payload of formula (I) as previously described. The antibody may especially be monoclonal, polyclonal or multispecific. It may also be an antibody fragment. It may also be a murine, human, humanized or chimeric antibody. Besides trastuzumab, cetuximab and enoblituzumab the antibodies used in the examples of the present disclosure are:
Tusamitamab (CAS [2349294-95-5]), an antibody against CEACAM5 receptor. The sequence of Tusamitamab (CAS [2349294-95-5]) is represented by SEQ ID NO: 88 (light chain of antibody Tusamitamab (CAS [2349294-95-5])) and by SEQ ID NO: 87 (heavy chain of antibody Tusamitamab (CAS [2349294-95-5])) following the procedure described in WO2014/079886 (U.S. Pat. No. 9,617,345B2). For the amino acid sequence of tusamitamab, it may also be referred to SEQ ID NO. 1 (light chain) and SEQ ID NO. 2 (heavy chain) disclosed herein after,
hu2H11_R35R74, an antagonist antibody against EphA2 receptor. The sequence of hu2H11_R35R74 is represented by SEQ ID NO: 16 (light chain of antibody hu2H11_R35R74) and SEQ ID NO:18 (heavy chain of antibody hu2H11_R35R74) following the procedure described in WO2011039724 A1 (U.S. Pat. No. 8,668,910). For the amino acid sequence of hu2H11_R35R74, it may also be referred to SEQ ID NO. 3 (light chain) and SEQ ID NO. 4 (heavy chain) disclosed herein after,
CEACAM5_Tusamitamab_E152C according to EU numbering, also named CEACAM5_Tusamitamab_E150C according to Kabat numbering, an antibody against CEACAM5 receptor. It corresponds to the antibody Tusamitamab (CAS [2349294-95-5]) in which Glu has been replaced by Cys at the indicated position,
CEACAM5_Tusamitamab_S239C according to EU numbering, also named CEACAM5_Tusamitamab_S252C according to Kabat numbering, an antibody against CEACAM5 receptor. It corresponds to the antibody Tusamitamab (CAS [2349294-95-5]) in which Ser has been replaced by Cys at the indicated position,
CEACAM5_Tusamitamab_K274C according to EU numbering, also named CEACAM5_Tusamitamab_K287C according to Kabat numbering, an antibody against CEACAM5 receptor. It corresponds to the antibody Tusamitamab (CAS [2349294-95-5]) in which Lys has been replaced by Cys at the indicated position,
CEACAM5_Tusamitamab_K290C according to EU numbering, also named CEACAM5_Tusamitamab_K307C according to Kabat numbering, an antibody against CEACAM5 receptor. It corresponds to the antibody Tusamitamab (CAS [2349294-95-5]) in which Lys has been replaced by Cys at the indicated position,
CEACAM5_Tusamitamab_K326C according to EU numbering, also named CEACAM5_Tusamitamab_K345C according to Kabat numbering, an antibody against CEACAM5 receptor. It corresponds to the antibody Tusamitamab (CAS [2349294-95-5]) in which Lys has been replaced by Cys at the indicated position,
CEACAM5_Tusamitamab_K320C according to EU numbering, also named CEACAM5_Tusamitamab_K320C according to Kabat numbering, an antibody against CEACAM5 receptor. It corresponds to the antibody Tusamitamab (CAS [2349294-95-5]) in which Lys has been replaced by Cys at the indicated position,
CEACAM5_Tusamitamab_K340C according to EU numbering, also named CEACAM5_Tusamitamab_K340C according to Kabat numbering, an antibody against CEACAM5 receptor. It corresponds to the antibody Tusamitamab (CAS [2349294-95-5]) in which Lys has been replaced by Cys at the indicated position,
CEACAM5_Tusamitamab_S375C according to EU numbering, also named CEACAM5_Tusamitamab_K398C according to Kabat numbering, an antibody against CEACAM5 receptor. It corresponds to the antibody Tusamitamab (CAS [2349294-95-5]) in which Ser has been replaced by Cys at the indicated position,
CEACAM5_Tusamitamab_N361C according to EU numbering, also named CEACAM5_Tusamitamab_N361C according to Kabat numbering, an antibody against CEACAM5 receptor. It corresponds to the antibody Tusamitamab (CAS [2349294-95-5]) in which Asn has been replaced by Cys at the indicated position,
CEACAM5_Tusamitamab_K414C according to EU numbering, also named CEACAM5_Tusamitamab_K445C according to Kabat numbering, an antibody against CEACAM5 receptor. It corresponds to the antibody Tusamitamab (CAS [2349294-95-5]) in which Lys has been replaced by Cys at the indicated position,
CEACAM5_Tusamitamab_V422C according to EU numbering, also named CEACAM5_Tusamitamab_V453C according to Kabat numbering, an antibody against CEACAM5 receptor. It corresponds to the antibody Tusamitamab (CAS [2349294-95-5]) in which Val has been replaced by Cys at the indicated position,
CEACAM5_Tusamitamab_E152C_S375C according to EU numbering, also named CEACAM5_Tusamitamab_E150C_S398C according to Kabat numbering, an antibody against CEACAM5 receptor. It corresponds to the antibody Tusamitamab (CAS [2349294-95-5]) in which Glu and Ser have both been replaced by Cys at the indicated positions.

The above Tusamitamab variants comprise Fc mutations for site specific conjugation wherein the mutated amino acid is identified according to the EU numbering from Edelman, G. M. et al., Proc. Natl. Acad. USA, 63, 78-85 (1969). PMID: 5257969 and/or the Kabat numbering from Kabat, E. A. et al., Sequences of proteins of immunological interest. 5th Edition—US Department of Health and Human Services, NIH publication no 91-3242, pp 662,680,689 (1991).

### Compound/Conjugate of Formula (II) (Conjugate):

A compound/conjugate of formula (II) generally comprises from about 1 to 10 compound/payload of formula (I) covalently attached to the antibody (this is the degree of grafting or “drug-to-antibody ratio” or “DAR”). This number varies as a function of the nature of the antibody and of the compound/payload of formula (I), and also of the operating conditions used in the conjugation process (for example the number of equivalents of compound/payload of formula (I) relative to the antibody, the reaction time, the nature of the solvent and of any cosolvent). Placing of the antibody and the compound/payload of formula (I) in contact leads to a mixture comprising several compounds/conjugates of formula (II) that are individually distinguished from each other by different DARs; optionally the unreacted antibody; optionally aggregates. The DAR that is determined on the final solution thus corresponds to an average DAR. The DAR may be calculated from the deconvolution of the SEC-HRMS spectrum of the compound/conjugate of formula (II). The DAR (HRMS) is for example greater than 0.5, for instance ranging from 1 to 10, such as ranging from 2 to 7.

The compound/conjugate of formula (II) may be used as an anticancer agent. Owing to the presence of the antibody, the compound/conjugate of formula (II) is made highly selective towards tumor cells rather than healthy cells. This makes it possible to direct the compound/conjugate of formula (II) in an environment similar thereto or directly therein. It is possible to treat solid or liquid cancers. The compound/conjugate of formula (II) may be used alone or in combination with at least one other anticancer agent.

The compound/conjugate of formula (II) is formulated in the form of a buffered aqueous solution at a concentration generally ranging from 1 to 10 mg/mL. This solution may be injected in perfusion form per se or may be re-diluted to form a perfusion solution.

The examples which follow describe the preparation of some compounds of formulae (I) and (II) in accordance with the disclosure. The numbers of the compounds exemplified below match those given above. All reactions are performed under inert atmosphere, unless otherwise stated.

In the following examples, when the source of the starting products is not specified, it should be understood that said products are known compounds.

### EXAMPLES

The examples which follow describe the preparation of certain compounds in accordance with the disclosure. These examples are not limitative, and merely illustrate the present disclosure. The Tusamitamab variants are named according to the EU numbering.

### Analytical Methods Used

### High Pressure Liquid Chromatography-Mass Spectrometry (LCMS)

### Method M1

The spectra were acquired on a Waters UPLC-SQD.

Ionization: electrospray in positive and/or negative mode (ES+/−).
Column ACQUITY CSH C18-1.7 μm-2.1×50 mm
Solvents: A: H₂O (0.1% formic acid) B: CH₃CN (0.1% formic acid)
Column temperature: 50° C.
Flow rate: 0.90 mL/min
Gradient (2.5 min): from 5 to 100% B in 1.8 min; 2.4 min: 100% B; 2.45 min: 5% B

### Method M2

The spectra were acquired on a Waters UPLC-SQD.

Ionization: electrospray in positive and/or negative mode (ES+/−).
Column ACQUITY CSH C18-1.7 μm-2.1×50 mm
Solvents: A: H₂O (0.1% formic acid) B: CH₃CN (0.1% formic acid)
Column temperature: 50° C.
Flow rate: 0.80 mL/min
Gradient (10 min): from 5 to 100% B in 8.6 min; 9.6 min: 100% B; 9.8 min: 5% B

### Method M3

The spectra were acquired on a Waters UPLC-SQD.

Ionization: electrospray in positive and/or negative mode (ES+/−).
Column ACQUITY CSH C18-1.7 μm-2.1×50 mm
Solvents: A: H₂O (0.1% formic acid) B: CH₃CN (0.1% formic acid)
Column temperature: 50° C.
Flow rate: 0.80 mL/min
Gradient (5 min): 0.2: 5% B; from 5 to 98% B in 3.4 min; 3.7 min: 98% B; 4.1 min: 5% B

### Method M4

The spectra were acquired on a Waters UPLC-SQD.

Ionization: electrospray in positive and/or negative mode (ES+/−).
Column ACQUITY CSH C₁₈-1.7 μm-2.1×30 mm
Solvents: A: H₂O (0.1% formic acid) B: CH₃CN (0.1% formic acid)
Column temperature: 35° C.
Flow rate: 0.70 mL/min
Gradient (4 min): 0.18: 5% B; from 5 to 99% B in 3.02 min; 3.4 min: 99% B; 3.55 min: 5% B

### Method M5

The spectra were acquired on a Waters UPLC-SQD.

Ionization: electrospray in positive and/or negative mode (ES+/−).
Column ACQUITY CSH C18-1.7 μm-2.1×50 mm
Solvents: A: H₂O (0.1% formic acid) B: CH₃CN (0.1% formic acid)
Column temperature: 35° C.
Flow rate: 0.70 mL/min
Gradient (5 min): 0.15: 3% B; from 3 to 100% B in 3.3 min; 3.45 min: 100% B; 3.85 min: 3% B

### ¹H Nuclear Magnetic Resonance (NMR)

The ¹H NMR spectra were acquired on a Bruker Avance spectrometer, either of model DRX-400 or DRX-500. The chemical shifts (δ) are given in ppm.

### Size Exclusion Chromatography-High Resolution Mass Spectrometry (RP-HRMS)

The analyses were performed on an UPLC-XEVO-G2-XS-QTOF (Waters). The reverse phase chromatographic analysis was performed using an Agilent BioHPLC PLRP-S 4000 Å 5 μm column (2.1×50 mm) at 80° C. with a flow rate of 0.5 mL/min and the following elution gradient: 5% B at 1 min, 50% B at 4 min, 50% B at 5 min, 5% B at 6 min, 5% B at 7 min (solvents: A: H₂O (0.1% formic acid); B: CH₃CN (0.1% formic acid)). The mass spectrometry was performed with electrospray ionization in positive mode (ES+). The mass spectra were deconvoluted with the Waters MaxEnt1 software. The observed molecular masses correspond respectively to the mass of the naked antibody, if present, and to the conjugation of 1 (D1), 2 (D2)n (Dn) drugs on the antibody.

When needed, preliminary ADC reduction was performed using the following conditions: ADC was treated with 1/10 (in volume) of a 0.5M TCEP solution in water and left for 30 min at RT before SEC HRMS analysis.

### Analytical Size Exclusion Chromatography (SEC)

The analysis was performed on a Hitachi Labchrom system equipped with a photodiode array detector and a Tosoh Bioscience TSK-GEL SuperSW 4 μm (4.6×150 mm) column with a flow rate of 0.2 mL/min or a Tosoh Bioscience TSKgel G3000 SWXL 5 μm (7.8×300 mm) column with a flow rate of 0.5 mL/min and an isocratic elution of 15 minutes with a pH 7 buffer containing 0.2 M of KCl, 0.052 M of KH₂PO₄, 0.107 M of K₂HPO₄ and 20% by volume of isopropanol.

### Buffers

Buffer A (pH 5.5): sodium acetate (20 mM), 10% (w/v) sucrose
Buffer B (pH 5.5): sodium acetate (20 mM), 5% (w/v) sucrose, 0.01% PS80
Buffer C (pH 7.4): NaPi (10 mM), NaCl (140 mM)
Buffer D (pH 6.5): KPi (50 mM), NaCl (50 mM), EDTA (2 mM)
Buffer E (pH 6.5): 10 mM Histidine, 10% (w/v) sucrose
Buffer F (pH 5.5): 10 mM Histidine, 8% (w/v) sucrose
DPBS: Na₂HPO₄ (8.10 mM), KH₂PO₄ (1.47 mM), NaCl (136.9 mM), KCl (2.67 mM)

### General Method Used for the Preparation of Cyclic PEGn Sulfates (M1)

Under argon, the appropriate ethylene glycol (1 equiv) was dissolved in anhydrous DCM (225 mL). At 0° C., TEA (4.9 equiv) and DMAP (0.001 equiv) were added. A solution of thionyl chloride (2 equiv) in DCM (10 mL) was added dropwise over 2 h. The reaction mixture was then stirred at RT until completion of the reaction. The brown solution was cooled in an ice bath and water (25 mL) was slowly added. The organic layer was washed with water (2×25 mL), dried over MgSO₄, filtered and concentrated in vacuo to provide the expected cyclic PEGn sulfite as a brown oil.

The cyclic PEGn sulfite was taken up in 20 mL of a mixture DCM/CH₃CN (1:1). At 0° C., were added, under vigorous stirring, a sodium periodate (1.5 equiv) solution in water (15 mL) and a ruthenium(III) chloride trihydrate (0.001 equiv) solution in water (1 mL). After stirring 1 h at 0° C., the reaction mixture was allowed to warm up to RT overnight then filtered over Clarcel. The filtrate was washed with water (3×25 mL), stirred with MgSO₄ and Charcoal, filtered again over Clarcel and concentrated in vacuo. The crude oil was stirred and extracted with diethyl ether (3×15 mL). The ethereal phases were concentrated in vacuo to provide the expected cyclic PEGn sulfate as a colorless oil.

### General Method Used for the O-Alkylation Reaction (M2)

To a suspension of sodium hydride (2 equiv) in anhydrous DMF (2 mL) under argon, a solution of trityl-R848, compound 1 (1 equiv) in DMF (2 mL) was added dropwise at 0° C. After 45 minutes of stirring at 0° C., the reaction mixture was stirred 30 minutes to RT. The yellow solution was cooled at 0° C., then a solution of cyclic PEGn sulfate (1.6 equiv) in DMF (2 mL) was added dropwise. The reaction mixture was allowed to warm up to RT overnight. The reaction mixture was quenched with 1.5 mL of water at 0° C. and stirred 10 minutes. After concentration in vacuo and co-evaporation with toluene the solid residue was extracted with DCM (3×20 mL), concentrated in vacuo and purified by flash chromatography on silica gel (gradient elution DCM to DCM/MeOH/H₂O 80:20:1) to provide the expected compound.

### General Method Used for the Desulfatation Reaction (M3)

The sulfate compound resulting from O-alkylation reaction M2 was heated in a mixture of pyridine and 1,4-dioxane (4:1) (50 mL/g) at 80° C. After completion of the reaction, the colorless solution was concentrated in vacuo. The residue was taken up with DCM (50 mL), washed with water (3×10 mL) and sat. brine (3×10 mL), dried over MgSO₄, filtered and concentrated in vacuo to provide the expected compound.

### General Method Used for the PEGn Chain Elongation Reaction (M4)

Under argon, to a suspension of sodium hydride (2 equiv) in anhydrous DMF (2-4 mL), a solution of trityl-R848-PEGn-OH resulting from the desulfatation reaction M3 (1 equiv, 130-250 mg) in DMF (3-6 mL) was added dropwise at 0° C. After 45 minutes, the reaction mixture was stirred (30-60 minutes) to RT. The yellow suspension was cooled at T<+4° C., then a solution of cyclic PEGn sulfate (1.6 equiv) in DMF (3-6 mL) was added dropwise. The reaction mixture was allowed to warm up to RT overnight. The reaction mixture was quenched with 5 mL of water at 0° C. and stirred 30 minutes. After concentration in vacuo and co-evaporation with toluene, the solid residue was extracted with DCM (3×50 mL) and filtered. The combined organic layers were concentrated in vacuo and purified by flash chromatography on silica gel (gradient elution DCM to DCM/MeOH/H₂O 80:20:1) to provide the expected compound as a colorless oil.

### General Method Used for the Trityl Group Deprotection (M5)

To a solution of trityl compound in 1,4-dioxane in an ice bath was added a solution of a 4N HCl solution in 1,4-dioxane. The ice bath was removed after 30 minutes and the reaction mixture was stirred at RT until completion of the reaction. MeOH was then added to dissolve the mixture and the solution was concentrated in vacuo. The residue was dissolved in water (10 mL), washed with EtOAc (3×10 mL) and Et₂O (10 mL). The pH of the aqueous layer was adjusted to pH 10 with 0.1N NaOH under stirring in an ice bath. The product was extracted with DCM or EtOAc (6×10 mL). The combined organic layers were dried over MgSO₄, filtered, concentrated in vacuo and then purified by flash chromatography on silica gel (gradient elution DCM to DCM/MeOH/H₂O 80:20:1) to provide the expected compound as a colorless oil.

### General Method Used for the Preparation of PEGn Aldehydes (M6)

Under argon, to a solution of oxalyl chloride (1.2 equiv) in anhydrous DCM (2 mL) was added DMSO (3.6 equiv) at −75° C. After 30 minutes at −75° C., a solution of azido-PEGn-OH (1 equiv) in DCM (2 mL) was added, the reaction mixture was stirred for 30 minutes at −75° C., followed by the addition of TEA (7.2 equiv). The reaction mixture was stirred at −75° C. for another 30 minutes and allowed to warm up to RT. The reaction mixture was quenched with water (4 mL), washed with sat. brine, dried over Na₂SO₄, filtered and concentrated in vacuo to provide the expected PEGn aldehyde.

### General Method Used for the Reductive Amination Reaction (M7)

To a solution of PEGn aldehyde resulting from general method M6 (1 equiv) in THF (2 mL) were added the imidazoquinoline 3M-012 (1 equiv) and sodium triacetoxyborohydride (1.3 equiv). The reaction mixture was stirred for 2 h at RT, quenched with water and extracted with EtOAc three times. The combined organic layers were washed with sat. brine, dried over Na₂SO₄, filtered, concentrated in vacuo and purified by flash chromatography on silica gel (gradient elution DCM/MeOH) to provide the expected compound.

General method used for the preparation of compounds/conjugates of formula (II):
General Method Used for the Preparation of mAb-DBCO Using NHS Ester (M8 and M9)

M8: To a solution of antibody in an aqueous buffer eventually diluted with DPBS was added 1 N HEPES (composition varying from 96:4 to 100:0). The solution was treated with an excess of a solution at approximatively 20 mM of NHS ester DBCO linker in DMA or DMSO such that the final antibody concentration is 1-11 mg/mL and the percentage of DMA or DMSO in the aqueous buffer is 2-20%. After stirring at RT for 1-3 hours, the mixture was analyzed by SEC HPLC so as to determine the linker-to-antibody ratio (LAR) on the population of monomeric antibodies. If the LAR was found insufficient, the mixture was treated with a further excess of linker solution in DMA or DMSO for up to 2 additional hours at RT under stirring.

M9: To a solution of antibody in an aqueous buffer eventually diluted with DPBS was added 1 N HEPES (composition varying from 99:1 to 100:0, in other terms from 0% to 1% HEPES (v/v)). The solution was treated with an excess of a solution at approximatively 20 mM of NHS ester DBCO linker in DMA such that the final antibody concentration is ˜10 mg/mL and the percentage of DMA in the aqueous buffer is 20%. After stirring at RT for 1 hour, the mixture was analyzed by SEC HPLC to determine the LAR on the population of monomeric antibodies. If the LAR was found insufficient, the mixture was treated with a further excess of linker solution in DMA for up to 2 additional hours at RT under stirring. The mixture was then purified by gel filtration using a Sephadex™ G25 matrix (Hiprep™ 26/10 desalting column, GE Healthcare) pre-equilibrated with DPBS buffer with 20% of DMA.

General Method Used for the Preparation of mAb-DBCO Using Water Soluble Activated Esters (M10)

A solution of antibody in an aqueous buffer diluted with 30 mM K₂HPO₄ buffer to adjust the pH between 7 to 8 was treated with an excess of a solution at approximatively 50 mM of water-soluble activated ester in DMSO or sterile water such that the final antibody concentration is ˜10 mg/mL. After stirring at RT for 1 to 3 hours, the mixture was analyzed by SEC HPLC and/or RP-HRMS to determine the LAR on the population of monomeric antibodies.

### General Method Used for the Preparation of Compounds/Conjugates of Formula (II) (M11 to M13)

M11: A solution of mAb-DBCO in an aqueous buffer containing 20% DMA (eventually diluted with DPBS), was treated with an excess (6 to 12 equivalents) of a solution at 7 to 20 mM of azido payload in DMA such that the final antibody concentration is 1-6 mg/mL and the percentage of DMA in the aqueous buffer is 20%. After overnight stirring at RT, the reaction mixture was analyzed by RP-HRMS to determine the drug-to-antibody ratio (DAR) on the population of monomeric antibodies. The mixture was purified by gel filtration using Sephadex™ G25 matrix (NAP® or Hiprep™ 26/10 desalting columns, GE Healthcare) pre-equilibrated with the final aqueous buffer (buffer A or DPBS). The compound/conjugate of formula (II) was finally sterile filtered (Millex®-SV 0.22 μm, PVDF, Durapore®, Millipore). The final compound/conjugate of formula (II) was assayed by UV spectrometry or SEC HPLC to measure the compound/conjugate of formula (II) concentration, by SEC HPLC to determine the monomeric purity and by RP-HRMS to determine the DAR from the deconvolution of the mass spectrum of the compound/conjugate of formula (II).

M12: A solution of mAb-DBCO in an aqueous buffer containing 20% DMA (eventually diluted with DPBS) was treated with an excess (7 to 14 equivalents) of a solution at 10 to 20 mM of azido payload in DMA such that the final antibody concentration is 1-4 mg/mL and the percentage of DMA in the aqueous buffer is -20%. After overnight stirring at RT, the reaction mixture was analyzed by RP-HRMS to determine the DAR on the population of monomeric antibodies. The mixture was eventually purified by ultrafiltration with ultrafiltration spin column (Vivaspin®, PES membrane 50K, Sartorius) with DPBS buffer containing 20% of DMA. The mixture was then purified by gel filtration using a Superdex™ 200 pg matrix (HiLoad® 16/60 or 26/60 desalting column, GE Healthcare) pre-equilibrated in DPBS buffer containing up to 10% of DMA. The fractions containing the monomeric conjugated antibody were pooled and eventually concentrated by ultrafiltration on Vivaspin® (PES membrane 50K, Sartorius) to reach a concentration between 2 and 5 mg/mL. In some cases, the conjugate was formulated by gel filtration using a Sephadex™ G25 matrix (NAP® or Hitrap™ desalting columns, GE Healthcare) pre-equilibrated with the final aqueous buffer. The conjugate was finally sterile filtered (Millex®-SV 0.22 μm, PVDF, Durapore®, Millipore). then assayed by UV spectrometry or SEC HPLC so as to measure the conjugate concentration, by SEC HPLC so as to determine the monomeric purity and by RP-HRMS so as to determine the DAR from the deconvolution of the mass spectrum of the conjugate.

M13: A solution of mAb-DBCO in an aqueous buffer containing 20% DMA (eventually diluted with DPBS) was treated with an excess (12 to 14 equivalents) of a solution at 10 to 20 mM of azido payload in DMA such that the final antibody concentration is 1-4 mg/mL and the percentage of DMA in the aqueous buffer is -20%. After overnight stirring at RT, the mixture was analyzed by RP-HRMS so as to determine the DAR on the population of monomeric antibodies. The mixture was purified by diafiltration with ultrafiltration spin columns (Vivaspin®: PES membrane 50K, Sartorius or Amicon® Ultra: Ultracel membrane, 50K, Millipore) then formulated using Sephadex™ G25 matrix (Hitrap™ desalting GE Healthcare) pre-equilibrated with the final aqueous buffer. Depending on the residual freedrug content as assessed by HRMS, additional purification by diafiltration with final buffer was performed. The conjugate was finally sterile filtered (Millex®-SV 0.22 μm (PVDF, Durapore, Millipore)). The final conjugate was assayed by UV spectrometry or SEC HPLC to measure the conjugate concentration, by SEC-HPLC so as to determine the monomeric purity and by RP-HRMS to determine the DAR from the deconvolution of the mass spectrum of the conjugate.

### General Method Used for the Reduction of Azido-PEGn-R848 Payload (M14)

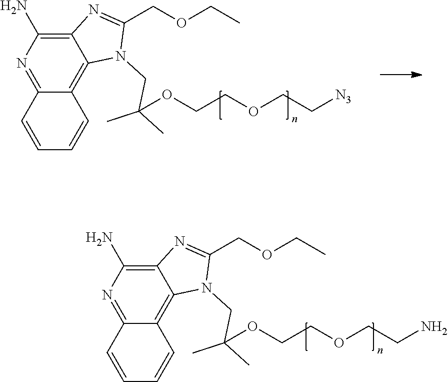

To a solution of 215 μmol of azido-PEGn-R848 in 10 ml MeOH were added ammonium formiate (10 equiv) and 5% (w/w) of 10% Pd/C. The mixture was heated under reflux for 1 hour then filtered over Clarcel Flo. The filtrate was concentrated in vacuo and the crude was purified by flash chromatography on silica gel (gradient elution DCM to DCM/MeOH/H₂O 40:5:0.5) to provide the expected compound.

### General Method Used for the Synthesis of NHS-Maleimido Linker (M15)

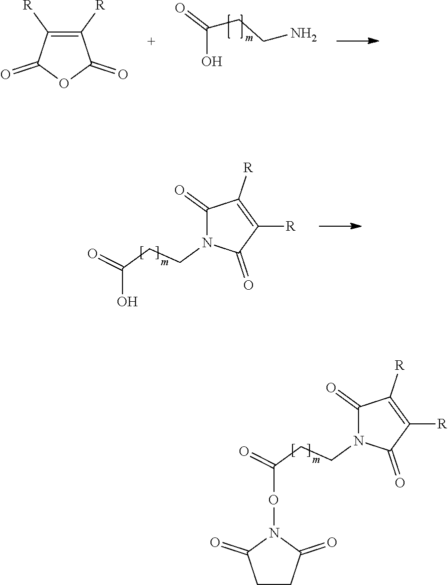

Under argon a maleic anhydride derivative (1.2 equiv, R═H or Me) was added to a solution of amino acid (1 equiv, m=1 to 6) in 10 volumes of AcOH. The mixture was stirred at 120° C. for 6 hours, poured into water after cooling to RT and extracted with EtOAc. The combined organic layers were washed with brine, dried over anhydrous MgSO₄, and concentrated in vacuo to give the crude product that was purified by flash chromatography on silica gel (gradient elution DCM to DCM/MeOH 9:1) to provide the expected compound. Under argon, to a solution of the previous intermediate in 20 volumes of anhydrous DCM were added DSC (1 equiv) and DIEA (1 equiv). The reaction mixture was stirred overnight at RT and then purified by flash chromatography on silica gel (gradient elution DCM to DCM/Acetone 8:2) to provide the expected compound.

### General Method Used for the Synthesis of Maleimido-PEGn-R848 Payload (M16)

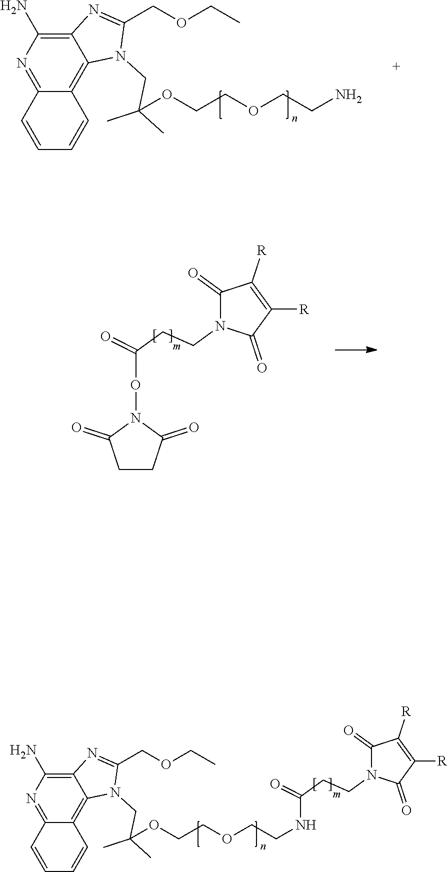

Under argon, to a solution of amino-PEGn-R848 (1 equiv) in 10 volumes of anhydrous DMF were added NHS-substituted maleimido linker (1 equiv) and DIEA (2-3 equiv). The reaction mixture was stirred overnight at RT then concentrated in vacuo and the crude compound was purified on C18 silica gel (Chromabond RS40 C18ec) using a gradient CH₃CN/H₂O with 0.1% formic or trifluoroacetic acid to provide the expected compound after lyophilization.

### General Cys Conjugation Method Used for the Preparation of Compounds/Conjugates of Formula (II) (M17)

To a solution of thiomAb at 1-11 mg/mL in DPBS was added DTT (64 equiv of a 100-300 mM solution in H₂O or DPBS). The mixture was stirred at 37° C. during 45 min to 1 h and then purified by gel filtration using a Sephadex™ G25 matrix (NAP® or Hiprep™ 26/10 desalting columns, GE Healthcare) pre-equilibrated with DPBS buffer. To the solution of reduced thiomAb was added DHAA (14-15 equiv of a 25-100 nM solution in DPBS). After stirring at RT for 3 hours and addition of DMA (10-20% v/v), the mixture was treated with an excess of maleimido compound (1.5 to 2 equiv per uncapped thiols of a 10 to 20 mM solution in DMA) such that the final antibody concentration is 1-4 mg/mL and the percentage of DMA in the aqueous buffer is -10-20%. After overnight stirring at RT, the mixture was analyzed by RP-HRMS so as to determine the DAR on the population of monomeric antibodies. The mixture was concentrated by diafiltration with ultrafiltration spin columns (Amicon® Ultra: PES membrane, 50K, Millipore) before or after purification by gel filtration using a Superdex 200 pg matrix (HiLoad® 16/60 or 26/60 desalting column, GE Healthcare) pre-equilibrated in aqueous buffer such as DPBS containing 5 to 20% of an organic solvent such as DMA or EtOH and then formulated by gel filtration using a Sephadex™ G25 matrix NAP® or Hiprep™ 26/10 desalting columns, GE Healthcare) pre-equilibrated with the final aqueous buffer A, buffer E or buffer F. Depending on the residual free drug content as assessed by HRMS, additional purification by diafiltration with final buffer was performed. The conjugate was finally sterile filtered (Millex®-SV 0.22 μm, PVDF, Durapore, Millipore). The conjugate was assayed by UV spectrometry or SEC HPLC so as to measure the conjugate concentration, by SEC-HPLC so as to determine the monomeric purity and by RP-HRMS so as to determine the DAR from the deconvolution of the mass spectrum of the conjugate.

### Synthesis of Compound 1: 1-(2-(ethoxymethyl)-4-(tritylamino)-1H-imidazo[4,5-c]quinolin-1-yl)-2-methylpropan-2-ol

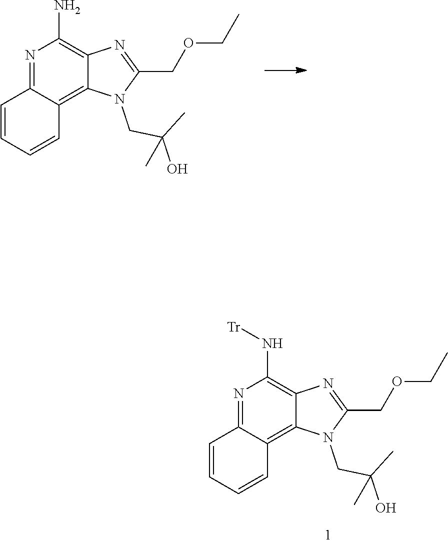

To a solution of R848 ([144875-48-9], 250 mg, 0.755 mmol) in CH₃CN (9.5 mL) were added TEA (263 μL, 1.89 mmol) and trityl chloride (253 mg, 0.906 mmol). The reaction mixture was heated to reflux for 45 minutes, cooled to RT and then placed in an ice bath. The solid was collected by filtration, washed with cold acetonitrile and dried in vacuo at RT to give 320 mg of compound 1 as a white powder (76%).

NMR ¹H (400 MHz, δ in ppm, chloroform-d): 1.24 to 1.37 (m, 9H); 3.36 (s, 1H); 3.68 (q, J=7 Hz, 2H); 4.71 (s large, 2H); 4.87 (s, 2H); 7.02 (s, 1H); 7.15 to 7.30 (m, 12H); 7.45 to 7.50 (m, 6H); 7.93 (d, J=8 Hz, 1H).

### Synthesis of Cyclic PEGn Sulfates

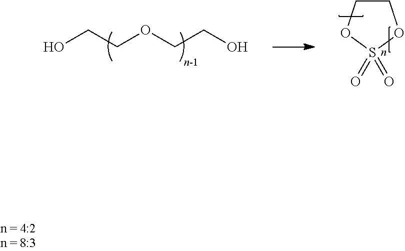

### Compound 2: 1,3,6,9,12-pentaoxa-2-thiacyclotetradecane 2,2-dioxide

Following the procedure described in general method M1, the cyclic PEG4 sulfate compound 2 was prepared from tetraethylene glycol (1.52 mL, 8.71 mmol) and obtained as a white solid (553 mg, 24%).

NMR ¹H (400 MHz, δ in ppm, DMSO-d6): 3.54 (m, 4H); 3.61 (m, 4H); 3.76 (t, J=5 Hz, 4H); 4.42 (t, J=5 Hz, 4H); LCMS (M1): t_{R}=0.60, [M+H]⁺=257; IR: 1397; 1195; 1103; 1004; 939; 910; 828; 603 and 508 cm⁻¹.

### Compound 3: 1,3,6,9,12,15,18,21,24-nonaoxa-2-thiacyclohexacosane 2,2-dioxide

Following the procedure described in general method M1, the cyclic PEG8 sulfate was prepared from octaethylene glycol (1.69 g, 4.33 mmol) and obtained as a white solid (880 mg, 42%).

NMR ¹H (400 MHz, δ in ppm, DMSO-d6): 3.50 to 3.58 (m, 24H); 3.71 (m, 4H); 4.39 (m, 4H); LCMS (M1): t_{R}=0.75, [M+H]⁺=433; IR: 2886; 1184; 1097; 1008; 920; 918; 589 and 519 cm⁻¹.

### Synthesis of Dibenzylcyclooctyne Linkers

### Synthesis of Compound 5a: ADIBO-glutaryl-ONHS

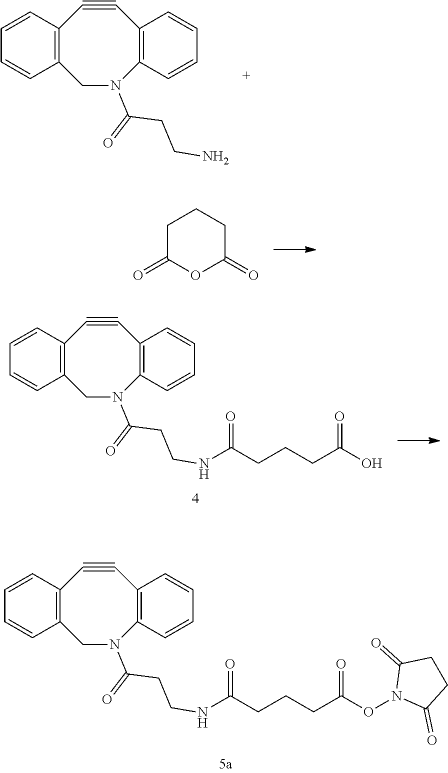 [SMILES: NCCC(=O)N1Cc2ccccc2C#Cc2ccccc21 ; O=C1CCCC(=O)O1 ; O=C(O)CCCC(=O)NCCC(=O)N1Cc2ccccc2C#Cc2ccccc21 ; O=C(CCCC(=O)ON1C(=O)CCC1=O)NCCC(=O)N1Cc2ccccc2C#Cc2ccccc21]

### Compound 4: 5-[[3-(2-azatricyclo[10.4.0.04,9]hexadeca-1(12),4(9),5,7,13,15-hexaen-10-yn-2-yl)-3-oxo-propyl]amino]-5-oxo-pentanoic acid

To a solution of dibenzocyclooctyne-amine ([1255942-06-3], 77.5 mg, 280 μmol) in anhydrous DCM (2 mL) was added glutaric anhydride (42.5 mg, 364 μmol). The colorless reaction mixture was stirred overnight at RT and purified by flash chromatography on 5 g of silica gel (gradient elution EtOAc to EtOAc/MeOH/H₂O 7:2:1) to give 94 mg of compound 4 as a white foam (86%).

NMR ¹H (400 MHz, δ in ppm, DMSO-d6): 1.59 (quin, J=7 Hz, 2H); 1.83 (ddd, J=6, 8 and 16 Hz, 1H); 1.94 (t, J=8 Hz, 2H); 2.11 (t, J=7 Hz, 2H); 2.40 (ddd, J=6, 8 and 16 Hz, 1H); 2.87 (m, 1H); 3.09 (m, 1H); 3.63 (d, J=14 Hz, 1H); 5.04 (d, J=14 Hz, 1H); 7.26 to 7.42 (m, 3H); 7.43 to 7.52 (m, 3H); 7.54 to 7.67 (m, 3H); 12.01 (large s, 1H).

### Compound 5a: (2,5-dioxopyrrolidin-1-yl)-5-[[3-(2-azatricyclo[10.4.0.04,9]hexadeca-1(12),4(9),5,7,13,15-hexaen-10-yn-2-yl)-3-oxo-propyl]amino]-5-oxo-pentanoate

Under argon, to a solution of compound 4 (31 mg, 79 μmol) in anhydrous DCM (1.7 mL) was added N,N′-disuccinimidyl carbonate (43 mg, 159 μmol) followed by DIEA (28 μL, 159 μmol). The reaction mixture was stirred overnight at RT and then purified by flash chromatography on 4.3 g of diol silica gel (gradient elution heptane/EtOAC) to give 18.5 mg of compound 5a as a white solid (47%).

NMR ¹H (400 MHz, δ in ppm, DMSO-d6): 1.65 to 1.77 (m, 2H); 1.85 (ddd, J=6, 8 and 16 Hz, 1H); 2.04 (t, J=7 Hz, 2H); 2.40 (m, 1H); 2.59 (t, J=7 Hz, 2H); 2.81 (s, 4H); 2.95 (m, 1H); 3.09 (m, 1H); 3.63 (d, J=14 Hz, 1H); 5.05 (d, J=14 Hz, 1H); 7.26 to 7.54 (m, 6H); 7.55 to 7.71 (m, 3H); LCMS (M1): t_{R}=1.03, ES, m/z=488 [M+H]⁺, m/z=486 [M−H]⁻, m/z=532 [M−H+HCO₂H]⁻.

### Synthesis of Water Soluble Dibenzylcyclooctyne Linkers Compounds 5b, 5c and 5d

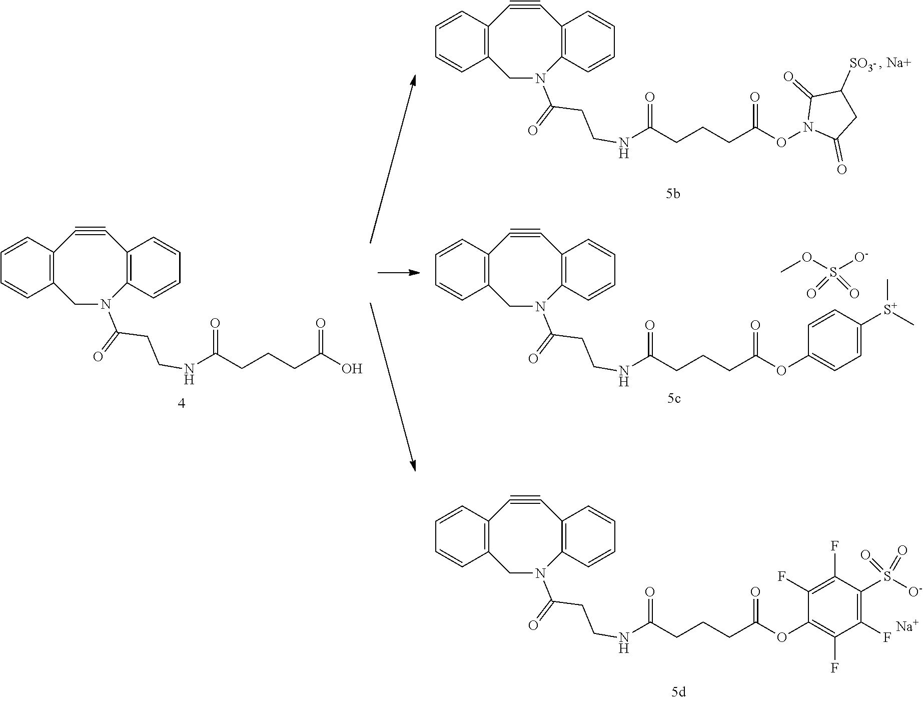

Under argon, to a solution of compound 4 (1 eq) in anhydrous DMF or CH₃CN (1.5 mL) were added water soluble activated ester (1.0-1.2 eq) and a coupling agent such as DIC or DCC (1.2-1.9 eq). The reaction mixture was stirred overnight at RT and then purified by trituration with MTBE in ice bath and sedimentation. After filtration and drying in vacuo expected activated esters were obtained as a white solid.

### Compound 5b: Sodium;1-[5-[[3-(2-azatricyclo[10.4.0.04,9]hexadeca-1(16),4(9),5,7,12,14-hexaen-10-yn-2-yl)-3-oxo-propyl]amino]-5-oxo-pentanoyl]oxy-2,5-dioxo-pyrrolidine-3-sulfonate

Following the procedure described in the general procedure above, compound 5b was prepared from compound 4 (78.5 mg, 0.2 mmol), N-hydroxysulfosuccinimide sodium salt (45.1 mg, 0.21 mmol) and DCC (74.8 mg, 0.36 mmol) in DMF (0.25 mL) and obtained as a white solid (35 mg, 35%).

¹H NMR (400 MHz, δ in ppm DMSO-d6): 1.72 (m, 2H); 1.85 (m, 2H); 2.05 (t, J=7 Hz, 2H); 2.42 (m, 2H); 2.56 (t, J=7 Hz, 2H); 2.85 (m, 2H); 2.95 (m, 1H); 3.11 (m, 1H); 3.63 (d, J=15 Hz, 1H); 3.91 (m, 1H); 5.05 (d, J=15 Hz, 1H); 7.30-7.70 (m, 9H); LCMS (M): t_{R}=3.83. ES, m/z=568.

### Compound 5c: [4-[5-[[3-(2-azatricyclo[10.4.0.04,9]hexadeca-1(16),4(9),5,7,12,14-hexaen-10-yn-2-yl)-3-oxo-propyl]amino]-5-oxo-pentanoyl]oxyphenyl]-dimethyl-sulfonium;methyl sulfate

Following the procedure described in the general procedure above, compound 5c was prepared from compound 4 (97.6 mg, 0.25 mmol), 4-hydroxyphenyldimethylsulfonium methyl sulfate (76.6 mg, 0.29 mmol) and DIC (40.4 mg, 0.32 mmol) in CH₃CN (2.5 mL) and obtained as a white solid (96 mg, 60%).

¹H NMR (400 MHz, δ in ppm, DMSO-d6): 1.75 (m, 2H); 1.85 (m, 2H); 2.05 (t, J=7 Hz, 2H); 2.42 (m, 2H); 2.68 (t, J=7 Hz, 2H); 2.95 (m, 1H); 3.11 (m, 1H); 3.63 (d, J=15 Hz, 1H); 5.05 (d, J=15 Hz, 1H); 7.30-7.70 (m, 9H); LCMS (M): t_{R}=1.16, ES, m/z=527.

### Compound 5d: Sodium;4-[5-[[3-(2-azatricyclo[10.4.0.04,9]hexadeca-1(16),4(9),5,7,12,14-hexaen-10-yn-2-yl)-3-oxo-propyl]amino]-5-oxo-pentanoyl]oxy-2,3,5,6-tetrafluoro-benzenesulfonate

Following the procedure described in the general procedure above, compound 5d was prepared from compound 4 (100.5 mg, 0.26 mmol), 2,3,5,6-tetrafluoro-4-hydroxybenzenesulfonic acid sodium salt (74.8 mg, 0.28 mmol) and DIC (61 mg, 0.48 mmol) in CH₃CN (3.5 mL) and obtained as a colorless oil (67 mg, 40%).

¹H NMR (400 MHz, δ in ppm, DMSO-d6): 1.76 (m, 2H); 1.85 (m, 2H); 2.05 (t, J=7 Hz, 2H); 2.42 (m, 2H); 2.55 (t, J=7 Hz, 2H); 2.95 (m, 1H); 3.11 (m, 1H); 3.19 (s, 3H); 3.28 (s, 6H); 3.65 (d, J=15 Hz, 1H); 5.04 (d, J=15 Hz, 1H); 7.25-7.52 (m, 8H); 7.57-7.70 (m, 3H); 8.02 (d, J=8 Hz, 2H); LCMS (M): t_{R}=1.79, ES, m/z=619.

### Synthesis of mAb 1a: CEACAM5_Tusamitamab-DBCO

mAb 1a was prepared following general method M9. To 4 mL of anti-CEACAM5 Tusamitamab (CAS [2349294-95-5]) antibody (22.5 mg/mL in buffer B) were added DPBS (2.8 mL) and DMA (1.5 mL). This solution was treated with 255 μL of a solution of Compound 5a in DMA (20.89 mM) and reacted for 2 h. The crude was purified by gel filtration using a Sephadex™ G25 matrix (Hiprep™ 26/10 desalting column, GE Healthcare) pre-equilibrated with 80:20 DPBS/DMA; mAb 1a was obtained as a colorless limpid solution (93 mg at 5.84 mg/mL) with a LAR (SEC) of 4.32 and a monomeric purity of 98.6%.

### Synthesis of mAb 1b: CEACAM5_Tusamitamab-DBCO

mAb 1b was prepared following general method M8. To 1.6 mL of anti-CEACAM5 Tusamitamab (CAS [2349294-95-5]) antibody (19.7 mg/mL in buffer A) were added DPBS (0.78 mL), 1M HEPES (19 μL) and DMA (0.52 mL). This solution was treated with 80 μL of a solution of Compound 5a in DMA (21.6 mM) and reacted for 2 h; mAb 1b was obtained crude as a colorless limpid solution (32 mg at 10.5 mg/mL) with a LAR (SEC) of 4.4 and a monomeric purity of 97.7%.

### Synthesis of mAb 1c: CEACAM5_Tusamitamab-DBCO

mAb 1c was prepared following general method M10. To 5 mL of anti-CEACAM5 Tusamitamab (CAS [2349294-95-5]) antibody (21.3 mg/mL in buffer A) was added 30 mM K₂HPO₄ (5 mL). This solution was treated with 147 μL of a solution of Compound 5b in DMSO (50 mM) and reacted for 3 h. The crude was purified by gel filtration using Sephadex™ G25 matrix (Hitrap™ desalting column, GE Healthcare); mAb 1c was obtained as a colorless limpid solution (106.5 mg at 10.495 mg/mL) with a LAR (SEC) of 4.17 and a monomeric purity of 97.4%.

### Synthesis of mAb 1d: CEACAM5_Tusamitamab-DBCO

mAb 1d was prepared following general method M10. To 5 mL of anti-CEACAM5 Tusamitamab (CAS [2349294-95-5]) antibody (21.3 mg/mL in buffer A) was added 30 mM K₂HPO₄ (5 mL). This solution was treated with 147 μL of a solution of Compound 5c in water (50 mM) and reacted for 3 h. The crude was purified by gel filtration using Sephadex™ G25 matrix (Hitrap™ desalting column, GE Healthcare); mAb 1d was obtained as a colorless limpid solution (106 mg at 10.495 mg/mL) with a LAR (HRMS) of 1.94 and a monomeric purity of 98.8%.

### Synthesis of mAb 1e: CEACAM5_Tusamitamab-DBCO

mAb 1e was prepared following general method M10. To 5 mL of anti-CEACAM5 Tusamitamab (CAS [2349294-95-5]) antibody (21.3 mg/mL in buffer A) was added 30 mM K₂HPO₄ (5 mL). This solution was treated with 147 μL of a solution of Compound 5d in water (50 mM) and reacted for 3 h. The crude was purified by gel filtration using Sephadex™ G25 matrix (Hitrap™ desalting column, GE Healthcare); mAb 1e was obtained as a colorless limpid solution (106 mg at 10.495 mg/mL) with a LAR (HRMS) of 5 and a monomeric purity of 97.5%.

### Synthesis of mAb 2: EphA2_hu2H11_R35R74-DBCO

mAb 2 was prepared following general method M8. To 4 mL of anti-EphA2 hu2H11_R35R74 antibody (11.1 mg/mL in buffer D) were added 1.1 mL of DPBS and 1.2 mL of DMA. This solution was treated with 120 μL of a solution of Compound 5a in DMA (20 mM) and reacted for ˜3 h; mAb 2 was obtained crude as a colorless limpid solution (45 mg at 6.9 mg/mL) with a LAR (SEC) of 4.1 and a monomeric purity of 96.6%.

### Synthesis of mAb 3: HER2_trastuzumab-DBCO

mAb 3 was prepared following general method M8. To 5.86 mL of anti-HER2 antibody Trastuzumab, Herceptin® (17.78 mg/mL in DPBS) were added 1.365 mL of DMA. This solution was treated with 126 μL of a solution of Compound 5a in DMA (20 mM) and reacted for 5 h; mAb3 was obtained crude as a colorless limpid solution (45 mg at 6.1 mg/mL) with a LAR (SEC) of 4.1 and a monomeric purity of 98%.

### Synthesis of mAb 4: EGFR_cetuximab-DBCO

mAb 4 was prepared following general method M8. To 23 mL of anti-EGFR antibody Cetuximab (2 mg/mL in buffer C) were added 3 mL of DMA. This solution was treated with 126 μL of a solution of Compound 5a in DMA (20 mM) and reacted for 3 h; mAb 4 was obtained crude as a colorless limpid solution (46 mg at 1.6 mg/mL) with a LAR (SEC) of 4.13 and a monomeric purity of 97.7%.

### Synthesis of mAb 5: B7H3_enoblituzumab-mAb-DBCO

mAb 5 was prepared following general method M8. To 27 mL of anti-B7H3 antibody Enoblituzumab (1.7 mg/mL in DPBS) were added 6.6 mL of DMA. This solution was treated with 126 μL of a solution of Compound 5a in DMA (20 mM) and reacted for 3 h; mAb 5 was obtained crude as a colorless limpid solution (45 mg at 1.36 mg/mL) with a LAR (SEC) of 4.14 and a monomeric purity of 98.8%.

### Synthesis of Examples 1 to 6: azido-PEG4-R848 and Corresponding ADCs

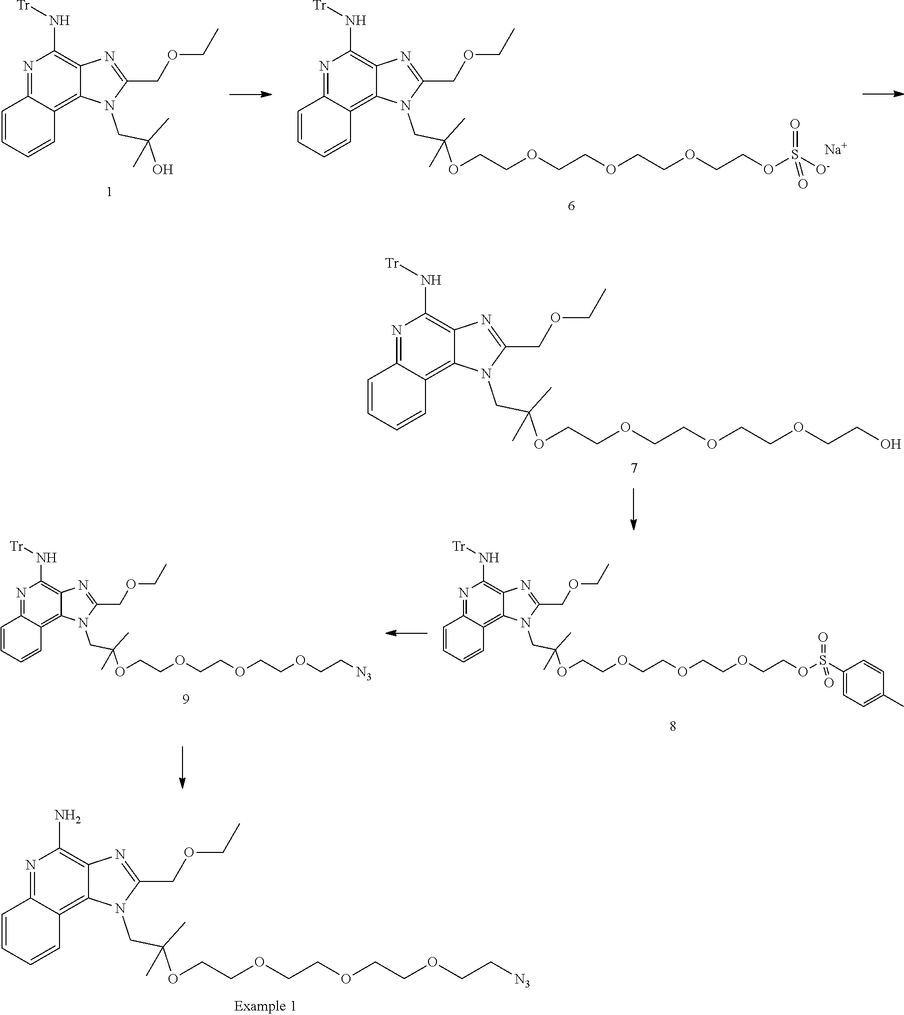

### Compound 6: sodium 14-(2-(ethoxymethyl)-4-(tritylamino)-1H-imidazo[4,5-c]quinolin-1-yl)-13,13-dimethyl-3,6,9,12-tetraoxatetradecyl sulfate

Following the procedure described in general method M2, compound 6 was prepared from compound 1 (100 mg, 0.179 mmol) and compound 2 (73 mg, 0.287 mmol) and obtained as a white solid (122 mg, 81%).

NMR ¹H (400 MHz, δ in ppm, DMSO-d6): 1.11 to 1.26 (m, 9H); 3.31 to 3.58 (m, 18H); 3.75 to 3.81 (m, 2H); 4.68 to 5.09 (m, 2H); 7.01 (s, 1H); 7.09 (d, J=8 Hz, 1H); 7.16 to 7.31 (m, 11H); 7.40 (d, J=8 Hz, 6H); 8.24 (d, J=8 Hz, 1H); LCMS (M1): t_{R}=1.30, ES, m/z=813 [M+H]⁺, m/z=811 [M−H]⁻.

### Compound 7: 14-(2-(ethoxymethyl)-4-(tritylamino)-1H-imidazo[4,5-c]quinolin-1-yl)-13,13-dimethyl-3,6,9,12-tetraoxatetradecan-1-ol

Following the procedure described in general method M3, compound 7 was prepared from compound 6 (118 mg, 0.141 mmol) in 6 mL of a 4:1 mixture of pyridine and 1,4-dioxane and obtained as a colorless oil (105 mg, quant.).

NMR ¹H (400 MHz, δ in ppm, DMSO-d6): 1.07 to 1.28 (m, 9H); 3.34 to 3.56 (m, 20H); 4.59 (t, J=5 Hz, 1H); 4.75 (large s, 2H); 7.03 (s, 1H); 7.09 (d, J=8 Hz, 1H); 7.16 to 7.31 (m, 10H); 7.40 (d, J=8 Hz, 7H); 8.24 (d, J=8 Hz, 1H). LCMS (M1): t_{R}=1.10, ES, m/z=733 [M+H]⁺.

### Compound 8: 14-(2-(ethoxymethyl)-4-(tritylamino)-1H-imidazo[4,5-c]quinolin-1-yl)-13,13-dimethyl-3,6,9,12-tetraoxatetradecyl 4-methylbenzenesulfonate

Under argon, to a solution of compound 7 (120 mg, 0.164 mmol) in anhydrous DCM (6 mL) were added DMAP (3 mg, 0.024 mmol) and TEA (114 μL, 0.818 mmol) followed by tosyl chloride (93 mg, 0.491 mmol) at 0° C. The reaction mixture was allowed to warm up to RT. After completion of the reaction, the mixture was diluted with DCM (50 mL), washed with water (3×10 mL) and sat. brine (3×13 mL), dried over MgSO₄, filtered and concentrated in vacuo. The crude yellow oil was purified by flash chromatography on 10 g of silica gel (gradient elution DCM/MeOH) to give 90 mg of compound 8 as a colorless oil (62%).

NMR ¹H (400 MHz, δ in ppm, chloroform-d): 1.10 to 1.36 (m, 15H); 2.42 (s, 3H); 3.36 to 3.60 (m, 16H); 3.64 (m, 2H); 4.13 (m, 2H); 4.74 (s, 2H); 7.00 (s, 1H); 7.07 to 7.35 (m, 14H); 7.40 to 7.57 (m, 6H); 7.78 (d, J=8 Hz, 2H); 7.99 (d, J=8 Hz, 1H).

### Compound 9: 1-(14-azido-2,2-dimethyl-3,6,9,12-tetraoxatetradecyl)-2-(ethoxymethyl)-N-trityl-1H-imidazo[4,5-c]quinolin-4-amine

Under argon, to a solution of compound 8 (90 mg, 0.101 mmol) in anhydrous DMF (6 mL) was added sodium azide (33 mg, 0.507 mmol). After stirring overnight at 80° C., the yellow reaction mixture was concentrated in vacuo and co-evaporated with toluene. The residue was taken up in a mixture of DCM (50 mL) and water (10 mL). The organic layer was washed with water (2×10 mL) and sat. brine (2×10 mL), dried over MgSO₄, filtered and concentrated in vacuo. The residue was purified by flash chromatography on 10 g of silica gel (gradient elution DCM/MeOH) to give 77 mg of compound 9 as a yellow oil (quant.).

NMR ¹H (400 MHz, δ in ppm, DMSO-d6): 1.12 to 1.31 (m, 9H); 3.34 to 3.41 (m, 8H); 3.44 to 3.59 (m, 12H); 4.76 (large s, 2H); 7.01 (s, 1H); 7.10 (d, J=8 Hz, 1H); 7.18 to 7.32 (m, 11H); 7.41 (d, J=8 Hz, 6H); 8.25 (d, J=8 Hz, 1H); LCMS (M1): t_{R}=1.28, ES, m/z=758 [M+H]; IR: 3419; 2873; 2104; 1591; 1534; 1490; 1096 and 699 cm⁻¹.

### Example 1: 1-(14-azido-2,2-dimethyl-3,6,9,12-tetraoxatetradecyl)-2-(ethoxymethyl)-1H-imidazo[4,5-c]quinolin-4-amine

Following the procedure described in general method M5, compound 9 (85 mg, 0.112 mmol) in 1,4-dioxane (2.4 mL) was treated with 4N HCl in 1,4-dioxane (4.8 mL) to give Example 1 as a colorless oil (49 mg, 84%).

NMR ¹H (400 MHz, δ in ppm, DMSO-d6): 1.10 to 1.26 (m, 9H); 3.32 to 3.70 (m, 20H); 4.77 (large s, 2H); 6.54 (large s, 2H); 7.22 (t, J=7 Hz, 1H); 7.41 (t, J=8 Hz, 1H); 7.59 (d, J=8 Hz, 1H); 8.29 (d, J=8 Hz, 1H); LCMS (M1): t_{R}=0.70, ES, m/z=516 [M+H]⁺.

### Example 2: CEACAM5_Tusamitamab-PEG4-R848 ADC

Example 2 was prepared following general method M11; mAb 1a (15 mg at 5.84 mg/mL) in 80:20 DPBS/DMA was reacted overnight with 53.06 μL of a solution of Example 1 in DMA (12.48 mM). The crude mixture was purified and formulated by gel filtration using a Sephadex™ G25 matrix (Hiprep™ 26/10 desalting column, GE Healthcare) pre-equilibrated with DPBS buffer. Example 2 (13.05 mg at 1.45 mg/mL, overall yield 87%) was finally obtained after sterile filtration as a colorless limpid solution with a DAR (HRMS) of 4.3 and a monomeric purity of 98.1%.

RP-HRMS: 147417 (naked mAb); 148305 (D1); 149195 (D2); 150082 (D3); 150970 (D4); 151857 (D5); 152747 (D6); 153634 (D7); 154521 (D8); 155411 (D9).

### Example 3: EphA2_hu2H11_R35R74-PEG4-R848 ADC

Example 3 was prepared following general method M13; mAb 2 (10.5 mg at 7 mg/mL) in 80:20 DPBS/DMA was reacted overnight with 85 μL of a solution of Example 1 in DMA (10 mM). The mixture was diafiltrated on Vivaspin® (PES membrane, 50K, Sartorius) using 80:20 DPBS/DMA, then formulated by gel filtration using a Sephadex™ G25 matrix (Hitrap™ desalting column, GE Healthcare) pre-equilibrated with the final aqueous buffer A. Example 3 (6.4 mg at 2.65 mg/mL, overall yield 60%) was finally obtained after sterile filtration as a colorless limpid solution with a DAR (HRMS) of 3.92 and a monomeric purity of 97.6%.

RP-HRMS: 149355 (naked mAb); 150241 (D1); 151130 (D2); 152018 (D3); 152905 (D4); 153793 (D5); 154681 (D6); 155569 (D7); 156454 (D8); 157347 (D9).

### Example 4: HER2_trastuzumab-PEG4-R848 ADC

Example 4 was prepared following general method M13; mAb 3 (11 mg at 10 mg/mL) in 80:20 DPBS/DMA was reacted overnight with 91 μL of a solution of Example 1 in DMA (10 mM). The mixture was diafiltrated on Vivaspin® (PES membrane, 50K, Sartorius) using 80:20 DPBS/DMA, then formulated by gel filtration using a Sephadex™ G25 matrix (Hitrap™ desalting column, GE Healthcare) pre-equilibrated with the final aqueous buffer A. Example 4 (6.67 mg at 2.78 mg/mL, overall yield 59%) was finally obtained after sterile filtration as a colorless limpid solution with a DAR (HRMS) of 3.84 and a monomeric purity of 98.2%.

RP-HRMS: 148059 (naked mAb); 148945 (D1); 149832 (D2); 150720 (D3); 151608 (D4); 152495 (D5); 53384 (D6); 154269 (D7); 155158 (D8); 156048 (D9).

### Example 5: EGFR_cetuximab-PEG4-R848 ADC

Example 5 was prepared following general method M12; mAb 4 (12 mg at 1.6 mg/mL) in 80:20 buffer C/DMA was reacted overnight with 99 μL of a solution of Example 1 in DMA (10 mM). The mixture was concentrated on Vivaspin® (PES membrane, 50K, Sartorius), then purified by gel filtration using a Superdex™ 200 pg matrix (HiLoad® 16/60 desalting column, GE Healthcare). After concentration on Vivaspin® (PES membrane, 50K, Sartorius) and formulation by gel filtration using a Sephadex™ G25 matrix (NAP®, GE Healthcare) pre-equilibrated with the final aqueous buffer A, Example 5 (6.9 mg at 2.48 mg/mL, overall yield 60%) was finally obtained after sterile filtration as a colorless limpid solution with a DAR (HRMS) of 4.3 and a monomeric purity of 100%.

RP-HRMS: 153690 (D1); 154566 (D2); 155448 (D3); 156348 (D4); 157229 (D5); 158106 (D6); 159002 (D7).

### Example 6: B7H3_enoblituzumab-PEG4-R848 ADC

Example 6 was prepared following general method M13; mAb 5 (11.3 mg at 1.36 mg/mL) in 80:20 DPBS/DMA was reacted overnight with 93 μL of a solution of Example 1 in DMA (10 mM). The reaction mixture was diafiltrated on Vivaspin® (PES membrane, 50K, Sartorius) using 80:20 DPBS/DMA, then formulated by gel filtration using a Sephadex™ G25 matrix (Hitrap™ desalting column, GE Healthcare) pre-equilibrated with the final aqueous buffer A. Example 6 (5.67 mg at 3.07 mg/mL, overall yield 50%) was finally obtained after sterile filtration as a colorless limpid solution with a DAR (HRMS) of 4.3 and a monomeric purity of 98.4%.

RP-HRMS: 147998 (naked mAb); 148895 (D1); 149780 (D2); 150669 (D3); 151558 (D4); 152447 (D5); 153332 (D6); 154219 (D7); 155106 (D8).

### Synthesis of Examples 7 to 12: azido-PEG8-R848 and Corresponding ADCs

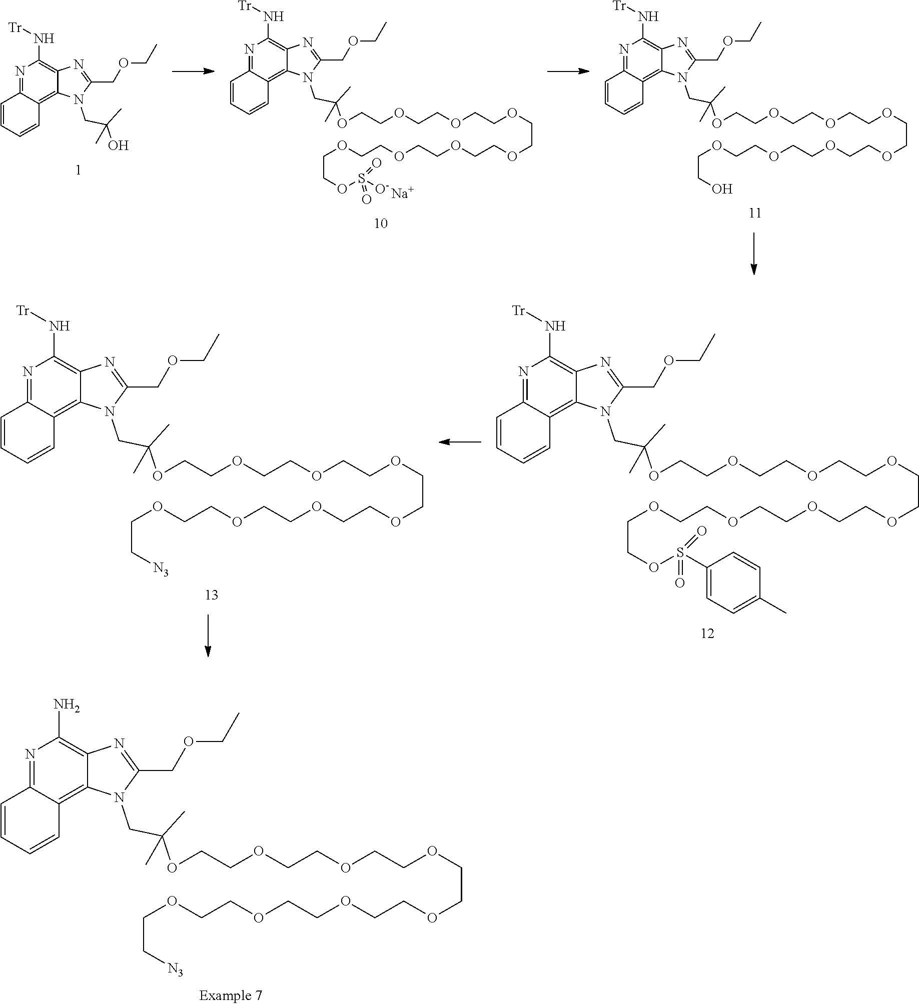

### Compound 10: sodium 26-(2-(ethoxymethyl)-4-(tritylamino)-1H-imidazo[4,5-c]quinolin-1-yl)25,25-dimethyl-3,6,9,12,15,18,21,24-octaoxahexacosyl sulfate

Following the procedure described in general method M2, compound 10 was prepared from compound 1 (200 mg, 0.359 mmol) and compound 3 (279 mg, 0.646 mmol) and obtained as a colorless oil (363 mg, 90%).

NMR ¹H (400 MHz, δ in ppm, DMSO-d6): 1.11 to 1.29 (m, 9H); 3.33 to 3.58 (m, 34H); 3.77 (m, 2H); 4.75 (large s, 2H); 7.00 (s, 1H); 7.09 (d, J=8 Hz, 1H); 7.16 to 7.32 (m, 11H); 7.39 (d, J=8 Hz, 6H); 8.24 (d, J=8 Hz, 1H); LCMS (M1): t_{R}=1.33, ES, m/z=989 [M+H]⁺, m/z=987 [M−H]⁻, m/z=455 [M+2H—SO₃H]²⁺.

### Compound 11: 26-(2-(ethoxymethyl)-4-(tritylamino)-1H-imidazo[4,5-c]quinolin-1-yl)-25,25-dimethyl-3,6,9,12,15,18,21,24-octaoxahexacosan-1-ol

Following the procedure described in general method M3, compound 11 was prepared from compound 10 (325 mg, 0.321 mmol) in 19.5 mL of a 4:1 mixture of pyridine and 1,4-dioxane and obtained as a white solid (211 mg, 72%).

NMR ¹H (400 MHz, δ in ppm, DMSO-d6): 1.11 to 1.33 (m, 9H); 3.35 to 3.56 (m, 36H); 4.54 (s, 1H); 4.75 (large s, 2H); 7.00 (s, 1H); 7.09 (d, J=8 Hz, 1H); 7.17 to 7.31 (m, 11H); 7.39 (m, 6H); 8.24 (d, J=9 Hz, 1H); LCMS (M1): t_{R}=1.11, ES, m/z=909 [M+H]⁺, m/z=455 [M+2H]²⁺, m/z=953 [M−H+HCOOH]⁻.

### Compound 12: 26-(2-(ethoxymethyl)-4-(tritylamino)-1H-imidazo[4,5-c]quinolin-1-yl)-25,25-dimethyl-3,6,9,12,15,18,21,24-octaoxahexacosyl 4-methylbenzenesulfonate

Under argon, to a solution of compound 11 (98 mg, 0.107 mmol) in anhydrous DCM (7 mL) were added DMAP (2.6 mg, 0.021 mmol) and TEA (105 μL, 0.754 mmol) followed by tosyl chloride (102 mg, 0.539 mmol) at 0° C. The reaction mixture was allowed to warm up to RT. After completion of the reaction, the reaction mixture was diluted with DCM (50 mL), washed with water (3×10 mL) and sat. brine (2×10 mL), dried over MgSO₄, filtered and concentrated in vacuo. The crude yellow oil was purified by flash chromatography on 10 g of silica gel (gradient elution DCM/MeOH) to give 71 mg of compound 12 as a colorless oil (62%).

NMR ¹H (400 MHz, δ in ppm, DMSO-d6): 1.12 to 1.29 (m, 9H); 2.40 (s, 3H); 3.33 to 3.65 (m, 34H); 4.10 (large s, 2H); 4.74 (large s, 2H); 6.99 (s, 1H); 7.09 (d, J=8 Hz, 1H); 7.16 to 7.31 (m, 11H); 7.39 (d, J=8 Hz, 6H); 7.43 to 7.50 (m, J=8 Hz, 2H); 7.75 to 7.80 (m, J=8 Hz, 2H); 8.23 (d, J=7 Hz, 1H); LCMS (M1): t_{R}=1.35, ES, m/z=1063 [M+H]⁺, m/z=532 [M+2H]²⁺.

### Compound 13: 1-(26-azido-2,2-dimethyl-3,6,9,12,15,18,21,24-octaoxahexacosyl)-2-(ethoxy-methyl)-N-trityl-1H-imidazo[4,5-c]quinolin-4-amine

Under argon, to a solution of compound 12 (69 mg, 0.065 mmol) in anhydrous DMF (5 mL) was added sodium azide 21 mg, 0.324 mmol). After stirring overnight at 80° C., the yellow solution was concentrated in vacuo and co-evaporated with toluene. The residue was taken up in a mixture of DCM (50 mL) and water (10 mL). The organic layer was washed with water (2×10 mL) and sat. brine (2×10 mL), dried over MgSO₄, filtered and concentrated in vacuo. The residue was purified by flash chromatography on 5 g of silica gel (gradient elution DCM/MeOH) to give 60 mg of compound 13 as a white solid (quant.).

NMR ¹H (400 MHz, δ in ppm, DMSO-d6): 1.09 to 1.26 (m, 9H); 3.32 to 3.62 (m, 36H); 4.75 (large s, 2H); 7.00 (s, 1H); 7.09 (d, J=8 Hz, 1H); 7.14 to 7.32 (m, 11H); 7.39 (d, J=7 Hz, 6H); 8.24 (d, J=8 Hz, 1H); LCMS (M1): t_{R}=1.26, ES, m/z=934 [M+H]⁺.

### Example 7: 1-(14-azido-2,2-dimethyl-3,6,9,12-tetraoxatetradecyl)-2-(ethoxymethyl)-1H-imidazo[4,5-c]quinolin-4-amine

Following the procedure described in general method M5, compound 13 (51 mg, 0.054 mmol) in 1,4-dioxane (1.5 mL) was treated with 4N HCl in 1,4-dioxane (3 mL) to give example 7 as a colorless oil (33 mg, 87%).

NMR ¹H (400 MHz, δ in ppm, DMSO-d6): 1.10 to 1.28 (m, 9H); 3.32 to 3.60 (m, 36H); 4.77 (large s, 2H); 6.55 (large s, 2H); 7.23 (t, J=8 Hz, 1H); 7.41 (t, J=8 Hz, 1H); 7.59 (d, J=8 Hz, 1H); 8.29 (d, J=8 Hz, 1H); LCMS (M1): t_{R}=0.74, ES, m/z=692 [M+H]⁺.

### Example 8: CEACAM5_Tusamitamab-PEG8-R848 ADC

Example 8 was prepared following general method M11; mAb 1b (52 mg at 10.5 mg/mL) in 80:20 DPBS/DMA was reacted overnight with 430 μL of a solution of Example 7 in DMA (9.96 mM). The crude mixture was purified and formulated by gel filtration using a Sephadex™ G25 matrix (Hiprep™ 26/10 desalting column, GE Healthcare) pre-equilibrated with the final aqueous buffer A. Example 8 (44.72 mg at 2.6 mg/mL, overall yield 85%) was finally obtained after sterile filtration as a colorless limpid solution with a DAR (HRMS) of 4.2 and a monomeric purity of 98.3%.

RP-HRMS: 147826 (naked mAb); 148492 (D1); 149554 (D2); 150619 (D3); 151683 (D4); 152748 (D5); 153814 (D6); 154880 (D7); 155943 (D8); 157007 (D9).

### Example 9: EphA2_hu2H11_R35R74-PEG8-R848 ADC

Example 9 was prepared following general method M13; mAb 2 (10.5 mg at 6.95 mg/mL) in 80:20 DPBS/DMA was reacted overnight with 45 μL of a solution of Example 7 in DMA (18.9 mM). The mixture was diafiltrated on Vivaspin® (PES membrane, 50K, Sartorius) using 80:20 DPBS/DMA, then formulated by gel filtration using a Sephadex™ G25 matrix (Hitrap™ desalting column, GE Healthcare) pre-equilibrated with the final aqueous buffer A. Example 9 (6.3 mg at 2.47 mg/mL, overall yield 61%) was finally obtained after sterile filtration as a colorless limpid solution with a DAR (HRMS) of 3.82 and a monomeric purity of 97.8%.

RP-HRMS: 149352 (naked mAb); 150419 (D1); 151482 (D2); 152546 (D3); 153611 (D4); 154676 (D5); 155739 (D6); 156802 (D7); 157870 (D8); 158934 (D9).

### Example 10: HER2_trastuzumab-PEG8-R848 ADC

Example 10 was prepared following general method M13; mAb 3 (11 mg at 6.14 mg/mL) in 80:20 DPBS/DMA was reacted overnight with 49 μL of a solution of Example 7 in DMA (18.92 mM). The mixture was diafiltrated on Vivaspin® (PES membrane, 50K, Sartorius) using 80:20 DPBS/DMA, then formulated by gel filtration using a Sephadex™ G25 matrix (Hitrap™ desalting column, GE Healthcare) pre-equilibrated with the final aqueous buffer A. Example 10 (6.7 mg at 2.4 mg/mL, overall yield 61%) was finally obtained after sterile filtration as a colorless limpid solution with a DAR (HRMS) of 3.92 and a monomeric purity of 98.2%.

RP-HRMS: 148056 (naked mAb); 149120 (D1); 150184 (D2); 151248 (D3); 152314 (D4); 153376 (D5); 154439 (D6); 155502 (D7); 156580 (D8); 157633 (D9); 158697 (D10).

### Example 11: EGFR_cetuximab-PEG8-R848 ADC

Example 11 was prepared following general method M12; mAb 4 (12 mg at 1.6 mg/mL) in 80:20 buffer C/DMA was reacted overnight with 52 μL of a solution of Example 7 in DMA (18.9 mM). The mixture was concentrated on Vivaspin® (PES membrane, 50K, Sartorius), then purified by gel filtration using a Superdex™ 200 pg matrix (HiLoad® 16/60 desalting column, GE Healthcare). After concentration on Vivaspin® (PES membrane, 50K, Sartorius) and formulation by gel filtration using a Sephadex™ G25 matrix (NAP®, GE Healthcare) pre-equilibrated with the final aqueous buffer A, Example 11 (7.3 mg at 2.62 mg/mL, overall yield 63%) was finally obtained after sterile filtration as a colorless limpid solution with a DAR (HRMS) of 3.8 and a monomeric purity of 100%.

RP-HRMS: 152781 (naked mAb); 153849 (D1); 154920 (D2); 155980 (D3); 157052 (D4); 158109 (D5); 159170 (D6); 160224 (D7); 161303 (D8).

### Example 12: B7H3_enoblituzumab-PEG8-R848 ADC

Example 12 was prepared following general method M13; mAb 5 (11.3 mg at 1.36 mg/mL) in 80:20 DPBS/DMA was reacted overnight with 49 μL of a solution of Example 7 in DMA (18.92 mM). The mixture was diafiltrated on Vivaspin® (PES membrane, 50K, Sartorius) using 80:20 DPBS/DMA, then formulated by gel filtration using a Sephadex™ G25 matrix (Hitrap™ desalting column, GE Healthcare) pre-equilibrated with the final aqueous buffer A. Example 12 (6.5 mg at 3.42 mg/mL, overall yield 59%) was finally obtained after sterile filtration as a colorless limpid solution with a DAR (HRMS) of 4.45 and a monomeric purity of 97.6%.

RP-HRMS: 149071 (D1); 150131 (D2); 151196 (D3); 152261 (D4); 153324 (D5); 154389 (D6); 155450 (D7); 156516 (D8); 157582 (D9).

### Synthesis of Examples 13 to 18: azido-PEG12-R848 and Corresponding ADCs

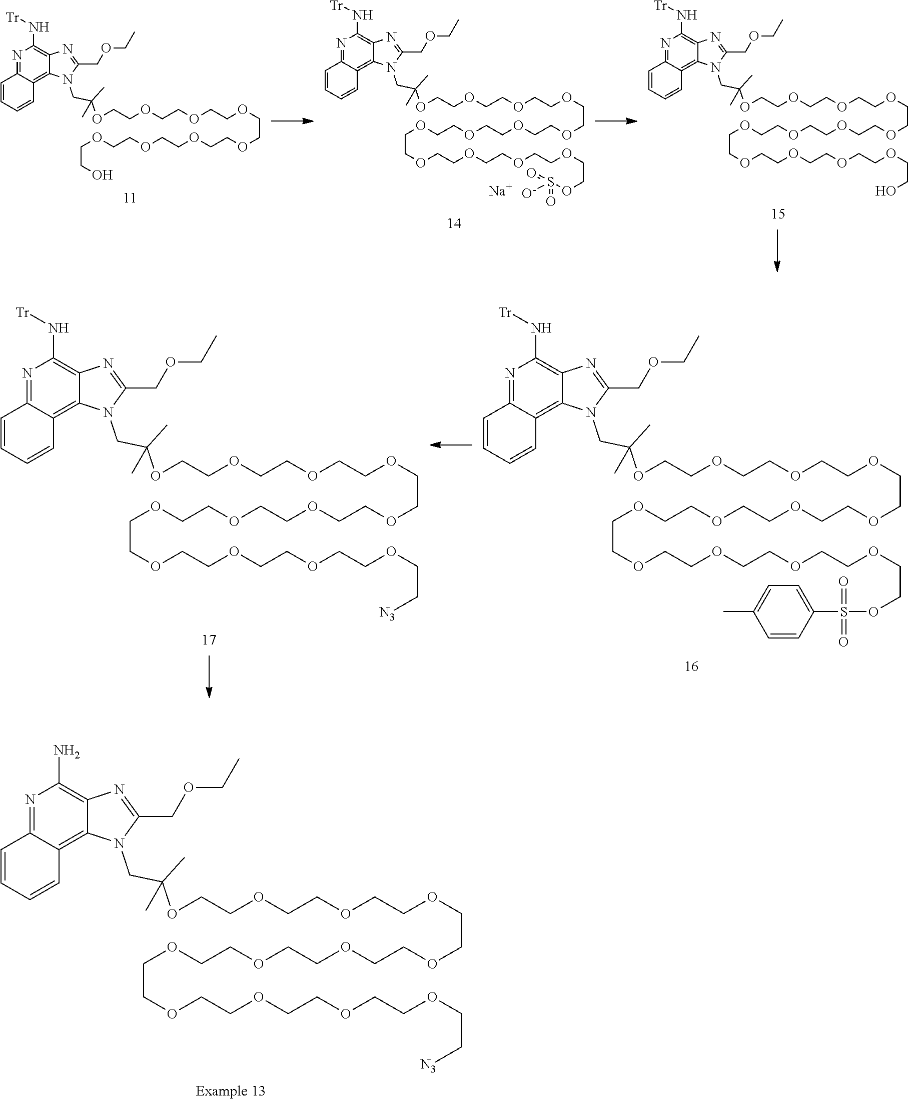

### Compound 14: sodium 38-(2-(ethoxymethyl)-4-(tritylamino)-1H-imidazo[4,5-c]quinolin-1-yl)37,37-dimethyl-3,6,9,12,15,18,21,24,27,30,33,36-dodecaoxaoctatriacontyl sulfate

Following the procedure described in general method M4, compound 14 was prepared from compound 11 (138 mg, 0.151 mmol) and obtained as a colorless oil (178 mg, 98%).

NMR ¹H (400 MHz, δ in ppm, DMSO-d6): 1.10 to 1.28 (m, 9H); 3.25 to 3.59 (m, 50H); 3.78 (dd, J=4 and 6 Hz, 2H); 4.64 to 4.82 (large s, 2H); 7.00 (s, 1H); 7.09 (dd, J=1 and 8 Hz, 1H); 7.14 to 7.32 (m, 11H); 7.35 to 7.44 (m, 6H); 8.24 (d, J=8 Hz, 1H); LCMS (M1): t_{R}=1.35, ES, m/z=1165 [M+H]⁺, m/z=1163 [M−H]⁻, m/z=543 [M+2H—SO₃H]²⁺.

### Compound 15: 38-(2-(ethoxymethyl)-4-(tritylamino)-1H-imidazo[4,5-c]quinolin-1-yl)-37,37-dimethyl-3,6,9,12,15,18,21,24,27,30,33,36-dodecaoxaoctatriacontan-1-ol

Following the procedure described in general method M3, compound 15 was prepared from compound 14 (176 mg, 0.148 mmol) in 13.5 mL of a 4:1 mixture of pyridine and 1,4-dioxane and obtained as a colorless oil (100 mg, 62%).

NMR ¹H (400 MHz, δ in ppm, DMSO-d6): 1.03 to 1.32 (m, 9H); 3.31 to 3.55 (m, 52H); 4.54 (s, 1H); 4.75 (large s, 2H); 7.00 (s, 1H); 7.09 (d, J=8 Hz, 1H); 7.16 to 7.31 (m, 11H); 7.39 (d, J=8 Hz, 6H); 8.24 (d, J=8 Hz, 1H); LCMS (M1): t_{R}=1.11, ES, m/z=1085 [M+H]⁺, m/z=543 [M+2H]²⁺, m/z=1129 [M−H+HCOOH]⁻.

### Compound 16: 38-(2-(ethoxymethyl)-4-(tritylamino)-1H-imidazo[4,5-c]quinolin-1-yl)-37,37-dimethyl-3,6,9,12,15,18,21,24,27,30,33,36-dodecaoxaoctatriacontyl 4-methylbenzenesulfonate

Under argon, to a solution of compound 15 (97 mg, 0.089 mmol) in anhydrous DCM (7 mL) were added DMAP (2.2 mg, 0.018 mmol) and TEA (87 μL, 0.625 mmol) followed by tosyl chloride (85 mg, 0.446 mmol) at 0° C. The reaction mixture was allowed to warm up to RT. After completion of the reaction, the mixture was diluted with DCM (50 mL), washed with water (3×10 mL) and sat. brine (2×10 mL), dried over MgSO₄, filtered and concentrated in vacuo. The crude yellow oil was purified by flash chromatography on 10 g of silica gel (gradient elution DCM/MeOH) to give 95 mg of compound 16 as a colorless oil (85%).

NMR ¹H (400 MHz, δ in ppm, DMSO-d6): 1.03 to 1.30 (m, 9H); 2.41 (s, 3H); 3.32 to 3.61 (m, 50H); 4.10 (m, 2H); 4.65 to 5.03 (large m, 2H); 6.99 (s, 1H); 7.09 (d, J=8 Hz, 1H); 7.16 to 7.31 (m, 11H); 7.36 to 7.42 (m, 6H); 7.47 (d, J=8 Hz, 2H); 7.78 (d, J=1 Hz, 2H); 8.24 (d, J=8 Hz, 1H); LCMS (M1): t_{R}=1.33, ES, m/z=1239 [M+H]⁺, m/z=1237 [M−H]⁻, m/z=620 [M+2H]²⁺, m/z=1283 [M−H+HCOOH]⁻.

### Compound 17: 1-(38-azido-2,2-dimethyl-3,6,9,12,15,18,21,24,27,30,33,36-dodecaoxaoctatriacontyl)-2-(ethoxymethyl)-N-trityl-1H-imidazo[4,5-c]quinolin-4-amine

Under argon, to a solution of compound 16 (94 mg, 0.075 mmol) in anhydrous DMF (5 mL) was added sodium azide (24 mg, 0.379 mmol). After stirring overnight at 80° C., the yellow reaction mixture was concentrated in vacuo and co-evaporated with toluene. The residue was taken up in a mixture of DCM (50 mL) and water (10 mL). The organic layer was washed with water (2×10 mL) and sat. brine (2×10 mL), dried over MgSO₄, filtered and concentrated in vacuo. The residue was purified by flash chromatography on 5 g of silica gel (gradient elution DCM/MeOH) to give 81 mg of compound 17 as a colorless oil (96%).

NMR ¹H (400 MHz, δ in ppm, DMSO-d6): 1.08 to 1.28 (m, 9H); 3.30 to 3.70 (m, 52H); 4.62 to 5.09 (m large, 2H); 6.99 (s, 1H); 7.09 (d, J=8 Hz, 1H); 7.15 to 7.33 (m, 11H); 7.39 (d, J=7 Hz, 6H); 8.24 (d, J=8 Hz, 1H); LCMS (M1): t_{R}=1.25, ES, m/z=1110 [M+H]⁺, m/z=541 [M+2H—C₂H₅]²⁺, m/z=1154 [M−H+HCOOH]⁻.

### Example 13: 1-(38-azido-2,2-dimethyl-3,6,9,12,15,18,21,24,27,30,33,36-dodecaoxaocta-triacontyl)-2-(ethoxymethyl)-1H-imidazo[4,5-c]quinolin-4-amine

Following the procedure described in general method M5, compound 17 (71 mg, 0.064 mmol) in 1,4-dioxane (2 mL) was treated with 4N HCl in 1,4-dioxane (4 mL) to give example 13 as a colorless oil (51 mg, 92%).

NMR ¹H (400 MHz, δ in ppm, DMSO-d6): 1.07 to 1.29 (m, 9H); 3.35 to 3.66 (m, 52H); 4.78 (large s, 2H); 6.53 (large s, 2H); 7.23 (t, J=7 Hz, 1H); 7.42 (t, J=8 Hz, 1H); 7.60 (d, J=8 Hz, 1H); 8.30 (d, J=8 Hz, 1H); LCMS (M1): t_{R}=0.77, ES, m/z=868 [M+H]⁺, m/z=420 [M+2H—C₂H₅]²⁺, m/z=912 [M−H+HCOOH]⁻.

### Example 14: CEACAM5_Tusamitamab-PEG12-R848 ADC

Example 14 was prepared following general method M11; mAb 1b (31.5 mg at 10.5 mg/mL) in 74:6:20 DPBS/1M HEPES/DMA was reacted overnight with 188 μL of a solution of Example 13 in DMA (13.6 mM). The crude mixture was purified and formulated by gel filtration using a Sephadex™ G25 matrix (Hiprep™ 26/10 desalting column, GE Healthcare) pre-equilibrated with the final aqueous buffer A. Example 14 (28.8 mg at 3.6 mg/mL, overall yield 91%) was finally obtained after sterile filtration as a colorless limpid solution with a DAR (HRMS) of 4.1 and a monomeric purity of 97.5%.

RP-HRMS: 147422 (naked mAb); 148669 (D1); 149906 (D2); 151148 (D3); 152385 (D4); 153624 (D5); 154869 (D6); 156105 (D7); 157340 (D8); 158560 (D9).

### Example 15: EphA2_hu2H11_R35R74-PEG12-R848 ADC

Example 15 was prepared following general method M13; mAb 2 (10.5 mg at 6.95 mg/mL) in 80:20 DPBS/DMA was reacted overnight with 63 μL of a solution of Example 13 in DMA (13.6 mM). The mixture was diafiltrated on Vivaspin® (PES membrane, 50K, Sartorius) using 80:20 DPBS/DMA, then formulated by gel filtration using a Sephadex™ G25 matrix (Hitrap™ desalting column, GE Healthcare) pre-equilibrated with the final aqueous buffer A. Example 15 (5.8 mg at 2.77 mg/mL, overall yield 58%) was finally obtained after sterile filtration as a colorless limpid solution with a DAR (HRMS) of 3.58 and a monomeric purity of 97.7%.

RP-HRMS: 149353 (naked mAb); 150594 (D1); 151836 (D2); 153076 (D3); 154317 (D4); 155556 (D5); 156797 (D6); 158036 (D7); 159274 (D8).

### Example 16: HER2_trastuzumab-PEG12-R848 ADC

Example 16 was prepared following general method M13; mAb 3 (11 mg at 6.14 mg/mL) in 80:20 DPBS/DMA was reacted overnight with 67 μL of a solution of Example 13 in DMA (13.62 mM). The mixture was diafiltrated on Vivaspin® (PES membrane, 50K, Sartorius) using 80:20 DPBS/DMA, then formulated by gel filtration using a Sephadex™ G25 matrix (Hitrap™ desalting column, GE Healthcare) pre-equilibrated with the final aqueous buffer A. After another diafiltration on Vivaspin® (PES membrane, 50K, Sartorius) using a buffer A, Example 16 (5.7 mg at 2.7 mg/mL, overall yield 57%) was finally obtained after sterile filtration as a colorless limpid solution with a DAR (HRMS) of 3.22 and a monomeric purity of 98.3%.

RP-HRMS: 148057 (naked mAb); 149295 (D1); 150538 (D2); 151776 (D3); 153014 (D4); 154253 (D5); 155493 (D6); 156735 (D7); 157966 (D8).

### Example 17: EGFR_cetuximab-PEG12-R848 ADC

Example 17 was prepared following general method M12; mAb 4 (11.4 mg at 1.6 mg/mL) in 80:20 buffer C/DMA was reacted overnight with 69 μL of a solution of Example 13 in DMA (13.6 mM). The mixture was diafiltrated on Vivaspin® (PES membrane, 50K, Sartorius) with 80:20 DPBS/DMA, then purified by gel filtration using a Superdex™ 200 pg matrix (HiLoad® 16/60 desalting column, GE Healthcare). After concentration on Vivaspin® (PES membrane, 50K, Sartorius) and formulation by gel filtration using a Sephadex™ G25 matrix (NAP®, GE Healthcare) pre-equilibrated with the final aqueous buffer A, Example 17 (5 mg at 2.73 mg/mL, overall yield 44%) was finally obtained after sterile filtration as a colorless limpid solution with a DAR (HRMS) of 3.9 and a monomeric purity of 99.7%.

RP-HRMS: 154043 (D1); 155283 (D2); 156515 (D3); 157765 (D4); 159004 (D5); 160250 (D6); 161488 (D7)

### Example 18: B7H3_enoblituzumab-PEG12-R848 ADC

Example 18 was prepared following general method M13; mAb 5 (11.3 mg at 1.36 mg/mL) in 80:20 DPBS/DMA was reacted overnight with 49 μL of a solution of Example 18 in DMA (18.92 mM). The reaction mixture was diafiltrated on Vivaspin® (PES membrane, 50K, Sartorius) using 80:20 DPBS/DMA, then formulated by gel filtration using a Sephadex™ G25 matrix (Hitrap™ desalting column, GE Healthcare) pre-equilibrated with the final aqueous buffer A. Example 18 (6.3 mg at 2.86 mg/mL, overall yield 57%) was finally obtained after sterile filtration as a colorless limpid solution with a DAR (HRMS) of 3.83 and a monomeric purity of 98.5%.

RP-HRMS: 148003 (naked mAb); 149244 (D1); 150484 (D2); 151723 (D3); 152966 (D4); 154206 (D5); 155444 (D6); 156684 (D7); 157929 (D8).

### Synthesis of Examples 19 to 24: azido-PEG24-R848 and Corresponding ADCs

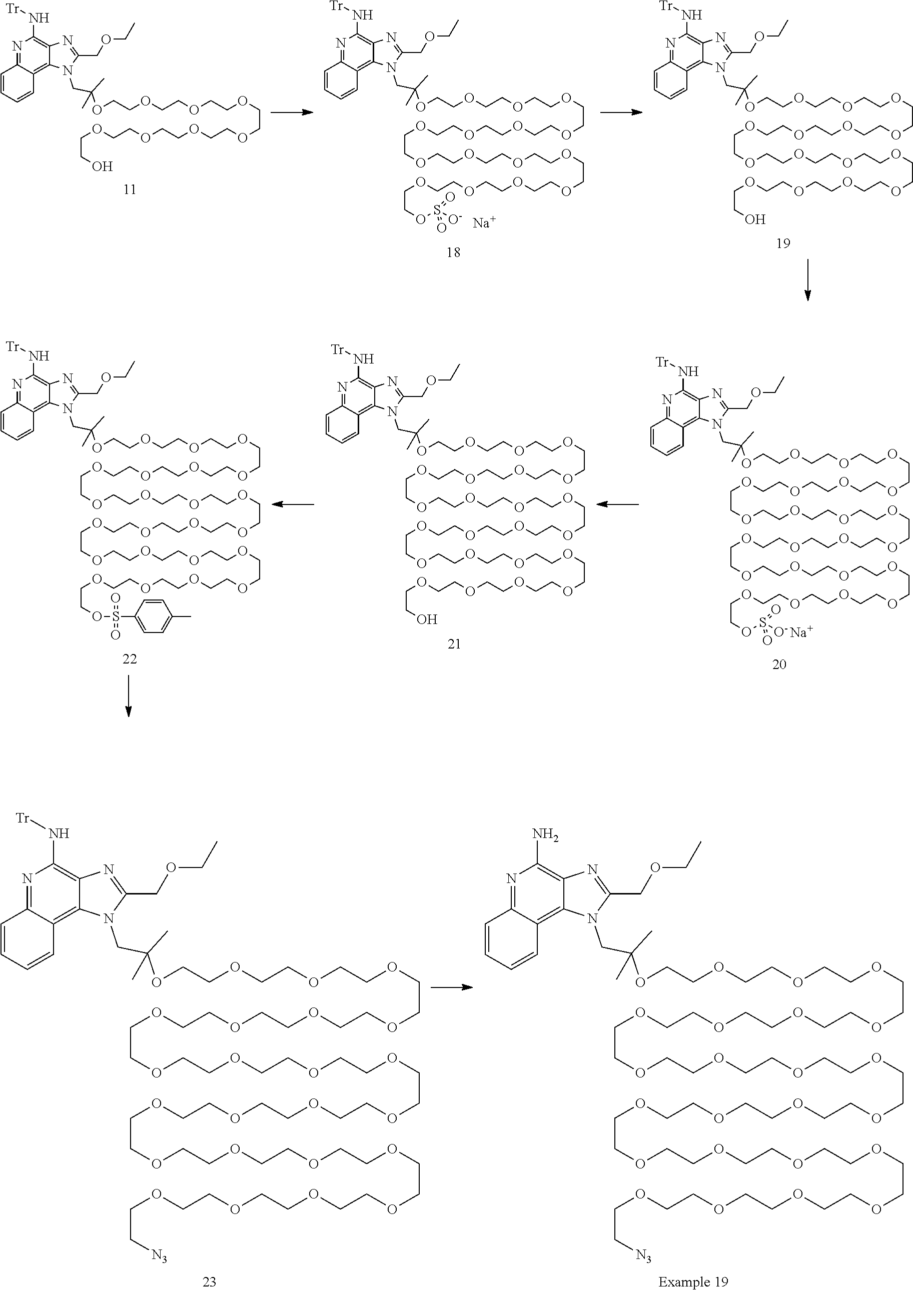

### Compound 18: sodium 50-(2-(ethoxymethyl)-4-(tritylamino)-1H-imidazo[4,5-c]quinolin-1-yl)49,49-dimethyl-3,6,9,12,15,18,21,24,27,30,33,36,39,42,45,48-hexadecaoxapentacontyl sulfate

Following the procedure described in general method M4, compound 18 was prepared from compound 11 (222 mg, 0.244 mmol) and compound 3 (158 mg, 0.366 mmol) and obtained as a colorless oil (354 mg, quant.).

NMR ¹H (400 MHz, δ in ppm, DMSO-d6): 1.04 to 1.26 (m, 9H); 3.31 to 3.68 (m, 66H); 3.78 (m, 2H); 4.66 to 4.99 (m large, 2H); 6.99 (s, 1H); 7.09 (dd, J=1 and 8 Hz, 1H); 7.15 to 7.32 (m, 11H); 7.39 (d, J=8 Hz, 6H); 8.24 (d, J=9 Hz, 1H); LCMS (M1): t_{R}=1.36, ES, m/z=1341 [M+H]⁺, m/z=1339 [M−H]⁻, m/z=631 [M+2H—SO₃H]²⁺.

### Compound 19: 50-(2-(ethoxymethyl)-4-(tritylamino)-1H-imidazo[4,5-c]quinolin-1-yl)-49,49-dimethyl-3,6,9,12,15,18,21,24,27,30,33,36,39,42,45,48-hexadecaoxapentacontan-1-ol

Following the procedure described in general method M3, compound 19 was prepared from compound 18 (350 mg, 0.256 mmol) in 30 mL of a 4:1 mixture of pyridine and 1,4-dioxane and obtained as a colorless oil (258 mg, 79%).

NMR ¹H (400 MHz, δ in ppm, DMSO-d6): 1.03 to 1.31 (m, 9H); 3.34 to 3.55 (m, 68H); 4.54 (t, J=6 Hz, 1H); 4.75 (large s, 2H); 7.00 (s, 1H); 7.09 (dd, J=1 and 8 Hz, 1H); 7.16 to 7.31 (m, 11H); 7.39 (d, J=8 Hz, 6H); 8.24 (d, J=8 Hz, 1H); LCMS (M1): t_{R}=1.12, ES, m/z=1261[M+H]⁺, m/z=631 [M+2H]²⁺, m/z=1305 [M−H+HCOOH]⁻.

### Compound 20: sodium 74-(2-(ethoxymethyl)-4-(tritylamino)-1H-imidazo[4,5-c]quinolin-1-yl)73,73-dimethyl-3,6,9,12,15,18,21,24,27,30,33,36,39,42,45,48,51,54,57, 60,63,66,69,72-tetracosaoxatetraheptacontyl sulfate

Following the procedure described in general M4, compound 20 was prepared from compound 19 (256 mg, 0.203 mmol) and obtained as a colorless oil (308 mg, 88%).

NMR ¹H (400 MHz, δ in ppm, DMSO-d6): 1.01 to 1.33 (m, 9H); 3.33 to 3.66 (m, 98H); 3.78 (t, J=5 Hz, 2H); 4.75 (large s, 2H); 7.00 (s, 1H); 7.09 (d, J=8 Hz, 1H); 7.14 to 7.34 (m, 11H); 7.39 (d, J=8 Hz, 6H); 8.24 (d, J=8 Hz, 1H); LCMS (M1): t_{R}=1.38, ES, m/z=1692 [M−H]⁻, m/z=847 [M+2H]²⁺.

### Compound 21: 74-(2-(ethoxymethyl)-4-(tritylamino)-1H-imidazo[4,5-c]quinolin-1-yl)-73,73-dimethyl-3,6,9,12,15,18,21,24,27,30,33,36,39,42,45,48,51,54,57,60,63,66,69,72-tetracosaoxatetraheptacontan-1-ol

Following the procedure described in general method M3, compound 21 was prepared from compound 20 (305 mg, 0.177 mmol) in 30 mL of a 4:1 mixture of pyridine and 1,4-dioxane and obtained as a colorless oil (199 mg, 69%).

NMR ¹H (400 MHz, δ in ppm, DMSO-d6): 1.05 to 1.31 (m, 9H); 3.33 to 3.61 (m, 100H); 4.55 (t, J=6 Hz, 1H); 4.76 (large s, 2H); 7.01 (s, 1H); 7.10 (dd, J=1 and 8 Hz, 1H); 7.16 to 7.33 (m, 11H); 7.40 (d, J=7 Hz, 6H); 8.25 (d, J=8 Hz, 1H), LCMS (M1): t_{R}=1.13, ES, m/z=1613 [M+H]⁺,

m/z=807 [M+2H]²⁺, m/z=1657 [M−H+HCOOH]⁻.

### Compound 22: 74-(2-(ethoxymethyl)-4-(tritylamino)-1H-imidazo[4,5-c]quinolin-1-yl)-73,73-dimethyl-3,6,9,12,15,18,21,24,27,30,33,36,39,42,45,48,51,54,57,60,63,66,69,72-tetracosaoxatetraheptacontyl 4-methylbenzenesulfonate

Under argon, to a solution of compound 21 (197 mg, 0.122 mmol) in anhydrous DCM (14 mL) were added DMAP (7.5 mg, 0.061 mmol) and TEA (170 μL, 1.22 mmol) followed by tosyl chloride (186 mg, 0.976 mmol.) at 0° C. The reaction mixture was allowed to warm up to RT. After completion of the reaction, the reaction mixture was diluted with DCM (50 mL), washed with water (3×10 mL) and sat. brine (2×10 mL), dried over MgSO₄, filtered and concentrated in vacuo. The crude yellow oil was purified by flash chromatography on 15 g of silica gel (gradient elution DCM/MeOH/H₂O) to give 194 mg of compound 22 as a colorless oil (90%).

NMR ¹H (400 MHz, δ in ppm, DMSO-d6): 1.06 to 1.30 (m, 9H); 2.42 (s, 3H); 3.34 to 3.78 (m, 98H); 4.11 (m, 2H); 4.76 (large s, 2H); 7.00 (s, 1H); 7.10 (d, J=8 Hz, 1H); 7.15 to 7.34 (m, 11H); 7.40 (d, J=8 Hz, 6H); 7.49 (d, J=8 Hz, 2H); 7.79 (d, J=8 Hz, 2H); 8.25 (d, J=8 Hz, 1H). LCMS (M2): t_{R}=4.83, ES, m/z=1768 [M+H]⁺, m/z=884 [M+2H]²⁺, m/z=1812 [M−H+HCOOH]⁻.

### Compound 23: 1-(74-azido-2,2-dimethyl3,6,9,12,15,18,21,24,27,30,33,36,39,42,45,48, 51,54,57,60,63,66,69,72-tetracosaoxatetraheptacontyl)-2-(ethoxymethyl)-N-trityl-1H-imidazo[4,5-c]quinolin-4-amine

Under argon, to a solution of compound 22 (189 mg, 0.106 mmol) in anhydrous DMF (10 mL) was added sodium azide (34 mg, 0.534 mmol). After stirring overnight at 80° C., the yellow reaction mixture was concentrated in vacuo and co-evaporated with toluene. The residue was taken up in a mixture of DCM (75 mL) and water (10 mL). The organic layer was washed with water (2×10 mL) and sat. brine (2×10 mL), dried over MgSO₄, filtered and concentrated in vacuo. The residue was purified by flash chromatography on 15 g of silica gel (gradient elution DCM/MeOH/H₂O) to give 143 mg of compound 23 as a colorless oil (81%).

NMR ¹H (400 MHz, δ in ppm, DMSO-d6): 1.12 to 1.27 (m, 9H); 3.34 to 3.70 (m, 100H); 4.75 (large s, 2H); 7.00 (s, 1H); 7.10 (dd, J=2 and 8 Hz, 1H); 7.16 to 7.34 (m, 11H); 7.40 (d, J=7 Hz, 6H); 8.25 (d, J=7 Hz, 1H), LCMS (M1): t_{R}=1.23, ES, m/z=1638 [M+H]⁺, m/z=819 [M+2H]²⁺, m/z=1682 [M−H+HCOOH]⁻.

### Example 19: 1-(74-azido-2,2-dimethyl-3,6,9,12,15,18,21,24,27,30,33,36,39,42,45,48,51, 54,57,60,63,66,69,72-tetracosaoxatetraheptacontyl)-2-(ethoxymethyl)-1H-imidazo[4,5-c]quinolin-4-amine

Following the procedure described in general method M5, compound 23 (141 mg, 0.086 mmol) in 1,4-dioxane (4 mL) was treated with 4N HCl in 1,4-dioxane (8 mL) to give example 19 as a colorless oil (118 mg, 98%).

NMR ¹H (400 MHz, δ in ppm, DMSO-d6): 0.89 to 1.03 (m, 9H); 3.10 to 3.48 (m, 100H); 4.55 (large s, 2H); 6.32 (m, 2H); 7.00 (t, J=8 Hz, 1H); 7.20 (t, J=8 Hz, 1H); 7.36 (t, J=8 Hz, 1H); 8.06 (d, J=8 Hz, 1H), LCMS (M1): t_{R}=0.83, ES, m/z=1396 [M+H]⁺, m/z=698.5 [M+2H]²⁺.

### Example 20a: CEACAM5_Tusamitamab-PEG24-R848 ADC

Example 20a was prepared following general method M12; mAb 1a (58 mg at 5.09 mg/mL) in 80:20 DPBS/DMA was reacted overnight with 309 μL of a solution of Example 19 in DMA (9.55 mM) then 62 μL of Example 19 were added and the reaction mixture was stirred for 4 h. It was purified and formulated by gel filtration using a Superdex™ 200 pg matrix (HiLoad® 26/60 desalting column, GE Healthcare) pre-equilibrated with DPBS. Example 20a (45 mg at 1.41 mg/mL, overall yield 88%) was finally obtained after sterile filtration as a colorless limpid solution with a DAR (HRMS) of 4.2 and a monomeric purity of 100%.

RP-HRMS: 149171 (D1); 150936 (D2); 152706 (D3); 154478 (D4); 156247 (D5); 158015 (D6); 159782 (D7); 161541 (D8).

### Example 20b: CEACAM5_Tusamitamab-PEG24-R848 ADC

mAb 1c (10.15 mL at 10.495 mg/mL) was diluted in 20 mM K₂HPO₄ pH 7.5 (10.96 mL) and reacted overnight with 192 μL of a solution of Example 19 in DMSO (50 mM). The crude reaction mixture was filtered on Steriflip® (0.22 μm, PVDF membrane, Millipore) and purified by gel filtration using Sephadex™ G25 matrix (Hiprep™ desalting column, GE Healthcare); Example 20b (106 mg at 5 mg/mL, quant.) was finally obtained as a colorless limpid solution with a DAR (HRMS) of 3.69 and a monomeric purity of 97.6%.

SEC-HRMS: 147401 (naked mAb); 149170 (D1); 150939 (D2); 152709 (D3); 154479 (D4); 156246 (D5); 158015 (D6); 159786 (D7); 161536 (D8).

### Example 20c: CEACAM5_Tusamitamab-PEG24-R848 ADC

mAb 1d (10.15 mL at 10.495 mg/mL) was diluted in 20 mM K₂HPO₄ pH 7.5 (10.9 mL) and reacted overnight with 221 μL of a solution of Example 19 in DMSO (50 mM). The crude reaction mixture was filtered on Steriflip® (0.22 μm, PVDF membrane, Millipore) and purified by gel filtration using Sephadex™ G25 matrix (Hiprep™ desalting column, GE Healthcare); Example 20c (106 mg at 5 mg/mL, quant.) was finally obtained as a colorless limpid solution with a DAR (HRMS) of 1.5 and a monomeric purity of 98.8%.

RP-HRMS: 147414 (naked mAb); 149183 (D1); 150952 (D2); 152721 (D3); 154490 (D4); 156256 (D5).

### Example 20d: CEACAM5_Tusamitamab-PEG24-R848 ADC

mAb 1e (10.15 mL at 10.495 mg/mL) was diluted in 20 mM K₂HPO₄ pH 7.5 (10.9 mL) and reacted overnight with 221 μL of a solution of Example 19 in DMSO (50 mM). The crude reaction mixture was filtered on Steriflip® (0.22 μm, PVDF membrane, Millipore) and purified by gel filtration using Sephadex™ G25 matrix (Hiprep™ desalting column, GE Healthcare); Example 20d (106 mg at 5 mg/mL, quant.) was finally obtained as a colorless limpid solution with a DAR (HRMS) of 3.6 and a monomeric purity of 97.5%.

RP-HRMS: 147406 (naked mAb); 149181 (D1); 150943 (D2); 152718 (D3); 154488 (D4); 156249 (D5); 158029 (D6); 159799 (D7).

### Example 21: EphA2_hu2H11_R35R74-PEG24-R848 ADC

Example 21 was prepared following general method M13; mAb 2 (10.5 mg at 7 mg/mL) in 80:20 DPBS/DMA was reacted overnight with 85 μL of a solution of Example 19 in DMA (10 mM). The mixture was diafiltrated on Vivaspin® (PES membrane, 50K, Sartorius) using 80:20 DPBS/DMA, then formulated bygel filtration using a Sephadex™ G25 matrix (Hitrap™ desalting column, GE Healthcare) pre-equilibrated with the final aqueous buffer A. After another diafiltration using buffer A, Example 21 (5.5 mg at 3.24 mg/mL, overall yield 52%) was finally obtained after sterile filtration as a colorless limpid solution with a DAR (HRMS) of 3.43 and a monomeric purity of 98%.

RP-HRMS: 149343 (naked mAb); 151110 (D1); 152879 (D2); 154647 (D3); 156415 (D4); 158185 (D5); 159952 (D6); 161718 (D7); 163496 (D8); 165278 (D9).

### Example 22: HER2_trastuzumab-PEG24-R848 ADC

Example 22 was prepared following general method M13; mAb 3 (11 mg at 6.14 mg/mL) in 80:20 DPBS/DMA was reacted overnight with 91 μL of a solution of Example 19 in DMA (10 mM). The mixture was diafiltrated on Vivaspin® (PES membrane, 50K, Sartorius) using 80:20 DPBS/DMA, then formulated by gel filtration using a Sephadex™ G25 matrix (Hitrap™ desalting column, GE Healthcare) pre-equilibrated with the final aqueous buffer A. After another diafiltration on Vivaspin® (PES membrane, 50K, Sartorius), Example 22 (5.85 mg at 3.55 mg/mL, overall yield 53%) was finally obtained after sterile filtration as a colorless limpid solution with a DAR (HRMS) of 3.4 and a monomeric purity of 98.3%. RP-HRMS: 148059 (naked mAb); 149836 (D1); 151605 (D2); 153374 (D3); 155143 (D4); 156909 (D5); 158674 (D6); 160442 (D7); 162224 (D8).

### Example 23: EGFR_cetuximab-PEG24-R848 ADC

Example 23 was prepared following general method M12; mAb 4 (11.4 mg at 1.6 mg/mL) in 80:20 buffer C/DMA was reacted overnight with 94 μL of a solution of Example 19 in DMA (10 mM). The mixture was diafiltrated on Vivaspin® (PES membrane, 50K, Sartorius) with 80:20 DPBS/DMA, then purified by gel filtration using a Superdex™ 200 pg matrix (HiLoad® 16/60 desalting column, GE Healthcare). After concentration on Vivaspin® (PES membrane, 50K, Sartorius) and formulation by gel filtration using a Sephadex™ G25 matrix (NAP®, GE Healthcare) pre-equilibrated with the final aqueous buffer A; Example 23 (6.7 mg at 3.7 mg/mL, overall yield 59%) was finally obtained after sterile filtration as a colorless limpid solution with a DAR (HRMS) of 4.4 (rebuilt DAR following analysis on reduced ADC) and a monomeric purity of 100%.

RP-HRMS: DAR_{LC}=0.21; 23423 (naked LC); 25192 (LC1); DAR_{HC}=1.99; 52764 (HC); 54525 (HC1); 56298 (HC2); 58065 (HC3); 59832 (HC4).

### Example 24: B7H3_enoblituzumab-PEG24-R848 ADC

Example 24 was prepared following general method M13; mAb 5 (11.3 mg at 1.36 mg/mL) in 80:20 DPBS/DMA was reacted overnight with 93 μL of a solution on Example 19 in DMA (10 mM). The mixture was diafiltrated on Vivaspin® (PES membrane, 50K, Sartorius) using 80:20 DPBS/DMA, then formulated by gel filtration using a Sephadex™ G25 matrix (Hitrap™ desalting column, GE Healthcare) pre-equilibrated with the final aqueous buffer A. After another diafiltration on Vivaspin® (PES membrane, 50K, Sartorius) using buffer A, Example 24 (4.4 mg at 2.68 mg/mL, overall yield 39%) was finally obtained after sterile filtration as a colorless limpid solution with a DAR (HRMS) of 4.2 and a monomeric purity of 99.3%.

RP-HRMS: 149788 (D1); 151554 (D2); 153321 (D3); 155089 (D4); 156859 (D5); 158625 (D6); 160389 (D7); 162174 (D8).

### Synthesis of Azido PEGn aldehydes

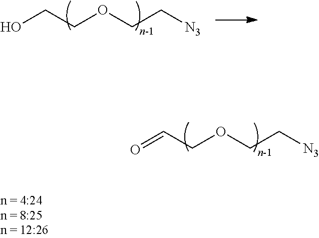

### Compound 24: 2-(2-(2-(2-azidoethoxy)ethoxy)ethoxy)acetaldehyde

Following the procedure described in general method M6, compound 24 was obtained from 2-(2-(2-(2-azidoethoxy)ethoxy)ethoxy)ethan-1-ol or PEG4-azide (200 mg, 0.912 mmol) and obtained as a colorless oil (161 mg, 81%).

NMR ¹H (400 MHz, δ in ppm, DMSO-d6): 3.40 (t, J=5 Hz, 2H); 3.50 to 3.65 (m, 10H); 4.19 (s, 2H); 9.57 (s, 1H).

### Compound 25: 23-azido-3,6,9,12,15,18,21-heptaoxatricosanal

Alternative synthesis of aldehyde using Dess-Martin reagent. Sodium bicarbonate (159 mg, 0.625 mmol) and Dess-Martin periodinane (273 mg, 0.625 mmol) were added to a solution of 2-(2-(2-(2-(2-(2-(2-(2-azidoethoxy)ethoxy)ethoxy)ethoxy)ethoxy)ethoxy)ethoxy)ethan-1-ol or PEG8-azide (250 mg, 0.625 mmol) in dry DCM (5 mL) under argon. The reaction mixture thus obtained was stirred at RT for 2 h. An aqueous solution of Na₂S₂O₃ (4 mL) was added and the organic layer was washed with sat. brine, dried over MgSO₄, filtered and concentrated in vacuo to give 300 mg of compound 25 (quant.) as a colorless oil.

### Compound 26: 35-azido-3,6,9,12,15,18,21,24,27,30,33-undecaoxapentatriacontanal

Following the procedure described in general method M6, compound 26 was obtained from 2-(2-(2-(2-(2-(2-(2-(2-(2-(2-(2-(2-azidoethoxy)ethoxy)ethoxy)ethoxy)ethoxy)ethoxy)ethoxy)ethoxy)ethoxy) ethoxy)ethoxy)-ethan-1-ol or PEG12-azide (1.69 g, 4.33 mmol) and obtained as a colorless oil (178 mg, 90%).

### Synthesis of Examples 25 and 26: azido-PEG4-3M012 and Corresponding ADC

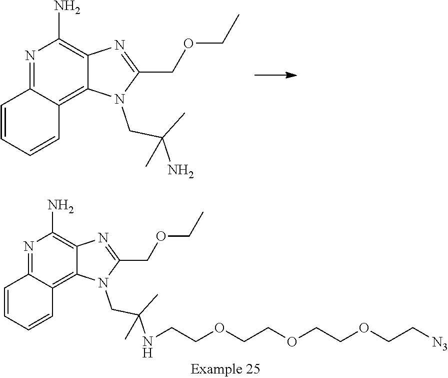 [SMILES: CCOCc1nc2c(N)nc3ccccc3c2n1CC(C)(C)N ; CCOCc1nc2c(N)nc3ccccc3c2n1CC(C)(C)NCCOCCOCCOCCN=[N+]=[N-]]

### Example 25: 1-(14-azido-2,2-dimethyl-6,9,12-trioxa-3-azatetradecyl)-2-(ethoxymethyl)-1H-imidazo[4,5-c]quinolin-4-amine

Following the procedure described in general method M7, from 3M-012 ([12244966-68-4], 46 mg, 0.148 mmol) and compound 24 (32 mg, 0.147 mmol), example 25 was obtained as a colorless oil (40 mg, 53%).

NMR ¹H (400 MHz, δ in ppm, DMSO-d6): 0.92 to 1.20 (m, 9H); 3.39 (masked m, 21H); 6.56 (s, 2H); 6.66 (large s, 2H); 7.23 (t, J=8 Hz, 1H); 7.42 (t, J=8 Hz, 1H); 7.59 (d, J=8 Hz, 1H); 8.31 (d, J=8 Hz, 1H). LCMS (M1): t_{R}=0.37, ES, m/z=515 [M+H]⁺.

### Example 26: CEACAM5_Tusamitamab-PEG4-3M-012 ADC

Example 26 was prepared following general method M12; mAb 1a (15.13 mg at 5.84 mg/mL) in 80/20 DPBS/DMA was reacted overnight with 47 μL of a solution of Example 25 in DMA (14.09 mM). The mixture was purified by gel filtration using a Sephadex™ G25 matrix (Hiprep™ 26/10 desalting columns, GE Healthcare) pre-equilibrated with DPBS. Example 26 (10.1 mg at 1 mg/mL, overall yield 66%) was finally obtained after sterile filtration as a colorless limpid solution with a DAR (HRMS) of 4 and a monomeric purity of 100%.

RP-HRMS: 148309 (D1); 149195 (D2); 150083 (D3); 150970 (D4); 151857 (D5); 152744 (D6); 153631 (D7); 154532 (D8).

### Synthesis of Examples 27 and 28: azido-PEG8-3M012 and corresponding ADC

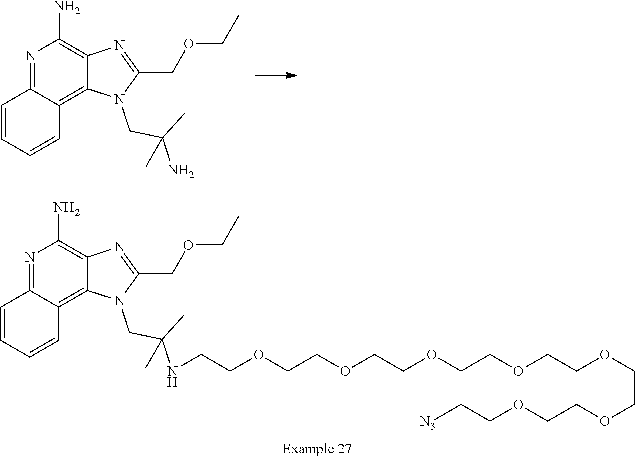 [SMILES: CCOCc1nc2c(N)nc3ccccc3c2n1CC(C)(C)N ; CCOCc1nc2c(N)nc3ccccc3c2n1CC(C)(C)NCCOCCOCCOCCOCCOCCOCCOCCN=[N+]=[N-]]

### Example 27: 1-(38-azido-2,2-dimethyl-6,9,12,15,18,21,24,27,30,33,36-undecaoxa-3-azaoctatriacontyl)-2-(ethoxymethyl)-1H-imidazo[4,5-c]quinolin-4-amine

Following the procedure described in general method M7, from 3M-012 ([12244966-68-4], 200 mg, 0.638 mmol) and compound 26 (251 mg, 0.638 mmol.), example 27 was obtained as a colorless oil (133 mg, 30%).

NMR ¹H (400 MHz, δ in ppm, DMSO-d6): 1.05 (large s, 6H); 1.14 (t, J=7 Hz, 3H); 3.33 to 3.63 (m, 37H); 4.66 (large m, 2H); 6.54 (large s, 2H); 7.23 (t, J=7 Hz, 1H); 7.41 (t, J=8 Hz, 1H); 7.59 (d, J=8 Hz, 1H); 8.31 (d, J=8 Hz, 1H), LCMS (M1): t_{R}=0.39 to 0.43, ES, m/z=691 [M+H]⁺, m/z=346 [M+2H]²⁺.

### Example 28: CEACAM5_Tusamitamab-PEG8-3M012 ADC

Example 28 was prepared following general method M12; mAb 1a (15.13 mg at 5.84 mg/mL) in 80/20 DPBS/DMA was reacted overnight with 52 μL of a solution of Example 27 in DMA (12.83 mM). The mixture was purified by gel filtration using a Sephadex™ G25 matrix (Hiprep™ 26/10 desalting columns, GE Healthcare) pre-equilibrated with DPBS. Example 28 (13.5 mg at 1.69 mg/mL, overall yield 89%) was finally obtained after sterile filtration as a colorless limpid solution with a DAR (HRMS) of 4 and a monomeric purity of 97.7%.

RP-HRMS: 148482 (D1); 149545 (D2); 150607 (D3); 151667 (D4); 152733 (D5); 153797 (D6); 154862 (D7); 155921 (D8); 156892 (D9).

### Synthesis of Examples 29 and 30: azido-PEG12-3M012 and Corresponding ADC

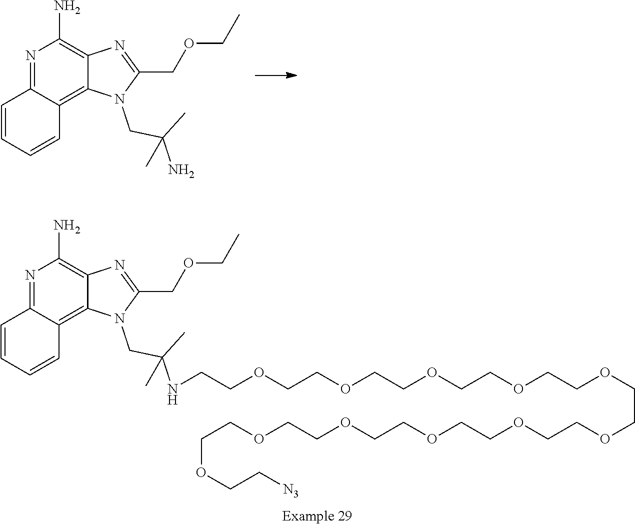 [SMILES: CCOCc1nc2c(N)nc3ccccc3c2n1CC(C)(C)N ; CCOCc1nc2c(N)nc3ccccc3c2n1CC(C)(C)NCCOCCOCCOCCOCCOCCOCCOCCOCCOCCOCCOCCN=[N+]=[N-]]

### Example 29: 1-(26-azido-2,2-dimethyl-6,9,12,15,18,21,24-heptaoxa-3-azahexacosyl)-2-(ethoxy-methyl)-1H-imidazo[4,5-c]quinolin-4-amine

Following the procedure described in general method M7, from 3M-012 ([12244966-68-4], 76 mg, 0.242 mmol) and compound 25 (138 mg, 0.242 mmol), example 29 was obtained as a colorless oil (63 mg, 30%).

NMR ¹H (500 MHz, δ in ppm, DMSO-d6): 1.05 (m, 6H); 1.14 (t, J=7 Hz, 3H); 2.59 (m, 2H); 3.35 to 3.57 (m, 49H); 3.59 (m, 2H); 4.53 to 5.06 (large m, 2H); 6.59 (large s, 2H); 7.23 (t, J=8 Hz, 1H); 7.41 (t, J=8 Hz, 1H); 7.60 (dd, J=1 and 8 Hz, 1H); 8.31 (dd, J=1 and 8 Hz, 1H), LCMS (M1): t_{R}=0.49, ES, m/z=867 [M+H]⁺, m/z=434 [M+2H]²⁺.

### Example 30: CEACAM5_Tusamitamab-PEG12-3M-012 ADC

Example 30 was prepared following general method M12; mAb 1a (15.13 mg at 5.84 mg/mL) in 80/20 DPBS/DMA was reacted overnight with 89.79 μL of a solution of Example 29 in DMA (7.38 mM). The mixture was purified by gel filtration using a Sephadex™ G25 matrix (Hiprep™ 26/10 desalting columns, GE Healthcare) pre-equilibrated with DPBS. Example 30 (12.1 mg at 1.18 mg/mL, overall yield 80%) was finally obtained after sterile filtration as a colorless limpid solution with a DAR (HRMS) of 4.15 and a monomeric purity of 99.8%.

RP-HRMS: 147420 (naked mAb); 148660 (D1); 149900 (D2); 151139 (D3); 152378 (D4); 153618 (D5); 154856 (D6); 156099 (D7); 157338 (D8); 158570 (D9).

### Synthesis of Examples 31 to 43: maleimidopropionyl-PEG24-R848 and corresponding ADCs

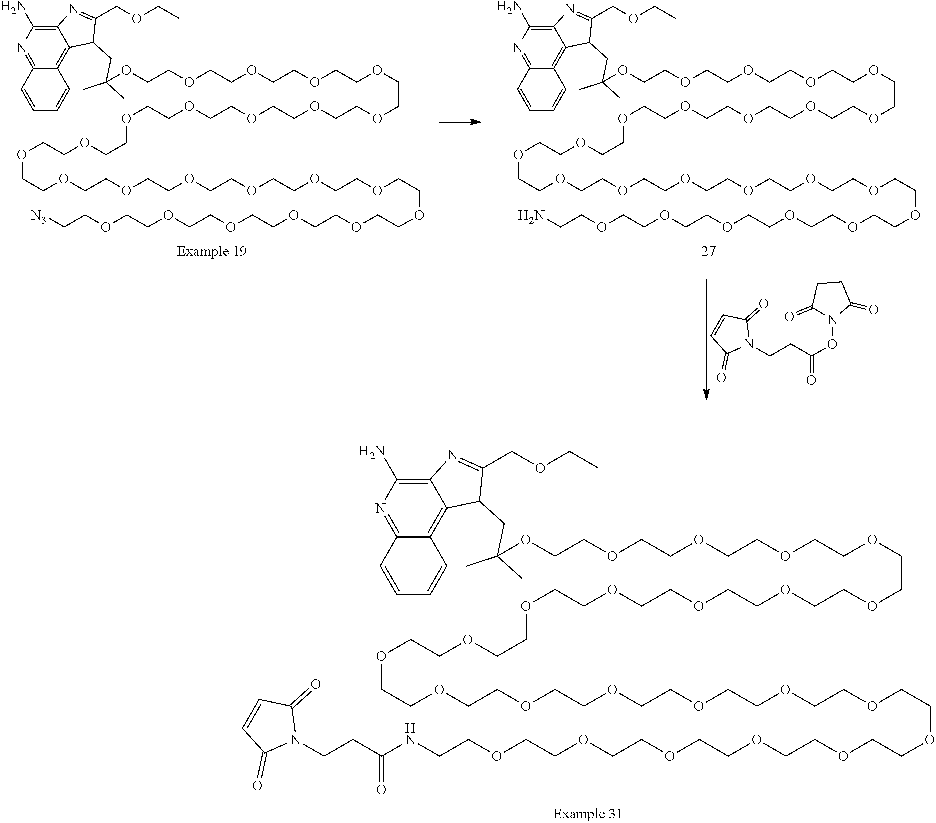 [SMILES: CCOCC1=Nc2c(N)nc3ccccc3c2C1CC(C)(C)OCCOCCOCCOCCOCCOCCOCCOCCOCCOCCOCCOCCOCCOCCOCCOCCOCCOCCOCCOCCOCCOCCOCCOCCN=[N+]=[N-] ; CCOCC1=Nc2c(N)nc3ccccc3c2C1CC(C)(C)OCCOCCOCCOCCOCCOCCOCCOCCOCCOCCOCCOCCOCCOCCOCCOCCOCCOCCOCCOCCOCCOCCOCCOCCN ; O=C(CCN1C(=O)C=CC1=O)ON1C(=O)CCC1=O ; CCOCC1=Nc2c(N)nc3ccccc3c2C1CC(C)(C)OCCOCCOCCOCCOCCOCCOCCOCCOCCOCCOCCOCCOCCOCCOCCOCCOCCOCCOCCOCCOCCOCCOCCOCCNC(=O)CCN1C(=O)C=CC1=O]

### Compound 27: 1-(74-amino-2,2-dimethyl-3,6,9,12,15,18,21,24,27,30,33,36,39,42,45,48, 51,54,57,60,63,66,69,72-tetracosaoxatetraheptacontyl)-2-(ethoxymethyl)-1H-imidazo[4,5-c]quinolin-4-amine

Following the procedure described in general method M14, compound 27 was prepared from example 19 (300 mg, 0.21 mmol) and obtained as a white foam (270 mg, 92%).

NMR ¹H (400 MHz, δ in ppm, DMSO-d6): 1.05-1.30 (m, 6H); 1.14 (t, J=7 Hz, 3H); 2.87 (t, J=5 Hz, 2H); 3.20-3.70 (m partially hidden, 96H); 4.50-5.20 (m, 4H); 6.40-6.65 (m, 2H); 7.22 (td, J=8, 1 Hz, 1H); 7.41 (td, J=8, 1 Hz, 1H); 7.59 (dd, J=8, 1 Hz, 1H); 8.28 (br d, J=8 Hz, 1H); LCMS (M1): t_{R}=1.06, ES, m/z=1414 [M−H+HCOOH]⁻.

### Example 31: N-(74-(4-amino-2-(ethoxymethyl)-1H-imidazo[4,5-c]quinolin-1-yl)-73,73-dimethyl-3,6,9,12,15,18,21,24,27,30,33,36,39,42,45,48,51,54,57,60,63,66,69,72-tetracosaoxatetraheptacontyl)-3-(2,5-dioxo-2,5-dihydro-1H-pyrrol-1-yl)propanamide formate

Following the procedure described in general method M16, example 31 was prepared from compound 27 (270 mg, 197 μmol) and succinimidyl 3-maleimidopropionate (CAS [55750-62-4], 55 mg, 197 μmol) and obtained as a white foam formiate salt (165 mg, 53%).

NMR ¹H (400 MHz, δ in ppm, DMSO-d6): 1.05-1.30 (m, 6H); 1.15 (t, J=7 Hz, 3H); 2.28-2.37 (m, 2H); 3.15 (q, J=6 Hz, 2H); 3.25-3.70 (m, 96H); 4.60-5.20 (m, 4H); 6.55 (br s, 2H); 7.01 (s, 2H); 7.23 (br t, J=7 Hz, 1H); 7.41 (br t, J=7 Hz, 1H); 7.60 (br d, J=8 Hz, 1H); 8.01 (t, J=6 Hz, 1H); 8.19 (s, 1H); 8.29 (br d, J=8 Hz, 1H); LCMS (M1): t_{R}=1.23, ES, m/z=1521 [M+H]⁺.

### Example 32: CEACAM5_Tusamitamab_E152C-PEG24-R848 ADC

Example 32 was prepared following general method 17. A solution of CEACAM5_Tusamitamab_E152C (41.6 mg at 10.4 mg/mL) in DPBS was reacted with 66 μL of a 280 mM DTT solution for 45 min at 37° C. Following gel filtration using a Sephadex™ G25 matrix (Hiprep™ 26/10 desalting column, GE Healthcare) pre-equilibrated with DPBS buffer, the reduced antibody was reacted with 69 μL of a 50 mM DHA solution for 3 hours at RT then were added DMA (2.6 mL, 20% v/v) and 34 μL of a solution of Example 31 in DMA (20 mM) and the reaction mixture was stirred overnight at RT. Following concentration (9 mL, 2.57 mg/mL) by dialfiltration (Amicon® Ultra: PES membrane, 50K, Millipore), it was purified by gel filtration using a Superdex™ 200 pg matrix (HiLoad® 26/60 desalting column, GE Healthcare) pre-equilibrated with DPBS/10% DMA and formulated by gel filtration using a Sephadex™ G25 matrix (Hitrap™ desalting, GE Healthcare) pre-equilibrated with buffer E. Example 32 (21.4 mg at 3.1 mg/mL, overall yield 49%) was finally obtained after sterile filtration as a colorless limpid solution with a DAR (HRMS) of 1.85 and a monomeric purity of 99.8%.

RP-HRMS: 148973 (D1); 150458 (D2).

### Example 33: CEACAM5_Tusamitamab_S239C-PEG24-R848 ADC

Example 33 was prepared following general method 17. A solution of CEACAM5_Tusamitamab_S239C (37 mg at 9.24 mg/mL) in DPBS was reacted with 58 μL of a 280 mM DTT solution for 45 min at 37° C. Following gel filtration using a Sephadex™ G25 matrix (Hiprep™ 26/10 desalting column, GE Healthcare) pre-equilibrated with DPBS buffer, the reduced antibody was reacted with 69 μL of a 50 mM DHA solution for 3 hours at RT then were added DMA (1.67 mL, 20% v/v) and 34 μL of a solution of Example 31 in DMA (20 mM) and the reaction mixture was stirred overnight at RT. It was then purified and formulated using Sephadex™ G25 matrix (Hitrap™ desalting, GE Healthcare) pre-equilibrated with buffer A and concentrated by diafiltration (Amicon® Ultra: PES membrane, 50K, Millipore). Example 33 (20.0 mg at 4.4 mg/mL, overall yield 54%) was finally obtained after sterile filtration as a colorless limpid solution with a DAR (HRMS) of 1.8 and a monomeric purity of 98.8%.

RP-HRMS: 147455 (naked mAb); 148976 (D1); 150499 (D2).

### Example 34: CEACAM5_Tusamitamab_K274C-PEG24-R848 ADC

Example 34 was prepared following general method 17. A solution of CEACAM5_Tusamitamab_K274C (34.7 mg at 8.68 mg/mL) in DPBS was reacted with 53 μL of a 280 mM DTT solution for 45 min at 37° C. Following gel filtration using a Sephadex™ G25 matrix (Hiprep™ 26/10 desalting column, GE Healthcare) pre-equilibrated with DPBS buffer, the reduced antibody was reacted with 66 μL of a 50 mM DHA solution for 3 hours at RT then were added DMA (1.86 mL, 20% v/v) and 33 μL of a solution of Example 31 in DMA (20 mM) and the reaction mixture was stirred overnight at RT. It was then purified and formulated using Sephadex™ G25 matrix (Hitrap™ desalting, GE Healthcare) pre-equilibrated with buffer A and concentrated by diafiltration (Amicon® Ultra: PES membrane, 50K, Millipore). Example 34 (23.0 mg at 3.06 mg/mL, overall yield 66%) was finally obtained after sterile filtration as a colorless limpid solution with a DAR (HRMS) of 2 and a monomeric purity of 99.6%.

RP-HRMS: 150412 (D2).

### Example 35: CEACAM5_Tusamitamab_K290C-PEG24-R848 ADC

Example 35 was prepared following general method 17. A solution of CEACAM5_Tusamitamab_K290C (42 mg at 10.51 mg/mL) in DPBS was reacted with 65.1 μL of a 280 mM DTT solution for 45 min at 37° C. Following gel filtration using a Sephadex™ G25 matrix (Hiprep™ 26/10 desalting column, GE Healthcare) pre-equilibrated with DPBS buffer, the reduced antibody was reacted with 71 μL of a 50 mM DHA solution for 3 hours at RT then were added DMA (1.98 mL, 20% v/v) and 41 μL of a solution of Example 31 in DMA (20 mM) and the reaction mixture was stirred overnight at RT. It was then purified and formulated using Sephadex™ G25 matrix (Hitrap™ desalting, GE Healthcare) pre-equilibrated with buffer A and concentrated by diafiltration (Amicon® Ultra: PES membrane, 50K, Millipore). Example 35 (28 mg at 4.07 mg/mL, overall yield 68%) was finally obtained after sterile filtration as a colorless limpid solution with a DAR (HRMS) of 2 and a monomeric purity of 99.7%.

RP-HRMS: 150417 (D2).

### Example 36: CEACAM5_Tusamitamab_K326C-PEG24-R848 ADC

Example 36 was prepared following general method 17. A solution of CEACAM5_Tusamitamab_K326C (42 mg at 10.47 mg/mL) in DPBS was reacted with 115 μL of a 158 mM DTT solution for 45 min at 37° C. Following gel filtration using a Sephadex™ G25 matrix (Hiprep™ 26/10 desalting column, GE Healthcare) pre-equilibrated with DPBS buffer, the reduced antibody was reacted with 55 μL of a 71 mM DHA solution for 3 hours at RT then were added DMA (1.86 mL, 20% v/v) and 60 μL of a solution of Example 31 in DMA (20 mM) and the reaction mixture was stirred overnight at RT. Following concentration by dialfiltration (Amicon® Ultra: PES membrane, 50K, Millipore), it was then purified and formulated using Sephadex™ G25 matrix (Hitrap™ desalting, GE Healthcare) pre-equilibrated with buffer A. Example 36 (19.4 mg at 2.99 mg/mL, overall yield 46%) was finally obtained after sterile filtration as a colorless limpid solution with a DAR (HRMS) of 2 and a monomeric purity of 98.6%.

RP-HRMS: 150418 (D2).

### Example 37: CEACAM5_Tusamitamab_K320C-PEG24-R848 ADC

Example 37 was prepared following general method 17. A solution of CEACAM5_Tusamitamab_K320C (43.4 mg at 9.65 mg/mL) in DPBS was reacted with 108 μL of a 180 mM DTT solution for 45 min at 37° C. Following gel filtration using a Sephadex™ G25 matrix (Hiprep™ 26/10 desalting column, GE Healthcare) pre-equilibrated with DPBS buffer, the reduced antibody was reacted with 57 μL of a 68 mM DHA solution for 3 hours at RT then were added DMA (2.2 mL, 20% v/v) and 39 μL of a solution of Example 31 in DMA (20 mM) and the reaction mixture was stirred overnight at RT. It was then purified and formulated using Sephadex™ G25 matrix (Hitrap™ desalting, GE Healthcare) pre-equilibrated with buffer A and concentrated by diafiltration (Amicon® Ultra: PES membrane, 50K, Millipore). Example 37 (27.2 mg at 3.2 mg/mL, overall yield 63%) was finally obtained after sterile filtration as a colorless limpid solution with a DAR (HRMS) of 1.92 and a monomeric purity of 99.6%.

RP-HRMS: 149003 (D1); 150533 (D2).

### Example 38: CEACAM5_Tusamitamab_K340C-PEG24-R848 ADC

Example 38 was prepared following general method 17. A solution of CEACAM5_Tusamitamab_K340C (40.6 mg at 9.67 mg/mL) in DPBS was reacted with 63 μL of a 280 mM DTT solution for 45 min at 37° C. Following gel filtration using a Sephadex™ G25 matrix (Hiprep™ 26/10 desalting column, GE Healthcare) pre-equilibrated with DPBS buffer, the reduced antibody was reacted with 65 μL of a 50 mM DHA solution for 3 hours at RT then were added DMA (1.75 mL, 20% v/v) and 32.4 μL of a solution of Example 31 in DMA (20 mM) and the reaction mixture was stirred overnight at RT. It was then purified and formulated using Sephadex™ G25 matrix (Hitrap™ desalting, GE Healthcare) pre-equilibrated with buffer A and concentrated by diafiltration (Amicon® Ultra: PES membrane, 50K, Millipore). Example 38 (28.7 mg at 4.29 mg/mL, overall yield 71%) was finally obtained after sterile filtration as a colorless limpid solution with a DAR (HRMS) of 2 and a monomeric purity of 97.1%.

RP-HRMS: 150413 (D2).

### Example 39: CEACAM5_Tusamitamab_S375C-PEG24-R848 ADC

Example 39 was prepared following general method 17. A solution of CEACAM5_Tusamitamab_S375C (40 mg at 9.96 mg/mL) in DPBS was reacted with 63 μL of a 280 mM DTT solution for 45 min at 37° C. Following gel filtration using a Sephadex™ G25 matrix (Hiprep™ 26/10 desalting column, GE Healthcare) pre-equilibrated with DPBS buffer, the reduced antibody was reacted with 74 μL of a 50 mM DHA solution for 3 hours at RT then were added DMA (1.73 mL, 20% v/v) and 37 μL of a solution of Example 31 in DMA (20 mM) and the reaction mixture was stirred overnight at RT. It was then purified by gel filtration using a Superdex 200 pg matrix (HiLoad® 16/60 or 26/60 desalting column, GE Healthcare) pre-equilibrated in DPBS/10% DMA (v/v), concentrated by diafiltration (Amicon® Ultra: PES membrane, 50K, Millipore) and formulated using Sephadex™ G25 matrix (Hitrap™ desalting, GE Healthcare) pre-equilibrated with buffer E. Example 39 (25 mg at 3 mg/mL, overall yield 63%) was finally obtained after sterile filtration as a colorless limpid solution with a DAR (HRMS) of 2 and a monomeric purity of 99.8%.

RP-HRMS: 150540 (D2).

### Example 40: CEACAM5_Tusamitamab_N361C-PEG24-R848 ADC

Example 40 was prepared following general method 17. A solution of CEACAM5_Tusamitamab_N361C (60 mg at 10.77 mg/mL) in DPBS was reacted with 150 μL of a 100 mM DTT solution for 45 min at 37° C. Following gel filtration using a Sephadex™ G25 matrix (Hiprep™ 26/10 desalting column, GE Healthcare) pre-equilibrated with DPBS buffer, the reduced antibody was reacted with 152 μL of a 25 mM DHA solution for 3 hours at RT then were added DMA (3.6 mL, 20% v/v) and 110 μL of a solution of Example 31 in DMA (20 mM) and the reaction mixture was stirred overnight at RT. It was then concentrated to 7 mL (˜8 mg/mL) by diafiltration with ultrafiltration (Amicon® Ultra: PES membrane, 50K, Millipore) and formulated using Sephadex™ G25 matrix (Hitrap™ desalting GE Healthcare) pre-equilibrated with buffer A. Example 40 (43 mg at 4.67 mg/mL, overall yield 73%) was finally obtained after sterile filtration as a colorless limpid solution with a DAR (HRMS) of 2 and a monomeric purity of 97.9%.

RP-HRMS: 150446 (D2).

### Example 41: CEACAM5_Tusamitamab_K414C-PEG24-R848 ADC

Example 41 was prepared following general method 17. A solution of CEACAM5_Tusamitamab_K414C (49.2 mg at 9.84 mg/mL) in DPBS was reacted with 142 μL of a 100 mM DTT solution for 45 min at 37° C. Following gel filtration using a Sephadex™ G25 matrix (Hiprep™ 26/10 desalting column, GE Healthcare) pre-equilibrated with DPBS buffer, the reduced antibody was reacted with 94 μL of a 25 mM DHA solution for 3 hours at RT then were added DMA (4.1 mL, 20% v/v) and 250 μL of a solution of Example 31 in DMA (20 mM) and the reaction mixture was stirred overnight at RT. It was then concentrated to 9 mL (6.06 mg/mL) by diafiltration with ultrafiltration (Amicon® Ultra: PES membrane, 50K, Millipore) and formulated using Sephadex™ G25 matrix (Hitrap™ desalting GE Healthcare) pre-equilibrated with buffer A. Example 41 (32.8 mg at 3.64 mg/mL, overall yield 66.6%) was finally obtained after sterile filtration as a colorless limpid solution with a DAR (HRMS) of 2 and a monomeric purity of 97.7%.

RP-HRMS: 150419 (D2).

### Example 42: CEACAM5_Tusamitamab_V422C-PEG24-R848 ADC

Example 42 was prepared following general method 17. A solution of CEACAM5_Tusamitamab_V422C (51.35 mg at 10.27 mg/mL) in DPBS was reacted with 140 μL of a 100 mM DTT solution for 45 min at 37° C. Following gel filtration using a Sephadex™ G25 matrix (Hiprep™ 26/10 desalting column, GE Healthcare) pre-equilibrated with DPBS buffer, the reduced antibody was reacted with 200 μL of a 25 mM DHA solution for 3 hours at RT then were added DMA (3.6 mL, 20% v/v) and 200 μL of a solution of Example 31 in DMA (20 mM) and the reaction mixture was stirred overnight at RT. It was then concentrated by diafiltration with ultrafiltration (Amicon® Ultra: PES membrane, 50K, Millipore) and formulated using Sephadex™ G25 matrix (Hitrap™ desalting GE Healthcare) pre-equilibrated with buffer A. Example 42 (36.45 mg at 5.36 mg/mL, overall yield 71%) was finally obtained after sterile filtration as a colorless limpid solution with a DAR (HRMS) of 2 and a monomeric purity of 99%.

RP-HRMS: 150478 (D2).

### Example 43: CEACAM5_Tusamitamab_E152C_S375C-PEG24-R848 ADC

Example 43 was prepared following general method 17. A solution of CEACAM5_Tusamitamab_E152C_S375C (34.3 mg at 11.42 mg/mL) in DPBS was reacted with 66.2 μL of a 280 mM DTT solution for 45 min at 37° C. Following gel filtration using a Sephadex™ G25 matrix (Hiprep™ 26/10 desalting column, GE Healthcare) pre-equilibrated with DPBS buffer, the reduced antibody was reacted with 118 μL of a 50 mM DHA solution for 3 hours at RT then were added DMA (1.73 mL, 20% v/v) and 37 μL of a solution of Example 31 in DMA (20 mM) and the reaction mixture was stirred overnight at RT. It was then purified by gel filtration using a Superdex 200 pg matrix (HiLoad® 16/60 or 26/60 desalting column, GE Healthcare) pre-equilibrated in DPBS/10% DMA (v/v), concentrated by diafiltration (Amicon® Ultra: PES membrane, 50K, Millipore) and formulated using Sephadex™ G25 matrix (Hitrap™ desalting, GE Healthcare) pre-equilibrated with buffer E. Example 43 (12.2 mg at 2.41 mg/mL, overall yield 46%) was finally obtained after sterile filtration as a colorless limpid solution with a DAR (HRMS) of 3.75 and a monomeric purity of 100%.

RP-HRMS: 151984 (D3); 153536 (D4).

### Synthesis of Examples 44 to 47: monomethyl- and dimethyl-maleimidopropionyl-PEG24-R848 and Corresponding ADCs

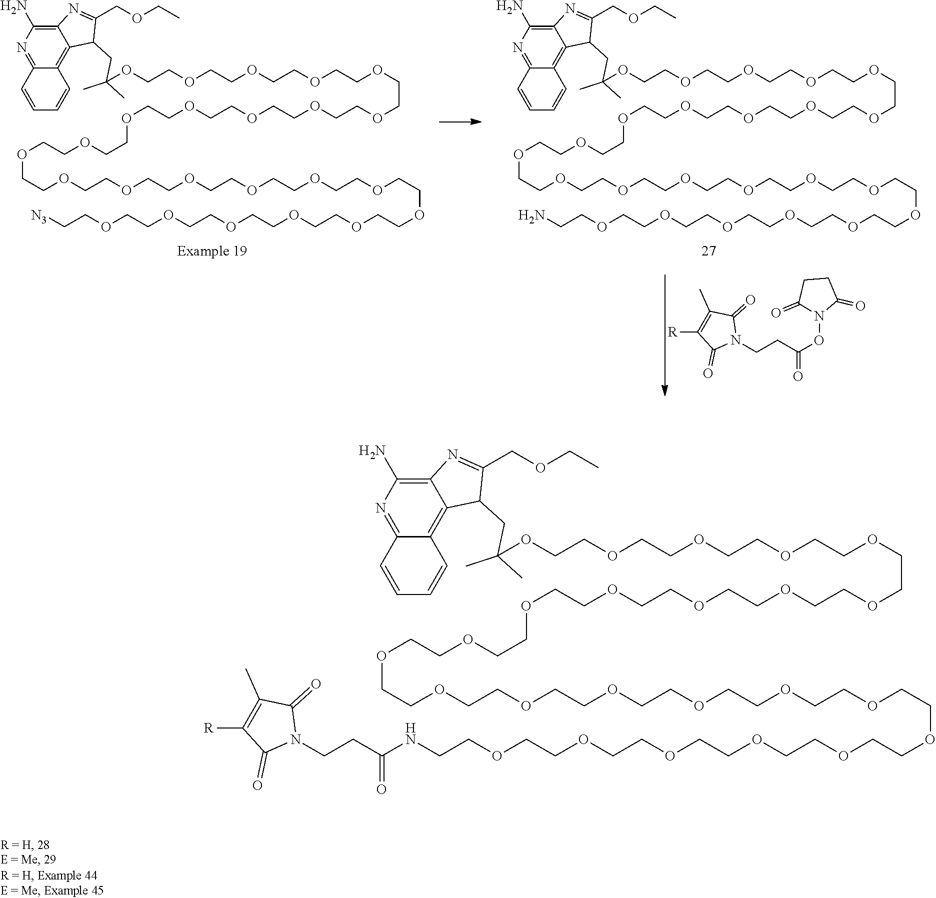

### Compound 28: (2,5-dioxopyrrolidin-1-yl) 3-(3-methyl-2,5-dioxo-pyrrol-1-yl)propanoate

Following the procedure described in general method M15, compound 28 was prepared from citraconic anhydride (350 mg, 3.13 mmol) and obtained as a white powder (74 mg, 13%).

NMR ¹H (400 MHz, δ in ppm, DMSO-d6): 1.9 (s, 6H); 2.5 (t, 2H); 3.6 (t, 2H); 12.4 (s, 1H); LCMS (M1): t_{R}=0.96, ES, m/z=281 [M+H]⁺.

### Compound 29: (2,5-dioxopyrrolidin-1-yl) 3-(3,4-dimethyl-2,5-dioxo-pyrrol-1-yl)propanoate

Following the procedure described in general method M15, compound 29 was prepared from 2,3-Dimethylmaleic anhydride (255 mg, 2.02 mmol) and obtained as a white powder (133 mg, 27%).

NMR ¹H (400 MHz, δ in ppm, DMSO-d6): 2.8 (s, 4H); 3.5 (t, 2H); 3.75 (t, 2H); 7 (d, 2H); LCMS (M1): t_{R}=1.11, ES, m/z=295 [M+H]⁺.

### Example 44: N-[2-[2-[2-[2-[2-[2-[2-[2-[2-[2-[2-[2-[2-[2-[2-[2-[2-[2-[2-[2-[2-[2-[2-[2-[2-[4-amino-2-(ethoxymethyl)imidazo[4,5-c]quinolin-1-yl]-1,1-dimethyl-ethoxy]ethoxy]ethoxy]ethoxy]ethoxy]ethoxy]ethoxy]ethoxy]ethoxy]ethoxy]ethoxy]eth oxy]ethoxy]ethoxy]ethoxy]ethoxy]ethoxy]ethoxy]ethoxy]ethoxy]ethoxy]ethoxy]ethoxy]ethoxy]ethyl]-3-(3-methyl-2,5-dioxo-pyrrol-1-yl)propanamide

Following the procedure described in general method M16, Example 44 was prepared from compound 27 (91.7 mg, 65.2 μmol) and compound 28 (18.8 mg, 65.2 μmol) and obtained as a colorless oil and a trifluoroacetate salt (80 mg, 53%).

NMR ¹H (400 MHz, δ in ppm, DMSO-d6): 1.05-1.35 (m, 6H); 1.16 (t, J=7 Hz, 3H); 1.98 (d, J=2 Hz, 3H); 2.32 (t, J=7 Hz, 2H); 3.15 (q, J=6 Hz, 2H); 3.19-3.70 (m, 98H); 4.40-5.40 (m, 4H); 6.60 (m, 1H); 7.52 (t, J=8 Hz, 1H); 7.69 (t, J=8 Hz, 1H); 7.80 (d, J=8 Hz, 1H); 7.97 (t, J=5 Hz, 1H); 8.20-9.50 (m, 2H); 8.53 (d, J=8 Hz, 1H); 13.30 (br s, 1H); LCMS (M1): t_{R}=1.28, ES, m/z=512 [M+3H]³⁺.

### Example 45: N-[2-[2-[2-[2-[2-[2-[2-[2-[2-[2-[2-[2-[2-[2-[2-[2-[2-[2-[2-[2-[2-[2-[2-[2-[2-[4-amino-2-(ethoxymethyl)imidazo[4,5-c]quinolin-1-yl]-1,1-dimethyl-ethoxy]ethoxy]ethoxy]ethoxy]ethoxy]ethoxy]ethoxy]ethoxy]ethoxy]ethoxy]ethoxy]eth oxy]ethoxy]ethoxy]ethoxy]ethoxy]ethoxy]ethoxy]ethoxy]ethoxy]ethoxy]ethoxy]ethoxy]ethoxy]ethyl]-3-(3,4-dimethyl-2,5-dioxo-pyrrol-1-yl)propanamide

Following the procedure described in general method M16, Example 45 was prepared from compound 27 (84.6 mg, 61.7 μmol) and compound 29 (18.2 mg, 61.72 μmol) and obtained as a colorless oil and as a trifluoroacetate salt (82 mg, 80%).

NMR ¹H (400 MHz, δ in ppm, DMSO-d6): 1.08-1.30 (m, 6H); 1.17 (t, J=7 Hz, 3H); 1.88 (s, 6H); 2.28-2.36 (m, 2H); 3.15 (q, J=6 Hz, 2H); 3.18-3.70 (m, 98H); 4.40-5.30 (m, 4H); 7.52 (t, J=8 Hz, 1H); 7.69 (t, J=8 Hz, 1H); 7.80 (d, J=8 Hz, 1H); 7.96 (br t, J=5 Hz, 1H); 8.20-9.40 (m, 2H); 8.53 (d, J=8 Hz, 1H); 13.31 (br s, 1H); LCMS (M1): t_{R}=1.31, ES, m/z=1549 [M+H]⁺.

### Example 46: CEACAM5_Tusamitamab_K290C-Example 44 ADC

Example 46 was prepared following general method 17. A solution of CEACAM5_Tusamitamab_K290C (26 mg at 10.4 mg/mL) in DPBS was reacted with 25.5 μL of a 280 mM DTT solution for 45 min at 37° C. Following gel filtration using a Sephadex™ G25 matrix (PD10™ desalting column, GE Healthcare) pre-equilibrated with DPBS buffer, the reduced antibody was reacted with 36 μL of a 50 mM DHA solution for 3 h at RT then were added DMA (453 μL, 10% v/v) and 25.5 μL of a solution of Example 44 in DMA (20 mM) and the reaction mixture was stirred overnight at RT. It was then concentrated by diafiltration with ultrafiltration (Amicon® Ultra: PES membrane, 50K, Millipore), purified by gel filtration using a Superdex 200 pg matrix (HiLoad® 16/60, GE Healthcare) pre-equilibrated in DPBS/10% DMA (v/v), and formulated using Sephadex™ G25 matrix (PD10™ desalting GE Healthcare) pre-equilibrated with buffer F. Example 46 (7.3 mg at 2.42 mg/mL, overall yield 28%) was finally obtained after sterile filtration as a colorless limpid solution with a DAR (HRMS) of 2 and a monomeric purity of 99%.

RP-HRMS: 150439 (D2).

### Example 47: CEACAM5_Tusamitamab_K274C-Example 44 ADC

Example 47 was prepared following general method 17. A solution of CEACAM5_Tusamitamab_K274C (11.5 mg at 7.58 mg/mL) in DPBS was reacted with 17.9 μL of a 280 mM DTT solution for 45 min at 37° C. Following gel filtration using a Sephadex™ G25 matrix (PD10™ desalting column, GE Healthcare) pre-equilibrated with DPBS buffer, the reduced antibody was reacted with 20 μL of a 50 mM DHA solution for 3 h at RT then were added DMA (335 μL, 10% v/v) and 32.3 μL of a solution of Example 44 in DMA (20 mM) and the reaction mixture was stirred overnight at RT. It was then concentrated by diafiltration with ultrafiltration (Amicon® Ultra: PES membrane, 50K, Millipore), purified by gel filtration using a Superdex 200 pg matrix (HiLoad® 16/60, GE Healthcare) pre-equilibrated in DPBS/10% DMA (v/v), and formulated using Sephadex™ G25 matrix (PD10™ desalting GE Healthcare) pre-equilibrated with buffer F. Example 47 (7.7 mg at 0.96 mg/mL, overall yield 67%) was finally obtained after sterile filtration as a colorless limpid solution with a DAR (HRMS) of 1.57 and a monomeric purity of 97.6%.

RP-HRMS: 150438 (D2).

### Synthesis of Examples 48 and 49: PODS-PEG24-R848 and Corresponding ADC

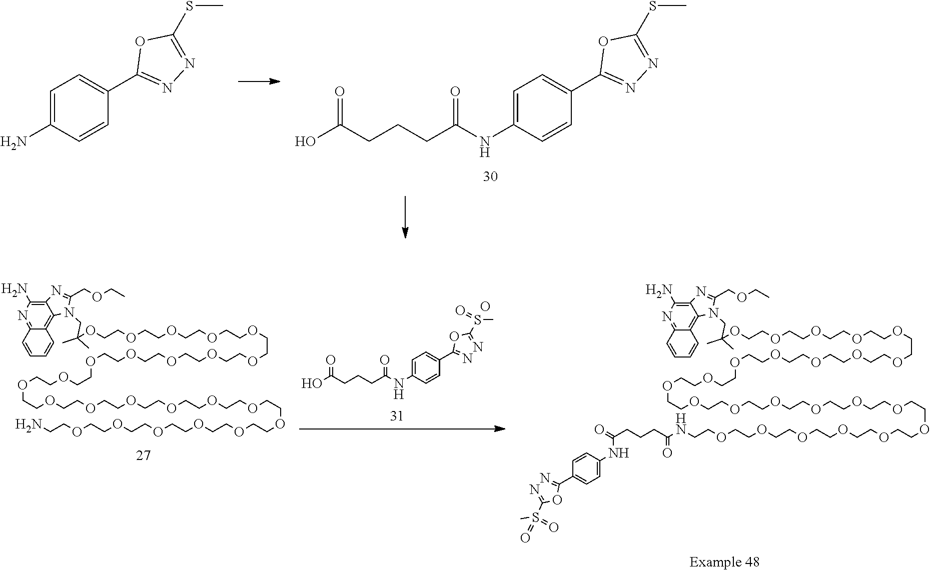 [SMILES: CSc1nnc(-c2ccc(N)cc2)o1 ; CSc1nnc(-c2ccc(NC(=O)CCCC(=O)O)cc2)o1 ; CCOCc1nc2c(N)nc3ccccc3c2n1CC(C)(C)OCCOCCOCCOCCOCCOCCOCCOCCOCCOCCOCCOCCOCCOCCOCCOCCOCCOCCOCCOCCOCCOCCOCCOCCN ; CS(=O)(=O)c1nnc(-c2ccc(NC(=O)CCCC(=O)O)cc2)o1 ; CCOCc1nc2c(N)nc3ccccc3c2n1CC(C)(C)OCCOCCOCCOCCOCCOCCOCCOCCOCCOCCOCCOCCOCCOCCOCCOCCOCCOCCOCCOCCOCCOCCOCCOCCNC(=O)CCCC(=O)Nc1ccc(-c2nnc(S(C)(=O)=O)o2)cc1]

### Compound 30: 5-[4-(5-methylsulfanyl-1,3,4-oxadiazol-2-yl)anilino]-5-oxo-pentanoic acid

To a solution of 4-(5-methylsulfanyl-1,3,4-oxadiazol-2-yl)aniline (70 mg, 338 μmol), prepared as described in Bioconjugate Chemistry 2018, 29 (4), 1364-1372, in DCM (7 mL) were added glutaric anhydride (46 mg, 405 μmol) in DCM (1 mL) and THF (a few drops). The mixture was stirred at RT for 48 h then DIEA (6 μL, 338 μmol) was added. After stirring for 1 h at RT, the reaction mixture was concentrated under reduced pressure. The residue was diluted with DCM, washed with 1M hydrochloric acid solution and concentrated under reduced pressure. Compound 30 (96 mg, 89% yield) was obtained as a pale yellow solid.

NMR ¹H (400 MHz, δ in ppm, DMSO-d6): 1.82 (quin, J=7.32 Hz, 2H); 2.28 (t, J=7.36 Hz, 2H); 2.40 (t, J=7.40 Hz, 2H); 2.76 (s, 3H); 7.80 (d, J=8.84 Hz, 2H); 7.87 (d, J=8.80 Hz, 2H); 10.29 (s, 1H); 12.11 (s, 1H); LCMS (M5): t_{R} (min)=1.26, ES, m/z=322 [M+H+]⁺.

### Compound 31: 5-[4-(5-methylsulfonyl-1,3,4-oxadiazol-2-yl)anilino]-5-oxo-pentanoic acid

To a solution of compound 30 (41 mg, 0.128 mmol) in DCM (3 mL) and DMF (0.5 mL) was added mCPBA (a total of 243 mg, 1.41 mmol) in solution in DCM (4 mL) in 4 times over 24 h. The reaction mixture was stirred at RT for 48 h and concentrated under reduced pressure. The residue was taken up in EtOAc, filtrated and air dried. Compound 31 (23 mg, 51% yield) was obtained as a pale yellow solid.

NMR ¹H (400 MHz, δ in ppm, DMSO-d6): 1.82 (quin, J=7.20 Hz, 2H); 2.29 (t, J=7.32 Hz, 2H); 2.42 (t, J=7.40 Hz, 2H); 3.70 (s, 3H); 7.86 (d, J=8.72 Hz, 2H); 8.04 (d, J=8.72 Hz, 2H); 10.36 (s, 1H); 12.11 (s, 1H); LCMS (M3): t_{R} (min)=1.59, ES, m/z=354 [M+H+]⁺.

### Example 48: N-[2-[2-[2-[2-[2-[2-[2-[2-[2-[2-[2-[2-[2-[2-[2-[2-[2-[2-[2-[2-[2-[2-[2-[2-[2-[4-amino-2-(ethoxymethyl)imidazo[4,5-c]quinolin-1-yl]-1,1-dimethyl-ethoxy]ethoxy]ethoxy]ethoxy]ethoxy]ethoxy]ethoxy]ethoxy]ethoxy]ethoxy]ethoxy]eth oxy]ethoxy]ethoxy]ethoxy]ethoxy]ethoxy]ethoxy]ethoxy]ethoxy]ethoxy]ethoxy]ethoxy]ethoxy]ethyl]-N′-[4-(5-methylsulfonyl-1,3,4-oxadiazol-2-yl)phenyl]pentanediamide

To a solution of compound 31 (11.4 mg, 32.3 μmol) in THF (2 mL) were added EDC (5.2 μL, 29.4 μmol) and HOBT (3.97 mg, 29.4 μmol) in THF (1 mL). The resulting solution was stirred at RT for 30 min before addition of compound 27 (40.3 mg, 29.4 μmol) in THF (2.5 mL) and DIEA (5.1 μL, 29.4 μmol). The reaction mixture was stirred at RT for 18 h then concentrated under reduced pressure. The residue was diluted with DCM, washed with 0.1M hydrochloric acid solution then with a saturated sodium bicarbonate solution, dried over MgSO₄ and concentrated under reduced pressure. The residue was purified by flash chromatography on silica gel (4 g Interchim column) eluted with DCM//DCM/MeOH/H₂O (9/1/0.1) from to 1//0 to 0//1 to provide Example 48 as a pale yellow oil (20 mg, 40%).

NMR ¹H (400 MHz, δ in ppm, DMSO-d6): 1.82 (quint, J=7.44 Hz, 2H); 2.15 (t, J=7.36 Hz, 2H); 2.37 (t, J=7.40 Hz, 2H); 3.34-3.60 (m, 98H); 3.70 (s, 3H); 6.58 (s, 2H) 7.86 (d, J=8.80 Hz, 2H); 7.89 (t, J=5.68 Hz, 1H); 8.04 (d, J=8.80 Hz, 2H); 10.33 (s, 1H). LCMS (M3): t_{R} (min)=2.04, ES, m/z=854 [M+2H+]²⁺.

### Example 49: CEACAM5_Tusamitamab_K274C-Example 48 ADC

Example 49 was prepared following general method 17. A solution of CEACAM5_Tusamitamab_K274C (34.7 mg at 8.68 mg/mL) in DPBS was reacted with 59 μL of a 272 mM DTT solution for 45 min at 37° C. Following gel filtration using a Sephadex™ G25 matrix (PD10™ desalting column, GE Healthcare) pre-equilibrated with DPBS buffer, the reduced antibody was reacted with 69 μL of a 50 mM DHA solution for 3 h at RT; to 2.3 mL of this crude reaction mixture at 4.79 mg/mL were then added DMA (515 μL) and 60 μL of a solution of Example 48 in DMA (5 mM) and the reaction mixture was stirred overnight at RT. It was then purified by gel filtration using a Superdex 200 pg matrix (HiLoad® 16/60, GE Healthcare) pre-equilibrated in DPBS/10% DMA (v/v) and formulated using Sephadex™ G25 matrix (NAP10™ desalting GE Healthcare) pre-equilibrated with buffer F. Example 49 (6.7 mg at 4.47 mg/mL, overall yield 61%) was finally obtained after sterile filtration as a colorless limpid solution with a DAR (HRMS) of 1.96 and a monomeric purity of 99.6%.

RP-HRMS: 148962 (D1), 150616 (D2), 152240 (D3).

### Synthesis of Examples 50 and 51: Iodoacetamido-PEG24-R848 and Corresponding ADC

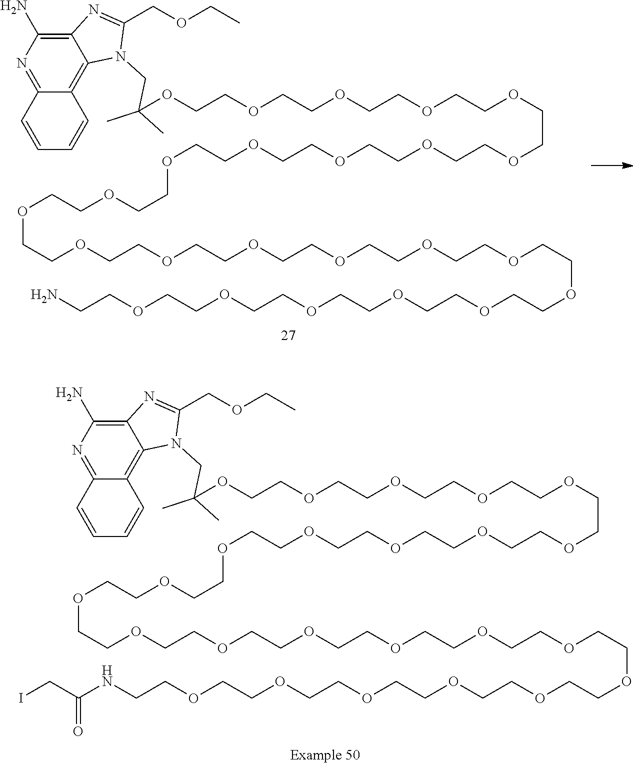 [SMILES: CCOCc1nc2c(N)nc3ccccc3c2n1CC(C)(C)OCCOCCOCCOCCOCCOCCOCCOCCOCCOCCOCCOCCOCCOCCOCCOCCOCCOCCOCCOCCOCCOCCOCCOCCN ; CCOCc1nc2c(N)nc3ccccc3c2n1CC(C)(C)OCCOCCOCCOCCOCCOCCOCCOCCOCCOCCOCCOCCOCCOCCOCCOCCOCCOCCOCCOCCOCCOCCOCCOCCNC(=O)CI]

### Example 50: N-[2-[2-[2-[2-[2-[2-[2-[2-[2-[2-[2-[2-[2-[2-[2-[2-[2-[2-[2-[2-[2-[2-[2-[2-[2-[4-amino-2-(ethoxymethyl)imidazo[4,5-c]quinolin-1-yl]-1,1-dimethyl-ethoxy]ethoxy]ethoxy]ethoxy]ethoxy]ethoxy]ethoxy]ethoxy]ethoxy]ethoxy]ethoxy]eth oxy]ethoxy]ethoxy]ethoxy]ethoxy]ethoxy]ethoxy]ethoxy]ethoxy]ethoxy]ethoxy]ethoxy]ethoxy]ethyl]-2-iodo-acetamide

To a solution of compound 27 (20 mg, 14.6 μmol) in DMF (1 mL) were added DIPEA (3 μL, 17.5 μmol) and iodoacetic anhydride (6.4 mg, 17.5 μmol) in DCM (0.5 mL). The reaction mixture was stirred at RT for 2 h then loaded onto a Sephadex LH20 column (8 g of Sephadex LH-20m resin, previously washed with EtOAc). The column was eluted with EtOAc (3 mL/min), the compound containing fractions were combined, and concentrated. The crude product was further purified by chromatography using Macherey Nagel C18 cartridge (25 g) and eluted with CH₃CN//H₂O/TFA (99.9/0.1) from to 5//95 to 90//10 to provide Example 50 was obtained as a colorless oil (13 mg, 60%).

NMR ¹H (400 MHz, δ in ppm, DMSO-d6): 6.54 (s, 2H); 3.34 to 3.60 (m, 98H); 3.19 (q, J=5.56 Hz, 2H); 3.65 (s, 2H); LCMS (M3): t_{R} (min)=1.97, ES, m/z=770 [M+2H⁺]²⁺.

### Example 51: CEACAM5_Tusamitamab_K274C-Example 50 ADC

Example 51 was prepared following general method 17. A solution of CEACAM5_Tusamitamab_K274C (34.7 mg at 8.68 mg/mL) in DPBS was reacted with 59 μL of a 272 mM DTT solution for 45 min at 37° C. Following gel filtration using a Sephadex™ G25 matrix (PD10™ desalting column, GE Healthcare) pre-equilibrated with DPBS buffer, the reduced antibody was reacted with 69 μL of a 50 mM DHA solution for 3 h at RT; to 2.3 mL of this crude reaction mixture at 4.79 mg/mL were then added DMA (560 μL), 1N HEPES (57 μL) and 15 μL of a solution of Example 50 in DMA (20 mM) and the reaction mixture was stirred overnight at RT. It was then purified by gel filtration using a Superdex 200 pg matrix (HiLoad® 16/60, GE Healthcare) pre-equilibrated in DPBS/10% DMA (v/v) and formulated using Sephadex™ G25 matrix (NAP10™ desalting GE Healthcare) pre-equilibrated with buffer F. Example 49 (5.7 mg at 3.79 mg/mL, overall yield 52%) was finally obtained after sterile filtration as a colorless limpid solution with a DAR (HRMS) of 1.79 and a monomeric purity of 99.6%.

RP-HRMS: 148745 (D1), 150187 (D2).

### Synthesis of Examples 52 and 53: Phenylacrylamide-PEG24-R848 and Corresponding ADC

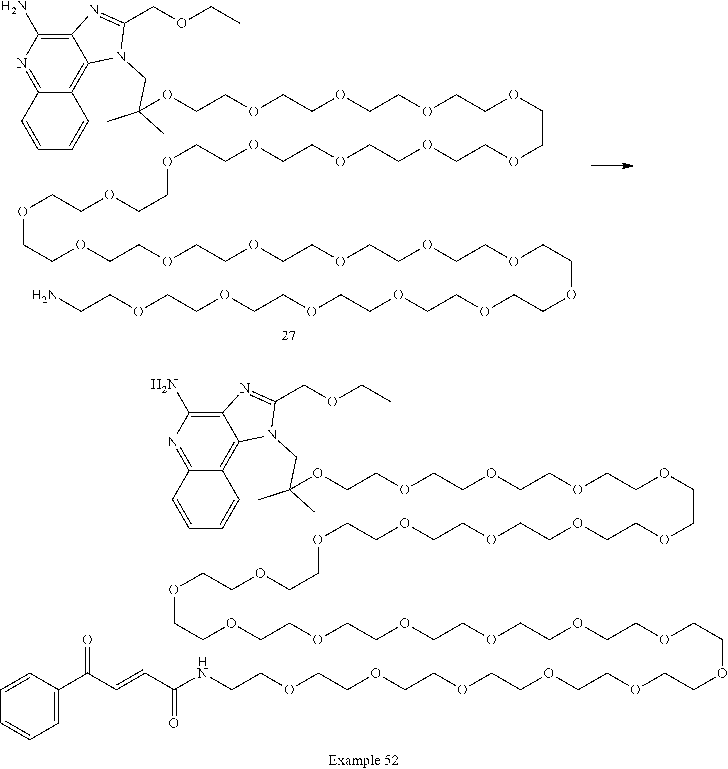 [SMILES: CCOCc1nc2c(N)nc3ccccc3c2n1CC(C)(C)OCCOCCOCCOCCOCCOCCOCCOCCOCCOCCOCCOCCOCCOCCOCCOCCOCCOCCOCCOCCOCCOCCOCCOCCN ; CCOCc1nc2c(N)nc3ccccc3c2n1CC(C)(C)OCCOCCOCCOCCOCCOCCOCCOCCOCCOCCOCCOCCOCCOCCOCCOCCOCCOCCOCCOCCOCCOCCOCCOCCNC(=O)/C=C/C(=O)c1ccccc1]

### Example 52: (E)-N-[2-[2-[2-[2-[2-[2-[2-[2-[2-[2-[2-[2-[2-[2-[2-[2-[2-[2-[2-[2-[2-[2-[2-[2-[2-[4-amino-2-(ethoxymethyl)imidazo[4,5-c]quinolin-1-yl]-1,1-dimethyl-ethoxy]ethoxy]ethoxy]ethoxy]ethoxy]ethoxy]ethoxy]ethoxy]ethoxy]ethoxy]ethoxy]eth oxy]ethoxy]ethoxy]ethoxy]ethoxy]ethoxy]ethoxy]ethoxy]ethoxy]ethoxy]ethoxy]ethoxy]ethoxy]ethyl]-4-oxo-4-phenyl-but-2-enamide

To (E)-4-oxo-4-phenyl-but-2-enoic acid (14.5 mg, 81 μmol) were added, at −10° C. under Ar, DMF (2 mL), isobutyl chloroformate (10.7 μL, 81 μmol) and 4-methylmorpholine (6.5 μL, 59 μmol). The reaction mixture was stirred at −10° C. for 10 min and let warmed to RT for 15 min to provide the mixed anhydride. To a solution of compound 27 (20 mg, 14.6 μmol) in DMF (2 mL) was added 4-methylmorpholine (2.4 μL, 22 μmol) and the solution was stirred at RT for 5 min under Ar before addition, at −10° C., of 575 μL of the solution of mixed anhydride. The reaction mixture was stirred at −10° C. for 15 min then at RT for 30 min and concentrated under pressure. The crude product was purified by flash chromatography on diol silica gel (4 g Macherey-Nagel) eluted with DCM/MeOH from 100/0 to 95/5 to provide Example 52 as a colorless oil (14.4 mg, 65%).

NMR ¹H (400 MHz, δ in ppm, DMSO-d6): 6.52 (s, 2H); 3.34 to 3.60 (m, 98H); 8.68 (t, J=5.36 Hz, 1H); 8.01 (d, J=7.20 Hz, 2H); 7.59 (d, J=8.00 Hz, 2H); 7.70 (t, J=7.40 Hz, 2H); LCMS (M4): t_{R} (min)=1.47, ES, m/z=1530 [M+H⁺]⁺.

### Example 53: CEACAM5_Tusamitamab_K274C-Example 52 ADC

Example 53 was prepared following general method 17. A solution of CEACAM5_Tusamitamab_K274C (34.7 mg at 8.68 mg/mL) in DPBS was reacted with 59 μL of a 272 mM DTT solution for 45 min at 37° C. Following gel filtration using a Sephadex™ G25 matrix (PD10™ desalting column, GE Healthcare) pre-equilibrated with DPBS buffer, the reduced antibody was reacted with 69 μL of a 50 mM DHA solution for 3 h at RT; to 2.3 mL of this crude reaction mixture at 4.79 mg/mL were then added DMA (560 μL) and 15 μL of a solution of Example 52 in DMA (20 mM) and the reaction mixture was stirred overnight at RT. It was then purified by gel filtration using a Superdex 200 pg matrix (HiLoad® 16/60, GE Healthcare) pre-equilibrated in DPBS/10% DMA (v/v) and formulated using Sephadex™ G25 matrix (NAP10™ desalting GE Healthcare) pre-equilibrated with buffer F. Example 49 (7.2 mg at 3.79 mg/mL, overall yield 65%) was finally obtained after sterile filtration as a colorless limpid solution with a DAR (HRMS) of 1.94 and a monomeric purity of 99.2%.

RP-HRMS: 148887 (D1), 150421 (D2), 151950 (D3).

### Pharmacological Activities

### Example A: Evaluation of Stimulation of Human TLR7 or Human TLR8 by the Compounds/Conjugates of Formula (II) by In-Vitro Assay

The compounds/conjugates of formula (II) according to the disclosure, R848 and 3M012 were subjected to pharmacological testing for determining their activity as TLR7 and TLR8 agonists. R848 and 3M012 are well known TLR7/8 agonists.

The below tests involving measuring the in vitro activity of some compounds of the disclosure on human TLR7 pathway were carried out using parental HEK-Blue™ hTLR7 cells and engineered tumor antigen-expressing HEK-Blue™ hTLR7 cells respectively as reporter cells.

The tests involving measuring the in vitro activity of some compounds of the disclosure on human TLR8 pathway were carried out using parental HEK-Blue™ hTLR8 (#hkb-htlr8) cells and engineered tumor antigen-expressing HEK-Blue™ hTLR8 cells respectively as reporter cells.

The activity described below, with respect to human TLR7 or human TLR8, is given by the half maximal effective concentration (EC50), which corresponds to the concentration required to obtain a 50% of the activation. The lower the EC50, the less the concentration of the compound is required to produce 50% of maximum activation and the higher the potency. The below EC50 between 1 to 300 nM are considered as demonstrating a good pathway activation and EC50≥1000 nM are considered as demonstrating an absence of pathway activation.

The EC50 of R848 and 3M012 have been
tested according to the below test.

| | EC50 on HEK-Blue ™ | EC50 on HEK-Blue ™ |
|---|---|---|
| | hTLR7 cells | hTLR8 cells |
| R848 | 182 nM | 1.01 μM |
| 3M012 | 518 nM | 2.54 μM |

HEK-Blue™ hTLR7 Cells (#hkb-htlr7) and HEK-Blue™ hTLR8 Cells (#hkb-htlr8) cells from InvivoGen and the engineered tumor antigen expressing cells were used according to the manufacturer's instructions. The HEK-Blue™ hTLR7 and HEK-Blue™ hTLR8 cells were obtained by co-transfection of the hTLR7 or hTLR8 gene in HEK293 cells and an optimized Secreted Embryonic Alkaline Phosphatase (SEAP) reporter gene which is placed under the control of the IFN-β minimal promoter fused to five NF-κB and AP-1-binding sites. The human tumor antigen expressing HEK-Blue™ hTLR7 and hTLR8 cells were obtained by transfection of the tumor antigen genes in the parental cells followed by a polyclonal selection to enrich in cells overexpressing the tumor antigen. Stimulation with a TLR7/8 ligand activates NF-kB and AP-1 which induce the production of SLAP. The cells are seeded in 96-well plate at 3.10E4 cells/well and incubated 24 hours at 37C, 5% CO₂ before adding the conjugates to be evaluated. The cells are subsequently incubated with 3-fold serial dilutions of each of the compounds (0.01 nM to 1 μM) for 96 hours at 37° C., 5% CO₂. Next, 20 μL of the supernatant is harvested and incubated with 200 μL of QUANTI-Blue™ Solution. After a 3-hour incubation at 37° C., 5% CO₂, SLAP activity is assessed by reading the optical density (GD) at 620 nm with an Envision microplate reader and a half maximal effective concentration (EC50) is calculated and used to rank the compounds. The results are summarized in Tables below.

TABLE 1a

| Compounds/ | | | | EC50 | |
|---|---|---|---|---|---|
| Conjugates | | EC50 | | HEK-Blue- | |
| of formula (II) | | HEKBlue- | | hTLR7- | |
| or references | Name | hTLR7 | | tumor Ag | |
| Example 2 | CEACAM5_Tusamitamab- | >1000 | nM | 20.5 | nM |
| | PEG4-R848 | | | | |
| Example 3 | EphA2_hu2H11_R35R74- | >1000 | nM | 69.5 | nM |
| | PEG4-R848 | | | | |
| Example 4 | HER2_trastuzumab-PEG4- | >1000 | nM | 4.7 | nM |
| | R848 | | | | |
| Example 5 | EGFR_cetuximab-PEG4- | >1000 | nM | 3.9 | nM |
| | R848 | | | | |
| Example 8 | CEACAM5_Tusamitamab- | >1000 | nM | 35.6 | nM |
| | PEG8-R848 | | | | |
| Example 9 | EphA2_hu2H11_R35R74- | >1000 | nM | 45.8 | nM |
| | PEG8-R848 | | | | |
| Example 10 | HER2_trastuzumab-PEG8- | >1000 | nM | 3.8 | nM |
| | R848 | | | | |
| Example 11 | EGFR_cetuximab-PEG8- | >1000 | nM | 6.7 | nM |
| | R848 | | | | |
| Example 14 | CEACAM5_Tusamitamab- | NT | | 15.5 | nM |
| | PEG12-R848 | | | | |
| Example 15 | EphA2_hu2H11_R35R74- | >1000 | nM | 34.2 | nM |
| | PEG12-R848 | | | | |
| Example 16 | HER2_trastuzumab- | >1000 | nM | 2.76 | nM |
| | PEG12-R848 | | | | |
| Example 17 | EGFR_cetuximab-PEG12- | >1000 | nM | 4.8 | nM |
| | R848 | | | | |
| Example 20a | CEACAM5_Tusamitamab- | >1000 | nM | 5.5 | nM |
| | PEG24-R848 | | | | |
| Example 21 | EphA2_hu2H11_R35R74- | >1000 | nM | 20.2 | nM |
| | PEG24-R848 | | | | |
| Example 22 | HER2_trastuzumab- | >1000 | nM | 3.2 | nM |
| | PEG24-R848 | | | | |
| Example 23 | EGFR_cetuximab-PEG24- | >1000 | nM | 4.1 | nM |
| | R848 | | | | |
| TLR7/8 agonist | Resiquimod (R848) | 182 | nM | 302 | nM |

| | | | | |
|---|---|---|---|---|
| TLR7/8 agonist | 3M012 | 518 | nM | NT |

NT = not tested

TABLE 1b

| Compounds/ | | | | EC50 | |
|---|---|---|---|---|---|
| Conjugates of | | EC50 | | HEK-Blue- | |
| formula (II) | | HEKBlue- | | hTLR7- | |
| or references | Name | hTLR7 | | tumor Ag | |
| Example 20a | CEACAM5_Tusamitamab-PEG24-R848 | >1000 | nM | 10.2 | nM |
| Example 33 | CEACAM5_Tusamitamab_S239C-PEG24-R848 | >1000 | nM | 13.7 | nM |
| Example 34 | CEACAM5_Tusamitamab_K274C-PEG24-R848 | >1000 | nM | 11.3 | nM |
| Example 35 | CEACAM5_Tusamitamab_K290C-PEG24-R848 | >1000 | nM | 13.0 | nM |
| Example 36 | CEACAM5_Tusamitamab_K326C-PEG24-R848 | >1000 | nM | 10.5 | nM |
| Example 37 | CEACAM5_Tusamitamab_K320C-PEG24-R848 | >1000 | nM | 13.9 | nM |
| Example 38 | CEACAM5_Tusamitamab_K340C-PEG24-R848 | >1000 | nM | 13.2 | nM |
| Example 40 | CEACAM5_Tusamitamab_N361C-PEG24-R848 | >1000 | nM | 11.1 | nM |
| Example 41 | CEACAM5_Tusamitamab_K414C-PEG24-R848 | >1000 | nM | 7.9 | nM |
| Example 42 | CEACAM5_Tusamitamab_V422C-PEG24-R848 | >1000 | nM | 11.5 | nM |
| TLR7/8 agonist | Resiquimod (R848) | 188 | nM | 208 | nM |

TABLE 1c

| Compounds/ | | | | EC50 | |
|---|---|---|---|---|---|
| Conjugates of | | EC50 | | HEK-Blue- | |
| formula (II) | | HEKBlue- | | hTLR7- | |
| or references | Name | hTLR7 | | tumor Ag | |
| Example 20a | CEACAM5_Tusamitamab-PEG24-R848 | >1000 | nM | 16.4 | nM |
| Example 32 | CEACAM5_Tusamitamab_E152C-PEG24-R848 | >1000 | nM | 27.8 | nM |
| Example 38 | CEACAM5_Tusamitamab_K340C-PEG24-R848 | >1000 | nM | 9.9 | nM |
| Example 39 | CEACAM5_Tusamitamab_S375C-PEG24-R848 | >1000 | nM | 18.7 | nM |
| Example 41 | CEACAM5_Tusamitamab_K414C-PEG24-R848 | >1000 | nM | 15.2 | nM |
| Example 43 | CEACAM5_Tusamitamab_E152C_S375C-PEG24-R848 | >1000 | nM | 25.4 | nM |
| TLR7/8 agonist | Resiquimod (R848) | 199 | nM | 249 | nM |

TABLE 1d

| Compounds/ | | | | EC50 | |
|---|---|---|---|---|---|
| Conjugates of | | EC50 | | HEK-Blue- | |
| formula (II) or | | HEKBlue- | | hTLR7- | |
| references | Name | hTLR7 | | tumor Ag | |
| Example 20a | CEACAM5_Tusamitamab- | >667 | nM | 12.3 | nM |
| | PEG24-R848 | | | | |
| Example 49 | CEACAM5_Tusamitamab_{—} | >667 | nM | 14.0 | nM |
| | K274C-Example 48 | | | | |
| Example 51 | CEACAM5_Tusamitamab_{—} | >667 | nM | 12.1 | nM |
| | K274C-Example 50 | | | | |
| Example 53 | CEACAM5_Tusamitamab_{—} | >667 | nM | >667 | nM |
| | K274C-Example 52 | | | | |
| TLR7/8 agonist | Resiquimod (R848) | 97.0 | nM | 180.0 | nM |

The above results show that the compounds/conjugates of formula (II) of the disclosure well activate the TLR7 pathway in the engineered tumor antigen expressing HEK-Blue™ hTLR7 cells, but not in the parental cells that do not express tumor antigen. The TLR7 pathway activation by the compounds/conjugates of formula (II) of the disclosure is dependent on the expression of the tumor antigen at the surface membrane of the cells.

TABLE 2

| Compounds/ | | | | EC50 | |
|---|---|---|---|---|---|
| Conjugates of | | EC50 | | HEK Blue- | |
| formula (II) | | HEKBlue- | | hTLR8- | |
| or reference | Name | hTLR8 | | CEACAM5 | |
| Example 2 | CEACAM5_Tusamit amab-PEG4-R848 | >1000 | nM | >1000 | nM |
| Example 8 | CEACAM5_Tusamit amab-PEG8-R848 | >1000 | nM | >1000 | nM |
| Example 14 | CEACAM5_Tusamit amab-PEG12-R848 | >1000 | nM | >1000 | nM |
| Example 20a | CEACAM5_Tusamit amab-PEG24-R848 | >1000 | nM | >1000 | nM |
| TLR7/8 agonist | Resiquimod (R848) | 1.01 | μM | 0.905 | μM |

The above results show that the compounds/conjugates of the formula (II) of the disclosure do not activate the TLR8 pathway whether in the parental or engineered tumor antigen expressing HEK-Blue™ hTLR8 cells. These results are unexpected considering that R848 is able to activate the TLR8 pathway in parental HEK-Blue™ hTLR8 EC50: 1.01 μM and engineered tumor antigen expressing HEK-Blue™ hTLR8 EC50: 0.905 μM). It means that the TLR7/8 agonist under the compounds/conjugate of formula (II) of the disclosure do not activate the TLR8 pathway while they are able to activate the TLR7 pathway as exemplified in Table 1.

This particularity of the compounds/conjugates of formula (II) of the disclosure to activate TLR7 but not TLR8 pathways is notably advantageous for a therapeutical use knowing that TLR7 and TLR8 receptors trigger different signaling pathways that contribute to distinct cytokine secretion phenotypes.

The activation of TLR7 pathway preferentially induced secretion of type-1 interferon and IFN-regulated chemokines, such as I-TAC (CXCL11) and IP-10 (CXCL10), while the activation of TLR8 pathway predominantly induced proinflammatory cytokines and chemokines including IL6, TNFα, IL-12 and MIP-1α (CCL3) (Gorden et al., 2005). IL6 and TNFα are among the core cytokines found to be elevated in the serum of patients with cytokine release syndrome (CRS), which can cause flu-like symptoms but also nonspecific and systemic symptoms that could be life threatening according to the grade of the CRS. Moreover, intravenous antibody-TLR8 agonist conjugates have been described to induce acute anaphylaxis-like reaction in non-human primates (WO2020/056008).

Therefore, the compounds/conjugates of formula (II) of the disclosure by activating only TLR7 pathway and not TLR8 pathway are particularly advantageous for a therapeutical use to limit the adverse effects such as limiting CRS in treated patients and secure the intravenous administration.

### Example B: Evaluation of the Cellular Binding (Apparent Affinity) on Human CEACAM5-Expressing HEK-Blue™ hTLR7 and HEK-Blue™ hTLR8 Cells by In Vitro Assay

The compounds/conjugates of formula (II) according to the disclosure were subjected to pharmacological testing for determining their cellular binding (apparent affinity) to cells expressing human CEACAM5.

The below tests involving measuring the cellular binding of some compounds/conjugates of formula (II) of the disclosure were carried out using human CEACAM5-expressing HEK-Blue™ hTLR7 and HEK-Blue™ hTLR8 cells.

The activity described below is given by the half maximal effective concentration (EC50), which corresponds to the concentration required to obtain a 50% of the cellular binding. The lower the EC50, the less the concentration of the compound/conjugate of formula (II) is required to produce 50% of maximum binding and the higher the potency.

The below EC50 between 1 to 10 nM are considered as demonstrating a cellular binding.

The tested compound/conjugate was covalently labelled with Alexa Fluor 488 (AF488). The human CEACAM5-expressing HEK-Blue™ hTLR7 and HEK-Blue™ hTLR8 cells are seeded in 96-well plate at 2.10E5 cells/well and incubated with 2-fold serial dilutions of each of the AF488-labelled compounds (1.3 nM to 167 nM) for 1 hour at 4° C. Following a washing step with PBS, the cells are detached using trypsin/EDTA and analyzed on MACSQuant® VYB flow cytometer (Miltenyi Biotech). Mean fluorescent intensity values were obtained from the histograms and were used to plot the binding curves. A half maximal effective concentration (EC50) is calculated and used to rank the compounds.

TABLE 3

| | | EC50 | EC50 |
|---|---|---|---|
| Compound/ | | HEKBlue- | HEK Blue- |
| Conjugates of | | hTLR7- | hTLR8- |
| formula (II) | Name | CEACAM5 | CEACAM5 |
| Example 20a | CEACAM5_Tusamit amab-PEG24-R848 | 7.3 nM | 9.7 nM |

The above result shows that the compounds/conjugates of formula (II) of the disclosure well bind to the CEACAM5-expressing HEK-Blue™ hTLR7 and HEK-Blue™ hTLR8 cells.

### Example C: Evaluation of the Internalization of Compounds/Conjugates of Formula (II) by Human CEACAM5-Expressing HEK-Blue™ hTLR7 and HEK-Blue™ hTLR8 Cells by In-Vitro Assay

The compounds/conjugates of formula (II) according to the disclosure were subjected to pharmacological testing for determining their internalization capability.

The below tests involving measuring the internalization of some compounds/conjugates of formula (II) of the disclosure were carried out using human CEACAM5-expressing HEK-Blue™ hTLR7 and human CEACAM5-expressing HEK-Blue™ hTLR8 cells.

The activity described below is given by the quantity of internalized compounds/conjugates of formula (II) which corresponds to the median of the internalized fluorescence intensity (internal. MFI) measured at 4, 24, 48 and 72 hours of incubation at 37° C., 5% CO₂. The higher the internal. MFI, the greater the amount of internalized compounds/conjugates of formula (II).

The below internal. MFI values >1000 are considered as demonstrating an internalization of the tested compounds/conjugates of formula (II) in the cells. The below internal. MFI values <1000 are considered as demonstrating an absence of internalization.

The tested compounds/conjugates of formula (II) were covalently labelled with Alexa Fluor 488 (AF488). The human CEACAM5-expressing HEK-Blue™ hTLR7 and human CEACAM5-expressing HEK-Blue™ hTLR8 cells are seeded in 96-well plate at 1.10E5 cells/well and incubated with 10 μg/ml of the AF488-labelled compounds for 4, 24, 48 and 72 hours at 37° C., 5% CO₂. Then, the internalization was stopped by incubating the plate at 4° C.; an anti-AF488 antibody (Invitrogen, A11094) was added at 50 μg/ml in each well and incubated for 45 min at 4° C., in order to quench the fluorescent signal from the cell surface, preserving the fluorescence signal of compounds internalized by the cells during the incubation phase at 37° C., 5% CO₂. Following a washing step with PBS, the cells are detached using trypsin/EDTA and analyzed on MACSQuant® VYB flow cytometer (Miltenyi Biotech). The internalized fluorescent intensity (internal. MFI) median values were obtained from the histograms and used to determine the quantity of internalized compounds/conjugates of formula (II) by the human CEACAM5-expressing HEK-Blue™ hTLR7 and human CEACAM5-expressing HEK-Blue™ hTLR8 cells.

TABLE 4

| | |
|---|---|
| | Internalized fluorescent intensity |
| Time of | (Median) on HEKBlue-hTLR7-CEACAM5 |

| incubation at | CEACAM5_{—} | Irrelevant |
|---|---|---|
| 37° C., 5% CO₂ | Tusamitamab-PEG24- | human |
| (hours) | R848 (example 20a) | IgG1 |
| 4 | 1341 | 144 |
| 24 | 5443 | 525 |
| 48 | 7750 | 738 |
| 72 | 11169 | 833 |

TABLE 5

| | |
|---|---|
| | Internalized fluorescent intensity |
| Time of | (Median) on HEKBlue-hTLR8-CEACAM5 |

| incubation at | CEACAM5_{—} | Irrelevant |
|---|---|---|
| 37° C., 5% CO₂ | Tusamitamab-PEG24- | human |
| (hours) | R848 (example 20a) | IgG1 |
| 4 | 1669 | 68 |
| 24 | 5785 | 120 |
| 48 | 14251 | 151 |
| 72 | 14603 | 167 |

The above results show that the compounds/conjugate of formula (II) of the disclosure are well internalized by the CEACAM5-expressing HEK-Blue™ hTLR7 and HEK-Blue™ hTLR8 cells and accumulate into the cells with time of incubation. The internalization of the compounds/conjugates of formula (II) is dependent on CEACAM5 expressed at the surface of the cells as an irrelevant human IgG1 that does not recognize any membrane protein is not internalized and not accumulated with time of incubation This confirms that the internalization of the compounds/conjugates of formula (II) of the disclosure is CEACAM5-mediated in both cell lines.

### Example D: Evaluation of the Stimulation of Human THP-1 Monocytic Cells by In Vitro Assay

The compounds/conjugates of formula (II) according to the disclosure were subjected to pharmacological testing for determining their activity on human monocytic cells that express at their membrane surface Fc receptors that show binding specificity for the Fc part of the compounds/conjugates of formula (II).

The below tests involving measuring the in vitro activity of some compounds/conjugates of formula (II) of the disclosure were carried out using THP1-Dual™ cells as reporter monocyte cells.

The activity described below is given by the half maximal effective concentration (EC50), which corresponds to the concentration required to obtain a 50% of the activation. The lower the EC50, the less the concentration of the compound is required to produce 50% of maximum activation and the higher the potency.

The below EC50 between 0.3 to 400 nM are considered as demonstrating an activation; EC50≥1000 nM are considered as demonstrating an absence of activation.

THP1-Dual™ cells (#thpd-nfis) from InvivoGen were used according to the manufacturer's instructions. The cells were obtained by stable integration of two inducible reporter constructs in human THP-1 monocyte cells: an optimized Secreted Embryonic Alkaline Phosphatase (SEAP) reporter gene which is placed under the control of the IFN-β minimal promoter fused to five NF-κB and three c-Rel binding sites and a Secreted Lucia luciferase gene which is placed under the control of an ISG54 minimal promoter fused with five interferon (IFN)-stimulated response elements.

As a result, THP1-Dual™ cells allow to study the activation of the NF-κB pathway by monitoring the activity of SEAP, and the interferon regulatory factor pathway, by assessing the activity of Lucia luciferase. Stimulation of THP1-Dual™ cells with TLR7, TLR7/8 or TLR8 ligands activates NF-kB and c-Rel which induce the production of SEAP as documented below.

TABLE 6

| | EC50 NFkB pathway | |
|---|---|---|
| Ligand name | THP1-Dual cells | |
| Gardiquimod TLR7 agonist | >4000 | nM |
| R848 TLR7/8 agonist | 212 | nM |
| Motolimod TLR8 agonist | 394 | nM |

The cells are seeded in 96-well plate at 1.10E5 cells/well and subsequently incubated with 3-fold serial dilutions of each of the compounds (0.01 nM to 1 μM) for 96 hours at 37° C., 5% CO₂. Next, 20 μL of the supernatant is harvested and incubated with 200 μL of QUANTI-Blue™ Solution. After a 3 hours incubation at 37° C., 5% C₀₂, SEAP activity is assessed by reading the optical density (OD) at 620 nm with an Envision microplate reader and a half maximal effective concentration (EC50) is calculated and used to rank the compounds/conjugates of formula (II).

The results are summarized in Table below.

TABLE 7a

| | | EC50 NFkB |
|---|---|---|
| | | pathway THP1- |
| Conjugates | Name | Dual cells |
| Example 2 | CEACAM5_Tusamitamab-PEG4-R848 | 0.8 nM |
| Example 8 | CEACAM5_Tusamitamab-PEG8-R848 | 1.9 nM |
| Example 14 | CEACAM5_Tusamitamab-PEG12-R848 | 2.8 nM |
| Example 20a | CEACAM5_Tusamitamab-PEG24-R848 | 0.9 nM |

TABLE 7b

| Compounds/ | | | |
|---|---|---|---|
| Conjugates of | | EC50 NFkB | |
| formula (II) or | | pathway | |
| references | Name | THP1-Dual cells | |
| Example 20a | CEACAM5_Tusamitamab- | 1.6 | nM |
| | PEG24-R848 | | |
| Example 32 | CEACAM5_Tusamitamab_{—} | 1.6 | nM |
| | E152C-PEG24-R848 | | |
| Example 38 | CEACAM5_Tusamitamab_{—} | >1000 | nM |
| | K340C-PEG24-R848 | | |
| Example 39 | CEACAM5_Tusamitamab_{—} | >1000 | nM |
| | S375C-PEG24-R848 | | |
| Example 41 | CEACAM5_Tusamitamab_{—} | 2.6 | nM |
| | K414C-PEG24-R848 | | |
| Example 43 | CEACAM5_Tusamitamab_{—} | 2.0 | nM |
| | E152C_S375C-PEG24-R848 | | |
| TLR7/8 agonist | Resiquimod (R848) | 238 | nM |

The above results show that the compounds/conjugates of formula (II) of the disclosure activate the NFkB pathway in THP1 dual cells. It means that the compounds/conjugates of formula (II) of the disclosure bind the immune cell via the Fc part of the antibody, internalize and activate the NFkB pathway.

### Example E: In Vitro Assay for Evaluating the Immune Cell-Mediated Tumor Cell Killing Activity of Conjugates

The conjugates according to the disclosure were subjected to pharmacological testing for determining their tumor cell killing activity via immune cell activation.

The below tests involving measuring the in vitro tumor cell killing activity of some compounds of the disclosure were carried out using Incucyte® Immune Cell Killing Assay consisting in co-culture of human peripheral blood mononuclear cells (PBMC) and NucLight red-labelled human MKN-45 tumor cells. This Immune Cell Killing Assay allows for direct, measurements of immune cell-mediated killing of MKN-45 tumor cells with the combination of real-time, automated analysis of tumor cell number.

MKN-45 (stomach tumor cell line, DSMZ Germany) were used as they present a very high expression of CEACAM5 (Antigen Binding Capacity ≈3.10E5 receptors per cell). To count living MKN-45 cells in real time without altering their function, MKN45 cells have been stained thanks to a lentiviral-based labeling reagent enabling expression of a nuclear-restricted red (Nuclight Red) fluorescent protein.

NucLight red-labelled human MKN-45 cells were seeded in 96-well plate at 7.5.10E3 cells/well (80 μl) and subsequently incubated for 24 hours at 37° C., 5% CO₂. Then, conjugates of the disclosure were added in wells. All conjugates have been diluted in RPMI with 20% FBS and added in the wells at a final concentration of 10 nM.

PBMCs were isolated by Ficoll density gradient centrifugation from peripheral blood samples obtained from healthy donors. Freshly isolated PBMCs were washed twice with PBS and resuspended in RPMI-1640 medium supplement with 20% FBS and added to Nuclight red MKN-45 tumor cells at a ratio of 1:30, i.e. 2.25 10E5 PBMC in 80 μl.

The plates were then placed in the Incucyte® live-cell analysis apparatus. The Incucyte® Cell-by-Cell Analysis module was used to quantify the number of NucLight red-labelled MKN-45 tumor cells over-time.

The activity described below is given by the percentage of immune cell-mediated killing activity after a 120-hour incubation. The higher the percentage, the higher the killing activity is. The below percentage values >20 are considered as demonstrating a significant killing activity of the tested compounds on the cells. The below percentage values <20 are considered as demonstrating an absence of activity.

| | % of Immune cell-mediated |
|---|---|
| | killing activity |
| Compounds | No compound |
| No compound | 0% |
| CEACAM5_Tusamitamab-PEG24-R848 | 57% |
| Irrelevant human antibody | 6.3% |

The above example shows that the conjugates of the disclosure elicit immune-mediated tumor cell killing and therefore show a therapeutic effect especially as a promising treatment of cancer.

The compounds/payloads of formula (I) of the present disclosure are able to be conjugated, and the compounds/conjugates of formula (II) show a good solubility and a good stability.

The compounds/conjugates of formula (II) of the present disclosure are able to selectively stimulate TLR7 pathway without stimulating TLR8 pathway, bind to the tumoral target through the antibody and to internalize into the targeted cell, stimulate the immune cells in a FcyR-dependent binding and TLR7 activation, and finally provide tumor cell killing by activated immune cells.

Furthermore, the compounds/conjugates of the formula (II), by not stimulating TLR8, are good candidates for therapeutic uses with less adverse-events, such as adverse-events linked to the NFK-β pathway for instance CRS and specially to prevent and/or treat cancer.

It is understood that the examples and embodiments described herein are for illustrative purposes only and that various modifications or changes in light thereof will be suggested to persons skilled in the art and are to be included within the spirit and purview of this application and scope of the appended claims.

Accordingly, in another of its aspects, the disclosure provides medicaments which comprise at least one compound/conjugate of formula (II) according to the present disclosure, or a pharmaceutically acceptable salt thereof.

The present disclosure, according to another of its aspects, also relates to the compounds/conjugates of formula (II) according to the present disclosure, or a pharmaceutically acceptable salt thereof, for use as medicaments.

The present disclosure, according to another of its aspects, also relates to the compounds/conjugates of formula (II) according to the present disclosure, or a pharmaceutically acceptable salt thereof, for use in therapy.

These medicaments are employed therapeutically, especially in the prevention and/or treatment of diseases or disorders that could benefit from an activation of the immune system.

These medicaments are employed therapeutically, especially in the prevention and/or treatment of a disease or a disorder that could benefit from an activation of the immune system, for instance in the prevention and/or treatment of a cell-proliferative disease, a cancer, a chronic myelogenous, a hairy cell leukemia, a dermatological disease such as a skin lesion or a skin cancer (for example an external genital and perianal warts/condyloma acuminate, a genital herpes, an actinic keratosis, a basal cell carcinoma, a cutaneous T-cell lymphoma), an autoimmune disease, an inflammatory disease, a respiratory disease, a sepsis, an allergy (for example an allergic rhinitis or a respiratory allergy), an asthma, a graft rejection, a graft-versus-host disease, and an immunodeficiency, for instance in the prevention and/or treatment of cancers.

In an embodiment, is a compound/conjugates of formula (II) of the instant disclosure, or a pharmaceutically acceptable salt thereof, for use in the prevention and/or treatment of a cancer.

It is possible to prevent and/or treat solid or liquid cancers.

In an embodiment, the cancers are selected from bone, brain, kidney, liver, adrenal gland, colorectal, urinary bladder, breast, stomach, ovaries, colon, rectum, prostate, pancreas, lung, vagina, thyroid, the neck and head cancers.

In another embodiment is a compound/conjugate of formula (II) of the instant disclosure or a pharmaceutically acceptable salt thereof, for use in an anti-tumoral vaccine.

The present disclosure, according to another of its aspects, also relates to a method of preventing and/or treating the pathological conditions indicated above, comprising administering to a subject in need thereof a therapeutically effective amount of a compound/conjugate of formula (II), or a pharmaceutically acceptable salt thereof.

In an embodiment of this method of treatment, the subject is a human.

The present disclosure also relates to the use of a compound/conjugate of formula (II), or a pharmaceutically acceptable salt thereof according to the present disclosure, for the manufacture of a medicament useful in preventing and/or treating any of the pathological conditions indicated above, for example useful as anti-tumoral vaccine or for preventing and/or treating cancer.

The compounds/conjugates of formula (II) of the present disclosure may also be used in monotherapy or combination with radiotherapy or chemotherapy. For example, targeted chemotherapy, molecularly targeted therapy such as drugs interfering with specific targeted molecules needed for carcinogenesis and tumor growth, drugs interfering with cancer cell metabolism, immunotherapy including but not limiting to checkpoint inhibitors, cellular immunotherapy, antibody therapy and cytokine therapy, radiotherapy-based modalities, antiangiogenic therapy or adjuvant or neoadjuvant therapy.

The compound/conjugate of formula (II) may be used alone or in combination with at least one other anticancer agent.

According to another of its aspects, the present disclosure relates to pharmaceutical compositions comprising an effective dose of at least one compound/conjugate of formula (II) according to the disclosure or a pharmaceutically acceptable salt thereof, and also at least one pharmaceutically acceptable excipient.

The said excipients are selected, in accordance with the pharmaceutical form and method of administration desired, from the customary excipients, which are known to a person skilled in the art.

The unit administration forms appropriate include intra-ocular and intra-nasal administration forms, forms for inhalative, topical, transdermal, subcutaneous, intra-muscular or intravenous administration, rectal administration forms and implants. For topical application it is possible to use the compounds according to the disclosure in creams, gels, ointments or lotions.

Sequence listing

-Tusamitamab
>LC, AntiCEACAM5_hyb_769_4DVL1c_4DVH1a_IgG1
DIQMTQSPASLSASVGDRVTITCRASENIFSYLAWYQQKPGKSPKLLVYNTRTLAEGVPS
RFSGSGSGTDFSLTISSLQPEDFATYYCQHHYGTPFTFGSGTKLEIKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC (SEQ ID NO. 1)
>HC, AntiCEACAM5_hyb_769_4DVL1c_4DVH1a_IgG1
EVQLQESGPGLVKPGGSLSLSCAASGFVFSSYDMSWVRQTPERGLEWVAYISSGGGITYA
PSTVKGRFTVSRDNAKNTLYLQMNSLTSEDTAVYYCAAHYFGSSGPFAYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPG (SEQ ID NO. 2)
-Hu2H11-R35-R74
>LC
DVVMTQTPLSLSVTLGQPASISCKSSQSLIHSDGRTYLNWLLQRPGQSPKRLIYLVSRLD
SGVPDRFTGSGAGTDFTLKISRVEAEDLGVYYCWQGSHFPRTFGGGTKLEIKRTVAAPSV
FIFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC (SEQ ID NO. 3)
>HC
QVQLVQSGAEVVKPGASVKISCKASGYTFTAYYMHWVKQSPVQSLEWIGLVNPYNGESSY
NQKFQGKASLTVDRSSSTAYMELHSLTSEDSAVYYCAREFYGYRYFDVWGQGTAVTVSSA
STKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYSKLTVDKSRWQ
QGNVFSCSVMHEALHNHYTQKSLSLSPG (SEQ ID NO. 4)
-Trastuzumab
>LC
IQMTQSPSSLSASVGDRVTITCRASQDVNTAVAWYQQKPGKAPKLLIYSASFLYSGVPS
RFSGSRSGTDFTLTISSLQPEDFATYYCQQHYTTPPTFGQGTKVEIKRTVAAPSVFIFP
PSDEQLKSGTASVVCLLNNFYPREAKVQWKVDNALQSGNSQESVTEQDSKDSTYSLSST
LTLSKADYEKHKVYACEVTHQGLSSPVTKSFNRGEC (SEQ ID NO. 5)
>HC
EVQLVESGGGLVQPGGSLRLSCAASGENIKDTYIHWVRQAPGKGLEWVARIYPTNGYTR
YADSVKGRFTISADTSKNTAYLQMNSLRAEDTAVYYCSRWGGDGFYAMDYWGQGTLVTV
SSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGK (SEQ ID NO. 6)
-Cetuximab
>LC
DILLTQSPVILSVSPGERVSFSCRASQSIGTNIHWYQQRINGSPRLLIKYASESISGIPS
RFSGSGSGTDFTLSINSVESEDIADYYCQQNNNWPTTFGAGTKLELKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC (SEQ ID NO. 7)
>HC
QVQLKQSGPGLVQPSQSLSITCTVSGFSLTNYGVHWVRQSPGKGLEWLGVIWSGGNTDYN
TPFTSRLSINKDNSKSQVFFKMNSLQSNDTAIYYCARALTYYDYEFAYWGQGTLVTVSAA
STKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYSKLTVDKSRWQ
QGNVFSCSVMHEALHNHYTQKSLSLSPGK (SEQ ID NO. 8)
-Enoblituzumab
>LC
DIQLTQSPSFLSASVGDRVTITCKASQNVDTNVAWYQQKPGKAPKALIYSASYRYSGVPS
RFSGSGSGTDFTLTISSLQPEDFATYYCQQYNNYPFTFGQGTKLEIKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC (SEQ ID NO. 9)
>HC
EVQLVESGGGLVQPGGSLRLSCAASGFTFSSFGMHWVRQAPGKGLEWVAYISSDSSAIYY
ADTVKGRFTISRDNAKNSLYLQMNSLRDEDTAVYYCGRGRENIYYGSRLDYWGQGTTVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTHTCPPCPAPELV
GGPSVFLLPPKPKDTLMISRTPEVTCVVVDVSHEDPEVKFNWYVDGVEVHNAKTKPPEEQ
YNSTLRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPLVLDSDGSFFLYSKLTVDKS
RWQQGNVFSCSVMHEALHNHYTQKSLSLSPGK (SEQ ID NO. 10)

In case of any discrepancy between the above list of sequences and sequences disclosed in an appended sequence listing, the correct sequences are those disclosed above in the present disclosure.

1. A compound of formula (I) or a pharmaceutically acceptable salt thereof 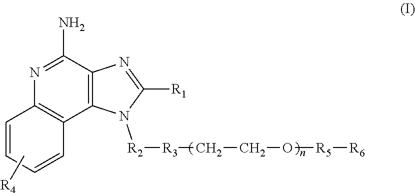
wherein
n is an integer from 1 to 50, for instance from 2 to 30, such as from 3 to 25;
R₁ represents a hydrogen atom, a —(C₁-C₆)alkylene-O—(C₁-C₆)alkyl group, a —(C₁-C₆)alkylene-NH—(C₁-C₆)alkyl group, or a —(C₁-C₆)alkyl group;
R₂ represents a —(C₁-C₆)alkylene- group;
R₃ represents —O—, —NH— or a —N((C₁-C₆)alkyl)- group;
R₄ represents a hydrogen atom, a —(C₁-C₆)alkyl group or a —(C₁-C₆)alkoxy group;
R₅ represents a —(C₁-C₆)alkylene- group; and
R₆ represents a -L₁-RCG₁ group or a —RCG₁ group;
L₁ representing:
—NH—;
—C(═O)—NH—, for instance the —C(═O)— being linked to RCG₁ and the —NH— being linked to R₅;
a —(C₁-C₆)alkylene-R₈—C(═O)—NH— group, R₈ being a —(C₃-C₁₀) cycloalkylene-group or a —(C₃-C₁₀)heterocycloalkylene- group comprising 2 to 9 carbon atoms and 1 to 4 heteroatom(s) selected from oxygen, nitrogen, sulfur, —S(O)— and —SO₂—, for instance the —(C₁-C₆)alkylene- group being linked to RCG₁ and the —NH— being linked to R₅;
a —NH—C(═O)—(C₁-C₆)alkylene-C(═O)—NH— group, one —NH— being linked to RCG₁ and the other —NH— being linked to R₅; or
a —(C₁-C₆)alkylene-C(═O)—NH— group, the —(C₁-C₆)alkylene- group being optionally substituted by a —NH₂ group, for instance the —(C₁-C₆)alkylene- group being linked to RCG₁ and the —NH— being linked to R₅;
RCG₁ representing:
(i) a RₐZₐ—C(═O)— reactive group for which:
Zₐ represents a single bond, —O— or —NH, such as —O—, and
Rₐ represents a hydrogen atom, a —(C₁-C₆)alkyl group, a —(C₃-C₇)cycloalkyl group, a —(C₂-C₆)alkenyl group, a —(C₆-C₁₀)aryl group, a —(C₅-C₁₀)heteroaryl group comprising 4 to 9 carbon atoms and 1 to 4 heteroatom(s) selected from oxygen, nitrogen, and sulfur, or a —(C₃-C₇)heterocycloalkyl group comprising 2 to 6 carbon atoms and 1 to 4 heteroatom(s) selected from oxygen, nitrogen, sulfur, —S(O)— and —SO₂—, said —(C₆-C₁₀)aryl group, —(C₅-C₁₀)heteroaryl group and/or —(C₃-C₇)heterocycloalkyl group being optionally substituted by 1 to 5 atoms/groups chosen from a halogen atom, such as a fluorine atom, a —(C₁-C₆)alkyl group, a —(C₁-C₆)alkoxy group, a hydroxyl group, an oxo group, a nitro group and a cyano group; or
(ii) one of the following reactive groups: a maleimido 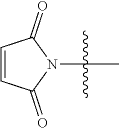 [SMILES: *N1C(=O)C=CC1=O]
group; a substituted maleimido group such as 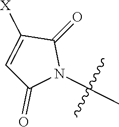
with X=Me, Br, 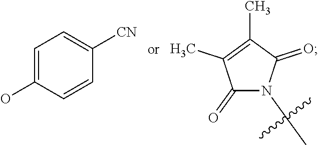
a haloacetamido 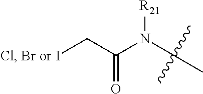
group with R₂₁ representing a hydrogen atom or a (C₁-C₆)alkyl group, such as a methyl group; Cl—; N₃—; HO—; HS—; an activated disulfide such as H or 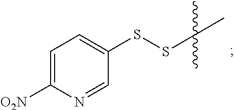 [SMILES: *SSc1ccc([N+](=O)[O-])nc1]
H₂N—; HC≡C— or an activated C≡C such as a cyclooctyne moiety for instance a DBCO-amine 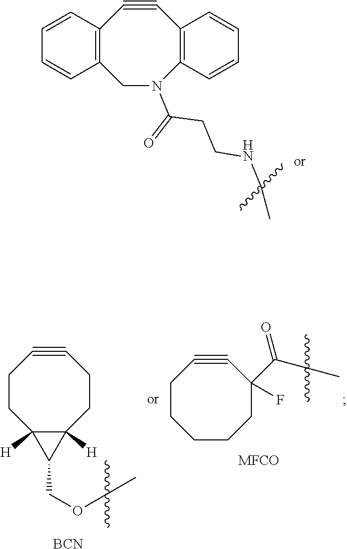
a phenyloxadiazolyl methylsulfone group (PODS) such as 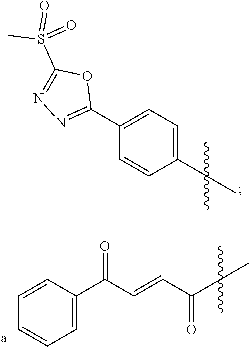 [SMILES: *c1ccc(-c2nnc(S(C)(=O)=O)o2)cc1 ; *C(=O)/C=C/C(=O)c1ccccc1]
group; a 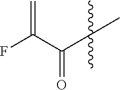 [SMILES: *C(=O)C(=C)F]
group, an O-alkyl hydroxylamine or a Pictet-Spengler reaction substrate such as 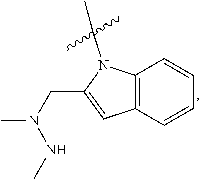 [SMILES: *n1c(CN(C)NC)cc2ccccc21]
for instance RCG₁ is N₃—; a maleimido group 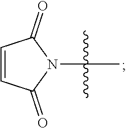 [SMILES: *N1C(=O)C=CC1=O]
a substituted maleimido group 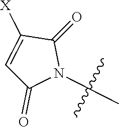
with X=Me, Br, 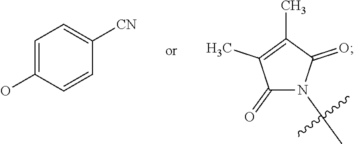
a phenyloxadiazolyl methylsulfone group 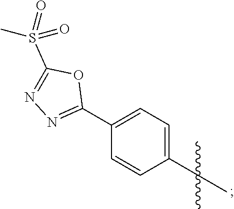 [SMILES: *c1ccc(-c2nnc(S(C)(=O)=O)o2)cc1]
a 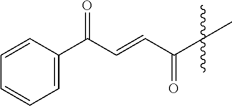 [SMILES: *C(=O)/C=C/C(=O)c1ccccc1]
group; a I—CH₂—C(═O)—NR₂₁— group with R₂₁ representing a hydrogen atom or a (C₁-C₆)alkyl group; or a 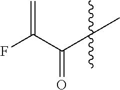 [SMILES: *C(=O)C(=C)F]
group.

2. The compound of formula (I) or a pharmaceutically acceptable salt thereof according to claim 1 wherein n is 3, 7, 11 or 23.

3. The compound of formula (I) or a pharmaceutically acceptable salt thereof according to claim 1, wherein R₁ represents a —CH₂—O—C₂H₅ group.

4. The compound of formula (I) or a pharmaceutically acceptable salt thereof according to anyone of claim 1, wherein R₂ represents a —CH₂—C(CH₃)₂-group.

5. The compound of formula (I) or a pharmaceutically acceptable salt thereof according to anyone of claim 1, wherein R₃ represents —O— or —NH—.

6. The compound of formula (I) or a pharmaceutically acceptable salt thereof according to claim 1, wherein R₄ represents a hydrogen atom.

7. The compound of formula (I) or a pharmaceutically acceptable salt thereof according to claim 1, wherein R₅ represents a —CH₂—CH₂— group.

8. The compound of formula (I) or a pharmaceutically acceptable salt thereof according to claim 1,
wherein R₆ represents a RCG₁ group, RCG₁ being a —N₃ group or a I—CH₂—C(═O)—NH— group, or wherein R₆ represents a -L₁-RCG₁ group, L₁ being a —(CH₂)₂—C(═O)—NH— group, a —NH—C(═O)—(CH₂)₃—C(═O)—NH— group or a —NH— group and RCG₁ being a maleimido 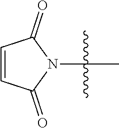 [SMILES: *N1C(=O)C=CC1=O]
group, a substituted maleimido group 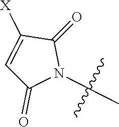
with X=Me, Br, 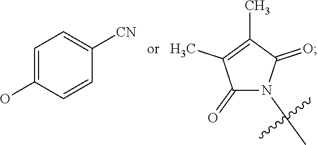
or 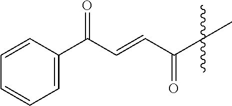 [SMILES: *C(=O)/C=C/C(=O)c1ccccc1]
group; or a phenyloxadiazolyl methylsulfone group (PODS) such as

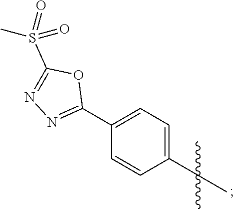 [SMILES: *c1ccc(-c2nnc(S(C)(=O)=O)o2)cc1]

9. The compound of formula (I) or a pharmaceutically acceptable salt thereof according to claim 1, wherein the compound is selected from the group consisting of:

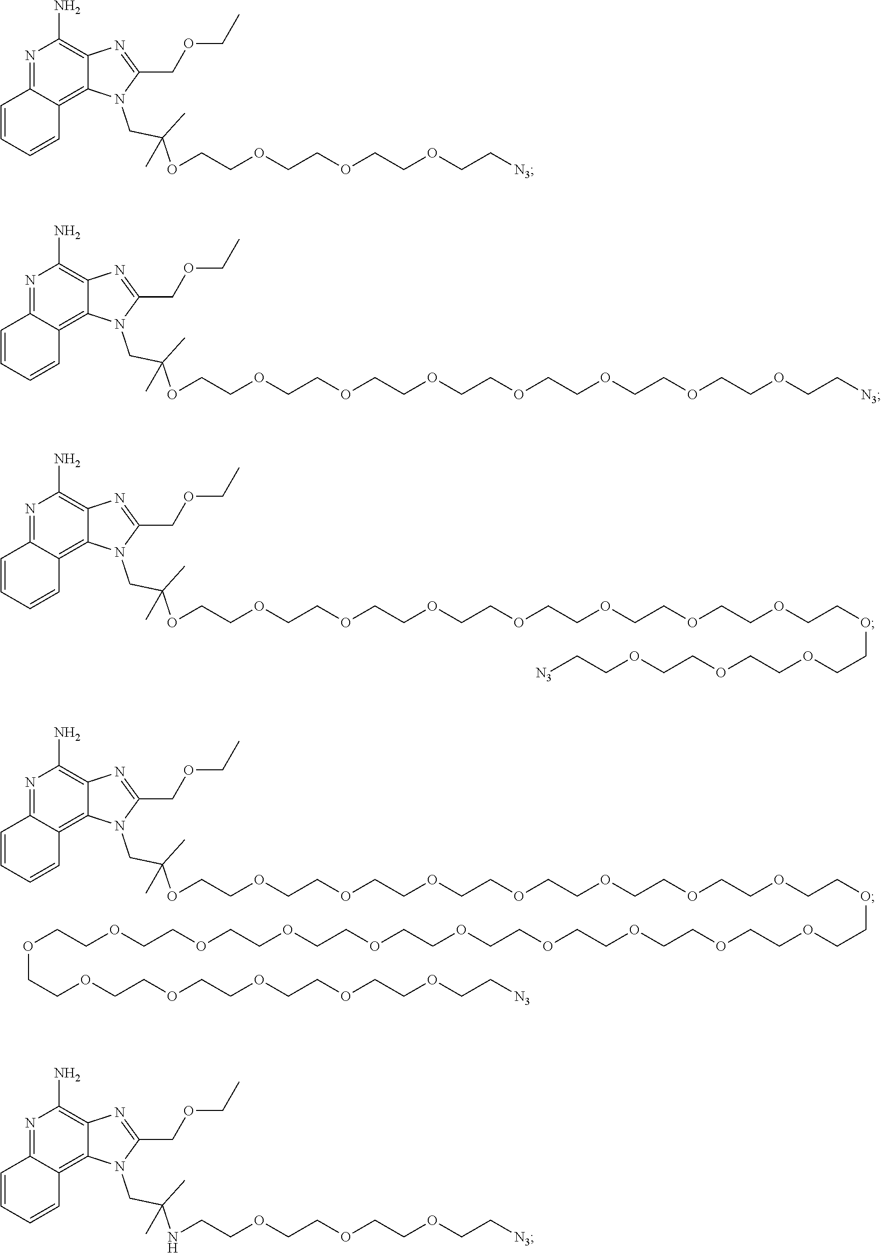 [SMILES: CCOCc1nc2c(N)nc3ccccc3c2n1CC(C)(C)OCCOCCOCCOCCN=[N+]=[N-] ; CCOCc1nc2c(N)nc3ccccc3c2n1CC(C)(C)OCCOCCOCCOCCOCCOCCOCCOCCN=[N+]=[N-] ; CCOCc1nc2c(N)nc3ccccc3c2n1CC(C)(C)OCCOCCOCCOCCOCCOCCOCCOCCOCCOCCOCCOCCN=[N+]=[N-] ; CCOCc1nc2c(N)nc3ccccc3c2n1CC(C)(C)OCCOCCOCCOCCOCCOCCOCCOCCOCCOCCOCCOCCOCCOCCOCCOCCOCCOCCOCCOCCOCCOCCOCCOCCN=[N+]=[N-] ; CCOCc1nc2c(N)nc3ccccc3c2n1CC(C)(C)NCCOCCOCCOCCN=[N+]=[N-]]

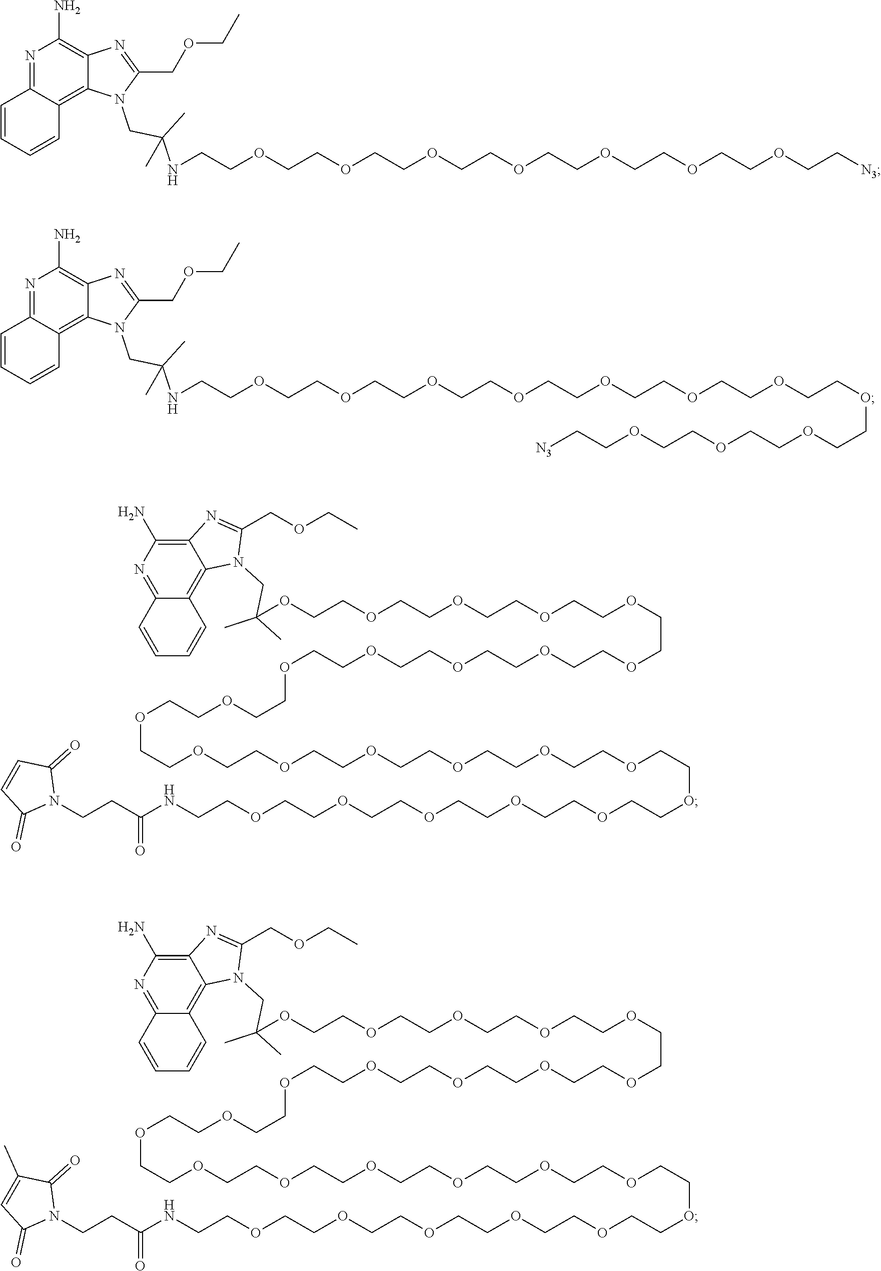 [SMILES: CCOCc1nc2c(N)nc3ccccc3c2n1CC(C)(C)NCCOCCOCCOCCOCCOCCOCCOCCN=[N+]=[N-] ; CCOCc1nc2c(N)nc3ccccc3c2n1CC(C)(C)NCCOCCOCCOCCOCCOCCOCCOCCOCCOCCOCCOCCN=[N+]=[N-] ; CCOCc1nc2c(N)nc3ccccc3c2n1CC(C)(C)OCCOCCOCCOCCOCCOCCOCCOCCOCCOCCOCCOCCOCCOCCOCCOCCOCCOCCOCCOCCOCCOCCOCCOCCNC(=O)CCN1C(=O)C=CC1=O ; CCOCc1nc2c(N)nc3ccccc3c2n1CC(C)(C)OCCOCCOCCOCCOCCOCCOCCOCCOCCOCCOCCOCCOCCOCCOCCOCCOCCOCCOCCOCCOCCOCCOCCOCCNC(=O)CCN1C(=O)C=C(C)C1=O]

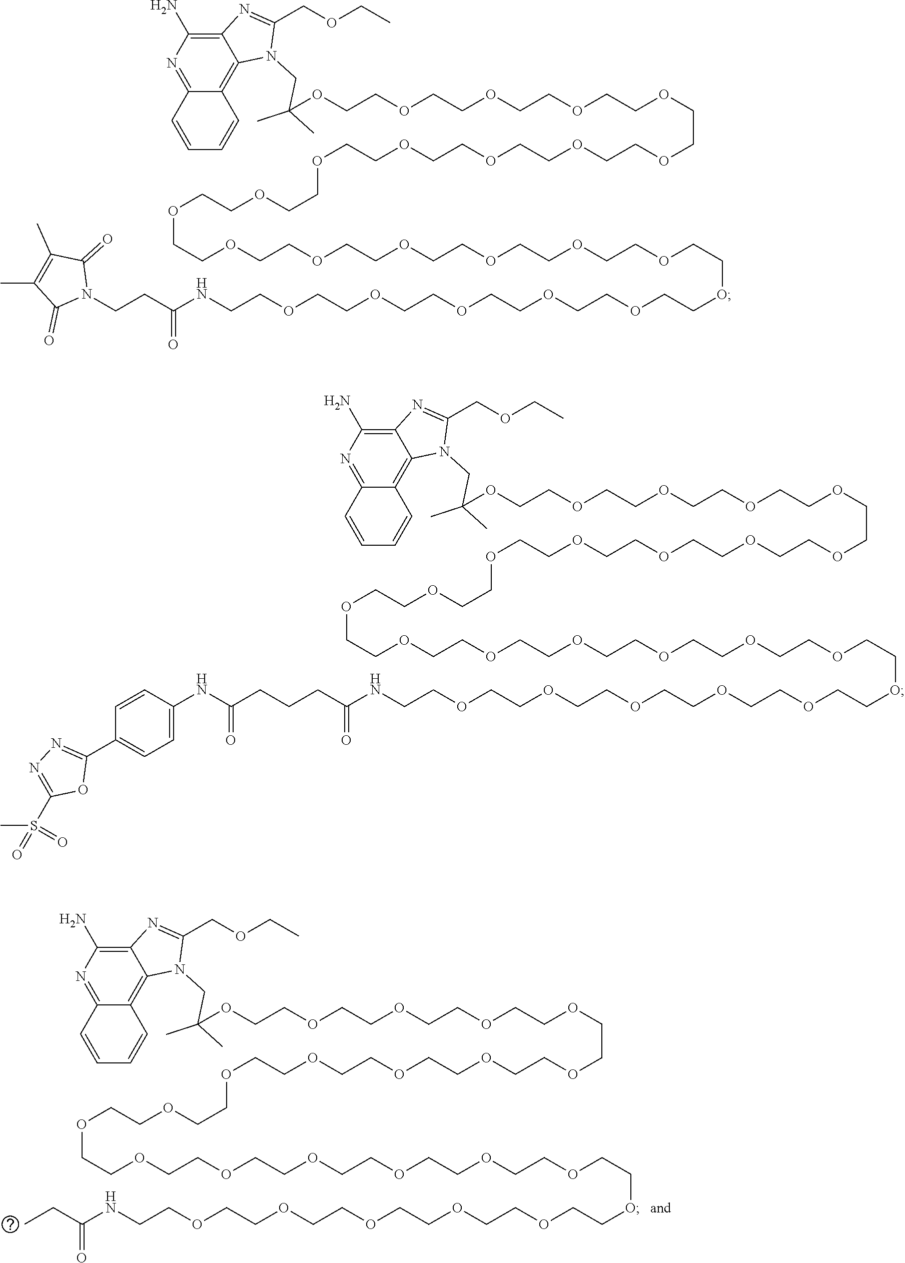 [SMILES: CCOCc1nc2c(N)nc3ccccc3c2n1CC(C)(C)OCCOCCOCCOCCOCCOCCOCCOCCOCCOCCOCCOCCOCCOCCOCCOCCOCCOCCOCCOCCOCCOCCOCCOCCNC(=O)CCN1C(=O)C(C)=C(C)C1=O ; CCOCc1nc2c(N)nc3ccccc3c2n1CC(C)(C)OCCOCCOCCOCCOCCOCCOCCOCCOCCOCCOCCOCCOCCOCCOCCOCCOCCOCCOCCOCCOCCOCCOCCOCCNC(=O)CCCC(=O)Nc1ccc(-c2nnc(S(C)(=O)=O)o2)cc1 ; CCOCc1nc2c(N)nc3ccccc3c2n1CC(C)(C)OCCOCCOCCOCCOCCOCCOCCOCCOCCOCCOCCOCCOCCOCCOCCOCCOCCOCCOCCOCCOCCOCCOCCOCCNC(=O)CC ; "and"]

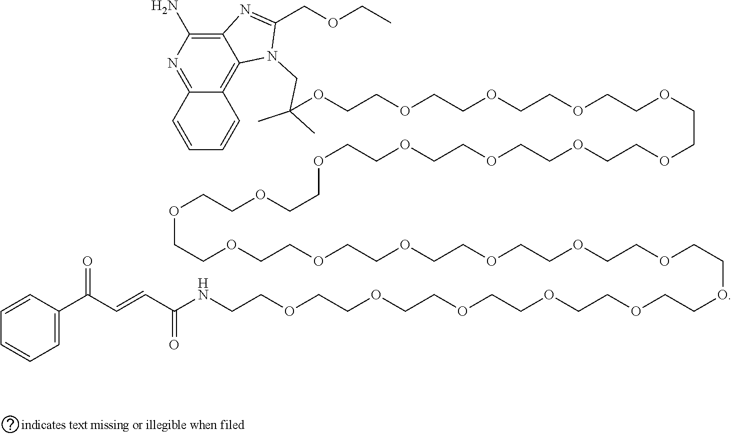 [SMILES: CCOCc1nc2c(N)nc3ccccc3c2n1CC(C)(C)OCCOCCOCCOCCOCCOCCOCCOCCOCCOCCOCCOCCOCCOCCOCCOCCOCCOCCOCCOCCOCCOCCOCCOCCNC(=O)/C=C/C(=O)c1ccccc1]

10. Compound of formula (II) or a pharmaceutically acceptable salt thereof 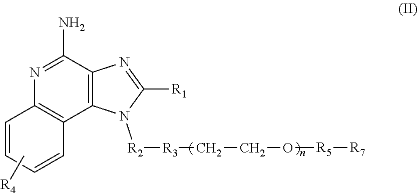
wherein
n is an integer from 1 to 50, for instance from 2 to 30, such as from 3 to 25, for instance 3, 7, 11 or 23;
R₁ represents a hydrogen atom, a —(C₁-C₆)alkylene-O—(C₁-C₆)alkyl group, a —(C₁-C₆)alkylene-NH—(C₁-C₆)alkyl group, or a —(C₁-C₆)alkyl group, for instance a —(C₁-C₆)alkylene-O—(C₁-C₆)alkyl group such as a —CH₂—O—C₂H₅ group;
R₂ represents a —(C₁-C₆)alkylene- group, for instance a —CH₂—C(CH₃)₂— group;
R₃ represents —O—, —NH— or a —N((C₁-C₆)alkyl)- group, for instance —O— or —NH—;
R₄ represents a hydrogen atom, a —(C₁-C₆)alkyl group or a —(C₁-C₆)alkoxy group, for instance a hydrogen atom;
R₅ represents a —(C₁-C₆)alkylene- group, for instance a —CH₂—CH₂— group; and
R₇ represents a -L₁-G-Ab group or a -G-Ab group,
L₁ representing:
—NH—;
—C(═O)—NH—, for instance the —C(═O)— being linked to G and the —NH— being linked to R5;
a —(C₁-C₆)alkylene-R₈—C(═O)—NH— group, R₈ being a —(C₃-C₁₀)cycloalkylene-group or a —(C₃-C₁₀)heterocycloalkylene- group comprising 2 to 9 carbon atoms and 1 to 4 heteroatom(s) selected from oxygen, nitrogen, sulfur, —S(O)— and —SO₂—, for instance L₁ is a —(CH₂)₂-piperidinyl-C(═O)—NH— group, for instance the —(C₁-C₆)alkylene- group being linked to G and the —NH— being linked to R₅;
a —NH—C(═O)—(C₁-C₆)alkylene-C(═O)—NH— group, for instance L1 is a —NH—C(═O)—(CH₂)₃—C(═O)—NH— group, one —NH— being linked to G and the other —NH— being linked to R₅; or
a —(C₁-C₆)alkylene-C(═O)—NH— group, the —(C₁-C₆)alkylene- group being optionally substituted by a NH₂ group, for instance a —CH₂—CH(CH₂—NH₂)—C(═O)—NH— group, a —(CH₂)₂—C(═O)—NH— group or a —(CH₂)₃—C(═O)—NH— group, for instance the —(C₁-C₆)alkylene-group being linked to G and the —NH— being linked to R₅;
Ab representing an antibody, for instance a monoclonal antibody; and
G representing the product of reaction between RCG₁ as defined in claim 1 and RCG₂, a reactive group present on the antibody, for instance G is selected from the group consisting of: 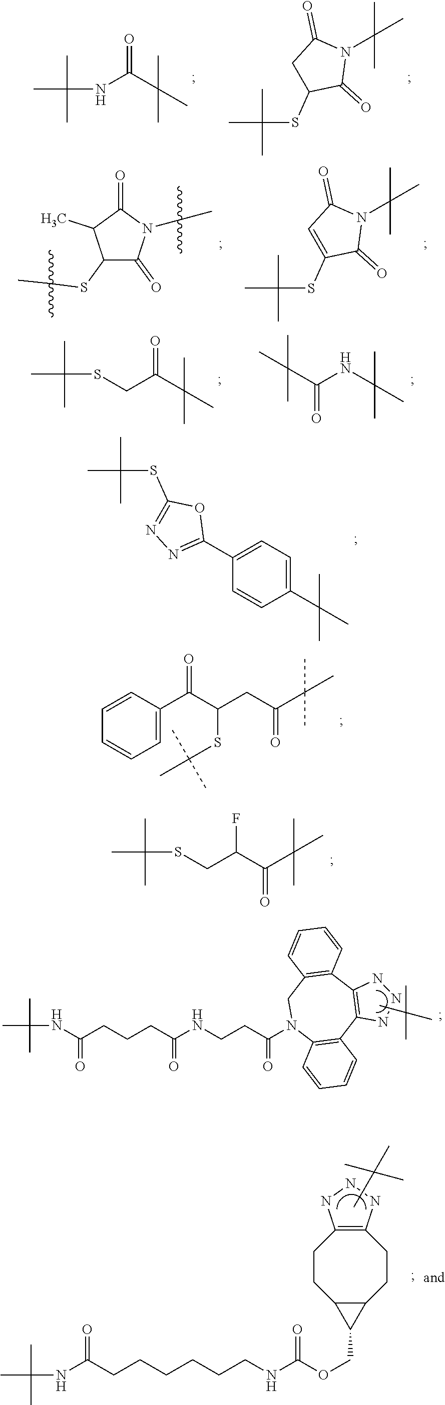 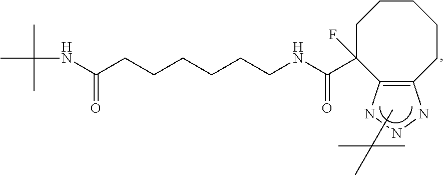
for instance the left side of the G groups being linked to Ab and the right side of the G groups being linked to L₁ when R₇ is -L₁-G-Ab or directly linked to R₅ when R₇ is -G-Ab, for instance, G represents the following groups:

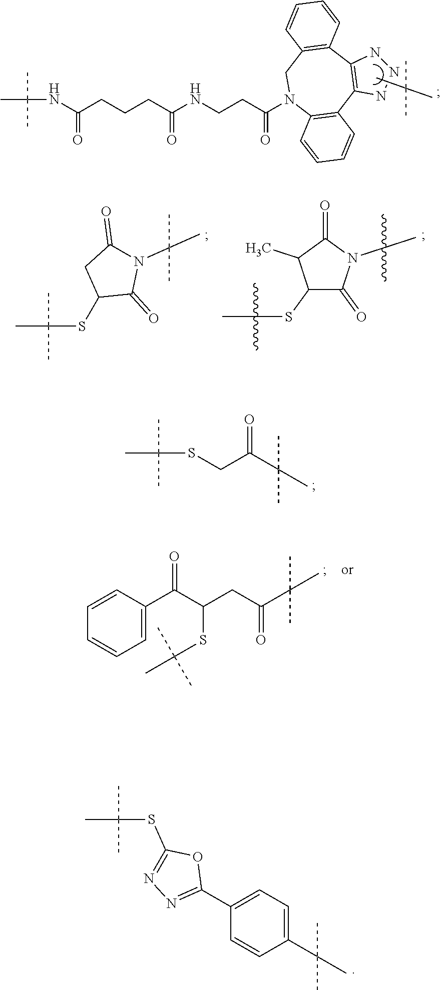

11. The compound of formula (II) or a pharmaceutically acceptable salt thereof according to claim 10, wherein RCG2 is selected from the group consisting of:
(i) a ε-amino group (ε—NH₂ group) borne by the side chain of a lysine residue that is present at the surface of the antibody;
(ii) an α-amino group (α-NH₂ group) of an N-terminal amino acid of a heavy chain or a light chain of the antibody;
(iii) the saccharide groups of the hinge region;
(iv) a thiol (a —SH group) of a cysteine residue generated by reducing an intra-chain disulfide bond of the antibody or a —SH group of an engineered cysteine residue of the antibody;
(v) an amide group (a —C(O)NH₂ group) borne by the side chains of a glutamine residue that is present at the surface of the antibody;
(vi) an aldehyde group (a —C(O)H group) introduced using formylglycine generating enzyme; and
(vii) RCG2 group, optionally introduced by means of a modifying agent.

12. The compound of formula (II) or a pharmaceutically acceptable salt thereof according to claim 10, wherein
when RCG1 represents a N-hydroxysuccinimidyl ester, RCG2 represents a —NH₂ group;
when RCG1 represents a maleimido function, a haloacetamido function, a chlorine atom or an activated disulfide, RCG2 represents a —SH group;
when RCG1 represents a —N₃ group, RCG2 represents a HC≡C— or an activated C≡C such as a cyclooctyne moiety;
when RCG1 represents a —OH or —NH₂ group, RCG2 represents a carboxylic acid or amide function;
when RCG1 represents a —SH group, RCG2 represents a maleimido function, a haloacetamido function or an activated disulfide function;
when RCG1 represents a HC≡C— or an activated C≡C, RCG2 represents a —N₃ group;
when RCG1 represents a O-alkyl hydroxylamine function or a Pictet-Spengler reaction substrate, RCG2 represents an aldehyde or a ketone function.

13. The compound of formula (II) or a pharmaceutically acceptable salt thereof according to claim 10, wherein the compound is selected from the group consisting of: 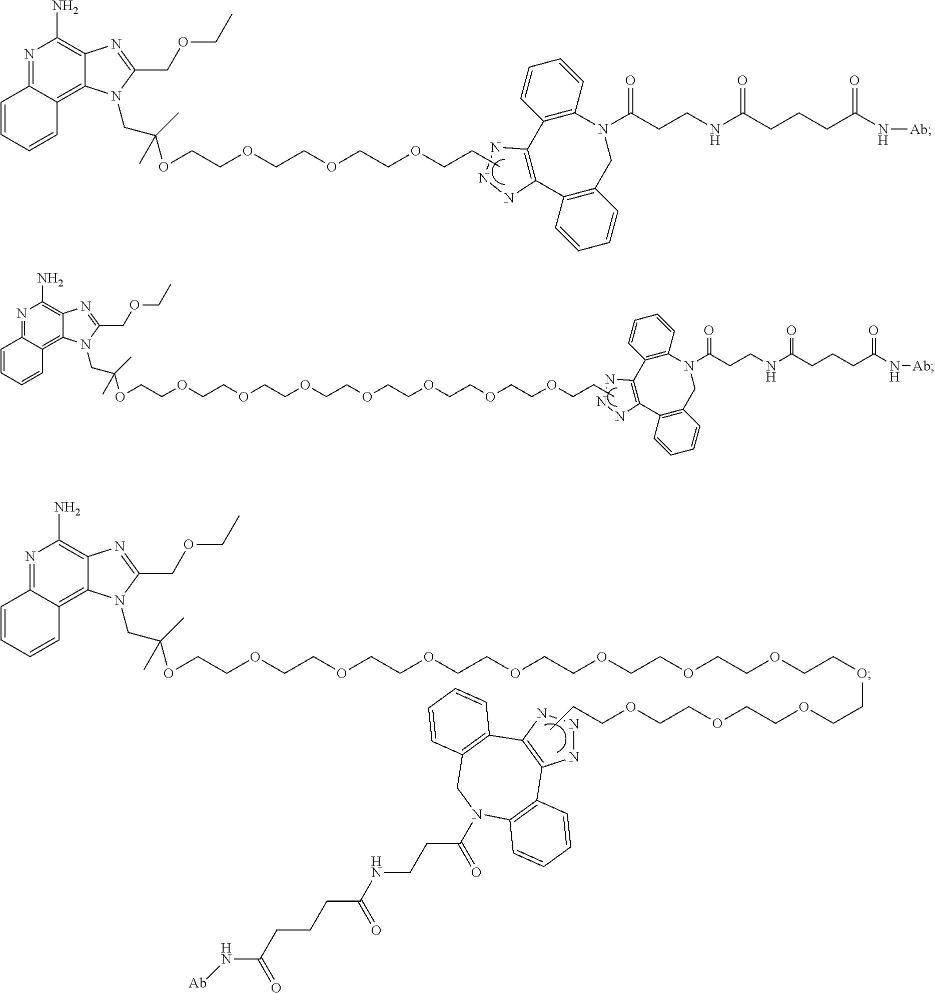 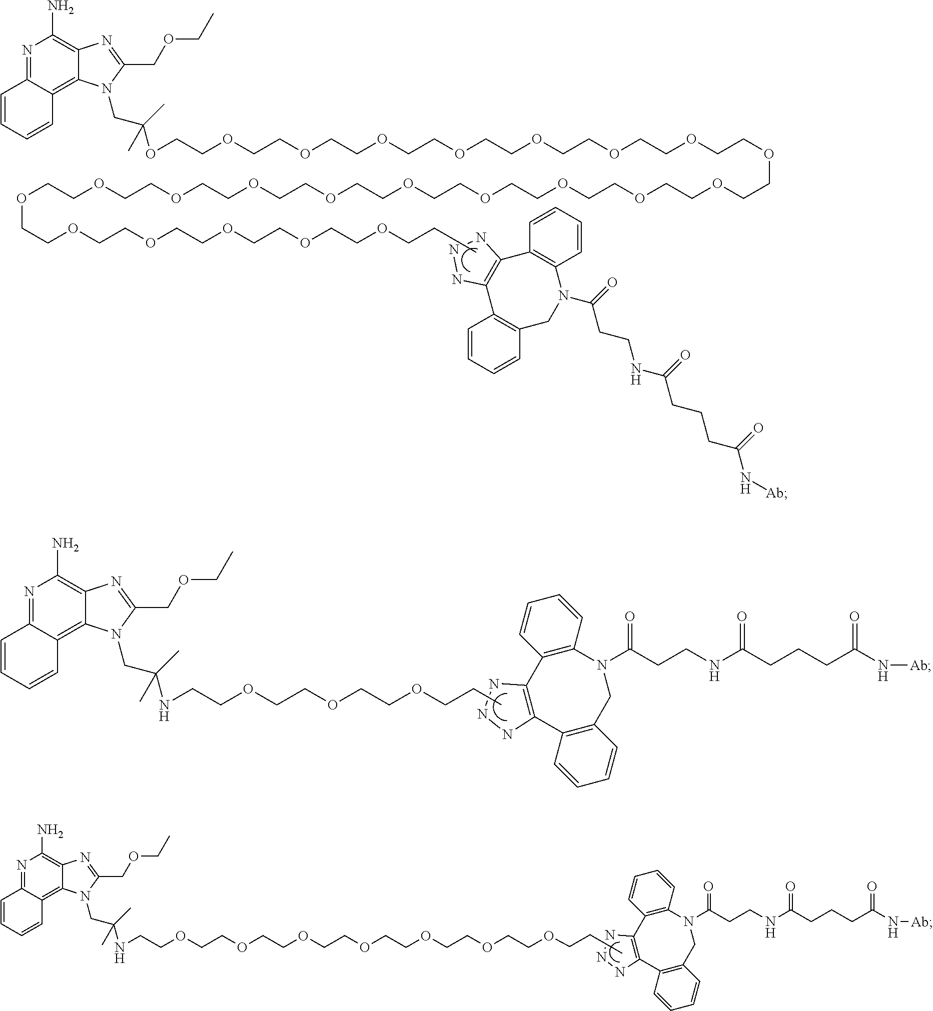 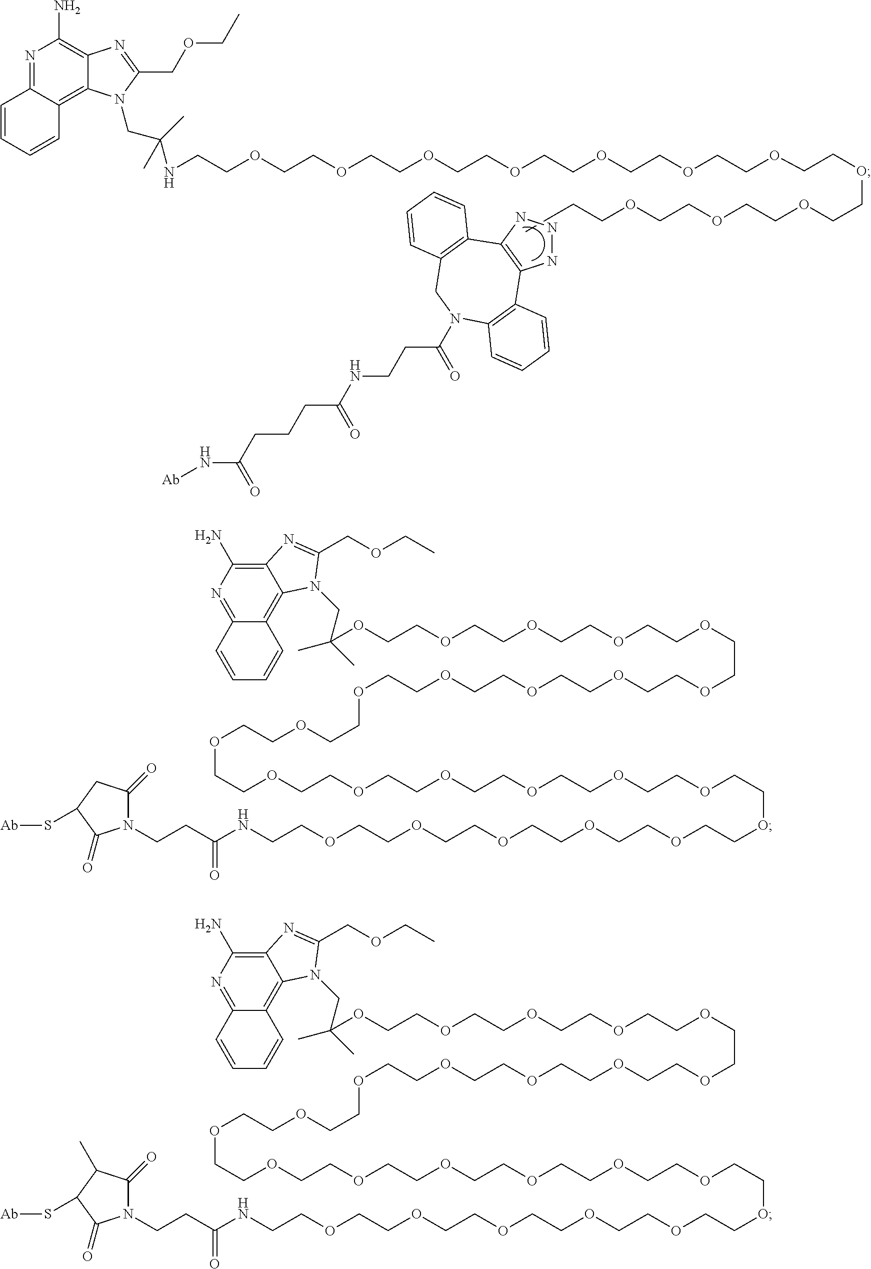 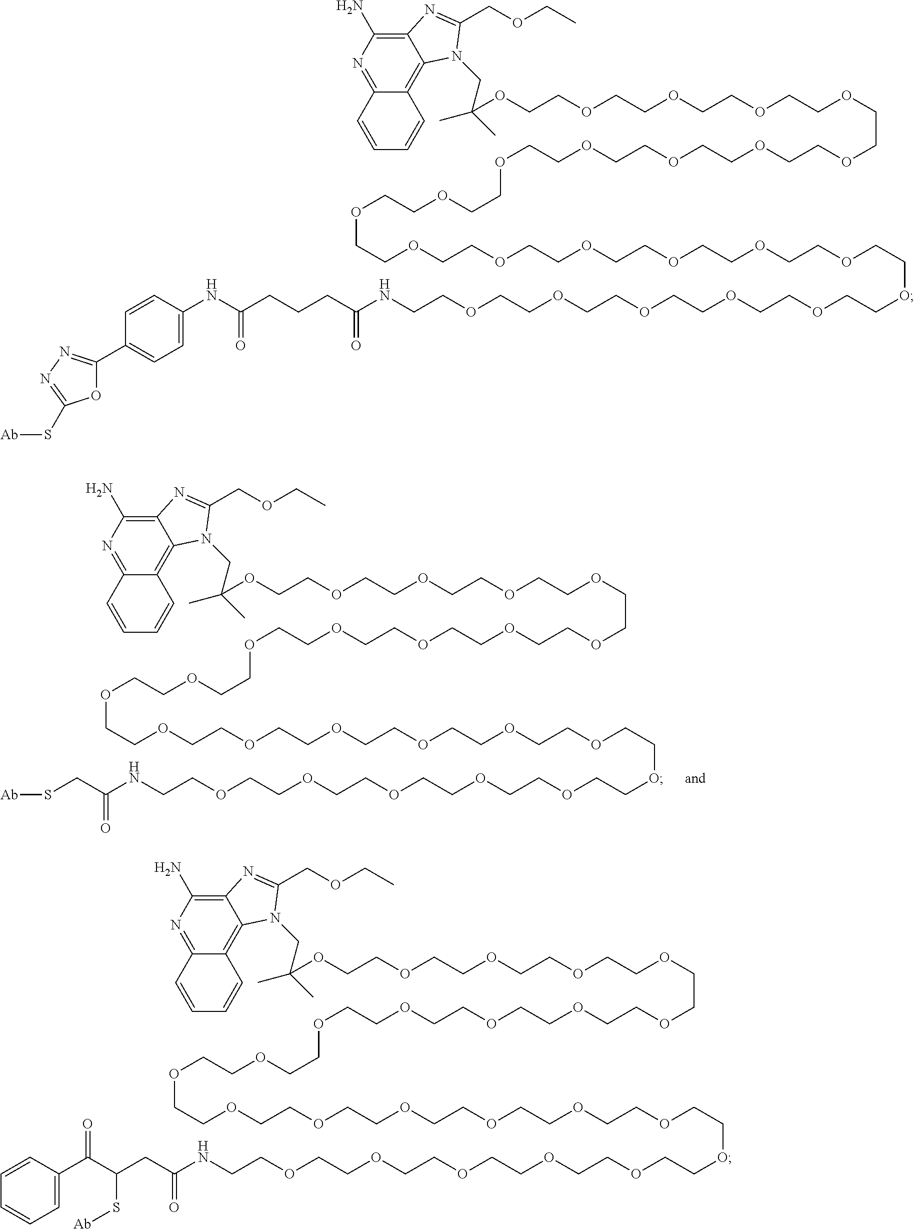
wherein Ab represents an antibody, for instance a monoclonal antibody.

14. Process for preparing a compound of formula (II) as defined in claim 10, comprising at least the steps of:
(i) placing in contact and leaving to react:
an, optionally buffered, aqueous solution of an antibody Ab, comprising a reactive RCG2 group, optionally modified by means of a modifying agent,
and
a solution of a compound of formula (I) comprising a reactive RCG1 group:
wherein formula (I) is 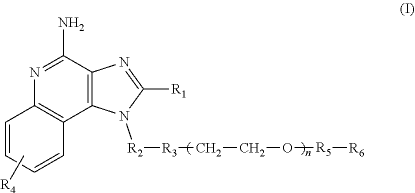
wherein
n is an integer from 1 to 50, for instance from 2 to 30, such as from 3 to 25;
R₁ represents a hydrogen atom, a —(C₁-C₆)alkylene-O—(C₁-C₆)alkyl group, a —(C₁-C₆)alkylene-NH—(C₁-C₆)alkyl group, or a —(C₁-C₆)alkyl group;
R₂ represents a —(C₁-C₆)alkylene- group;
R₃ represents —O—, —NH— or a —N((C₁-C₆)alkyl)- group;
R₄ represents a hydrogen atom, a —(C₁-C₆)alkyl group or a —(C₁-C₆)alkoxy group;
R₅ represents a —(C₁-C₆)alkylene- group; and
R₆ represents a -L₁-RCG₁ group or a —RCG₁ group:
L₁ representing:
—NH—;
—C(═O)—NH—, for instance the —C(═O)— being linked to RCG1 and the —NH— being linked to R5;
a —(C₁-C₆)alkylene-R₈—C(═O)—NH— group, R₈ being a —(C₃-C₁₀) cycloalkylene-group or a —(C₃-C₁₀)heterocycloalkylene- group comprising 2 to 9 carbon atoms and 1 to 4 heteroatom(s) selected from oxygen, nitrogen, sulfur, —S(O)— and —SO₂—, for instance the —(C₁-C₆)alkylene- group being linked to RCG1 and the —NH— being linked to R5;
a —NH—C(═O)—(C₁-C₆)alkylene-C(═O)—NH— group, one —NH— being linked to RCG₁ and the other —NH— being linked to R₅; or
a —(C₁-C₆)alkylene-C(═O)—NH— group, the —(C₁-C₆)alkylene- group being optionally substituted by a —NH₂ group, for instance the —(C₁-C₆)alkylene- group being linked to RCG₁ and the —NH— being linked to R₅;
RCG₁ representing:
(i) a RₐZₐ—C(═O)— reactive group for which:
Zₐ represents a single bond, —O— or —NH, such as —O—, and
Rₐ represents a hydrogen atom, a —(C₁-C₆)alkyl group, a —(C₃-C₇)cycloalkyl group, a —(C₂-C₆)alkenyl group, a —(C₆-C₁₀)aryl group, a —(C₅-C₁₀)heteroaryl group comprising 4 to 9 carbon atoms and 1 to 4 heteroatom(s) selected from oxygen, nitrogen, and sulfur, or a —(C₃-C₇)heterocycloalkyl group comprising 2 to 6 carbon atoms and 1 to 4 heteroatom(s) selected from oxygen, nitrogen, sulfur, —S(O)— and —SO₂—, said —(C₆-C₁₀)aryl group, —(C₅-C₁₀)heteroaryl group and/or —(C₃-C₇)heterocycloalkyl group being optionally substituted by 1 to 5 atoms/groups chosen from a halogen atom, such as a fluorine atom, a —(C₁-C₆)alkyl group, a —(C₁-C₆)alkoxy group, a hydroxyl group, an oxo group, a nitro group and a cyano group; or
(ii) one of the following reactive groups: a maleimido 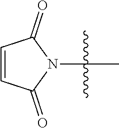 [SMILES: *N1C(=O)C=CC1=O]
group; a substituted maleimido group such as 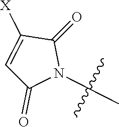
with X=Me, Br, 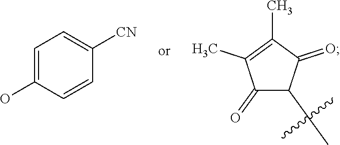
a haloacetamido 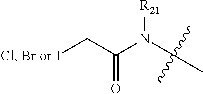
group with R₂₁ representing a hydrogen atom or a (C₁-C₆)alkyl group, such as a methyl group: Cl—; N₃—; HO—; HS—; an activated disulfide such as 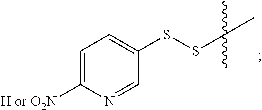
H₂N—; HC≡C— or an activated C≡C such as a cyclooctyne moiety for instance a DBCO-amine 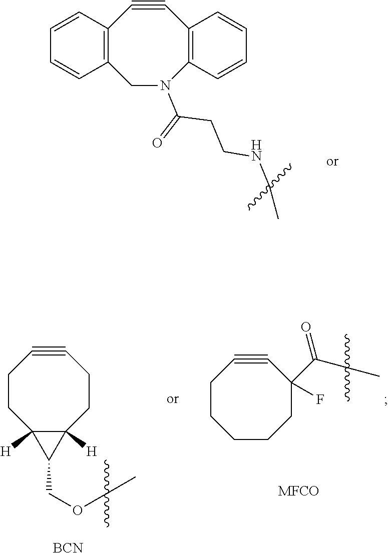
a phenyloxadiazolyl methylsulfone group (PODS) such as 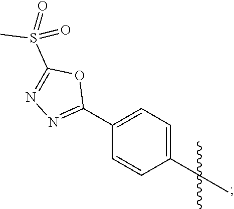 [SMILES: *c1ccc(-c2nnc(S(C)(=O)=O)o2)cc1] 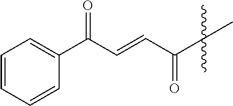 [SMILES: *C(=O)/C=C/C(=O)c1ccccc1]
group; 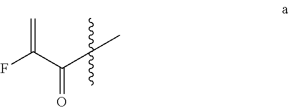 [SMILES: *C(=O)C(=C)F]
group; an O-alkyl hydroxylamine or a Pictet-Spengler reaction substrate such as 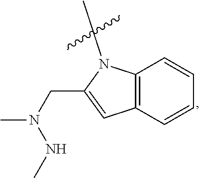 [SMILES: *n1c(CN(C)NC)cc2ccccc21]
for instance RCG₁ is N₃—; a maleimido group 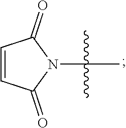 [SMILES: *N1C(=O)C=CC1=O]
a substituted maleimido group 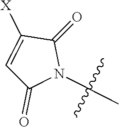
with X=Me, Br, 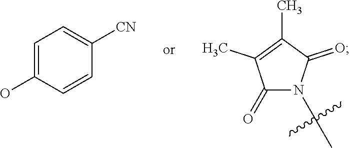
a phenyloxadiazolyl methylsulfone group 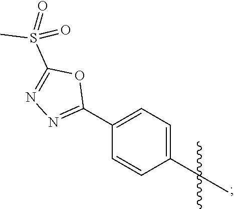 [SMILES: *c1ccc(-c2nnc(S(C)(=O)=O)o2)cc1]
a 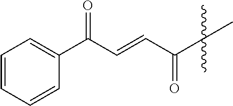 [SMILES: *C(=O)/C=C/C(=O)c1ccccc1]
group; a I—CH₂—C(═O)—NR₂₁— group with R₂₁ representing a hydrogen atom or a (C₁-C₆)alkyl group; or a 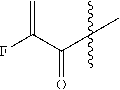 [SMILES: *C(=O)C(=C)F]
group,
RCG1 groups of the compound of formula (I) being reactive towards RCG2 groups of the antibody to form the G group by a covalent bond and forming compound of formula (II);
(ii) and then optionally separating the compound of formula (II) formed in step (i) from the unreacted compound of formula (I) and/or from the unreacted antibody and/or from any aggregates that may have been formed.

15. A pharmaceutical composition comprising a compound of formula (II) according to claim 10, and at least one pharmaceutically acceptable excipient.

16. A compound of formula (II) according to claim 10, for use as a medicine.

17. A compound of formula (II) according to claim 10, for use in the prevention and/or in the treatment of a disease or a disorder that may benefit of an activation of the immune system, for instance for use in the prevention and/or treatment of a cell-proliferative disease, a cancer, a chronic myelogenous, a hairy cell leukemia, a dermatological disease such as a skin lesion or a skin cancer for example an external genital and perianal warts/condyloma acuminate, a genital herpes, an actinic keratosis, a basal cell carcinoma, or a cutaneous T-cell lymphoma, an autoimmune disease, an inflammatory disease, a respiratory disease, a sepsis, an allergy for example an allergic rhinitis or a respiratory allergy, an asthma, a graft rejection, a graft-versus-host disease, and an immunodeficiency, for instance in the prevention and/or in the treatment of cancers.


SEQUENCE LISTING (10 sequences)

SEQ ID NO: 1 - protein, 214 aa, synthetic construct
DIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 449 aa, synthetic construct
EVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 219 aa, synthetic construct
DVVMTQTPLSLSVTLGQPASISCKSSQSLIHSDGRTYLNWLLQRPGQSPKRLIYLVSRLDSGVPDRFTGSGAGTDFTLKISRVEAEDLGVYYCWQGSHFPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 448 aa, synthetic construct
QVQLVQSGAEVVKPGASVKISCKASGYTFTAYYMHWVKQSPVQSLEWIGLVNPYNGFSSYNQKFQGKASLTVDRSSSTAYMELHSLTSEDSAVYYCAREFYGYRYFDVWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 213 aa, synthetic construct
IQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 214 aa, synthetic construct
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 452 aa, synthetic construct
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVMHEALHNHYTQKSLSLSPGK